(19) United States
(12) Patent Application Publication
(10) Pub. No.: US 2014/0056889 A1
(43) Pub. Date: Feb. 27, 2014

(54) COMPOSITIONS AND METHOD FOR TREATING AUTOIMMUNE DISEASES

(71) Applicants: Alyssa M. Morimoto, San Mateo, CA (US); Jacqueline McBride, Mountain View, CA (US); Richard Boismenu, Burlingame, CA (US); Romeo Maciuca, San Mateo, CA (US); William D. Kennedy, Redwood City, CA (US); Michael J. Townsend, San Jose, CA (US); Jorn Drappa, Washington, DC (US)

(72) Inventors: Alyssa M. Morimoto, San Mateo, CA (US); Jacqueline McBride, Mountain View, CA (US); Richard Boismenu, Burlingame, CA (US); Romeo Maciuca, San Mateo, CA (US); William D. Kennedy, Redwood City, CA (US); Michael J. Townsend, San Jose, CA (US); Jorn Drappa, Washington, DC (US)

(73) Assignee: Genentech, Inc., South San Francisco, CA (US)

(21) Appl. No.: 14/113,575

(22) PCT Filed: Apr. 26, 2012
(86) PCT No.: PCT/US12/35313

§ 371 Date: Oct. 23, 2013

Related U.S. Application Data
(60) Provisional application No. 61/479,314, filed on Apr. 26, 2011.
(60) Provisional application No. 61/582,179, filed on Dec. 30, 2011.

(51) Int. Cl. C07K 16/24; C12Q 1/68; A61K 39/395; G01N 33/68; A61K 45/06; A61K 31/573

(52) U.S. Cl. CPC C07K 16/249; A61K 45/06; A61K 31/573; A61K 39/3955; G01N 33/686; C12Q 1/6883
USPC 424/133.1; 435/7.24; 435/6.12; 506/9; 435/6.11; 435/7.92

(57) ABSTRACT

The invention provides methods and compositions for treating various autoimmune diseases (such as systemic lupus erythematousu) with an interferon inhibitor (such as an anti-intereron-alpha monoclonal antibody). More specifically, the invention provides a method of diagnosing, monitoring and adjusting the treatment of such a patient by way of an interferon signature metric (interferon response gene measurement value), a certain anti-dsDNA antibody titre or being ENA− (levels of extractable nuclear antigens lower than a healthy level). Furthermore, the invention provides articles of manufacture associated with such a diagnosis.

### CROSS-REFERENCE TO RELATED PATENT APPLICATIONS

This application claims the priority benefit of U.S. provisional application Ser. Nos. 61/479,314, filed Apr. 26, 2011, and 61/582,179, filed Dec. 30, 2011, which are incorporated herein by reference in their entirety.

### FIELD OF THE INVENTION

The invention involves methods and compositions for treating various autoimmune diseases (e.g., lupus) with an interferon inhibitor (e.g., an anti-Type I interferon antibody).

### BACKGROUND

Autoimmune diseases, such as systemic lupus erythematosus (SLE), myasthenia gravis (MG) and idiopathic thrombocytopenic purpura (ITP), among others, remain clinically important diseases in humans. As the name implies, autoimmune diseases wreak their havoc through the body's own immune system. While the pathological mechanisms differ between individual types of autoimmune diseases, one general mechanism involves the binding of certain antibodies (referred to herein as self-reactive antibodies or autoantibodies) present in the sera of patients to self-nuclear or cellular antigens.

Lupus is an autoimmune disease involving antibodies that attack connective tissue. The disease is estimated to affect nearly 1 million Americans, primarily women between the ages of 20-40. The principal form of lupus is a systemic one (systemic lupus erythematosus; SLE). SLE is associated with the production of antinuclear antibodies, circulating immune complexes, and activation of the complement system. SLE has an incidence of about 1 in 700 women between the ages of 20 and 60. SLE can affect any organ system and can cause severe tissue damage. Numerous autoantibodies of differing specificity are present in SLE. SLE patients often produce autoantibodies having anti-DNA, anti-Ro, anti-La, anti-Sm, anti-RNP, and anti-platelet specificity and that are capable of initiating clinical features of the disease, such as glomerulonephritis, arthritis, serositis, complete heart block in newborns, and hematologic abnormalities. These autoantibodies are also possibly related to central nervous system disturbances. Arbuckle et al. describes the development of autoantibodies before the clinical onset of SLE (Arbuckle et al. N. Engl. J. Med. 349(16): 1526-1533 (2003)).

Untreated lupus can be fatal as it progresses from attack of skin and joints to internal organs, including lung, heart, and kidneys (with renal disease being the primary concern). Lupus mainly appears as a series of flare-ups, with intervening periods of little or no disease manifestation.

Kidney damage, measured by the amount of proteinuria in the urine, is one of the most acute areas of damage associated with pathogenicity in SLE, and accounts for at least 50% of the mortality and morbidity of the disease.

The presence of antibodies immunoreactive with double-stranded native DNA is used as a diagnostic marker for SLE.

Currently, there are no really curative treatments for patients who have been diagnosed with SLE. From a practical standpoint, physicians generally employ a number of powerful immunosuppressive drugs such as high-dose corticosteroids, e.g., prednisone, or azathioprine or cyclophosphamide, which are given during periods of flare-ups, but may also be given persistently for those who have experienced frequent flare-ups. Even with effective treatment, which reduces symptoms and prolongs life, many of these drugs have potentially harmful side effects to the patients being treated. In addition, these immunosuppressive drugs interfere with the person's ability to produce all antibodies, not just the self-reactive anti-DNA antibodies. Immunosuppressants also weaken the body's defense against other potential pathogens, thereby making the patient extremely susceptible to infection and other potentially fatal diseases, such as cancer. In some of these instances, the side effects of current treatment modalities, combined with continued low-level manifestation of the disease, can cause serious impairment and premature death. Recent therapeutic regimens include cyclophosphamide, methotrexate, antimalarials, hormonal treatment (e.g., DHEA), and anti-hormonal therapy (e.g., the anti-prolactin agent bromocriptine).

Methods for treatment of SLE involving antibodies are also described. The method in Diamond et al. (U.S. Pat. No. 4,690,905) consists of generating monoclonal antibodies against anti-DNA antibodies (the monoclonal antibodies being referred to therein as anti-idiotypic antibodies) and then using these anti-idiotypic antibodies to remove the pathogenic anti-DNA antibodies from the patient's system. However, the removal of large quantities of blood for treatment can be a dangerous, complicated process. U.S. Pat. No. 6,726,909 discloses treating SLE wherein the antibody composition administered to the patient comprises purified anti-DNA anti-idiotypic antibodies and the administration requires an injection, or other equivalent mode of administration.

High-dose intravenous immune globulin (IVIG) infusions have also been used in treating certain autoimmune diseases. Up until the present time, treatment of SLE with WIG has provided mixed results, including both resolution of lupus nephritis (Akashi et al., J. Rheumatology 17:375-379 (1990)), and in a few instances, exacerbation of proteinuria and kidney damage (Jordan et al., Clin. Immunol. Immunopathol. 53: S164-169 (1989)).

Persons afflicted with lupus such as those with SLE who show clinical evidence for lupus nephritis and those with lupus nephritis need a cost-efficient and safe treatment that will help ameliorate the tissue damage that leads ultimately to kidney failure and the need for chronic hemodialysis and/or renal transplantation caused by their condition. Patients typically have several treatment options available to them including corticosteroids, non-steroidal anti-inflammatory drugs, and antibody-based drugs. Diagnostic methods useful for identifying patients likely to benefit from different treatment regimens would greatly benefit clinical management of these patients.

Thus, there is a need for objective, reproducible methods for the optimal diagnosis and/or treatment regimen for each patient. This invention meets these and other needs.

### SUMMARY OF THE INVENTION

The present invention involves, at least in part, a method of treating a systemic lupus erythematosus (SLE) patient with a type I interferon antibody. In one aspect, the invention involves methods and compositions for treating various autoimmune diseases (e.g., lupus) with an interferon inhibitor (e.g., anti-Type I interferon antibody). In any of the embodiments disclosed herein, an interferon inhibitor is an anti-Type I interferon antibody.

Accordingly, one embodiment of the invention provides methods of treating lupus in a patient, the method comprising administering an effective amount of an interferon type I antibody to a patient diagnosed with lupus, wherein the patient has an ENA status of ENA−. In some embodiments, the antibody specifically binds an interferon selected from the group consisting of: interferon α, interferon β, interferon ω, interferon λ and combinations thereof. In some embodiments, the antibody specifically binds interferon α. In some embodiments, the antibody specifically binds interferon β. In some embodiments, the antibody specifically binds interferon ω. In some embodiments, the antibody specifically binds interferon λ. In some embodiments, the antibody specifically binds interferon α, interferon β, interferon ω, and interferon λ. In some embodiments, the antibody specifically binds interferon α, interferon β and interferon ω but not interferon λ. In some embodiments, the antibody specifically binds interferon α, interferon λ and interferon ω, but not interferon β. In some embodiments, the antibody specifically binds interferon α, interferon β and interferon λ but not interferon ω. In some embodiments, the antibody specifically binds interferon α and interferon β but not interferon ω or interferon λ. In some embodiments, the antibody specifically binds interferon α and interferon ω but not interferon β or interferon λ. In some embodiments, the antibody specifically binds interferon α and interferon λ but not interferon β or interferon ω. In some embodiments, the antibody is rontalizumab. In some embodiments, the patient has been determined to be ENA−. In some embodiments, the ENA status of the patient is determined by detecting autoantibodies in a sample from the patient wherein the autoantibodies are selected from anti-Ro, anti-La, anti-SM, anti-RNP, and combinations thereof. In some embodiments, the sample is selected from whole blood, blood-derived cells, plasma, serum, and combinations thereof. In some embodiments, the antibody is administered intravenously. In some embodiments, the antibody is administered subcutaneously. In some embodiments, the lupus is systemic lupus erythematosus. In some embodiments, the patient has a baseline interferon signature metric (ISM) that is greater than or equal to the ISM of a healthy individual. In some embodiments, the patient has an ISM that is lower following administration of the antibody as compared to the patient's baseline ISM. In some embodiments, the ISM is determined by measuring the expression level of at least one gene selected from the group consisting of: CMPK2, EPST1, HERC5, and combinations thereof. In some embodiments, the ISM is determined by measuring the expression level of at least one gene selected from the group consisting of: IFI27, IFI44, IFIT1, MX1, OAS1, OAS2, OAS3, and combinations thereof. In some embodiments, the methods further comprise administering a second medicament to the subject. In some embodiments, the second medicament is elected from a corticosteroid, a non-steroidal anti-inflammatory drug (NSAID), an anti-malarial agent, a statin, and combinations thereof.

A further embodiment of the invention provides methods of identifying a lupus patient who may benefit from treatment with an interferon type I antibody, the method comprising determining ENA status of the patient, wherein a patient who is determined to have an ENA status of ENA− is identified as a patient who may benefit from treatment with the interferon type I antibody. In some embodiments, the antibody specifically binds an interferon selected from the group consisting of: interferon α, interferon β, interferon ω, interferon λ and combinations thereof. In some embodiments, the antibody specifically binds interferon α. In some embodiments, the antibody specifically binds interferon β. In some embodiments, the antibody specifically binds interferon ω. In some embodiments, the antibody specifically binds interferon λ. In some embodiments, the antibody specifically binds interferon α, interferon β, interferon ω and interferon λ. In some embodiments, the antibody specifically binds interferon α, interferon β and interferon ω, but not interferon λ. In some embodiments, the antibody specifically binds interferon α, interferon, interferon λ, and interferon ω, but not interferon β. In some embodiments, the antibody specifically binds interferon α, interferon β, and interferon λ, but not interferon ω. In some embodiments, the antibody specifically binds interferon α and interferon β but not interferon ω or interferon λ. In some embodiments, the antibody specifically binds interferon α and interferon ω but not interferon β or interferon λ. In some embodiments, the antibody specifically binds interferon α and interferon λ but not interferon β or interferon ω. In some embodiments, the antibody is rontalizumab. In some embodiments, the ENA status of the patient is determined by detecting autoantibodies in a sample from the patient wherein the autoantibodies are selected from anti-Ro, anti-La, anti-SM, anti-RNP, and combinations thereof. In some embodiments, the sample is selected from whole blood, blood-derived cells, plasma, serum, and combinations thereof. In some embodiments, the methods further comprise administering an effective amount of the interferon type I antibody to the patient. In some embodiments, the antibody is administered intravenously. In some embodiments, the antibody is administered subcutaneously. In some embodiments, the antibody is administered for at least 24 weeks. In some embodiments, the patient has a baseline interferon signature metric (ISM) that is greater than or equal to the ISM of a healthy individual. In some embodiments, the patient has an ISM that is lower following administration of the antibody as compared to the patient's baseline ISM. In some embodiments, the ISM is determined by measuring the expression level of at least one gene selected from the group consisting of: CMPK2, EPST1, HERC5, and combinations thereof. In some embodiments, the ISM is determined by measuring the expression level of at least one gene selected from the group consisting of: IFI27, IFI44, IFIT1, MX1, OAS1, OAS2, OAS3, and combinations thereof. In some embodiments, the methods further comprise administering a second medicament to the subject. In some embodiments, the second medicament is elected from a corticosteroid, a non-steroidal anti-inflammatory drug (NSAID), an anti-malarial agent, a statin, and combinations thereof.

Another embodiment of the invention provides methods of optimizing therapeutic efficacy for treatment of lupus, the method comprising determining ENA status of a lupus patient, wherein a patient who is determined to have an ENA status of ENA− has increased likelihood of benefit from treatment with the interferon type I antibody. In some embodiments, the antibody specifically binds an interferon selected from the group consisting of: interferon α, interferon β, interferon ω, interferon λ, and combinations thereof. In some embodiments, the antibody specifically binds interferon α. In some embodiments, the antibody specifically binds interferon β. In some embodiments, the antibody specifically binds interferon ω. In some embodiments, the antibody specifically binds interferon λ. In some embodiments, the antibody specifically binds interferon α, interferon β, interferon ω, and interferon λ. In some embodiments, the antibody specifically binds interferon α, interferon β, and interferon ω, but not interferon λ. In some embodiments, the antibody specifically binds interferon α, interferon λ, and interferon ω, but not interferon β. In some embodiments, the antibody specifically binds interferon α, interferon β, and interferon λ, but not interferon ω. In some embodiments, the antibody specifically binds interferon α and interferon β but not interferon ω or interferon λ. In some embodiments, the antibody specifically binds interferon α and interferon ω but not interferon β or interferon λ. In some embodiments, the antibody specifically binds interferon α and interferon λ but not interferon β or interferon ω. In some embodiments, the antibody is rontalizumab. In some embodiments, the ENA status of the patient is determined by detecting autoantibodies in a sample from the patient wherein the autoantibodies are selected from anti-Ro, anti-La, anti-SM, anti-RNP, and combinations thereof. In some embodiments, the sample is selected from whole blood, blood-derived cells, plasma, serum, and combinations thereof. In some embodiments, the methods further comprise administering an effective amount of the interferon type I antibody to the patient. In some embodiments, the antibody is administered intravenously. In some embodiments, the antibody is administered subcutaneously. In some embodiments, the antibody is administered for at least 24 weeks. In some embodiments, the patient has a baseline interferon signature metric (ISM) that is greater than or equal to the ISM of a healthy individual. In some embodiments, the patient has an ISM that is lower following administration of the antibody as compared to the patient's baseline ISM. In some embodiments, the ISM is determined by measuring the expression level of at least one gene selected from the group consisting of: CMPK2, EPST1, HERC5, and combinations thereof. In some embodiments, the ISM is determined by measuring the expression level of at least one gene selected from the group consisting of: IFI27, IFI44, IFIT1, MX1, OAS1, OAS2, OAS3, and combinations thereof. In some embodiments, the methods further comprise administering a second medicament to the subject. In some embodiments, the second medicament is elected from a corticosteroid, a non-steroidal anti-inflammatory drug (NSAID), an anti-malarial agent, a statin, and combinations thereof.

Even another embodiment of the invention provides methods of predicting responsiveness of a lupus patient to treatment with an interferon type I antibody, the method comprising determining ENA status of the patient, wherein a patient who is determined to have an ENA status of ENA− is identified as a patient who is likely to respond to treatment with the interferon type I antibody. In some embodiments, the antibody specifically binds an interferon selected from the group consisting of: interferon α, interferon β, interferon ω, interferon λ, and combinations thereof. In some embodiments, the antibody specifically binds interferon α. In some embodiments, the antibody specifically binds interferon β. In some embodiments, the antibody specifically binds interferon ω. In some embodiments, the antibody specifically binds interferon λ. In some embodiments, the antibody specifically binds interferon α, interferon β, interferon ω, and interferon λ. In some embodiments, the antibody specifically binds interferon α, interferon β, and interferon ω; but not interferon λ. In some embodiments, the antibody specifically binds interferon α, interferon λ, and interferon ω, but not interferon β. In some embodiments, the antibody specifically binds interferon α, interferon β, and interferon λ, but not interferon ω. In some embodiments, the antibody specifically binds interferon α and interferon β but not interferon ω or interferon λ. In some embodiments, the antibody specifically binds interferon α and interferon ω but not interferon β or interferon λ. In some embodiments, the antibody specifically binds interferon α and interferon λ but not interferon β or interferon ω. In some embodiments, the antibody is rontalizumab. In some embodiments, the ENA status of the patient is determined by detecting autoantibodies in a sample from the patient wherein the autoantibodies are selected from anti-Ro, anti-La, anti-SM, anti-RNP, and combinations thereof. In some embodiments, the sample is selected from whole blood, blood-derived cells, plasma, serum, and combinations thereof. In some embodiments, the methods further comprise administering an effective amount of the interferon type I antibody to the patient. In some embodiments, the antibody is administered intravenously. In some embodiments, the antibody is administered subcutaneously. In some embodiments, the antibody is administered for at least 24 weeks. In some embodiments, the patient has a baseline interferon signature metric (ISM) that is greater than or equal to the ISM of a healthy individual. In some embodiments, the patient has an ISM that is lower following administration of the antibody as compared to the patient's baseline ISM. In some embodiments, the ISM is determined by measuring the expression level of at least one gene selected from the group consisting of: CMPK2, EPST1, HERC5 and combinations thereof. In some embodiments, the ISM is determined by measuring the expression level of at least one gene selected from the group consisting of: IFI27, IFI44, IFIT1, MX1, OAS1, OAS2, OAS3, and combinations thereof. In some embodiments, the methods further comprise administering a second medicament to the subject. In some embodiments, the second medicament is elected from a corticosteroid, a non-steroidal anti-inflammatory drug (NSAID), an anti-malarial agent, a statin, and combinations thereof.

Yet another embodiment of the invention provides methods for determining the likelihood that a lupus patient will benefit from treatment with an interferon type I antibody, the method comprising determining ENA status of the patient, wherein a patient who is determined to have an ENA status of ENA− is identified as a patient who is likely to respond to treatment with the interferon type I antibody. In some embodiments, the antibody specifically binds an interferon selected from the group consisting of: interferon α, interferon β, interferon ω, interferon λ, and combinations thereof. In some embodiments, the antibody specifically binds interferon α. In some embodiments, the antibody specifically binds interferon β. In some embodiments, the antibody specifically binds interferon ω. In some embodiments, the antibody specifically binds interferon λ. In some embodiments, the antibody specifically binds interferon α, interferon β, interferon ω, and interferon λ. In some embodiments, the antibody specifically binds interferon α, interferon β, and interferon ω but not interferon λ. In some embodiments, the antibody specifically binds interferon α, interferon λ, and interferon ω, but not interferon β. In some embodiments, the antibody specifically binds interferon α, interferon β, and interferon λ but not interferon ω. In some embodiments, the antibody specifically binds interferon α and interferon β but not interferon ω or interferon λ. In some embodiments, the antibody specifically binds interferon α and interferon ω but not interferon β or interferon λ. In some embodiments, the antibody specifically binds interferon α and interferon λ but not interferon β or interferon ω. In some embodiments, the antibody is rontalizumab. In some embodiments, the ENA status of the patient is determined by detecting autoantibodies in a sample from the patient wherein the autoantibodies are selected from anti-Ro, anti-La, anti-SM, anti-RNP, and combinations thereof. In some embodiments, the sample is selected from whole blood, blood-derived cells, plasma, serum, and combinations thereof. In some embodiments, the methods further comprise administering an effective amount of the interferon type I antibody to the patient. In some embodiments, the antibody is administered intravenously. In some embodiments, the antibody is administered subcutaneously. In some embodiments, the antibody is administered for at least 24 weeks. In some embodiments, the patient has a baseline interferon signature metric (ISM) that is greater than or equal to the ISM of a healthy individual. In some embodiments, the patient has an ISM that is lower following administration of the antibody as compared to the patient's baseline ISM. In some embodiments, the ISM is determined by measuring the expression level of at least one gene selected from the group consisting of: CMPK2, EPST1, HERC5, and combinations thereof. In some embodiments, the ISM is determined by measuring the expression level of at least one gene selected from the group consisting of: IFI27, IFI44, IFIT1, MX1, OAS1, OAS2, OAS3, and combinations thereof. In some embodiments, the methods further comprise administering a second medicament to the subject. In some embodiments, the second medicament is elected from a corticosteroid, a non-steroidal anti-inflammatory drug (NSAID), an anti-malarial agent, a statin, and combinations thereof.

Accordingly, one embodiment of the invention provides methods of treating an autoimmune disease (e.g, lupus) in a patient, the method comprising administering an effective amount of an interferon type I antibody to a patient diagnosed with the autoimmune disease, wherein the patient has an anti-dsDNA antibody status of low (e.g. 200 IU). In some embodiments, the antibody specifically binds an interferon selected from the group consisting of: interferon α, interferon β, interferon ω, interferon λ and combinations thereof. In some embodiments, the antibody specifically binds interferon α. In some embodiments, the antibody specifically binds interferon β. In some embodiments, the antibody specifically binds interferon ω. In some embodiments, the antibody specifically binds interferon λ. In some embodiments, the antibody specifically binds interferon α, interferon β, interferon ω and interferon λ. In some embodiments, the antibody specifically binds interferon α, interferon β and interferon ω but not interferon λ. In some embodiments, the antibody specifically binds interferon α, interferon λ and interferon ω but not interferon β. In some embodiments, the antibody specifically binds interferon α, interferon β and interferon λ but not interferon ω. In some embodiments, the antibody specifically binds interferon α and interferon β but not interferon ω or interferon λ. In some embodiments, the antibody specifically binds interferon α and interferon ω but not interferon β or interferon λ. In some embodiments, the antibody specifically binds interferon α and interferon λ but not interferon β or interferon ω. In some embodiments, the antibody is rontalizumab. In some embodiments, the anti-dsDNA antibody status of the patient is determined by detecting autoantibodies in a sample from the patient by immunoassay. In some embodiments, the sample is selected from whole blood, blood-derived cells, plasma, serum, and combinations thereof. In some embodiments, the antibody is administered intravenously. In some embodiments, the lupus is systemic lupus erythematosus. In some embodiments, the patient has a baseline interferon signature metric (ISM) that is greater than or equal to the ISM of a healthy individual. In some embodiments, the patient has an ISM that is lower following administration of the antibody as compared to the patient's baseline ISM. In some embodiments, the ISM is determined by measuring the expression level of at least one gene selected from the group consisting of: CMPK2, EPST1, HERC5, and combinations thereof. In some embodiments, the ISM is determined by measuring the expression level of at least one gene selected from the group consisting of: IFI27, IFI44, IFIT1, MX1, OAS1, OAS2, OAS3, and combinations thereof. In some embodiments, prior to treatment with an interferon inhibitor, the IRG status of the patient has been determined. In one embodiment, any one or combination or all of the IRGs of the Enlarged ISM, the Enlarged ISM-A, the 24-gene ISM or the 3-gene ISM are used to evaluate the IRG status. In some embodiments, prior to treatment with an interferon inhibitor, the patient has anti-dsDNA antibody low status and an IRG status that is ISM^{lo}. In some embodiments, prior to treatment with an interferon inhibitor, the patient has anti-dsDNA antibody low status and an IRG status that is ISM^{hi}. In some embodiments, the methods further comprise administering a second medicament to the subject. In some embodiments, the second medicament is elected from a corticosteroid, a non-steroidal anti-inflammatory drug (NSAID), an anti-malarial agent, a statin, and combinations thereof.

A further embodiment of the invention provides methods of identifying an autoimmune disease patient (e.g., lupus patient) who may benefit from treatment with an interferon type I antibody, the method comprising determining anti-dsDNA antibody status of the patient, wherein a patient who is determined to have an anti-dsDNA antibody status of low (e.g. ≦200 IU) is identified as a patient who may benefit from treatment with the interferon type I antibody. In some embodiments, the antibody specifically binds an interferon selected from the group consisting of: interferon α, interferon β, interferon ω, interferon λ and combinations thereof. In some embodiments, the antibody specifically binds interferon α. In some embodiments, the antibody specifically binds interferon β. In some embodiments, the antibody specifically binds interferon ω. In some embodiments, the antibody specifically binds interferon λ. In some embodiments, the antibody specifically binds interferon α, interferon β, interferon ω and interferon λ. In some embodiments, the antibody specifically binds interferon α, interferon β and interferon ω but not interferon λ. In some embodiments, the antibody specifically binds interferon α, interferon λ and interferon ω but not interferon β. In some embodiments, the antibody specifically binds interferon α, interferon β and interferon λ but not interferon ω. In some embodiments, the antibody specifically binds interferon α and interferon β but not interferon ω or interferon λ. In some embodiments, the antibody specifically binds interferon α and interferon ω but not interferon β or interferon λ. In some embodiments, the antibody specifically binds interferon α and interferon λ but not interferon β or interferon ω. In some embodiments, the antibody is rontalizumab. In some embodiments, the anti-dsDNA antibody status of the patient is determined by detecting autoantibodies in a sample from the patient by immunoassay. In some embodiments, the sample is selected from whole blood, blood-derived cells, plasma, serum, and combinations thereof. In some embodiments, the methods further comprise administering an effective amount of the interferon type I antibody to the patient. In some embodiments, the antibody is administered intravenously. In some embodiments, the antibody is administered subcutaneously. In some embodiments, the antibody is administered for at least 24 weeks. In some embodiments, the patient has a baseline interferon signature metric (ISM) that is greater than or equal to the ISM of a healthy individual. In some embodiments, the patient has an ISM that is lower following administration of the antibody as compared to the patient's baseline ISM. In some embodiments, the ISM is determined by measuring the expression level of at least one gene selected from the group consisting of: CMPK2, EPST1, HERC5, and combinations thereof. In some embodiments, the ISM is determined by measuring the expression level of at least one gene selected from the group consisting of: IFI27, IFI44, IFIT1, MX1, OAS1, OAS2, OAS3, and combinations thereof. In some embodiments, prior to treatment with an interferon inhibitor, the IRG status of the patient has been determined. In one embodiment, any one or combination or all of the IRGs of the Enlarged ISM, the Enlarged ISM-A, the 24-gene ISM or the 3-gene ISM are used to evaluate the IRG status. In some embodiments, prior to treatment with an interferon inhibitor, the patient has been determined to have an anti-dsDNA antibody low status and an IRG status that is ISM^{lo}. In some embodiments, prior to treatment with an interferon inhibitor, the patient has been determined to have an anti-dsDNA antibody low status and an IRG status that is ISM^{hi}. In some embodiments, the methods further comprise administering a second medicament to the subject. In some embodiments, the second medicament is elected from a corticosteroid, a non-steroidal anti-inflammatory drug (NSAID), an anti-malarial agent, a statin, and combinations thereof.

Another embodiment of the invention provides methods of optimizing therapeutic efficacy for treatment of lupus, the method comprising determining anti-dsDNA antibody status of a lupus patient, wherein a patient who is determined to have an anti-dsDNA antibody status of low (e.g. ≦200 IU) has increased likelihood of benefit from treatment with the interferon type I antibody. In some embodiments, the antibody specifically binds an interferon selected from the group consisting of: interferon α, interferon β, interferon ω, interferon λ and combinations thereof. In some embodiments, the antibody specifically binds interferon α. In some embodiments, the antibody specifically binds interferon β. In some embodiments, the antibody specifically binds interferon ω. In some embodiments, the antibody specifically binds interferon λ. In some embodiments, the antibody specifically binds interferon α, interferon β, interferon ω and interferon λ. In some embodiments, the antibody specifically binds interferon α, interferon β and interferon ω but not interferon λ. In some embodiments, the antibody specifically binds interferon α, interferon λ and interferon ω but not interferon β. In some embodiments, the antibody specifically binds interferon α, interferon β and interferon λ but not interferon ω. In some embodiments, the antibody specifically binds interferon α and interferon β but not interferon ω or interferon λ. In some embodiments, the antibody specifically binds interferon α and interferon ω but not interferon β or interferon λ. In some embodiments, the antibody specifically binds interferon α and interferon λ but not interferon β or interferon ω. In some embodiments, the antibody is rontalizumab. In some embodiments, the anti-dsDNA antibody status of the patient is determined by immunoassay. In some embodiments, the sample is selected from whole blood, blood-derived cells, plasma, serum, and combinations thereof. In some embodiments, the methods further comprise administering an effective amount of the interferon type I antibody to the patient. In some embodiments, the antibody is administered intravenously. In some embodiments, the antibody is administered subcutaneously. In some embodiments, the antibody is administered for at least 24 weeks. In some embodiments, the patient has a baseline interferon signature metric (ISM) that is greater than or equal to the ISM of a healthy individual. In some embodiments, the patient has an ISM that is lower following administration of the antibody as compared to the patient's baseline ISM. In some embodiments, the ISM is determined by measuring the expression level of at least one gene selected from the group consisting of: CMPK2, EPST1, HERC5, and combinations thereof. In some embodiments, prior to treatment with an interferon inhibitor, the IRG status of the patient has been determined. In one embodiment, any one or combination or all of the IRGs of the Enlarged ISM, the Enlarged ISM-A, the 24-gene ISM or the 3-gene ISM are used to evaluate the IRG status. In some embodiments, prior to treatment with an interferon inhibitor, the patient has been determined to have an anti-dsDNA antibody low status and an IRG status that is ISM^{lo}. In some embodiments, prior to treatment with an interferon inhibitor, the patient has been determined to have an anti-dsDNA antibody low status and an IRG status that is ISM^{hi}. In some embodiments, the ISM is determined by measuring the expression level of at least one gene selected from the group consisting of: IFI27, IFI44, IFIT1, MX1, OAS1, OAS2, OAS3, and combinations thereof. In some embodiments, the methods further comprise administering a second medicament to the subject. In some embodiments, the second medicament is elected from a corticosteroid, a non-steroidal anti-inflammatory drug (NSAID), an anti-malarial agent, a statin, and combinations thereof.

Even another embodiment of the invention provides methods of predicting responsiveness of an autoimmune patient (e.g., lupus patient) to treatment with an interferon type I antibody, the method comprising determining anti-dsDNA antibody status of the patient, wherein a patient who is determined to have an anti-dsDNA antibody status of low (e.g. ≦200 IU) is identified as a patient who is likely to respond to treatment with the interferon type I antibody. In some embodiments, the antibody specifically binds an interferon selected from the group consisting of: interferon α, interferon β, interferon ω, interferon λ and combinations thereof. In some embodiments, the antibody specifically binds interferon α. In some embodiments, the antibody specifically binds interferon β. In some embodiments, the antibody specifically binds interferon ω. In some embodiments, the antibody specifically binds interferon λ. In some embodiments, the antibody specifically binds interferon α, interferon β, interferon ω and interferon λ. In some embodiments, the antibody specifically binds interferon α, interferon β and interferon ω but not interferon λ. In some embodiments, the antibody specifically binds interferon α, interferon λ and interferon ω but not interferon β. In some embodiments, the antibody specifically binds interferon α, interferon β and interferon λ; but not interferon ω. In some embodiments, the antibody specifically binds interferon α and interferon β but not interferon ω or interferon λ. In some embodiments, the antibody specifically binds interferon α and interferon ω but not interferon β or interferon λ. In some embodiments, the antibody specifically binds interferon α and interferon λ but not interferon β or interferon ω. In some embodiments, the antibody is rontalizumab. In some embodiments, the anti-dsDNA antibody status of the patient is determined by immunoassay. In some embodiments, the sample is selected from whole blood, blood-derived cells, plasma, serum, and combinations thereof. In some embodiments, the methods further comprise administering an effective amount of the interferon type I antibody to the patient. In some embodiments, the antibody is administered intravenously. In some embodiments, the antibody is administered subcutaneously. In some embodiments, the antibody is administered for at least 24 weeks. In some embodiments, the patient has a baseline interferon signature metric (ISM) that is greater than or equal to the ISM of a healthy individual. In some embodiments, the patient has an ISM that is lower following administration of the antibody as compared to the patient's baseline ISM. In some embodiments, the ISM is determined by measuring the expression level of at least one gene selected from the group consisting of: CMPK2, EPST1, HERC5, and combinations thereof. In some embodiments, the ISM is determined by measuring the expression level of at least one gene selected from the group consisting of: IFI27, IFI44, IFIT1, MX1, OAS1, OAS2, OAS3, and combinations thereof. In some embodiments, prior to treatment with an interferon inhibitor, the IRG status of the patient has been determined. In one embodiment, any one or combination or all of the IRGs of the Enlarged ISM, the Enlarged ISM-A, the 24-gene ISM or the 3-gene ISM are used to evaluate the IRG status. In some embodiments, prior to treatment with an interferon inhibitor, the patient has been determined to have an anti-dsDNA antibody low status and an IRG status that is ISM^{lo}, which status can be further useful in predicting which autoimmune patients are more likely to respond to the interferon inhibitor. In some embodiments, prior to treatment with an interferon inhibitor, the patient has been determined to have an anti-dsDNA antibody low status and an IRG status that is ISM^{hi} which status can be further useful in identifying which autoimmune patients are more likely to respond to the interferon inhibitor. In some embodiments, the methods further comprise administering a second medicament to the subject. In some embodiments, the second medicament is elected from a corticosteroid, a non-steroidal anti-inflammatory drug (NSAID), an anti-malarial agent, a statin, and combinations thereof.

Yet another embodiment of the invention provides methods for determining the likelihood that a lupus patient will benefit from treatment with an interferon type I antibody, the method comprising determining anti-dsDNA antibody status of the patient, wherein a patient who is determined to have an anti-dsDNA antibody status of low (e.g. ≦200 IU) is identified as a patient who is likely to respond to treatment with the interferon type I antibody. In some embodiments, the antibody specifically binds an interferon selected from the group consisting of: interferon α, interferon β, interferon ω, interferon λ and combinations thereof. In some embodiments, the antibody specifically binds interferon α. In some embodiments, the antibody specifically binds interferon β. In some embodiments, the antibody specifically binds interferon ω. In some embodiments, the antibody specifically binds interferon λ. In some embodiments, the antibody specifically binds interferon α, interferon β, interferon ω and interferon λ. In some embodiments, the antibody specifically binds interferon α, interferon β and interferon ω but not interferon λ. In some embodiments, the antibody specifically binds interferon α, interferon λ and interferon ω; but not interferon β. In some embodiments, the antibody specifically binds interferon α, interferon β and interferon λ but not interferon ω. In some embodiments, the antibody specifically binds interferon α and interferon β but not interferon ω or interferon λ. In some embodiments, the antibody specifically binds interferon α and interferon ω but not interferon β or interferon λ. In some embodiments, the antibody specifically binds interferon α and interferon λ but not interferon β or interferon ω. In some embodiments, the antibody is rontalizumab. In some embodiments, the anti-dsDNA antibody status of the patient is determined by immunoassay. In some embodiments, the sample is selected from whole blood, blood-derived cells, plasma, serum, and combinations thereof. In some embodiments, the methods further comprise administering an effective amount of the interferon type I antibody to the patient. In some embodiments, the antibody is administered intravenously. In some embodiments, the antibody is administered subcutaneously. In some embodiments, the antibody is administered for at least 24 weeks. In some embodiments, the patient has a baseline interferon signature metric (ISM) that is greater than or equal to the ISM of a healthy individual. In some embodiments, the patient has an IRG status of ISM^{lo}. In some embodiments, the patient has an ISM that is lower following administration of the antibody as compared to the patient's baseline ISM. In some embodiments, the ISM is determined by measuring the expression level of at least one gene selected from the group consisting of: CMPK2, EPST1, HERC5, and combinations thereof. In some embodiments, the ISM is determined by measuring the expression level of at least one gene selected from the group consisting of: IFI27, IFI44, IFIT1, MX1, OAS1, OAS2, OAS3, and combinations thereof. In some embodiments, prior to treatment with an interferon inhibitor, the IRG status of the patient has been determined. In one embodiment, any one or combination or all of the IRGs of the Enlarged ISM, the Enlarged ISM-A, the 24-gene ISM or the 3-gene ISM are used to evaluate the IRG status. In some embodiments, prior to treatment with an interferon inhibitor, the patient has been determined to have an anti-dsDNA antibody low status and an IRG status that is ISM^{lo}, which status can be further useful in predicting which autoimmune patients are more likely to benefit from treatment with the interferon inhibitor. In some embodiments, prior to treatment with an interferon inhibitor, the patient has been determined to have an anti-dsDNA antibody low status and an IRG status that is ISM^{hi} which status can be further useful in identifying which autoimmune patients are more likely to benefit from treatment with the interferon inhibitor. In some embodiments, the methods further comprise administering a second medicament to the subject. In some embodiments, the second medicament is elected from a corticosteroid, a non-steroidal anti-inflammatory drug (NSAID), an anti-malarial agent, a statin, and combinations thereof.

Another embodiment of the invention provides methods of treating autoimmune disease (e.g., lupus) in a patient, the method comprising administering an effective amount of an interferon type I antibody to a patient diagnosed with the autoimmune disease, wherein the patient has a baseline interferon signature metric (ISM) that is greater than or equal to the ISM of a healthy individual. Another embodiment of the invention provides methods of treating autoimmune disease (e.g., lupus) in a patient, the method comprising administering an effective amount of an interferon inhibitor to a patient diagnosed with the autoimmune disease, wherein the IRG status of the autoimmune patient has been determined to be equal to the IRG status of a healthy person or to be I ISM^{lo}. In some embodiments, the patient has an IRG status of ISM^{lo}. In some embodiments, the antibody specifically binds an interferon selected from the group consisting of: interferon α, interferon β, interferon ω, interferon λ and combinations thereof. In some embodiments, the antibody specifically binds interferon α. In some embodiments, the antibody specifically binds interferon β. In some embodiments, the antibody specifically binds interferon ω. In some embodiments, the antibody specifically binds interferon λ. In some embodiments, the antibody specifically binds interferon α, interferon β, interferon ω and interferon λ. In some embodiments, the antibody specifically binds interferon α, interferon β and interferon ω but not interferon λ. In some embodiments, the antibody specifically binds interferon α, interferon λ and interferon ω but not interferon β. In some embodiments, the antibody specifically binds interferon α, interferon β and interferon λ but not interferon ω. In some embodiments, the antibody specifically binds interferon α and interferon β but not interferon ω or interferon λ. In some embodiments, the antibody specifically binds interferon α and interferon ω but not interferon β or interferon λ. In some embodiments, the antibody specifically binds interferon α and interferon λ but not interferon β or interferon ω. In some embodiments, the antibody is rontalizumab. In some embodiments, the antibody is administered intravenously. In some embodiments, the antibody is administered subcutaneously. In some embodiments, the antibody is administered for at least 24 weeks. In some embodiments, the ISM is determined by measuring the expression level of at least one gene selected from the group consisting of: CMPK2, EPST1, HERC5, and combinations thereof. In some embodiments, the ISM is determined by measuring the expression level of at least one gene selected from the group consisting of: IFI27, IFI44, IFIT1, MX1, OAS1, OAS2, OAS3, and combinations thereof. In one embodiment, any one or combination or all of the IRGs of the Enlarged ISM, the Enlarged ISM-A, the 24-gene ISM or the 3-gene ISM are used to evaluate the IRG status. In some embodiments, the methods further comprise administering a second medicament to the subject. In some embodiments, the second medicament is elected from a corticosteroid, a non-steroidal anti-inflammatory drug (NSAID), an anti-malarial agent, a statin, and combinations thereof.

Even another embodiment of the invention provides methods of treating lupus in a patient, the method comprising administering an effective amount of an interferon type I antibody to a patient diagnosed with lupus, wherein the patient has an ISM that is lower following administration of the antibody as compared to the patient's baseline ISM. In yet another embodiment, the invention provides methods of treating an autoimmune disease in a patient, the method comprising administering an effective amount of an interferon inhibitor to a patient diagnosed with the autoimmune disease, wherein any one, combination or all IRGs are monitored as pharmacodynamic markers after treating the patient with the interferon inhibitor. In one embodiment, the IRGs are CMPK2, EPSTI1, HERC5, IFI27, IFI44, IFIT1, MX1, OAS1, OAS2, and OAS3. In some embodiments, the antibody specifically binds an interferon selected from the group consisting of: interferon α, interferon β, interferon ω, interferon λ and combinations thereof. In some embodiments, the antibody specifically binds interferon α. In some embodiments, the antibody specifically binds interferon β. In some embodiments, the antibody specifically binds interferon ω. In some embodiments, the antibody specifically binds interferon λ. In some embodiments, the antibody specifically binds interferon α, interferon β, interferon ω and interferon λ. In some embodiments, the antibody specifically binds interferon α, interferon β and interferon ω but not interferon λ. In some embodiments, the antibody specifically binds interferon α, interferon λ and interferon ω but not interferon β. In some embodiments, the antibody specifically binds interferon α, interferon β and interferon λ but not interferon ω. In some embodiments, the antibody specifically binds interferon α and interferon β but not interferon ω or interferon λ. In some embodiments, the antibody specifically binds interferon α and interferon ω but not interferon β or interferon λ. In some embodiments, the antibody specifically binds interferon α and interferon λ but not interferon β or interferon ω. In some embodiments, the antibody is rontalizumab. In some embodiments, the antibody is administered intravenously. In some embodiments, the antibody is administered subcutaneously. In some embodiments, the antibody is administered for at least 24 weeks. In some embodiments, the ISM is determined by measuring the expression level of at least one gene selected from the group consisting of: CMPK2, EPST1, HERC5, and combinations thereof. In some embodiments, the ISM is determined by measuring the expression level of at least one gene selected from the group consisting of: IFI27, IFI44, IFIT1, MX1, OAS1, OAS2, OAS3, and combinations thereof. In some embodiments, the methods further comprise administering a second medicament to the subject. In some embodiments, the second medicament is elected from a corticosteroid, a non-steroidal anti-inflammatory drug (NSAID), an anti-malarial agent, a statin, and combinations thereof.

Even a further embodiment of the invention provides methods of identifying an autoimmune patient (e.g., lupus patient) who may benefit from treatment with an interferon type I antibody, the method comprising determining the baseline ISM status of the patient, wherein a patient who has a baseline ISM greater than or equal to the ISM of a healthy individual is identified as a patient who may benefit from treatment with the interferon type I antibody. Another embodiment of the invention provides methods of identifying an autoimmune patient (e.g., lupus patient) who may benefit from treatment with an interferon inhibitor, the method comprising determining the IRG status of the autoimmune patient, wherein a patient who has an IRG status that is equal to the IRG status of a healthy individual or is ISM low is identified as a patient who is likely to benefit from treatment with the interferon inhibitor. In some embodiments, the patient has an IRG status of ISM^{lo}. In some embodiments, the antibody specifically binds an interferon selected from the group consisting of: interferon α, interferon β, interferon ω, interferon λ and combinations thereof. In some embodiments, the antibody specifically binds interferon α. In some embodiments, the antibody specifically binds interferon β. In some embodiments, the antibody specifically binds interferon ω. In some embodiments, the antibody specifically binds interferon λ. In some embodiments, the antibody specifically binds interferon α, interferon β, interferon ω and interferon λ. In some embodiments, the antibody specifically binds interferon α, interferon β and interferon ω but not interferon λ. In some embodiments, the antibody specifically binds interferon α, interferon λ and interferon ω; but not interferon β. In some embodiments, the antibody specifically binds interferon α, interferon β and interferon λ but not interferon ω. In some embodiments, the antibody specifically binds interferon α and interferon β but not interferon ω or interferon λ. In some embodiments, the antibody specifically binds interferon α and interferon ω but not interferon β or interferon λ. In some embodiments, the antibody specifically binds interferon α and interferon λ but not interferon β or interferon ω. In some embodiments, the antibody is rontalizumab. In some embodiments, the methods further comprise administering an effective amount of the interferon type I antibody to the patient. In some embodiments, the antibody is administered intravenously. In some embodiments, the antibody is administered subcutaneously. In some embodiments, the antibody is administered for at least 24 weeks. In some embodiments, the patient has a baseline interferon signature metric (ISM) that is greater than or equal to the ISM of a healthy individual. In some embodiments, the patient has an IRG status of ISM^{lo}. In some embodiments, the patient has an ISM that is lower following administration of the antibody as compared to the patient's baseline ISM. In some embodiments, the ISM is determined by measuring the expression level of at least one gene selected from the group consisting of: CMPK2, EPST1, HERC5, and combinations thereof. In some embodiments, the ISM is determined by measuring the expression level of at least one gene selected from the group consisting of: IFI27, IFI44, IFIT1, MX1, OAS1, OAS2, OAS3, and combinations thereof. In one embodiment, any one or combination or all of the IRGs of the Enlarged ISM, the Enlarged ISM-A, the 24-gene ISM or the 3-gene ISM are used to evaluate the IRG status. In some embodiments, the methods further comprise administering a second medicament to the subject. In some embodiments, the second medicament is elected from a corticosteroid, a non-steroidal anti-inflammatory drug (NSAID), an anti-malarial agent, a statin, and combinations thereof.

Yet another embodiment of the invention provides methods of optimizing therapeutic efficacy for treatment of lupus, the method comprising determining the baseline ISM status of the patient, wherein a patient who has a baseline ISM greater than or equal to the ISM of a healthy individual has increased likelihood of benefit from treatment with the interferon type I antibody. In some embodiments, the patient has an IRG status of ISM^{lo}. In some embodiments, the antibody specifically binds an interferon selected from the group consisting of: interferon α, interferon β, interferon ω, interferon λ and combinations thereof. In some embodiments, the antibody specifically binds interferon α. In some embodiments, the antibody specifically binds interferon β. In some embodiments, the antibody specifically binds interferon ω. In some embodiments, the antibody specifically binds interferon λ. In some embodiments, the antibody specifically binds interferon α, interferon β, interferon ω and interferon λ. In some embodiments, the antibody specifically binds interferon α, interferon β and interferon ω but not interferon λ. In some embodiments, the antibody specifically binds interferon α, interferon λ and interferon ω; but not interferon β. In some embodiments, the antibody specifically binds interferon α, interferon β and interferon λ; but not interferon ω. In some embodiments, the antibody specifically binds interferon α and interferon β but not interferon ω or interferon λ. In some embodiments, the antibody specifically binds interferon α and interferon ω but not interferon β or interferon λ. In some embodiments, the antibody specifically binds interferon α and interferon λ but not interferon β or interferon ω. In some embodiments, the methods further comprise administering an effective amount of the interferon type I antibody to the patient. In some embodiments, the antibody is administered intravenously. In some embodiments, the antibody is administered subcutaneously. In some embodiments, the antibody is administered for at least 24 weeks. In some embodiments, the patient has a baseline interferon signature metric (ISM) that is greater than or equal to the ISM of a healthy individual. In some embodiments, the patient has an ISM that is lower following administration of the antibody as compared to the patient's baseline ISM. In some embodiments, the ISM is determined by measuring the expression level of at least one gene selected from the group consisting of: CMPK2, EPST1, HERC5, and combinations thereof. In some embodiments, the ISM is determined by measuring the expression level of at least one gene selected from the group consisting of: IFI27, IFI44, IFIT1, MX1, OAS1, OAS2, OAS3, and combinations thereof. In some embodiments, the methods further comprise administering a second medicament to the subject. In some embodiments, the second medicament is elected from a corticosteroid, a non-steroidal anti-inflammatory drug (NSAID), an anti-malarial agent, a statin, and combinations thereof.

Yet a further embodiment of the invention provides methods of predicting responsiveness of a lupus patient to treatment with an interferon type I antibody, the method comprising determining the ISM status of the patient, wherein a patient who has an ISM greater than or equal to the ISM of a healthy individual is identified as a patient who is likely to respond to treatment with the interferon type I antibody. In another embodiment, the invention provides methods of predicting responsiveness of an autoimmune patient to treatment with an interferon inhibitor, the method comprising determining the IRG status of the patient, wherein a patient who has an IRG status that is equal to the IRG status of a healthy individual or is ISM low is identified as a patient who is likely to respond to treatment with the interferon inhibitor. In some embodiments, the patient has an IRG status of ISM^{lo}. In some embodiments, the antibody specifically binds an interferon selected from the group consisting of: interferon α, interferon β, interferon ω, interferon λ and combinations thereof. In some embodiments, the antibody specifically binds interferon α. In some embodiments, the antibody specifically binds interferon β. In some embodiments, the antibody specifically binds interferon ω. In some embodiments, the antibody specifically binds interferon λ. In some embodiments, the antibody specifically binds interferon α, interferon β, interferon ω and interferon λ. In some embodiments, the antibody specifically binds interferon α, interferon β and interferon ω; but not interferon λ. In some embodiments, the antibody specifically binds interferon α, interferon λ and interferon ω but not interferon β. In some embodiments, the antibody specifically binds interferon α, interferon β; and interferon λ; but not interferon ω. In some embodiments, the antibody specifically binds interferon α and interferon β but not interferon ω or interferon λ. In some embodiments, the antibody specifically binds interferon α and interferon ω but not interferon β or interferon λ. In some embodiments, the antibody specifically binds interferon α and interferon λ but not interferon β or interferon ω. In some embodiments, the antibody is rontalizumab. In some embodiments, the methods further comprise administering an effective amount of the interferon type I antibody to the patient. In some embodiments, the antibody is administered intravenously. In some embodiments, the antibody is administered subcutaneously. In some embodiments, the antibody is administered for at least 24 weeks. In some embodiments, the patient has a baseline interferon signature metric (ISM) that is greater than or equal to the ISM of a healthy individual. In some embodiments, the patient has an ISM that is lower following administration of the antibody as compared to the patient's baseline ISM. In some embodiments, the ISM is determined by measuring the expression level of at least one gene selected from the group consisting of: CMPK2, EPST1, HERC5, and combinations thereof. In some embodiments, the ISM is determined by measuring the expression level of at least one gene selected from the group consisting of: IFI27, IFI44, IFIT1, MX1, OAS1, OAS2, OAS3, and combinations thereof. In one embodiment, any one or combination or all of the IRGs of the Enlarged ISM, the Enlarged ISM-A, the 24-gene ISM or the 3-gene ISM are used to evaluate the IRG status. In some embodiments, the methods further comprise administering a second medicament to the subject. In some embodiments, the second medicament is elected from a corticosteroid, a non-steroidal anti-inflammatory drug (NSAID), an anti-malarial agent, a statin, and combinations thereof.

Another embodiment of the invention provides methods for determining the likelihood that a lupus patient will benefit from treatment with an interferon type I antibody, the method comprising determining the ISM status of the patient, wherein a patient who has an ISM greater than or equal to the ISM of a healthy individual is identified as a patient who is likely to respond to treatment with the interferon type I antibody. Another embodiment of this invention provides methods for determining the likelihood that an autoimmune patient will benefit from treatment with an interferon inhibitor, the method comprising determining the IRG status of the patient, wherein a patient who has an IRG status that is equal to the IRG status of a healthy individual or is ISM low is identified as a patient who is likely to respond benefit from treatment with the interferon inhibitor. In some embodiments, the patient has an IRG status of ISM^{lo}. In some embodiments, the antibody specifically binds an interferon selected from the group consisting of: interferon α, interferon β, interferon ω, interferon λ and combinations thereof. In some embodiments, the antibody specifically binds interferon α. In some embodiments, the antibody specifically binds interferon β. In some embodiments, the antibody specifically binds interferon ω. In some embodiments, the antibody specifically binds interferon λ. In some embodiments, the antibody specifically binds interferon α, interferon β, interferon ω and interferon λ. In some embodiments, the antibody specifically binds interferon α, interferon β and interferon ω; but not interferon λ. In some embodiments, the antibody specifically binds interferon α, interferon λ and interferon ω but not interferon β. In some embodiments, the antibody specifically binds interferon α, interferon β and interferon λ but not interferon ω. In some embodiments, the antibody specifically binds interferon α and interferon β but not interferon ω or interferon λ. In some embodiments, the antibody specifically binds interferon α and interferon ω but not interferon β or interferon λ. In some embodiments, the antibody specifically binds interferon α and interferon λ but not interferon β or interferon ω. In some embodiments, the antibody is rontalizumab. In some embodiments, the methods further comprise administering an effective amount of the interferon type I antibody to the patient. In some embodiments, the antibody is administered intravenously. In some embodiments, the antibody is administered subcutaneously. In some embodiments, the antibody is administered for at least 24 weeks. In some embodiments, the patient has a baseline interferon signature metric (ISM) that is greater than or equal to the ISM of a healthy individual. In some embodiments, the patient has an ISM that is lower following administration of the antibody as compared to the patient's baseline ISM. In some embodiments, the ISM is determined by measuring the expression level of at least one gene selected from the group consisting of: CMPK2, EPST1, HERC5, and combinations thereof. In some embodiments, the ISM is determined by measuring the expression level of at least one gene selected from the group consisting of: IFI27, IFI44, IFIT1, MX1, OAS1, OAS2, OAS3, and combinations thereof. In one embodiment, any one or combination or all of the IRGs of the Enlarged ISM, the Enlarged ISM-A, the 24-gene ISM or the 3-gene ISM are used to evaluate the IRG status. In some embodiments, the methods further comprise administering a second medicament to the subject. In some embodiments, the second medicament is elected from a corticosteroid, a non-steroidal anti-inflammatory drug (NSAID), an anti-malarial agent, a statin, and combinations thereof.

In one embodiments of the methods of this invention, the IRG status is determined by measuring the expression levels of one, combination or all of the following IRGs (e.g., Enlarged ISM): CHMP5, CIG5, EPSTI1, G1P2, HERC5, IFI44, IFI44L, IFIT1, IFIT4, IFIT5, IRF7, MX1, OAS1, OAS2, OAS3, OASL, RIG1, RIGE, SAMD9L, SP110, TYK1 (CMPK2), XIAP, ZBP1, PARP9, IFI27, SIGLEC1, DNAPTP6, USP18, IFI6, HSXIAPAF1, and LAMP3. In another embodiment of the methods of this invention, the IRG status is determined by measuring the expression levels of one, combination or all of the following IRGs (e.g., Enlarged ISM-A): IFI27, CIG5, IFI44L, IFI44, OAS1, OAS3, IFIT1, G1P2, HERC5, MX1, EPSTI1, IFIT3 and IFI6. In another embodiment of the methods of this invention, the IRG status is determined by measuring the expression levels of one, combination or all of the following IRGs (e.g., 24-gene ISM): CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP and ZBP1. In another embodiment of the methods of this invention, the IRG status is determined by measuring the expression levels of one, combination or all of the following IRGs (e.g., 3-gene ISM): EPSTI1, HERC5 and TYK1 (CMPK2). In some embodiments of the methods described herein, the IRG status is measured using qPCR. In one further embodiment, the qPCR is performed on a Roche Cobas® system.

In another aspect, the invention provides a method of treating an autoimmune disease in a patient, the method comprising administering an effective amount of an interferon inhibitor to a patient, wherein the patient has been diagnosed with the autoimmune disease and has been determined to be ISM^{lo} or has been selected for treatment based on being ISM^{lo}. In some embodiments, the ISM^{lo} is determined by measuring mRNA expression levels of one or more interferon response genes (IRGs) in a sample from the patient by RT-PCR. In some embodiments, the ISM^{lo} is determined by measuring mRNA expression levels of one or more interferon response genes (IRGs) in a sample from the patient by qPCR. In some embodiments, the qPCR is performed on a Roche Cobas® system. In some embodiments, the sample is a blood sample. In some embodiments, the IRG status is determined by measuring the mRNA expression levels of one, combination or all of the following IRGs (e.g., Enlarged ISM): CHMP5, CIG5, EPSTI1, G1P2, HERC5, IFI44, IFI44L, IFIT1, IFIT4, IFIT5, IRF7, MX1, OAS1, OAS2, OAS3, OASL, RIG1, RIGE, SAMD9L, SP110, TYK1 (CMPK2), XIAP, ZBP1, PARP9, IFI27, SIGLEC1, DNAPTP6, USP18, IFI6, HSXIAPAF1, and LAMP3. In some embodiments, the IRG status is determined by measuring the mRNA expression levels of one, combination or all of the following IRGs (e.g., 24-gene ISM): CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP and ZBP1. In some embodiments, the mRNA expression levels of EPSTI1, HERC5 and/or TYK1 (CMPK2) are determined. In some embodiments, the mRNA expression levels of the IRGs are normalized against mRNA expression level of a housekeeping gene. In some embodiments, the mRNA expression levels of EPSTI1, HERC5 and/or TYK1 (CMPK2) are normalized against mRNA expression level of transferrin receptor (TFRC).

In another aspect, the invention provides a method of treating an autoimmune disease in a patient, the method comprising administering an effective amount of an interferon inhibitor to a patient, wherein the patient has been diagnosed with the autoimmune disease and has been determined to have a pre-treatment anti-double stranded DNA antibody titer (anti-dsDNA) that is less than or equal to 200 IU as measured by immunoassay or selected for treatment based on having a pre-treatment anti-double stranded DNA antibody titer (anti-dsDNA) that is less than or equal to 200 IU as measured by immunoassay. In some embodiments, the immunoassay is an ELISA. In some embodiments, the patient has an anti-dsDNA titer that is less than or equal to 200 IU and is ISM^{hi}. In some embodiments, the ISM^{hi} is determined by measuring mRNA expression levels of one or more IRGs in a sample from the patient by RT-PCR. In some embodiments, the ISM^{hi} is determined by measuring mRNA expression levels of one or more IRGs in a sample from the patient by qPCR. In some embodiments, the qPCR is performed on a Roche Cobas® system. In some embodiments, the sample is a blood sample. In some embodiments, the IRG status is determined by measuring the mRNA expression levels of one, combination or all of the following IRGs (e.g., Enlarged ISM): CHMP5, CIG5, EPSTI1, G1P2, HERC5, IFI44, IFI44L, IFIT1, IFIT4, IFIT5, IRF7, MX1, OAS1, OAS2, OAS3, OASL, RIG1, RIGE, SAMD9L, SP110, TYK1 (CMPK2), XIAP, ZBP1, PARP9, IFI27, SIGLEC1, DNAPTP6, USP18, IFI6, HSXIAPAF1, and LAMPS. In some embodiments, the IRG status is determined by measuring the mRNA expression levels of one, combination or all of the following IRGs (e.g., 24-gene ISM): CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP and ZBP1. In some embodiments, the mRNA expression levels of EPSTI1, HERC5 and/or TYK1 (CMPK2) are determined. In some embodiments, the mRNA expression levels of the IRGs are normalized against mRNA expression level of a housekeeping gene. In some embodiments, the mRNA expression levels of EPSTI1, HERC5 and/or TYK1 (CMPK2) are normalized against TFRC.

In some embodiments, the autoimmune disease is selected from the group consisting of lupus, rheumatoid arthritis, psoriasis, psoriatic arthritis, insulin-dependent diabetes mellitus (IDDM), multiple sclerosis (MS), myositis, dermatomyositis, vasculitis, atherosclerosis, ankylosing spondylitis, and Sjogren's syndrome. In some embodiments, the patient has moderately to severely active lupus (such as moderately to severely active SLE). In some embodiments, the patient has lupus nephritis. In some embodiments, the patient has Class III-V lupus nephritis and is ISM^{lo}. In some embodiments, the patient has pediatric lupus.

In some embodiments, the interferon inhibitor in the methods described herein is an anti-interferon type I antibody. In some embodiments, the antibody specifically binds an interferon selected from the group consisting of: interferon α, interferon β, interferon ω, interferon λ, and combinations thereof. In some embodiments, the antibody specifically binds interferon α. In some embodiments, the antibody binds to at least IFNα subtypes 1, 2, 4, 5, 8, 10 and 21. In some embodiments, the antibody comprises a light chain comprising HVR-L1 comprising the amino acid sequence RASQSVSTSSYSYMH (SEQ ID NO:1), HVR-L2 comprising the amino acid sequence YASNLES (SEQ ID NO:2), and HVR-L3 comprising the amino acid sequence QHSWGIPRTF (SEQ ID NO: 3); and/or a heavy chain comprising HVR-H1 comprising the amino acid sequence GYTFTEYIIH (SEQ ID NO: 4), HVR-H2 comprising the amino acid sequence SINPDYDITNYNQRFKG (SEQ ID NO:5), and HVR-H3 comprising the amino acid sequence WISDFFDY (SEQ ID NO:6). In some embodiments, the antibody comprises a heavy chain variable region sequence of at least 95% sequence identity to the amino acid sequence of SEQ ID NO:7; and/or a light chain variable region sequence of at least 95% sequence identity to the amino acid sequence of SEQ ID NO:8. In some embodiments, the antibody comprises a heavy chain variable region comprising the amino acid sequence of SEQ ID NO:7; and/or a light chain variable region comprising the amino acid sequence of SEQ ID NO:8. In some embodiments, the antibody is rontalizumab having CAS registration number 948570-30-7. In some embodiments, the antibody has an amino acid sequence as disclosed in CAS 1006877-41-3.

In some embodiments, the anti-interferon type I antibody is administered intravenously or subcutaneously. In some embodiments, the antibody is administered at a flat dose of 100 to 2000 mg. In some embodiments, the antibody is administered at a flat dose of 100-500 mg weekly, 200-1000 mg biweekly, or 400-2000 mg monthly. In some embodiments, the antibody is administered at a flat does of 150 mg or 300 mg weekly, 300 mg or 600 mg biweekly, or 600 mg, 750 mg or 1200 mg monthly. In some embodiments, the antibody is administered subcutaneously at a flat dose of 150 mg or 300 mg weekly.

In some embodiments, the administration of the antibody is effective in one or more of the following: (1) reduction of the number and/or severity of lupus flares, (2) prevention of lupus flares, (3) reduction in lupus nephritis flares, (4) prevention of lupus nephritis flares, (5) induction of remission in lupus nephritis, (6) maintenance of lupus nephritis remission, (7) reduction in the number and/or severity of pediatric lupus flares, (8) prevention of pediatric lupus flares, (9) reduction in pediatric lupus nephritis flares, (10) prevention of pediatric lupus nephritis flares, (11) induction of remission in pediatric lupus nephritis, and (12) maintenance of pediatric lupus nephritis remission. In some embodiments, the administration of the antibody is effective in lowering the anti-dsDNA antibody titer in the patient. In some embodiments, the administration of the antibody is effective in reduction of flare(s) in the patient. In some embodiments, said flare(s) are moderate or severe. In some embodiments, the administration of the antibody is effective in reduction of Selena Flare Index (SFI) score or Selena Flare Index-Revised (SFI-R) score in the patient. In some embodiments, the administration of the antibody is effective in decreasing all pre-treatment BILAG A and B domains. In some embodiments, the patient has no new BILAG A organ domain score or no more than one new BILAG B organ domain score after the administration of the antibody. In some embodiments, the administration of the antibody is effective in decreasing in SELENA-SLEDAI score by at least four points from the patient's pre-treatment score. In some embodiments, the patient has no more than 0.3 points increase in Physician Global Assessment (PGA) from the pre-treatment score after the administration of the antibody. In some embodiments, said patient has a post-treatment decrease in disease activity in those organ systems with moderate or severe disease activity prior to treatment as measured by any one of the following assessment tools: SRI, BILAG, SELENA-SLEDAI, or Physician Global Assessment (PGA). In some embodiments, the patient has an SRI-4, SRI-5, SRI-6, or SRI-7 response to the administration of the antibody.

In some embodiments, the methods described herein further comprise administering a second medicament to the patient. In some embodiments, the second medicament is selected from the group consisting of: a corticosteroid, a non-steroidal anti-inflammatory drug (NSAID), an immunosuppressive, an anti-malarial agent, a statin, and combinations thereof. In some embodiments, the second medicament is a standard of care for lupus.

In some embodiments, the administration of the antibody results in corticosteroid sparing (CS) in a patient taking a corticosteroid prior to said administration of said antibody. In some embodiments, the administration of the antibody results in a decrease in the requirement for therapy with steroids and/or immunosuppressive regimens. In some embodiments, the patient has tapered their corticosteroid dose to a prednisone equivalent of 10 mg/day after the administration of the antibody. In some embodiments, the administration of the antibody results in reduction in corticosteroid use by at least 50% after about 24 to about 52 weeks of the administration of the antibody. In some embodiments, the administration of the antibody results in one or more of the following: reduction in the incidence of moderate and/or severe flares as measured by SELENA SLEDAI scores and/or Physicians Global Assessment; significantly delaying time to severe flare; BILAG A (severe) organ flare or more than one BILAG B (moderate) organ flare.

In another aspect, the invention provides a therapeutic regimen for the treatment of an ISM^{lo} SLE patient in need thereof comprising the administration of an interferon inhibitor. In some embodiments, the interferon inhibitor is an anti-IFNα antibody. In some embodiments, the antibody is administered at a flat dose of 100-2000 mg. In some embodiments, the antibody is administered at a flat dose of 100-500 mg weekly, 200-1000 mg biweekly, or 400-2000 mg monthly. In some embodiments, the antibody is administered at a flat dose of 150 mg or 300 mg weekly, 300 mg or 600 mg biweekly, or 600 mg, 750 mg or 1200 mg monthly. In some embodiments, the antibody is administered intravenously or subcutaneously. In some embodiments, the antibody is administered subcutaneously at a flat dose of 150 mg or 300 mg weekly. In some embodiments, the antibody comprises a light chain comprising HVR-L1 comprising the amino acid sequence RASQSVSTSSYSYMH (SEQ ID NO:1), HVR-L2 comprising the amino acid sequence YASNLES (SEQ ID NO:2), and HVR-L3 comprising the amino acid sequence QHSWGIPRTF (SEQ ID NO:3); and/or a heavy chain comprising HVR-H1 comprising the amino acid sequence GYTFTEYIIH (SEQ ID NO:4), HVR-H2 comprising the amino acid sequence SINPDYDITNYNQRFKG (SEQ ID NO:5), and HVR-H3 comprising the amino acid sequence WISDFFDY (SEQ ID NO:6). In some embodiments, the antibody comprises a heavy chain variable region sequence of at least 95% sequence identity to the amino acid sequence of SEQ ID NO:7; and/or a light chain variable region sequence of at least 95% sequence identity to the amino acid sequence of SEQ ID NO:8. In some embodiments, the antibody comprises a heavy chain variable region comprising the amino acid sequence of SEQ ID NO:7; and/or a light chain variable region comprising the amino acid sequence of SEQ ID NO:8. In some embodiments, the antibody is rontalizumab having CAS registration number 948570-30-7. In some embodiments, the antibody comprises an amino acid sequence as disclosed in CAS 1006877-41-3.

In another aspect, the invention provides a method of identifying a lupus patient who may benefit from an interferon inhibitor treatment, the method comprising determining the IRG status in a sample from the patient, wherein a patient who is ISM^{lo} is identified as a patient who may benefit from the interferon inhibitor treatment. In another aspect, in the invention provides a method of predicting responsiveness of a lupus patient to an interferon inhibitor treatment, the method comprising determining the IRG status in a sample from the patient, wherein a patient who is ISM^{lo} is identified as a patient who is likely to respond to the interferon inhibitor treatment. In some embodiments, the ISM^{lo} is determined by measuring mRNA expression levels of one or more interferon response genes (IRGs) in a sample from the patient by RT-PCR. In some embodiments, the ISM^{lo} is determined by measuring mRNA expression levels of one or more interferon response genes (IRGs) in a sample from the patient by qPCR. In some embodiments, the qPCR is performed on a Roche Cobas® system. In some embodiments, the sample is a blood sample. In some embodiments, the IRG status is determined by measuring the mRNA expression levels of one, combination or all of the following IRGs (e.g., Enlarged ISM): CHMP5, CIG5, EPSTI1, G1P2, HERC5, IFI44, IFI44L, IFIT1, IFIT4, IFIT5, IRF7, MX1, OAS1, OAS2, OAS3, OASL, RIG1, RIGE, SAMD9L, SP110, TYK1 (CMPK2), XIAP, ZBP1, PARP9, IFI27, SIGLEC1, DNAPTP6, USP18, IFI6, HSXIAPAF1, and LAMPS. In some embodiments, the IRG status is determined by measuring the mRNA expression levels of one, combination or all of the following IRGs (e.g., 24-gene ISM): CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP and ZBP1. In some embodiments, the mRNA expression levels of EPSTI1, HERC5 and/or TYK1 (CMPK2) are determined. In some embodiments, the mRNA expression levels of the IRGs are normalized against mRNA expression level of a housekeeping gene. In some embodiments, the mRNA expression levels of EPSTI1, HERC5 and/or TYK1 (CMPK2) are normalized against mRNA expression level of transferrin receptor (TFRC). In some embodiments, benefit includes any decrease in disease activity score as assessed by any one of the following: BILAG, SELENA-SLEDAI, SRI, PGA, SFI or SFI-R.

In another aspect, the invention provides a method of identifying a lupus patient who may benefit from an interferon inhibitor treatment, the method comprising determining the anti-dsDNA antibody status in a sample from the patient, wherein a patient who has an anti-dsDNA antibody titer that is less than or equal to 200 IU as measured by immunoassay is identified as a patient who may benefit from the interferon inhibitor treatment. In another aspect, the invention provides a method of predicting responsiveness of a lupus patient to an interferon inhibitor treatment, the method comprising determining the anti-dsDNA antibody status in a sample from the patient, wherein a patient who has an anti-dsDNA antibody titer that is less than or equal to 200 IU as measured by immunoassay is identified as a patient who is likely to respond to the interferon inhibitor treatment. In some embodiments, the immunoassay is an ELISA. In some embodiments, benefit includes any decrease in disease activity score as assessed by any one of the following: BILAG, SELENA-SLEDAI, SRI, PGA, SFI or SFI-R.

In some embodiments, the interferon inhibitor is an anti-interferon type I antibody. the antibody specifically binds an interferon selected from the group consisting of: interferon α, interferon β, interferon ω, interferon λ, and combinations thereof. In some embodiments, the antibody specifically binds interferon α. In some embodiments, the antibody binds to at least IFNα subtypes 1, 2, 4, 5, 8, 10 and 21. In some embodiments, the antibody comprises a light chain comprising HVR-L1 comprising the amino acid sequence RASQSVSTSSYSYMH (SEQ ID NO:1), HVR-L2 comprising the amino acid sequence YASNLES (SEQ ID NO:2), and HVR-L3 comprising the amino acid sequence QHSWGIPRTF (SEQ ID NO:3); and/or a heavy chain comprising HVR-H1 comprising the amino acid sequence GYTFTEYIIH (SEQ ID NO:4), HVR-H2 comprising the amino acid sequence SINPDYDITNYNQRFKG (SEQ ID NO:5), and HVR-H3 comprising the amino acid sequence WISDFFDY (SEQ ID NO:6). In some embodiments, the antibody comprises a heavy chain variable region sequence of at least 95% sequence identity to the amino acid sequence of SEQ ID NO:7; and/or a light chain variable region sequence of at least 95% sequence identity to the amino acid sequence of SEQ ID NO:8. In some embodiments, the antibody comprises a heavy chain variable region comprising the amino acid sequence of SEQ ID NO:7; and/or a light chain variable region comprising the amino acid sequence of SEQ ID NO:8. In some embodiments, the antibody is rontalizumab having CAS registration number 948570-30-7. In some embodiments, the antibody comprises an amino acid sequence as disclosed in CAS 1006877-41-3.

In another aspect, the invention provides a method for predicting the likelihood of a flare in a lupus patient, the method comprising determining the IRG status of the patient, wherein a significant increase of expression levels of IRGs indicates that the patient is likely to have a flare in the next 3 to 5 weeks. In some embodiments, the IRG status of the patient is monitored at baseline and/or after administration of an interferon inhibitor (such as an anti-interferon type I antibody described herein), and a significant increase of the expression levels of one or more of IRGs as compared the lowest level of the same IRG in a sample of the patient after the administration indicates that the patient is likely to have a flare in the next 3 to 5 weeks. In some embodiments, the increase is at least about 50%, at least about 75%, at least about 100%, or at least about 150%. In some embodiments, said IRGs are selected from the group consisting of EPSTI1, HERC5, TYK1 (CMPK2), IFI27, IFI44, IFIT1, MX1, OAS1, OAS2, OAS3, and a combination thereof. In some embodiments, the flare is determined by the SELENA-SLEDAI Flare Index (SFI) and/or SFI-Revised. In some embodiments, the flare is mild, moderate or severe based on the SELENA-SLEDAI Flare Index (SFI) and/or SFI-Revised.

In another aspect, the invention provides an article of manufacture comprising a subcutaneous administration device, which delivers to a patient a flat dose of an anti-interferon α antibody, wherein the flat dose is in the range of 50 mg to 2000 mg of the anti-interferon α antibody. In some embodiments, the flat dose is 100-500 mg weekly, 200-1000 mg biweekly, or 400-2000 mg monthly. In some embodiments, the flat dose is 150 mg or 300 mg weekly, 300 mg or 600 mg biweekly, or 600 mg, 750 mg or 1200 mg monthly. In some embodiments, the flat dose is 150 mg or 300 mg weekly. In some embodiments, the concentration of the antibody in the device is from about 50 to 250 mg/mL. In another aspect, the invention provides an article of manufacture comprising an anti-interferon α antibody in a concentration from about 50 to 250 mg/mL. In some embodiments, the antibody comprises a light chain comprising HVR-L1 comprising the amino acid sequence RASQSVSTSSYSYMH (SEQ ID NO:1), HVR-L2 comprising the amino acid sequence YASNLES (SEQ ID NO:2), and HVR-L3 comprising the amino acid sequence QHSWGIPRTF (SEQ ID NO:3); and/or a heavy chain comprising HVR-H1 comprising the amino acid sequence GYTFTEYIIH (SEQ ID NO:4), HVR-H2 comprising the amino acid sequence SINPDYDITNYNQRFKG (SEQ ID NO:5), and HVR-H3 comprising the amino acid sequence WISDFFDY (SEQ ID NO:6). In some embodiments, the antibody comprises a heavy chain variable region sequence of at least 95% sequence identity to the amino acid sequence of SEQ ID NO:7; and/or a light chain variable region sequence of at least 95% sequence identity to the amino acid sequence of SEQ ID NO:8. In some embodiments, the antibody comprises a heavy chain variable region comprising the amino acid sequence of SEQ ID NO:7; and/or a light chain variable region comprising the amino acid sequence of SEQ ID NO:8. In some embodiments, the antibody is rontalizumab having CAS registration number 948570-30-7. In some embodiments, the antibody comprises an amino acid sequence as disclosed in CAS 1006877-41-3. In some embodiments, the subcutaneous administration device is a pre-filled syringe, an autoinjector, or a large volume infusion device.

In another aspect, the invention provides an article of manufacture comprising a computerized system comprising a bio-assay module for detecting a gene expression of one or more IRGs from a biological sample and a processor module to calculate expression of the gene and to score the calculation of the gene against a cutoff value to provide a diagnosis, wherein the cutoff value is (1) less than 1.5 times the value of the expression levels of the IRGs of a healthy person (or control) or (2) less than two standard deviations over the median value of the expression levels of the IRGs in a healthy persons (or control). In some embodiments, the bio-assay module is the cobas z480 analyzer.

In another aspect, the invention provides a kit for identifying an autoimmune patient who may benefit for an interferon inhibitor treatment, comprising a vial for collecting a blood sample from an autoimmune patient and instructions for determining whether the autoimmune patient is ISM^{lo}. In some embodiments, the expression level of at least one gene selected from the group consisting of EPSTI1, HERC5, TYK1 (CMPK2), IFI27, IFI44, IFIT1, MX1, OAS1, OAS2, and OAS3 is used to determine whether the autoimmune patient is ISM^{lo}. In some embodiments, the autoimmune disease is lupus. In some embodiments, the interferon inhibitor is an anti-interferon α antibody.

In another aspect, the invention provides a stable liquid composition comprising an anti-interferon α antibody in an amount of about 50 to about 250 mg/mL, arginine-HCl in an amount of about 50 to about 200 mM, histidine in an amount of about 5 to about 100 mM, polysorbate in an amount of about 0.01 to about 0.1%, wherein the composition has a pH from about 5.5 to about 7.0.

In one embodiment, the cutoff for determining the upper boundaries of an ISM low score is about 1.5 times the value of the mean threshold cycle, or Ct, of healthy persons (or control) or two standard deviations above the mean value of healthy persons (or control). In one embodiment, autoimmune patients having a mean IRG DCt value less than the cut off will be more likely to respond to therapeutic agents of this invention (e.g., interferon inhibitors) than lupus patients having a mean DCt value greater than the cut off. In one aspect of the invention, the amount of the antibody specific to double stranded deoxyribonucleic acid (dsDNA) is determined by using AtheNa Multi-lyte ANA Test System and AtheNA Multi-Lyte® ANA-II PLUS Test System Kit (manufactured by Inverness Medical Inc., Raritan, N.J.). In another aspect, the IRG status can be determined by using RT-PCT with the cobas z480 analyzer (Roche Diagnostics). In one embodiment, the IRGs of the ISM, enlarged ISM, enlarged ISM-A, 24-gene ISM signature, 3-gene ISM signature are used to determine the IRG status.

It is to be understood that one, some, or all of the properties of the various embodiments described herein may be combined to form other embodiments of the present invention. These and other aspects of the invention will become apparent to one of skill in the art. These and other embodiments of the invention are further described in the detailed description that follows.

### BRIEF DESCRIPTION OF THE DRAWINGS

FIG. 1 depicts a diagram of the study design for the trial described in Example 1 below.

FIG. 2 is a table summarizing patient characteristics from the trial described in Example 1.

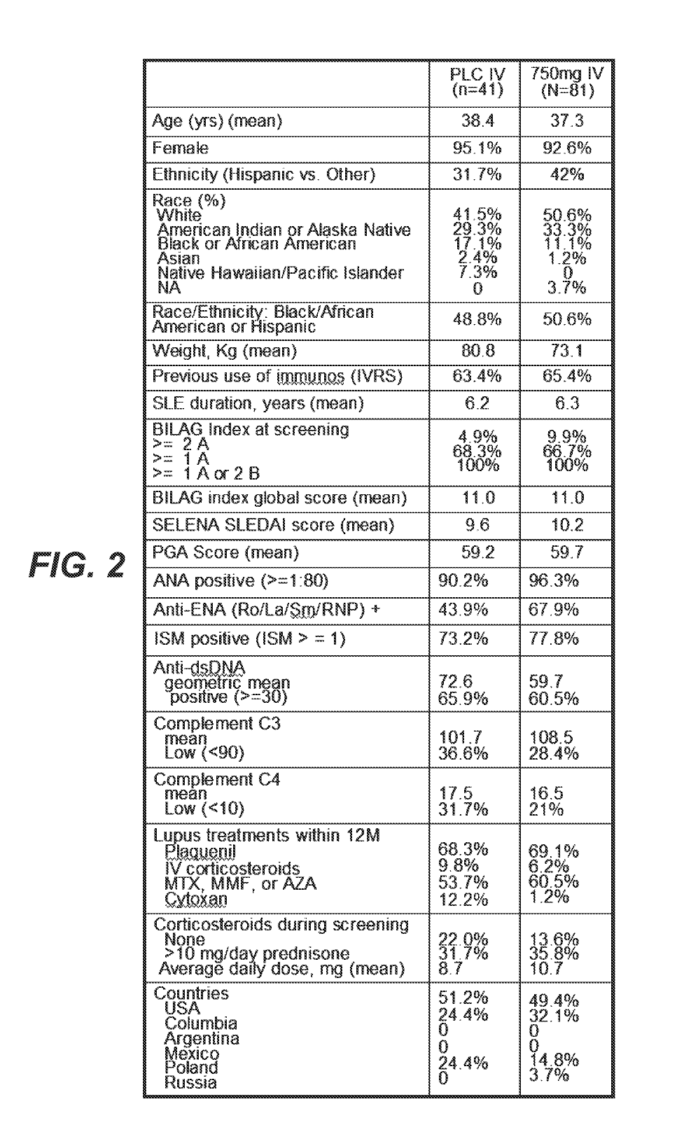

FIGS. 3A and 3B are tables summarizing patient characteristics by ISM levels.

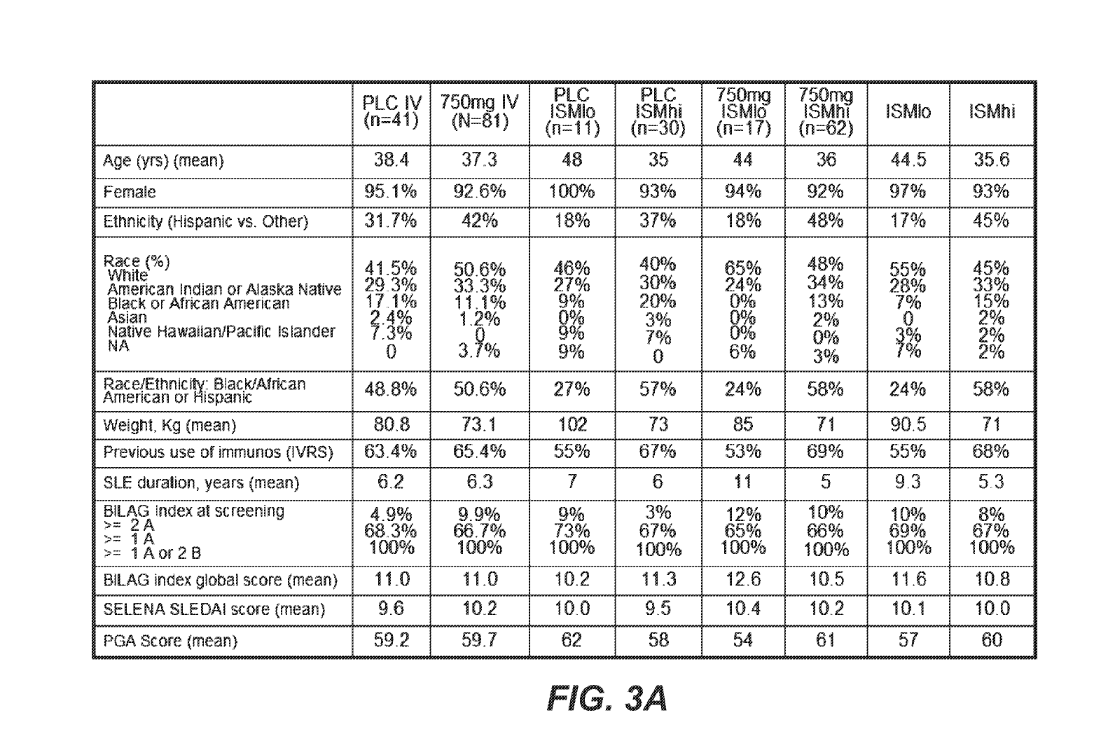

FIGS. 4A and 4B are tables summarizing patient characteristics by ENA status.

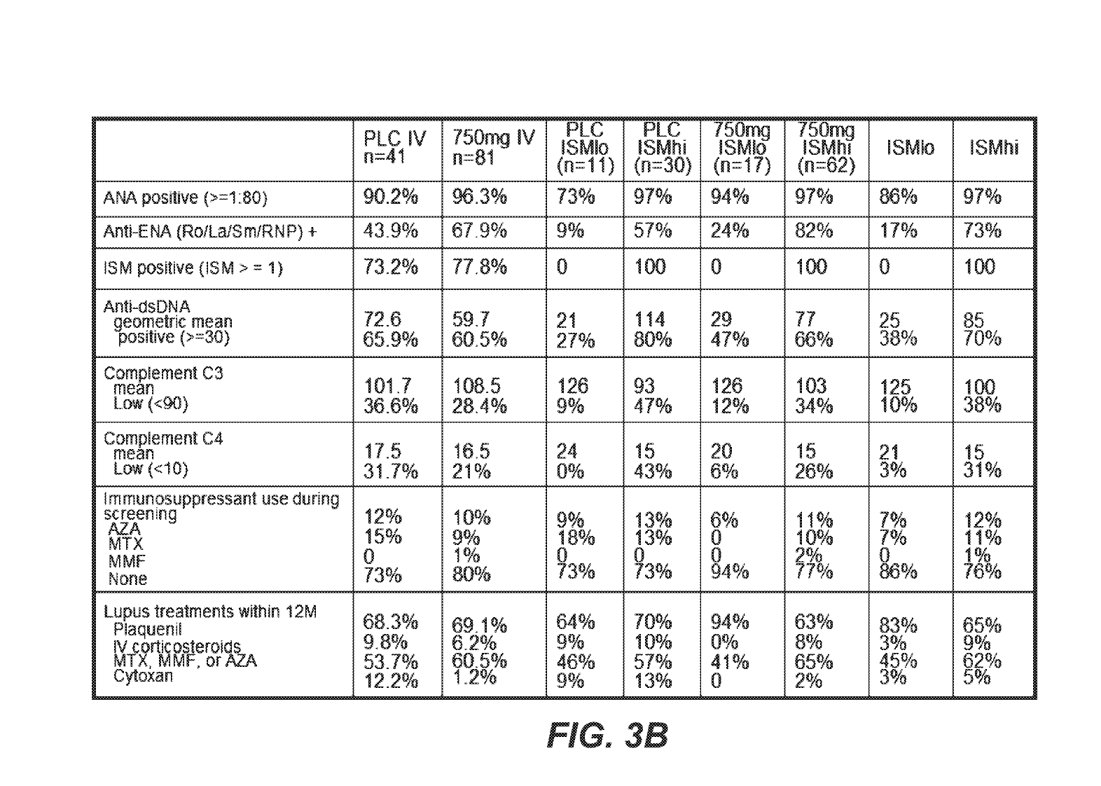

FIG. 5 depicts the mean reduction of IFI27 expression in lupus patients by ENA status and response over time. The grey box represents IFI27 expression in healthy individuals.

FIGS. 6A and 6B depict the mean BILAG Global Score and change from BILAG baseline by ENA status and time.

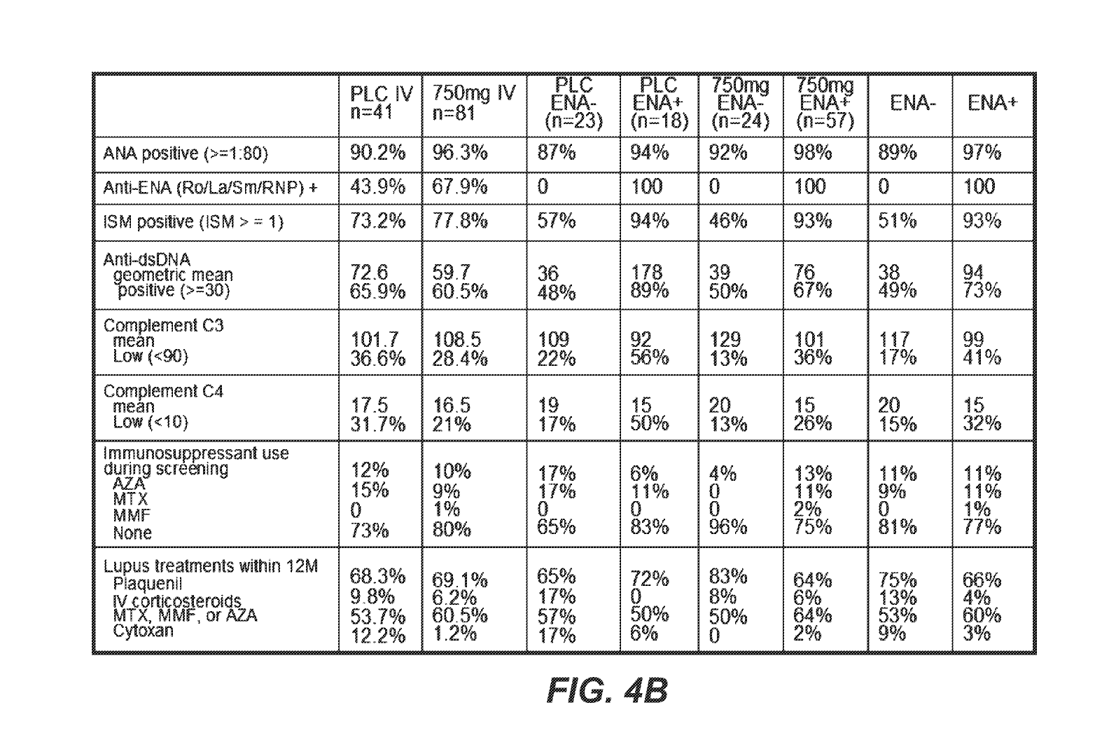

FIGS. 7A and 7B depict the mean SLEDAI Score and change from SLEDAI baseline by ENA status and time.

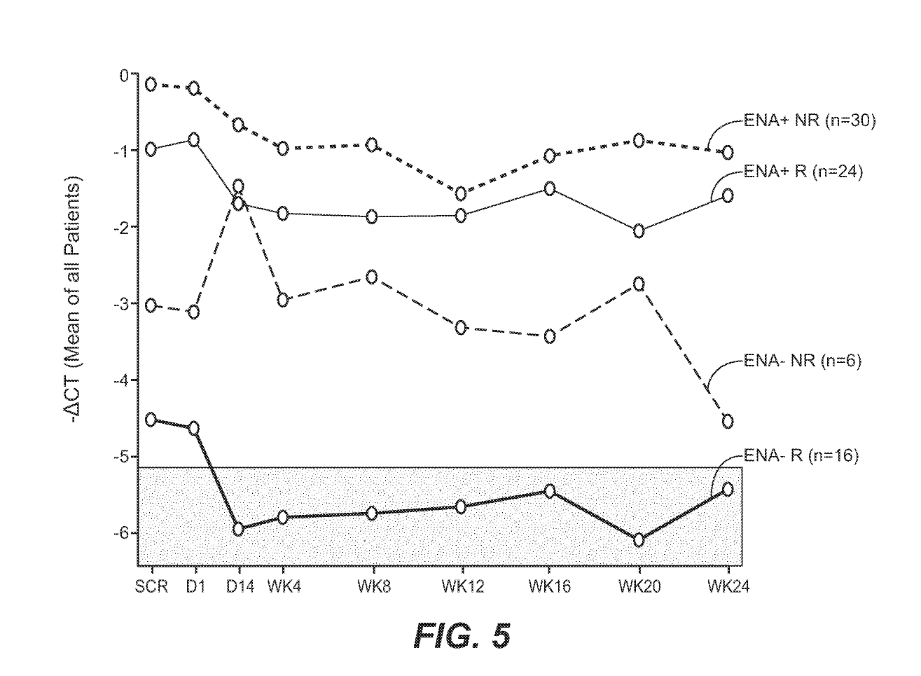

FIGS. 8A and 8B depict the mean percent change in BILAG Global Score and SLEDAI score by ENA status and time.

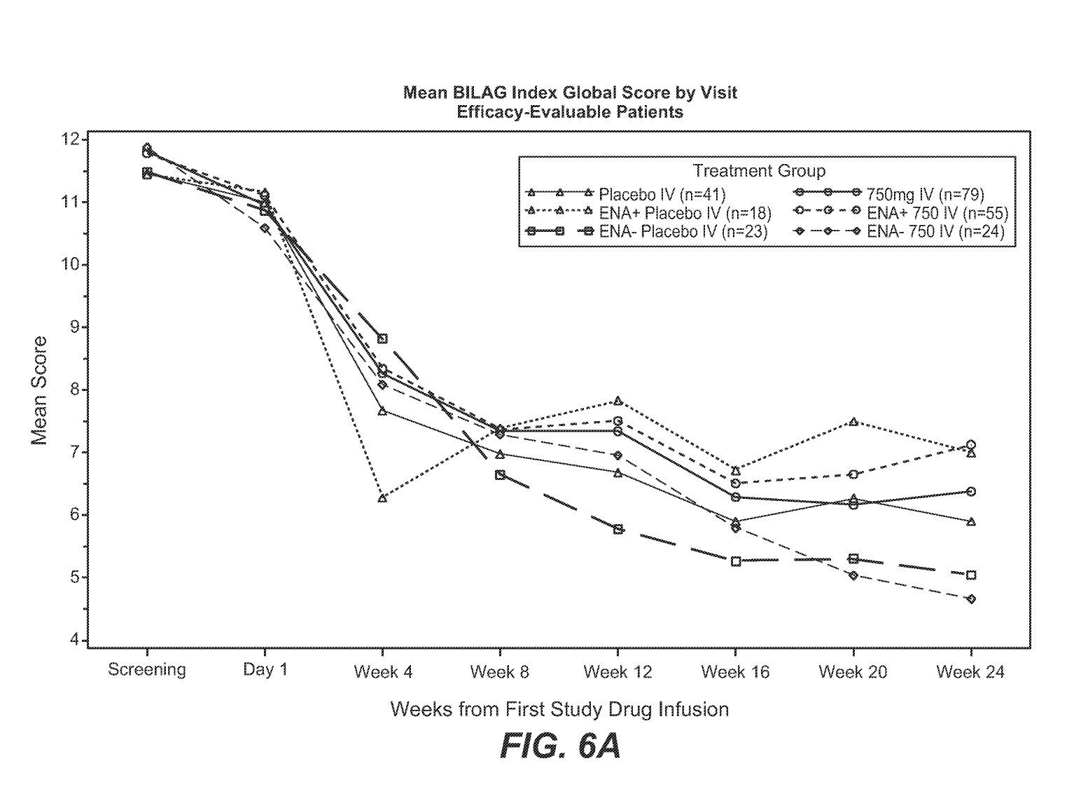

FIG. 9 show IFN Signature Metric (ISM) for healthy controls and SLE patients from various studies. The bimodal distribution of the patient population allows for selection of a specific population (or subpopulation) of ISM^{lo} lupus patients that are more responsive to anti-Type I interferon antibodies. “Ph I anti-IFNα” indicates Phase I study using rontalizumab.

FIG. 10 shows a plot of three populations in the ROSE study where the patients are defined by ISM and dsDNA antibody titer. As further detailed herein, ISM^{lo} patients as well as those who have a baseline double stranded DNA antibody titer (anti-dsDNA) that is less than or equal to 200 IU can be identified as good candidates for therapeutic treatment using anti-Type I interferon antibodies.

FIG. 11 shows graphs for IRG expression (−DCT) and Mod/severe SLE Flares

(Active ISM^{lo}±ISM^{Hi}). The upper curve shows the F=Flare, n=23. The upper left graph depicts IFI27. The upper right graph depicts IFI44. The lower left graph depicts MX1. The lower right graph shows IFIT1. A patient was considered to have had a moderate/severe flare if either of the following held: a severe flare was recorded on the SELENA FLARE INDEX (SFi), a moderate or severe flare was recorded on the SELENA Flare Index-Revised (SFI-R), or a new BILAG A or 2 new BILAG B scores were recorded at a visit.

FIGS. 12-21 show gene expression levels for various IRGs. The upper line (unfilled squares) represents patients who had week 16 moderate/severe flare. Interferon regulated genes expression levels (−delta CT or −DCT units) were measured at pre and post dose time points by quantitative PCR. Patients are separated into two categories; those that had a moderate to severe flare at week 16 (n=23, upper/unfilled square line) and the remainder of patients that did not present flares (lower/filled circle line). Preceding the flare at week 16, the mean IRG expression levels were elevated and these genes include but are not limited to IFI27, IFI44, MX1, IFIT1, HERC5, EPSTI1, and CMPK2. Lines shown represent the mean and standard errors of the mean of expression from baseline onwards and include patients from IV and SC cohorts of active groups only.

FIG. 12 shows the mean (+/−SE) of −Dct for HERC5.

FIG. 13 shows the mean (+/−SE) of −Dct for EPSTI1.

FIG. 14 shows the mean (+/−SE) of −Dct for CMPK2.

FIG. 15 shows the mean (+/−SE) of −Dct for IFI27.

FIG. 16 shows the mean (+/−SE) of −Dct for IFI44.

FIG. 17 shows the mean (+/−SE) of −Dct for IFIT1.

FIG. 18 shows the mean (+/−SE) of −Dct for MX1.

FIG. 19 shows the mean (+/−SE) of −Dct for OAS 1.

FIG. 20 shows the mean (+/−SE) of −Dct for OAS2.

FIG. 21 shows the mean (+/−SE) of −Dct for OAS3.

FIG. 22a shows the effects in endpoints support results based on the SRI. These data shows decrease in steroid use over time (week 8-24) and flare rate with treatment. Steroid use decrease was particularly pronounced in ISM^{lo} population. FIG. 22b shows the treatment effects in endpoints support results based on the SRI, in particular, the reduction in SELENA-SLEDAI flare rate.

FIG. 23 shows that Rontalizumab shows significant treatment effect in ISM^{lo} population using the SRI endpoint. SRI used was SRI-4. Using BILAG, there was inconclusive treatment difference (<10%) in all-corner and ISM subsets. Using SRI, there was inconclusive treatment difference between active for all-corner and ISM^{hi} population. For the high SRI response rate with ISM^{lo} patients (for IV+SC), the treatment difference was 35% (p=0.014).

FIG. 24 shows that the response in ISM^{lo} population is maintained using higher bar SRI thresholds. The reduction in baseline SELENA-SLEDAI by ≧X points (range tested: 4-7); SRI uses ≧4 points reduction in SELENA-SLEDAI. The treatment difference was adjusted by administration mode (IV or SC); CMH p-value.

FIG. 25 shows that anti-dsDNA^{lo} identifies another patient population with a positive treatment effect. There are twice as many patients in the ISM^{hi}/dsDNA^{lo} group (50%) as in the ISM^{lo} group (25%). The remaining 25% are the dsDNA^{hi} patients (upper right panel on slide 22) which do not appear to have benefited from rontaliumab treatment.

### DETAILED DESCRIPTION

### I. Introduction

The present invention provides, inter alia, methods of treating lupus patients with a type I interferon antibody and methods of identifying patients likely to benefit from such treatment, as well as methods of predicting flares. The present invention also provides methods and compositions for selection of a specific population of autoimmune patients (e.g., lupus) for treatment with an interferon inhibitor, such as an anti-Type I interferon antibody (e.g. rontalizumab).

### II. Definitions

“Lupus” as used herein is an autoimmune disease or disorder involving antibodies that attack connective tissue. The principal form of lupus is a systemic one, systemic lupus erythematosus (SLE), including cutaneous SLE and subacute cutaneous SLE, as well as other types of lupus (including nephritis, extrarenal, cerebritis, pediatric, non-renal, discoid, and alopecia).

“Lupus nephritis” is a serious consequence of end organ damage caused by lupus-associated inflammation of the kidney that occurs in up to 30% of lupus patients. In lupus nephritis patients, renal involvement is characterized by proteinuria (>0.5 g/24 hours), and/or red blood cells or casts in urine specimens. The histological classification of lupus nephritis based on revised classification criteria developed by the International Society of Nephrology and the Renal Pathology Society includes classes I-V: mesangial (I, II), proliferative (III, IV), and membranous (V) lesion. Kidney histology may have features of more than one class of disease. Classes III and IV are subdivided further depending on the activity or chronicity of the abnormalities seen. Class VI is reserved for widespread sclerotic disease.

“Pediatric lupus” is diagnosed with the same ACR criteria (4 out of 11 domains) as adult lupus. Comparing the clinical features of childhood- and adult-onset lupus reveals similarities as well as differences. In general, children with lupus tend to have more severe and more aggressive disease than adult SLE patients, and childhood-onset SLE often presents with major organ system involvement, including renal and neuropsychiatric disease.

The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity.

“Antibody fragments” comprise a portion of an intact antibody, preferably comprising the antigen-binding region thereof. Examples of antibody fragments include Fab, Fab′, F(ab′)₂, and Fv fragments; diabodies; linear antibodies; single-chain antibody molecules; and multispecific antibodies formed from antibody fragments.

For the purposes herein, an “intact antibody” is one comprising heavy- and light-variable domains as well as an Fc region.

As used herein, the terms “type I interferon” and “human type I interferon” are defined as all species of native human and synthetic interferon which fall within the human and synthetic interferon-α, interferon-ω and interferon-β classes and which bind to a common cellular receptor. Natural human interferon-α comprises 23 or more closely related proteins encoded by distinct genes with a high degree of structural homology (Weissmann and Weber, Prog. Nucl. Acid. Res. Mol. Biol., 33: 251 (1986); J. Interferon Res., 13: 443-444 (1993)). The human IFN-α locus comprises two subfamilies. The first subfamily consists of at least 14 functional, non-allelic genes, including genes encoding IFN-αA (IFN-α2), IFN-αB (IFN-α8), IFN-α (IFN-α10), IFN-αD (IFN-α1), IFN-αE (IFN-α22), IFN-αF (IFN-α21), IFN-αG (IFN-α5), IFN-α16, IFN-α17, IFN-α4, IFN-α6, IFN-α7, and IFN-αH (IFN-α14), and pseudogenes having at least 80% homology. The second subfamily, α_{II} or ω, contains at least 5 pseudogenes and 1 functional gene (denoted herein as “IFN-αs_{II1}” or “IFN-ω”) which exhibits 70% homology with the IFN-α genes (Weissmann and Weber (1986)). The human IFN-β is generally thought to be encoded by a single copy gene.

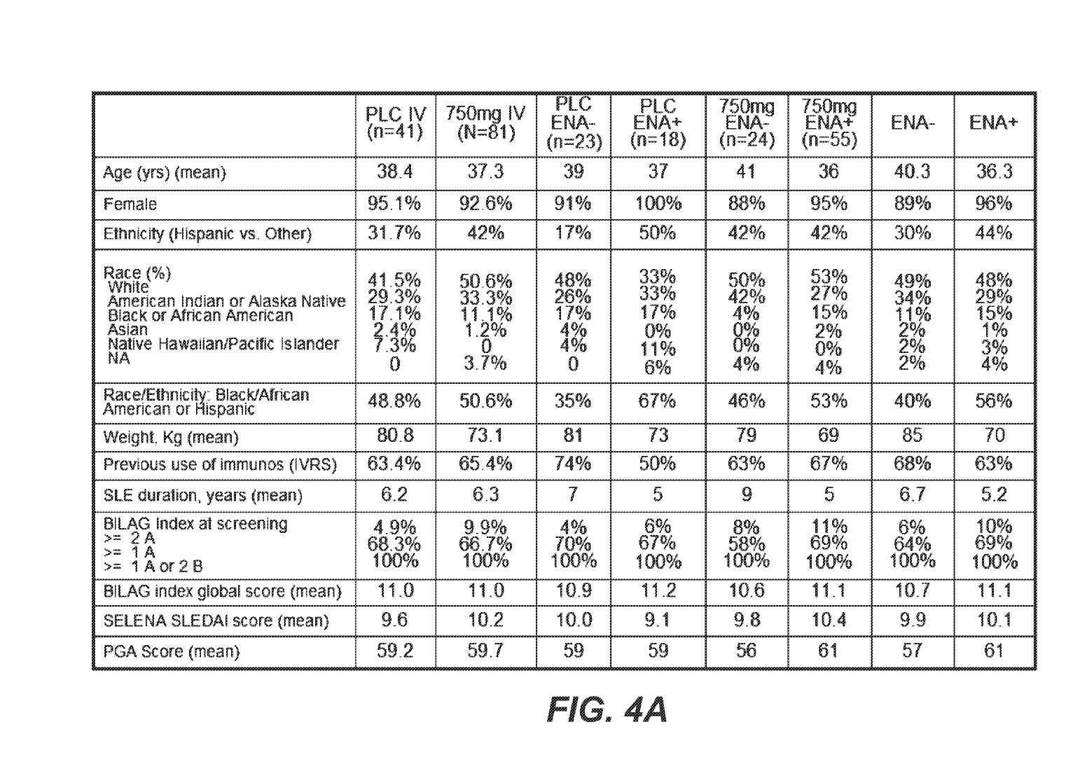

As used herein, the terms “human interferon-α (hIFN-α) receptor 1”, “IFN-βR”, “hIFNAR1”, “IFNAR1”, and “Uze chain” are defined as the 557 amino acid receptor protein cloned by Uze et al., Cell, 60: 225-234 (1990), including an extracellular domain of 409 residues, a transmembrane domain of 21 residues, and an intracellular domain of 100 residues, as shown in FIG. 5 on page 229 of Uze et al. In one embodiment, the foregoing terms include fragments of IFNAR1 that contain the extracellular domain (ECD) (or fragments of the ECD) of IFNAR1.

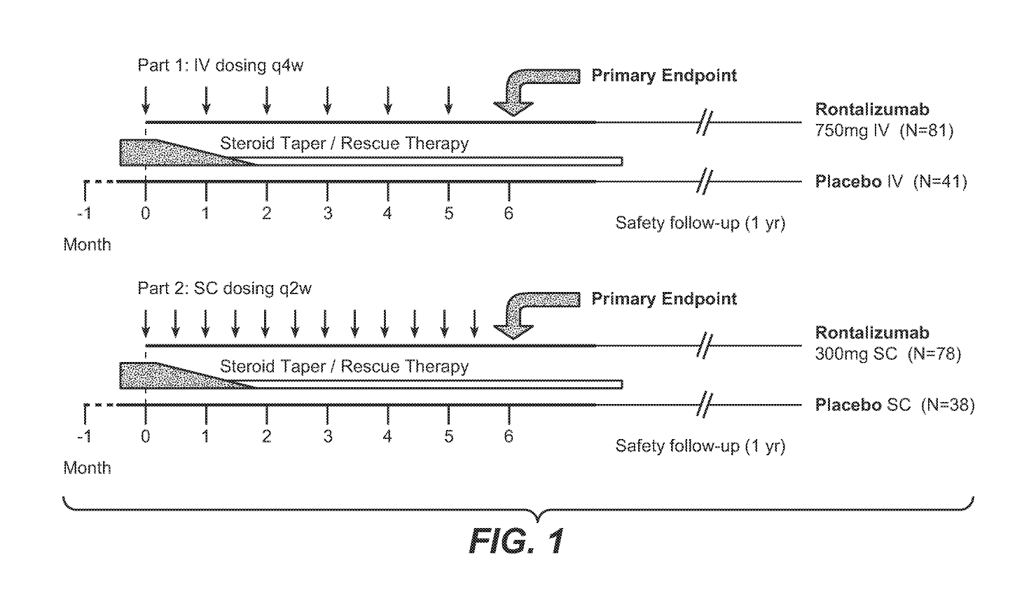

As used herein, the terms “human interferon-α (hIFN-α) receptor 2”, “IFN-αβR”, “hIFNAR2”, “IFNAR2”, and “Novick chain” also include the 515 amino acid receptor protein cloned by Domanski et al., J. Biol. Chem., 37: 21606-21611 (1995), including an extracellular domain of 217 residues, a transmembrane domain of 21 residues, and an intracellular domain of 250 residues, as shown in FIG. 1 on page 21608 of Domanski et al. In one embodiment, the foregoing terms include fragments of IFNAR2 that contain the extracellular domain (ECD) (or fragments of the ECD) of IFNAR2, and soluble forms of IFNAR2, such as IFNAR2ECD fused to at least a portion of an immunoglobulin sequence.

The term “interferon inhibitor” or “type I interferon inhibitor” as used herein refers to a molecule having the ability to inhibit a biological function of wild type or mutated Type 1 interferon. Accordingly, the term “inhibitor” is defined in the context of the biological role of Type 1 interferon. In one embodiment, an interferon inhibitor referred to herein specifically inhibits cell signaling via the Type 1 interferon/interferon receptor pathway. For example, an interferon inhibitor may interact with (e.g. bind to) interferon alpha receptor, or with a Type 1 interferon which normally binds to interferon receptor. In one embodiment, an interferon inhibitor binds to the extracellular domain of interferon alpha receptor. In one embodiment, an interferon inhibitor binds to the intracellular domain of interferon alpha receptor. In one embodiment, an interferon inhibitor binds to Type 1 interferon. In one embodiment, the Type 1 interferon is an interferon alpha subtype. In one embodiment, the Type 1 interferon is not interferon beta. In one embodiment, the Type 1 interferon is not interferon omega. In one embodiment, the Type 1 interferon is not interferon lambda. In one embodiment, the Type 1 interferon is not interferon beta or interferon omega.

In one embodiment, the Type 1 interferon is not interferon omega or interferon lambda. In one embodiment, the Type 1 interferon is not interferon beta or interferon lambda. In one embodiment, the Type 1 interferon is not interferon alpha, interferon beta or interferon lambda. In one embodiment, interferon biological activity inhibited by an interferon inhibitor is associated with an immune disorder, such as an autoimmune disorder. An interferon inhibitor can be in any form, so long as it is capable of inhibiting interferon/receptor activity; inhibitors include antibodies (e.g., monoclonal antibodies as defined hereinbelow and as described in U.S. Pat. Nos. 7,087,726 and 7,741,449 and U.S. Patent Publication No. 2009-0214565), small organic/inorganic molecules, antisense oligonucleotides, aptamers, inhibitory peptides/polypeptides, inhibitory RNAs (e.g., small interfering RNAs), combinations thereof, etc.

The term “biomarker” as used herein refers generally to a molecule, including a gene, protein, carbohydrate structure, or glycolipid, the expression of which in or on a mammalian tissue or cell can be detected by standard methods (or methods disclosed herein) and is predictive, diagnostic and/or prognostic for a mammalian cell's or tissue's sensitivity to treatment regimens based on inhibition of interferons, e.g. Type 1 interferons. Optionally, the expression of such a biomarker is determined to be higher than that observed for a control/reference tissue or cell sample. Optionally, for example, the expression of such a biomarker will be determined in a PCR or FACS assay to be at least about 5-fold, at least about 10-fold, at least about 20-fold, at least about 30-fold, at least about 40-fold, at least about 50-fold, or preferably at least about 100-fold higher in the test tissue or cell sample than that observed for a control tissue or cell sample. Optionally, the expression of such a biomarker will be determined in an IHC assay to score at least 2 or higher for staining intensity. Optionally, the expression of such a biomarker will be determined using a gene chip-based assay.

As used herein, the term “ENA” refers to Extractable Nuclear Antigens, i.e., a group of nuclear antigens including, e.g., RNP, Ro/SS-A, La/SS-B, Sm, SCL-70, Jo-1, as described in McNeilage et al., J., Clin. Lab. Immunol. 15:1-17 (1984); Whittingham, Ann. Acad. Med. 17(2):195-200 (1988); Wallace and Hahn, DUBOIS' LUPUS ERYTHEMATOSUS, 7^{TH} ED. LIPPINCOTT (2007); Tang et al., Medicine 89(1): 62-67 (2010). Antibodies to ENA have been correlated to lupus. McNeilage et al., 1984; Whittingham 1988; Asherson et al., Medicine 68(6): 366-374 (1989); and Tang et al., 2010. The term “ENA status” as used herein refers to the level of ENA antibodies in a sample from an individual. The term “ENA+” as used herein refers to a patient who has ENA antibodies at a level greater than the level of ENA antibodies found in a healthy individual. The term “ENA−” as used herein refers to a patient who has ENA antibodies at a level less than or equal to the level of ENA antibodies found in a healthy individual.

An “IRG” or “interferon response gene” or “interferon responsive gene,” as used herein, refers to one or more of the genes, and corresponding gene products, listed in Table 1, 2, 3 and/or 4 of U.S. Patent Publication No. 20080057503. As shown therein, aberrant expression levels/amounts of one or more of these genes are correlated with a variety of autoimmune disorders. As would be evident to one skilled in the art, depending on context, the term IRG can refer to nucleic acid (e.g., genes) or polypeptides (e.g., proteins) having the designation or unique identifier listed in Table 1, 2, 3 and/or 4 of U.S. Patent Publication No. 20080057503.

“ISM” or “interferon signature metric” as used herein refers to a measurement of the expression levels of one, two, three, four, five, six, seven, or more interferon response genes. The genes include, e.g., CMPK2, EPST1, HERC5, IFI27, IFI44, IFIT1, MX1, OAS1, OAS2, OAS3, and combinations thereof. The ISM may be determined at baseline, i.e., prior to any treatment with a type I interferon inhibitor, or at any time after administration. Gene expression may be detected by standard methods (or methods disclosed herein) in a biological sample from the individual (e.g., a lupus patient or a healthy individual). For example, gene expression may be determined qualitatively and/or quantitatively based on any suitable criterion known in the art, including but not limited to mRNA, cDNA, proteins, and/or protein fragments.

“Enlarged ISM” or “Enlarged ISM signature” as used herein refers to a measurement of the expression levels of one, two, three, four, five, six, seven, eight, nine, ten, eleven, twelve, thirteen or more interferon responsive genes, wherein at least one of the interferon responsive genes is CHMP5, CIG5, EPSTI1, G1P2, HERC5, IFI44, IFI44L, IFIT1, IFIT4, IFIT5, IRF7, MX1, OAS1, OAS2, OAS3, OASL, RIG1, RIGE, SAMD9L, SP110, TYK1 (CMPK2), XIAP, ZBP1, PARP9, IFI27, SIGLEC1, DNAPTP6, USP18, IFI6, HSXIAPAF1, and LAMP3, and any isoforms thereof.

TABLE A
Enlarged ISM genes.

| NO. | Gene | Alias (non-exhaustive) | Accession No |
|---|---|---|---|
| 1 | CHMP5 | chromatin modifying | NM_016410 |
| | | protein 5 | |
| 2 | CIG5 | RSAD2 or radical S- | NM_080657 |
| | | adenosyl methionine | |
| | | domain containing 2 | |
| 3 | EPSTI1 | epithelial stromal | NM_001002264 |
| | | interaction | |
| 4 | G1P2 | interferon, alpha- | NM_005101 |
| | | inducible protein IFI-15K | |
| | | or ISG15, IFI15; IP17; | |
| | | UCRP | |
| 5 | HERC5 | hect domain and RLD6 | NM_016323 |
| 6 | IFI44 | interferon-induced protein | NM_006417 |
| | | 44 | |
| 7 | IFI44L | interferon-induced protein | NM_006820 |
| | | 44-like | |
| 8 | IFIT1 | interferon-induced protein | NM_001001887 |
| | | with tetratricopeptide | |
| | | repeats 1 | |
| 9 | IFIT4 | interferon-induced protein | NM_001549.4 |
| | | with tetratricopeptide | NM_001031683.2 |
| | | repeats 4; interferon- | |
| | | induced protein with | |
| | | tetratricopeptide repeats 3 | |
| | | (IFIT3), CIG-49; GARG- | |
| | | 49; IFI60; IFIT4; IRG2; | |
| | | ISG60; RIG-G | |
| 10 | IFIT5 | interferon-induced protein | NM_012420 |
| | | with tetratricopeptide | |
| | | repeats 5 | |
| 11 | IRF7 | interferon regulatory | NM_001572 |
| | | factor 7 | |
| 12 | MX1 | myxovirus resistance 1 | NM_002462 |
| 13 | OAS1 | 2′-5′-oligoadenylate | NM_001032409 |
| | | synthase 1 | |
| 14 | OAS2 | 2′-5′-oligoadenylate | NM_001032731 |
| | | synthase 2 | |
| 15 | OAS3 | 2′-5′-oligoadenylate | NM_006187 |
| | | synthase 3 | |
| 16 | OASL | 2′-5′-oligoadenylate | NM_003733 |
| | | synthase-like | |
| 17 | PARP9 | poly ADP-ribose | NM_031458 |
| | | polymerase family, | |
| | | member 9 | |
| 18 | RIG1 | DDX58 or DEAD Asp- | NM_014314 |
| | | Glu-Ala-Asp box | |
| | | polypeptide 58 | |
| 19 | RIGE | lymphocyte antigen 6 | NM_002346 |
| | | complex, locus E or | |
| | | LY6E | |
| 20 | SAMD9L | sterile alpha motif domain | NM_152703 |
| | | containing 9-like | |
| 21 | SP110 | SP110 nuclear body | NM_004509 |
| | | protein | |
| 22 | TYK1 | thymidylate kinase family | NM_207315 |
| | | LPS-inducible; cytidine | |
| | | monophosphate (UMP- | |
| | | CMP) kinase 2 (CMPK2), | |
| | | mitochondrial, | |
| 23 | XIAP | X-linked inhibitor of | NM_001167 |
| | | apoptosis | |
| 24 | ZBP1 | Z-D binding protein 1 | NM_030776 |
| 25 | IFI27 | interferon, alpha- | NM_005532 |
| | | inducible protein 27) | |
| 26 | SIGLEC1 | SIGLEC1 (sialic acid | NM_023068 |
| | | binding Ig-like lectin 1, | |
| | | sialoadhesin | |
| 27 | DNAPTP6 | DNA polymerase- | NM_015535 |
| | | transactivated protein 6; | |
| | | SPATS2-like protein | |
| | | (spermatogenesis | |
| | | associated, serine-rich 2- | |
| | | like protein, SPATS2L) | |
| 28 | USP18 | ubiquitin specific | NM_017414 |
| | | peptidase 18 | |
| 29 | IFI6 | interferon, alpha- | NM_002038 |
| | | inducible protein 6; | |
| | | interferon, alpha- | |
| | | inducible protein clone | |
| | | IFI-6-16; G1P3; | |
| 30 | HSXIAPAF1 | XIAP associated factor-1; | NM_017523 |
| | | BIRC4BP, | |
| 31 | LAMP3 | Lysosomal-associated | NM_014398 |
| | | membrane protein 3 | |

In one embodiment, the Enlarged ISM comprises the measurement of the expression level of at least one of the interferon responsiveness genes IFI27, CIG5, IFI44L, IFI44, OAS1, OAS3, IFIT1, G1P2, HERC5, MX1, EPSTI1, IFIT3 and IFI6, or a combination thereof or all of such interferon responsive genes (“Enlarged ISM-A”).

“24-gene ISM signature” or “24-gene ISM” as used herein refers to a measurement of the expression levels of one, two, three, four, five six, seven, eight, nine, ten, eleven, twelve, thirteen, fourteen, fifteen, sixteen, seventeen, eighteen, nineteen, twenty, twenty-one, twenty-two, twenty-three or all interferon responsive genes, wherein at least one of the interferon responsive genes is CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP, ZBP1, or a combination or all of such interferon responsive genes.

“3-gene ISM signature” or “3-gene ISM” as used herein refers to a measurement of the expression levels of one, two or three interferon responsive genes, wherein at least one of the interferon responsive genes is EPSTI1, HERC5 or TYK1 (CMPK2). In one embodiment, the 3-gene ISM signature comprises a measurement of the expression levels of EPSTI1, HERC5 and TYK1. In another embodiment, the 3-gene ISM signature comprises a measurement of the expression levels of EPSTI1 and HERC5. In another embodiment, the 3-gene ISM signature comprises a measurement of the expression levels of EPSTI1 and TYK1. In another embodiment, the 3-gene ISM signature comprises a measurement of the expression levels of HERC5 and TYK1. In yet another embodiment, the 3-gene ISM signature comprises a measurement of the expression levels of TYK1, HERC5 or EPSTI1.

“IRG status” as used herein refers to the biological status of IRGs in a patient that is reflective of the gene expression levels of one or more IRGs in the patient. A patient can be ISM^{lo} or ISM^{hi}.

“ISM^{lo}” or “ISM Low” as used herein refers to the IRG status of an autoimmune disease patient that is reflective of the expression levels of his/her IRGs relative to the expression levels of the same IRGs in a healthy person(s) or a control, wherein the IRG expression levels of an ISM low autoimmune patient are generally (1) less than 1.5 times the mean value of the expression levels of the IRGs of a healthy person (or control) or (2) less than two standard deviations over the mean value of the expression levels of the same IRGs in a healthy persons (or control). The designation of ISM Low, unless otherwise specified, is not dependent on a particular assay or a particular set of IRG. In one embodiment, any one or combination of the IRGs of the Interferon Signature Metric, the Enlarged ISM, the Enlarged ISM-A, the 24-gene ISM or the 3-gene ISM are used to evaluate the IRG status of an autoimmune patient. In one embodiment, the ISM low is less than 1.4 times the mean value of the expression levels of the IRGs of healthy persons (or control). In other embodiments, the ISM low is less than 1.3, 1.2, or 1.1 times the mean value of the expression levels of the IRGs of healthy persons (or control). In other embodiments, the ISM low is the same value of the expression levels of the IRGs of a healthy person (or control). In other embodiments, the ISM low is less than 1.9, 1.8, 1.7, 1.6, 1.5, 1.4, 1.3, 1.2, or 1.1 standard deviations over the mean value of the expression levels of the same IRGs in a healthy persons (or control).

“ISM^{hi}” or “ISM high” as used herein refers to the biological status of an autoimmune patient reflective of the expression levels of his/her IRGs relative to the expression levels of the same IRG expression levels in a healthy persons(s) or a control, wherein the IRG expression levels of an ISM high autoimmune disease patient are generally (1) equal to or greater than 1.5 times the value of the expression of the IRGs of a healthy person (or control) or (2) greater than or equal to two standard deviations over the mean value of the expression levels of the same IRGs in healthy persons (or control).

The term “housekeeping gene” refers to a group of genes that codes for proteins whose activities are essential for the maintenance of cell function. These genes are typically similarly expressed in all cell types. Housekeeping genes include, without limitation, transferrin receptor (TFRC), ribosomal protein L19 (NP₋₋₀₀₀₉₇₂), glyceraldehyde-3-phosphate dehydrogenase (GAPDH), Cypl, albumin, actins (e.g. β-actin), tubulins, cyclophilin, hypoxantine phosphoribosyltransferase (HRPT), ribosomal protein L32 (NP₋₋₀₀₁₀₀₇₀₇₅), and ribosomal protein/genes 28S (e.g., Q9Y399) and 18S.

The term “sample,” or “test sample” as used herein, refers to a composition that is obtained or derived from a subject of interest that contains a cellular and/or other molecular entity that is to be characterized and/or identified, for example based on physical, biochemical, chemical and/or physiological characteristics. In one embodiment, the definition encompasses blood and other liquid samples of biological origin and tissue samples such as a biopsy specimen or tissue cultures or cells derived therefrom. The source of the tissue sample may be solid tissue as from a fresh, frozen and/or preserved organ or tissue sample or biopsy or aspirate; blood or any blood constituents; bodily fluids; and cells from any time in gestation or development of the subject or plasma. The term “sample,” or “test sample” includes biological samples that have been manipulated in any way after their procurement, such as by treatment with reagents, solubilization, or enrichment for certain components, such as proteins or polynucleotides, or embedding in a semi-solid or solid matrix for sectioning purposes. For the purposes herein a “section” of a tissue sample is meant a single part or piece of a tissue sample, e.g. a thin slice of tissue or cells cut from a tissue sample. Samples include, but are not limited to, whole blood, blood-derived cells, serum, plasma, lypmph fluid, synovial fluid, cellular extracts, and combinations thereof. In one embodiment, the sample is a clinical sample. In another embodiment, the sample is used in a diagnostic assay.

In one embodiment, a sample is obtained from a subject or patient prior to treatment with a type I interferon inhibitor. In another embodiment, a sample is obtained from a subject or patient following at least one treatment with a type I interferon inhibitor.

A “reference sample,” as used herein, refers to any sample, standard, or level that is used for comparison purposes. In one embodiment, a reference sample is obtained from a healthy and/or non-diseased part of the body (e.g., tissue or cells) of the same subject or patient. In another embodiment, a reference sample is obtained from an untreated tissue and/or cell of the body of the same subject or patient. In yet another embodiment, a reference sample is obtained from a healthy and/or non-diseased part of the body (e.g., tissues or cells) of an individual who is not the subject or patient. In even another embodiment, a reference sample is obtained from an untreated tissue and/or cell part of the body of an individual who is not the subject or patient.

In certain embodiments, a reference sample is a single sample or combined multiple samples from the same subject or patient that are obtained at one or more different time points than when the test sample is obtained. For example, a reference sample is obtained at an earlier time point from the same subject or patient than when the test sample is obtained. In certain embodiments, a reference sample includes all types of biological samples as defined above under the term “sample” that is obtained from one or more individuals who is not the subject or patient. In certain embodiments, a reference sample is obtained from one or more individuals with an angiogenic disorder (e.g., cancer) who is not the subject or patient.

In certain embodiments, a reference sample is a combined multiple samples from one or more healthy individuals who are not the subject or patient. In certain embodiments, a reference sample is a combined multiple samples from one or more individuals with a disease or disorder (e.g., an angiogenic disorder such as, for example, cancer) who are not the subject or patient. In certain embodiments, a reference sample is pooled RNA samples from normal tissues or pooled plasma or serum samples from one or more individuals who are not the subject or patient.

The standard of care in lupus management is based on current, accepted medical practice patterns, approved guidance documents developed by rheumatology societies (e.g. American College of Rheumatology, European League Against Rheumatism) and the discretion of treating physicians. Thus, the “standard of care” as used herein means the assessment and management of the signs and symptoms of lupus that depend on the lupus patient's particular constellation and severity of disease activity. Lupus patients continue to have disease activity long after the diagnosis is made, even with proper management, often involving new organ systems or specific organ system damage. There are three patterns of disease activity in lupus: the flare (or remitting, relapsing disease activity), chronically active disease, and long quiescence. These disease patterns are characterized using systematic clinical assessments, routine laboratory tests, standardized measures of disease activity, and integration of these assessments with the patient's own perceptions of health status and quality of life. As the patient's signs and symptoms of flare persist or worsen, the physician may find that a change in medications and/or dosages is warranted. The medications used to control lupus include, but is not limited to the following: (1) NSAIDs, including over-the-counter NSAIDs, e.g., naproxen (Aleve) and ibuprofen (Advil, Motrin, others), and stronger NSAIDs available by prescription; (2) Antimalarial drugs, e.g., hydroxychloroquine (Plaquenil); (3) Corticosteroids., e.g., Prednisone and other types of corticosteroids, and (4) Immune suppressants, e.g., cyclophosphamide (Cytoxan), azathioprine (Imuran, Azasan), mycophenolate (Cellcept), leflunomide (Arava) and methotrexate (Trexall).

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII, and FcγRIII FcR expression on hematopoietic cells is summarized in Table 3 on page 464 of Ravetch and Kinet Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

“Human effector cells” are leukocytes that express one or more FcRs and perform effector functions. Preferably, the cells express at least FcγRIII and carry out ADCC effector function. Examples of human leukocytes that mediate ADCC include peripheral blood mononuclear cells (PBMC), natural-killer (NK) cells, monocytes, cytotoxic T cells, and neutrophils, with PBMCs and NK cells being preferred.

The terms “Fc receptor” and “FcR” are used to describe a receptor that binds to the Fc region of an antibody. The preferred FcR is a native-sequence human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see Daëron Annu. Rev. Immunol. 15:203-234 (1997)). FcRs are reviewed in Ravetch and Kinet Annu. Rev. Immunol 9:457-92 (1991); Capel et al. Immunomethods 4:25-34 (1994); and de Haas et al. J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al, J. Immunol. 117:587 (1976) and Kim et al. J. Immunol. 24:249 (1994)).

“Complement-dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g., as described in Gazzano-Santoro et al. J. Immunol. Methods 202:163 (1996), may be performed.

“Growth-inhibitory” antibodies are those that prevent or reduce proliferation of a cell expressing an antigen to which the antibody binds. For example, the antibody may prevent or reduce proliferation of B cells in vitro and/or in vivo.

Antibodies that “induce apoptosis” are those that induce programmed cell death, e.g. of a B cell, as determined by standard apoptosis assays, such as binding of annexin V, fragmentation of DNA, cell shrinkage, dilation of endoplasmic reticulum, cell fragmentation, and/or formation of membrane vesicles (called apoptotic bodies).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light-chain and heavy-chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light-chain and the heavy-chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al. Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in ADCC.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment that contains a complete antigen-recognition and antigen-binding site. This region consists of a dimer of one heavy-chain and one light-chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy-chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains that enables the scFv to form the desired structure for antigen binding. For a review of scFv, see Plückthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 1993/11161; and Hollinger et al., Proc. Natl. Acad. Sci. USA, 90:6444-6448 (1993).

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable-domain antigen-binding sequences derived from a non-human primate (e.g. Old World Monkey, such as baboon, rhesus, or cynomolgus monkey) and human constant-region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH(H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. An HVR as used herein comprise any number of residues located within positions 24-36 (for L1), 46-56 (for L2), 89-97 (for L3), 26-35B (for H1), 47-65 (for H2), and 93-102 (for H3). Therefore, an HVR includes residues in positions described previously:
A) 24-34 (L1), 50-52 (L2), 91-96 (L3), 26-32 (H1), 53-55 (H2), and 96-101 (H3) (Chothia and Lesk, J. Mol. Biol. 196:901-917 (1987);
B) 24-34 of L1, 50-56 of L2, 89-97 of L3, 31-35B of H1, 50-65 of H2, and 95-102 of H3 (Kabat et al., Sequences of Proteins of Immunological Interest, 5th Ed. Public Health Service, National Institutes of Health, Bethesda, Md. (1991).
C) 30-36 (L1), 46-55 (L2), 89-96 (L3), 30-35 (H1), 47-58 (H2), 93-100a-j (H3) (MacCallum et al. J. Mol. Biol. 262:732-745 (1996).
   Unless otherwise indicated, HVR residues and other residues in the variable domain (e.g., FR residues) are numbered herein according to Kabat et al., supra.

A “naked antibody” is an antibody (as herein defined) that is not conjugated to a heterologous molecule, such as a cytotoxic moiety or radiolabel.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning-cup sequenator, or (3) to homogeneity by SDS-PAGE under reducing or nonreducing conditions using Coomassie blue or, preferably, silver stain. Isolated antibody includes the antibody in situ within recombinant cells, since at least one component of the antibody's natural environment will not be present. Ordinarily, however, isolated antibody will be prepared by at least one purification step.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

A “neutralizing antibody” is an antibody molecule which is able to eliminate or significantly reduce an effector function of a target antigen to which it binds. Accordingly, a “neutralizing” anti-IFN-α antibody is capable of eliminating or significantly reducing an effector function, such as receptor binding and/or elicitation of a cellular response, of IFN-α.

An exemplary assay is one that monitors the ability of an anti-IFN-α antibody to neutralize the receptor activation activity of IFN-α. See, for example, the Kinase Receptor Activation (KIRA) Assay as described in WO 95/14930, published Jun. 1, 1995, whereby neutralization is measured by the ability of a candidate antibody to reduce tyrosine phosphorylation (resulting from ligand binding) of the IFNAR1/R2 receptor complex.

Alternatively, the ability of the anti-IFN-α antibodies to neutralize the elicitation of a cellular response by IFN-α may be tested by monitoring the neutralization of the antiviral activity of IFN-α, as described by Kawade, J. Interferon Res. 1:61-70 (1980), or Kawade and Watanabe, J. Interferon Res. 4:571-584 (1984), or Yousefi, et al., Am. J. Clin. Pathol. 83: 735-740 (1985), or by testing the ability of an anti-IFN-α antibody to neutralize the ability of IFN-α to activate the binding of the signaling molecule, interferon-stimulated factor 3 (ISGF3), to an oligonucleotide derived from the interferon-stimulated response element (ISRE), in an electrophoretic mobility shift assay, as described by Kurabayashi et al., Mol. Cell. Biol., 15: 6386 (1995).

“Significant” reduction means at least about 60%, or at least about 70%, preferably at least about 75%, more preferably at least about 80%, even more preferably at least about 85%, still more preferably at least about 90%, still more preferably at least about 95%, most preferably at least about 99% reduction of an effector function of the target antigen (e.g. IFN-α), such as receptor (e.g. IFNAR2) binding and/or elicitation of a cellular response. Preferably, the “neutralizing” antibodies as defined herein will be capable of neutralizing at least about 60%, or at least about 70%, preferably at least about 75%, more preferably at least about 80%, even more preferably at least about 85%, still more preferably at least about 90%, still more preferably at least about 95%, most preferably at least about 99% of the anti-viral activity of IFN-α, as determined by the anti-viral assay of Kawade (1980), supra, or Yousefi (1985), supra. In another preferred embodiment, the “neutralizing” antibodies herein will be capable of reducing tyrosine phosphorylation, due to IFN-α binding, of the IFNAR1/IFNAR2 receptor complex, by at least about 60%, or at least about 70%, preferably at least about 75%, more preferably at least about 80%; even more preferably at least about 85%, still more preferably at least about 90%, still more preferably at least about 95%, most preferably at least about 99%, as determined in the KIRA assay referenced above. In a particularly preferred embodiment, the neutralizing anti-IFN-α antibodies herein will be able to neutralize all, or substantially all, subtypes of IFN-α and will not be able to neutralize IFN-β. In this context, the term “substantially all” means that the neutralizing anti-IFN-α antibody will neutralize at least IFN-α1, IFN-α2, IFN-α4, IFN-α5, IFN-α8, IFN-α10, and IFN-α21.

A “subject” or “patient” herein is a human subject or patient. Generally, such subject or patient is eligible for treatment for lupus. In one embodiment, such eligible subject or patient is one that is experiencing or has experienced one or more signs, symptoms, or other indicators of lupus or has been diagnosed with lupus, whether, for example, newly diagnosed, previously diagnosed with a new flare, or chronically steroid dependent with a new flare, or is at risk for developing lupus. In another embodiment, the patient to be treated can be screened using an assay to detect auto-antibodies, such as those noted below, wherein autoantibody production is assessed qualitatively, and preferably quantitatively. Exemplary such auto-antibodies associated with SLE are anti-nuclear antibody (ANA), anti-double-stranded DNA (dsDNA) antibody, anti-Sm antibody, anti-nuclear ribonucleoprotein antibody, anti-phospholipid antibody, anti-ribosomal P antibody, anti-Ro/SS-A antibody, anti-Ro antibody, anti-RNP antibody, and anti-La antibody.

A “stable” formulation is one in which the protein therein essentially retains its physical stability and/or chemical stability and/or biological activity upon storage. Preferably, the formulation essentially retains its physical and chemical stability, as well as its biological activity upon storage. The storage period is generally selected based on the intended shelf-life of the formulation. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected temperature for a selected time period. Preferably, the formulation is stable at about 40° C. for at least about 2-4 weeks, and/or stable at about 5° C. and/or 15° C. for at least 3 months, and/or stable at about −20° C. for at least 3 months or at least 1, 2, 3, or 4 years. Furthermore, the formulation is preferably stable following freezing (to, e.g., −70° C.) and thawing of the formulation, for example following 1, 2 or 3 cycles of freezing and thawing. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g. Asn deamidation), oxidation (e.g. Met oxidation), isomerization (e.g. Asp isomeriation), clipping/hydrolysis/fragmentation (e.g. hinge region fragmentation), succinimide formation, unpaired cysteine(s), N-terminal extension, C-terminal processing, glycosylation differences, etc.

A “histidine buffer” is a buffer comprising histidine ions. Examples of histidine buffers include histidine chloride, histidine acetate, histidine phosphate, histidine sulfate. The preferred histidine buffer identified in the examples herein was found to be histidine chloride. In one embodiment, the histidine chloride buffer is prepared by titrating L-histidine (free base, solid) with hydrochloric acid (liquid). In another embodiment, the histidine buffer is prepared by a mixture of histidine and histidine-hydrochloride salt to achieve the desired pH. Preferably, the histidine buffer or histidine chloride buffer is at pH 5.5 to 6.5, preferably pH 5.8 to 6.2.

A “saccharide” herein comprises the general composition (CH₂O)n and derivatives thereof, including monosaccharides, disaccharides, trisaccharides, polysaccharides, sugar alcohols, reducing sugars, nonreducing sugars, etc. Examples of saccharides herein include glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol, iso-maltulose, etc.

Herein, a “surfactant” refers to a surface-active agent, preferably a nonionic surfactant. Examples of surfactants herein include polysorbate (for example, polysorbate 20 and, polysorbate 80); poloxamer (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.); polyethyl glycol, polypropyl glycol, and copolymers of ethylene and propylene glycol (e.g. Pluronics, PF68 etc); etc. The preferred surfactant herein is polysorbate 20.

A flare is a measurable increase in disease activity in one or more organ systems involving new or worse clinical signs and symptoms and/or laboratory measurements. It must be considered clinically significant by the assessor and usually there would be at least consideration of a change or an increase in treatment. (See Ruperto et al., International consensus for a definition of disease flare in lupus. Lupus (2011) 20: 453-462.) “Flare” refers to onset of disease activity in a patient diagnosed with an immune disorder; in SLE, mild flare has been defined by an increase in systemic lupus erythematosus disease activity index (SLEDAI) by ≧4 units over a previous score for that patient and severe flare, as an increase in SLEDAI by ≧12 units. SLEDAI represents a composite assessment of disease activity based on 16 clinical manifestations and eight laboratory measures including two immunological tests with a possible range of overall score from 0 to 105.

“Reduction of lupus flare” or “reduction of flare” and grammatical equivalents thereof refers, depending on the context, either to a decrease in the number of flares relative to a placebo/control group, a decrease in the time to flare, or a decrease in the severity of flares as assessed using the SELENA Flare Index-Revised (SFI-R) (2009).

Diagnosis of SLE may be according to current American College of Rheumatology (ACR) criteria. Active disease may be defined by one British Isles Lupus Activity Group's (BILAG) “A” criteria or two BILAG “B” criteria; SLE Disease Activity Index (SLEDAI); or systemic lupus erythematosus (SLE) responder index (SRI) as noted in the Examples below and described in Furie et al., Arthritis Rheum. 61(9):1143-51 (2009). Some signs, symptoms, or other indicators used to diagnose SLE adapted from: Tan et al. “The Revised Criteria for the Classification of SLE” Arth Rheum 25 (1982) may be malar rash such as rash over the cheeks, discoid rash, or red raised patches, photosensitivity such as reaction to sunlight, resulting in the development of or increase in skin rash, oral ulcers such as ulcers in the nose or mouth, usually painless, arthritis, such as non-erosive arthritis involving two or more peripheral joints (arthritis in which the bones around the joints do not become destroyed), serositis, pleuritis or pericarditis, renal disorder such as excessive protein in the urine (greater than 0.5 gm/day or 3+ on test sticks) and/or cellular casts (abnormal elements derived from the urine and/or white cells and/or kidney tubule cells), neurologic signs, symptoms, or other indicators, seizures (convulsions), and/or psychosis in the absence of drugs or metabolic disturbances that are known to cause such effects, and hematologic signs, symptoms, or other indicators such as hemolytic anemia or leukopenia (white blood count below 4,000 cells per cubic millimeter) or lymphopenia (less than 1,500 lymphocytes per cubic millimeter) or thrombocytopenia (less than 100,000 platelets per cubic millimeter). The leukopenia and lymphopenia must be detected on two or more occasions. The thrombocytopenia must be detected in the absence of drugs known to induce it. The invention is not limited to these signs, symptoms, or other indicators of lupus.

“Treatment” of a subject herein refers to both therapeutic treatment and prophylactic or preventative measures. Those in need of treatment include those already with the lupus as well as those in which the lupus is to be prevented. Hence, the subject may have been diagnosed as having the lupus or may be predisposed or susceptible to the lupus.

A “symptom” of lupus is any morbid phenomenon or departure from the normal in structure, function, or sensation, experienced by the subject and indicative of disease.

The expression “effective amount” refers to an amount of the antibody that is effective for preventing, ameliorating, or treating the lupus.

Patients with “moderately to severely active SLE” are defined by a BILAG A score in at least one domain, or BILAG B scores in at least two domains. For BILAG B scores in the following domains, additional criteria apply: 1) Constitutional domain: BILAG B scores to which anorexia contributed do not count towards entry requirements; 2) Musculoskeletal domain: BILAG B scores to which arthritis (moderate)/tendonitis/tenosynovitis contributed do not count towards entry requirements unless objective signs of inflammation (i.e., tenderness, swelling or effusion) are observed in three or more joints. A patient-reported history of arthritis is not sufficient; and 3) Neuropsychiatric domain: BILAG B scores to which lupus headache contributed do not count towards entry requirements. Cognitive dysfunction cannot contribute to a B score unless it has been established using appropriate cognitive testing and is documented in the source document.

In some embodiments, the patient has at least one BILAG A score or three or more BILAG B scores. In some embodiments, the patient has one or two BILAG B scores.

The BILAG 2004 index is used for determining BILAG score. Yee, et al. Arthritis & Rheumatism 54:3300-3305, 2006; Isenberg et al., Rheumatology 44:902-906; 2005. The BILAG 2004 index assesses 97 clinical signs, symptoms, and laboratory parameters across nine organ system domains: constitutional, mucocutaneous, neuropsychiatric, musculoskeletal, cardiorespiratory, gastrointestinal, ophthalmic, renal, and hematological. The 97 symptoms are rated with respect to severity over the previous month (4 weeks) and with respect to any change from the previous examination (new, improving, stable, worsening, absent). A single alphabetic score (A through E) for each of the nine domains is then derived from the examination results in each organ category.

The scoring of SLE disease activity in Categories A-E is based on the principle of the physician's intention to treat. It is essential that clinical findings are only scored if there are attributable to SLE. BILAG assessments must be performed by a clinician with expertise in SLE and who can demonstrate adequate training in the use of the instrument. The BILAG assessment should be done in a consistent manner and/or by the same assessor at each visit. See Table 1 below. See Table 1 below.

TABLE 1

| Category | Definition |
|---|---|
| A | Severe disease activity requiring any of the following |
| | treatment: |
| | 1. Systemic high dose oral glucocorticoids (equivalent to |
| | prednisolone >20mg/day); |
| | 2. Intravenous pulse glucocorticoids (equivalent to pulse |
| | methylprednisolone ≧500 mg); |
| | 3. Systemic immunomodulators (include biologicals, |
| | immunoglobulins and plasmapheresis); |
| | 4. Therapeutic high dose anticoagulation in the |
| | presence of high dose steroids or |
| | immunomodulators, e.g., warfarin with target INR 3-4. |
| B | Moderate disease activity requiring any of the following |
| | treatment: |
| | 1. Systemic low dose oral glucocorticoids (equivalent to |
| | prednisolone ≦20mg/day); |
| | 2. Intramuscular or intra-articular or soft tissue |
| | glucocorticoids injection (equivalent to |
| | methylprednisolone <500 mg). |
| C | Stable mild disease. |
| D | Inactive disease but previously affected. |
| E | System never involved. |

The “SELENA-SLEDAI Index” is used as an instrument to assess disease activity. Table 2 below shows the criteria for determining the SELENA-SLEDAI score. Total score is sum of weights next to descriptors marked present.

TABLE 2

| Wt | Present | Descriptor | Definition |
|---|---|---|---|
| 8 | □ | Seizure | Recent onset (last 28 days). Exclude metabolic, infectious or |
| | | | drug cause, or seizure due to past irreversible CNS damage. |
| 8 | □ | Psychosis | Altered ability to function in normal activity due to severe |
| | | | disturbance in the perception of reality. Include |
| | | | hallucinations, incoherence, marked loose associations, |
| | | | impoverished thought content, marked illogical thinking, |
| | | | bizarre, disorganized, or catatonic behavior. Exclude uremia |
| | | | and drug causes. |
| 8 | □ | Organic Brain | Altered mental function with impaired orientation, memory |
| | | Syndrome | or other intellectual function, with rapid onset and |
| | | | fluctuating clinical features. Include clouding of |
| | | | consciousness with reduced capacity to focus and inability to |
| | | | sustain attention to environment, plus at least two of the |
| | | | following: perceptual disturbance, incoherent speech, |
| | | | insomnia or daytime drowsiness, or increased or decreased |
| | | | psychomotor activity. Exclude metabolic, infectious or drug |
| | | | causes. |
| 8 | □ | Visual | Retinal and eye changes of SLE. Include cytoid bodies, |
| | | Disturbance | retinal hemorrhages, serous exudate or hemorrhages in the |
| | | | choroid, optic neuritis, scleritis, or episcleritis. Exclude |
| | | | hypertension, infection, or drug causes. |
| 8 | □ | Cranial Nerve | New onset of sensory or motor neuropathy involving cranial |
| | | Disorder | nerves. Include vertigo due to lupus. |
| 8 | □ | Lupus Headache | Severe persistent headache: may be migrainous, but must be |
| | | | non-responsive to narcotic analgesia. |
| 8 | □ | CVA | New onset of cerebrovascular accident(s). Exclude |
| | | | arteriosclerosis or hypertensive causes. |
| 8 | □ | Vasculitis | Ulceration, gangrene, tender finger nodules, periungual |
| | | | infarction, splinter hemorrhages, or biopsy or angiogram |
| | | | proof of vasculitis. |
| 4 | □ | Arthritis | More than 2 joints with pain and signs of inflammation (i.e. |
| | | | Tenderness, swelling, or effusion). |
| 4 | □ | Myositis | Proximal muscle aching/weakness associated with elevated |
| | | | creatine phosphokinase/aldolase or electromyogram changes |
| | | | or a biopsy showing myositis. |
| 4 | □ | Urinary Casts | Heme-granular or red blood cell casts. |
| 4 | □ | Hematuria | >5 red blood cells/high power field. Exclude stone, infection |
| | | | or other causes. |
| 4 | □ | Proteinuria | New onset or recent increase of more than 0.5 g/24 hr |
| 4 | □ | Pyuria | >5 white blood cells/high power field. Exclude infection. |
| 2 | □ | Rash | Ongoing Inflammatory lupus rash |
| 2 | □ | Alopecia | Ongoing abnormal, patchy or diffuse loss of hair due to |
| | | | active lupus |
| 2 | □ | Mucosal Ulcers | Ongoing, oral or nasal ulcerations due to active lupus |
| 2 | □ | Pleurisy | Classic and severe pleuritic chest pain or pleural rub or |
| | | | effusion or new pleural thickening due to lupus |
| 2 | □ | Pericarditis | Classic and severe pericardial pain or rub or effusion, or |
| | | | electrocardiogram confirmation. |
| 2 | □ | Low Complement | Decrease in CH50, C3, or C4 below the lower limit of |
| | | | normal for testing laboratory |
| 2 | □ | Increased DNA | >25% binding by Farr assay or above normal range for |
| | | binding | testing laboratory |
| 1 | □ | Fever | >38° C. Exclude infectious cause. |
| 1 | □ | Thrombocytopenia | <100,000 platelets/mm³ |
| 1 | □ | Leukopenia | <3,000 white blood cells/mm³. Exclude drug causes |

The Physician's Global Assessment is also used to assess disease activity. Physicians are to rate the patient's disease activity over the past 28 days and place a vertical tick mark on a 100-mm analog scale marked from “none” to “severe” and graded from 0 to 3. Patient history, results of the physical examination, as well as pertinent laboratory values should be taken into account when rating the patient's disease activity. Physicians should also refer to the value recorded at the previous visit and move the tick mark as appropriate.

The SLE Responder Index (SRI) is a clinically meaningful endpoint that measures improvements in SLE disease activity while checking that no worsening occurs in any organ system or overall patient condition. SRI is a composite endpoint incorporating the SELENA-SLEDAI, the BILAG 2004 Index, and the Physician's Global Assessment (PGA).

An “SRI-4” response requires each of the following criteria to be met: 1)≧4 point reduction in the SELENA-SLEDAI score; 2) no new BILAG A organ domain score or no more than one new BILAG B organ domain scores; 3) no worsening (less than 10% increase) in PGA; and 4) no treatment failure.

An “SRI-5” response requires each of the following criteria to be met: 1)≧5 point reduction in the SELENA-SLEDAI score; 2) no new BILAG A organ domain score or no more than one new BILAG B organ domain scores; 3) no worsening (less than 10% increase) in PGA; and 4) no treatment failure.

An “SRI-6” response requires each of the following criteria to be met: 1)≧6 point reduction in the SELENA-SLEDAI score; 2) no new BILAG A organ domain score or no more than one new BILAG B organ domain scores; 3) no worsening (less than 10% increase) in PGA; and 4) no treatment failure.

An “SRI-7” response requires each of the following criteria to be met: 1)≧7 point reduction in the SELENA-SLEDAI score; 2) no new BILAG A organ domain score or no more than one new BILAG B organ domain scores; 3) no worsening (less than 10% increase) in PGA; and 4) no treatment failure.

Patients who require additional steroids exceeding a certain dose and duration or who require resumption/initiation of an immunosuppressive regimen are considered “treatment failures”. Patients who exceed the following steroid doses are considered treatment failures:
1) Patients unable to complete the steroid taper (not reaching target dose of 10 mg/day or less by the end of Week 8). Patients who cannot reach the target of ≦10 mg/day by the end of Week 6 according to the taper schedule but who achieve this target by the end of Week 8 are not be considered as treatment failures.
2) Prior to Week 20
   Any increase in steroids exceeding the lowest achieved dose by 20 mg or more for at least 14 days;
   Any increase in steroids exceeding the lowest achieved dose by 10 mg or more for at least 28 days.
3) From Week 20 to Week 24
   Received 20 mg or more of prednisone equivalent on any day during this 4-week period;
   Received more than 10 but less than 20 mg/day prednisone equivalent for more than 7 days (cumulative).

The SELENA-SLEDAI Flare Index (SFI) published in Lupus [1999] 8(8):685-91 and a SELENA-SLEDAI Flare Index-Revised (SFI-R) published in Arthritis & Rheumatology [2011] 63(12): 3918-30 are used as criteria for determining flares.

The “SFI-R” evaluates increases in SLE disease activity within eight organ systems: mucocutaneous, musculoskeletal, cardiopulmonary, hematological, constitutional, renal, neurological, and gastrointestinal as shown in tables below. Within each organ system, the investigators assess clinical manifestations and treatment recommendations to arrive at a flare categorization as no flare, mild flare, moderate flare, or severe flare. In the event that the assessment of a clinical manifestation and the recommendation for a treatment change are discrepant, the treatment choice takes precedence (in the direction of a higher flare definition). Treatment changes recommended because of intolerance, toxicity, or safety do not count towards a flare definition.

### 1. Mucocutaneous System

| None | Mild | Moderate | Severe |
|---|---|---|---|
| | Clinical: | Clinical: | Clinical: |
| | New/worse/recurrent malar | New/worse extensive | New/worse extensive |
| | rash | oral/nasal ulcers | and/or severe vasculitis, |
| | New/worse mild oral/nasal | New/worse discoid beyond a | panniculitis, bullous |
| | ulcers | very localized area, such as | lesions, large cutaneous |
| | New/worse discoid in a | new areas, enlargement, or | ulcers, desquamating, |
| | small existing lesion or a | deepening lesions | necrosis, gangrene, |
| | very localized area such as | New/worse moderate | angioedema |
| | ear | photosensitive or | |
| | New mild photosensitive or | maculopapular rash | |
| | maculopapular rash | New/worse marked alopecia | |
| | New mild alopecia | New/worse small cutaneous | |
| | New mild bullous lupus | ulcers, very limited | |
| | | periungual infarcts | |
| | | New/worse mild to moderate | |
| | | angioedema | |
| | | New/worse moderate bullous | |
| | | lupus | |
| | | New/worse mild to moderate | |
| | | panniculitis | |
| | AND/OR | AND/OR | AND/OR |
| | Treatment: any of | Treatment: any of | Treatment: any of |
| | No treatment or analgesic | New/increased prednisone | New/increased |
| | Topical treatment | to >7.5 mg/day but <0.5 | prednisone ≧0.5 |
| | New/increased | mg/kg/day for >3 days | mg/kg/day (including IV |
| | hydroxychloroquine or | Intramuscular corticosteroid | methylprednisolone) |
| | other antimalarial | New or increased dose of | Cyclophosphamide |
| | New/increased prednisone | immunosuppressive (not | Rituximab or other |
| | ≦7.5 mg/day | cyclophosphamide) | biologic |
| | | Two antimalarials | Hospitalization for |
| | | Thalidomide | severe activity |
| | | Dapsone | |
| | | New/increased retinoids | |

### 2. Musculoskeletal System

| None | Mild | Moderate | Severe |
|---|---|---|---|
| | Clinical: | Clinical: | Clinical: |
| | New/worse/recurrent | New/worse/recurrent | New/worse/polyarthritis |
| | polyarthralgias | polyarthritis (3 or more | (3 or more joints) with |
| | New/mild arthritis of 1 or 2 | joints) | marked reduction in |
| | joints | | range of motion or |
| | | | mobility |
| | AND/OR | AND/OR | AND/OR |
| | Treatment: any of | Treatment: any of | Treatment: any of |
| | No treatment or analgesia | New/increased prednisone | New/increased |
| | New/increased | to >7.5 mg/day but <0.5 | prednisone ≧0.5 |
| | hydroxychloroquine or | mg/kg/day for >3 days | mg/kg/day (including IV |
| | other antimalarial | Intramuscular corticosteroid | methylprednisolone) |
| | New/increased prednisone | Methotrexate ≦15 mg/wk | Methotrexate >15 mg/wk |
| | ≦7.5 mg/day | New or increased dose of | Cyclophosphamide |
| | New or increased NSAID | immunosuppressive (not | Rituximab or other |
| | New/increased | cyclophosphamide) | biologic |
| | dehydroepiandrosterone | Intraarticular corticosteroid | Hospitalization for |
| | (DHEA) | | severe activity |

### 3. Cardiopulmonary System

| None | Mild | Moderate | Severe |
|---|---|---|---|
| | Clinical: | Clinical: | Clinical: |
| | New/worse mild pleurisy | New/worse moderate | New/worse pleural or |
| | or pericarditis (symptoms | pleurisy, pericarditis, small | pericardial effusion |
| | sufficient) | pleural effusion (with | requiring tap or window, |
| | | physical examination | tamponade |
| | | findings, radiographs or | New/worse pulmonary |
| | | ECHO) | hemorrhage, shrinking |
| | | | lung |
| | | | New/worse myocarditis, |
| | | | coronary arteritis |
| | AND/OR | AND/OR | AND/OR |
| | Treatment: any of | Treatment: any of | Treatment: any of |
| | No treatment or analgesic | New/increased prednisone | New/increased prednisone |
| | New/increased | to >7.5 mg/day but <0.5 | ≧0.5 mg/kg/day (including |
| | hydroxychloroquine or | mg/kg/day for >3 days | IV methylprednisolone) |
| | other antimalarial | Intramuscular corticosteroid | Cyclophosphamide |
| | New/increased prednisone | New or increased dose of | Rituximab or other |
| | ≦7.5 mg/day | immunosuppressive (not | biologic |
| | New or increased NSAID | cyclophosphamide) | Hospitalization for severe |
| | | IV methylprednisolone if | activity |
| | | one dose | |

### 4. Hematological System

| None | Mild | Moderate | Severe |
|---|---|---|---|
| | Clinical: | Clinical: | Clinical: |
| | Leukopenia- | Leukopenia-<1500 but | Leukopenia-<1000 |
| | new/worse/recurrent | ≧1000 | Thrombocytopenia- |
| | <3,000 | Thrombocytopenia-30 to | <30,000 or thrombotic |
| | Thrombocytopenia- | 50,000 | microangiopathy |
| | New/worse/recurrent | Hemolytic anemia or anemia | Hemolytic anemia or |
| | 50 to 100,000 | of active SLE-HCT ≦30, but | anemia of active SLE- |
| | Hemolytic anemia or | >25 | HCT ≦25 |
| | anemia of active SLE- | | |
| | HCT >30 | | |
| | AND/OR | AND/OR | AND/OR |
| | Treatment: any of | Treatment: any of | Treatment: any of |
| | No treatment or analgesic | New/increased prednisone | New/increased prednisone |
| | New/increased | to >7.5 mg/day but <0.5 | ≧0.5 mg/kg/day (including |
| | hydroxychloroquine or | mg/kg/day for >3 days | IV methylprednisolone) |
| | other antimalarial | Intramuscular corticosteroid | Cyclophosphamide |
| | New/increased prednisone | New or increased dose of | Rituximab or other |
| | ≦7.5 mg/day | immunosuppressive (not | biologic |
| | | cyclophosphamide) | Hospitalization for severe |
| | | | activity |
| | | | Intravenous |
| | | | immunoglobulin |
| | | | Plasmapheresis |
| | | | Splenectomy |

### 5. Constitutional

| None | Mild | Moderate | Severe |
|---|---|---|---|
| | Clinical: | Clinical: | Clinical: |
| | Fever | Fever | Fever |
| | New/worse/recurrent up | New/worse >101° F. | New/worse >103° F. |
| | to 101° F. (38.3° C.) | (38.3° C.) but ≦103° F. | (39.4° C.) |
| | Lymphadenopathy | (39.4° C.) | Weight loss |
| | New/worse up to a few | Lymphadenopathy | >10% weight loss |
| | small cervical/axillary | New/worse lymph nodes | |
| | nodes (<1 cm) | outside cervical chain | |
| | Weight loss | Weight loss | |
| | New weight loss <5% | 5% to 10% weight loss | |
| | AND/OR | AND/OR | AND/OR |
| | Treatment: any of | Treatment: any of | Treatment: any of |
| | No treatment or analgesic | New/increased prednisone | New/increased prednisone |
| | New/increased | to >7.5 mg/day but <0.5 | ≧0.5 mg/kg/day |
| | hydroxychloroquine or | mg/kg/day for >3 days | (including IV |
| | other antimalarial | Intramuscular steroid | methylprednisolone) |
| | New/increased prednisone | New or increased dose of | Cyclophosphamide |
| | ≦7.5 mg/day | immunosuppressive (not | Rituximab or other biologic |
| | New/increased NSAID | cyclophosphamide) | Hospitalization for severe |
| | | | activity |

### 6. Renal System

| None | Mild | Moderate | Severe |
|---|---|---|---|
| | Clinical: | Clinical: | Clinical: |
| | New/worse protein/cr >0.2 | New/worse urine pr/cr ≧0.5 | Urine pr/cr ≧1.0 if baseline |
| | but <0.5 | but <1.0 | <0.3 Urine pr/cr doubled if |
| | | Increase in RBC/hpf | baseline is >1 |
| | | from <5 to >15 with >2 | Urine pr/cr >5.0 |
| | | acanthocytes/hpf | New RBC casts or mixed |
| | | | RBC casts |
| | | | Biopsy with new/worse |
| | | | aggressive lesions |
| | | | (necrosis, crescents) |
| | | | Biopsy with Class IV |
| | | | Rapidly progressive |
| | | | glomerulonephritis |
| | | | Decreased GFR in last 3 |
| | | | months If baseline Cr <2, |
| | | | increase of >0.2 mg/dL |
| | | | If baseline Cr >2, increase |
| | | | of ≧0.4 mg/dL |
| | AND/OR | AND/OR | AND/OR |
| | Treatment: any of | Treatment: any of | Treatment: any of |
| | New/increased | New/increased prednisone | New/increased prednisone |
| | hydroxychloroquine or | to >7.5 mg/day but <0.5 | >0.5 mg/kg/day (including |
| | other antimalarial | mg/kg/day for >3 days | IV methylprednisolone) |
| | New/increased prednisone | Intramuscular | Mycophenolate mofetil or |
| | ≦7.5 mg/day | corticosteroid | azathioprine for severe |
| | Angiotensin-converting | New or increased dose of | nephritis |
| | enzyme (ACE) inhibitor, | immunosuppressive (not | Cyclophosphamide |
| | angiotensin-receptor | cyclophosphamide) | Rituximab or other |
| | blocker (ARB), | | biologic |
| | spironolactone, low protein | | Hospitalization for severe |
| | diet, low sodium diet | | activity |
| | Statins | | |

### 7. Neurological System

| None | Mild | Moderate | Severe |
|---|---|---|---|
| | Clinical: | Clinical: | Clinical: |
| | Minimal/intermittent ACR | New/worsening persistent | Acute delirium or confusional |
| | neuropsychiatric SLE | ACR neuropsychiatric | state (organic brain |
| | syndrome | SLE syndrome | syndrome) |
| | | | Coma |
| | | | Status epilepticus |
| | | | Cranial nerve palsy (including |
| | | | optic) |
| | | | Stroke due to CNS vasculitis |
| | | | Aseptic meningitis |
| | | | Mononeuritis multiplex |
| | | | Longitudinal myelitis |
| | | | Chorea |
| | | | Cerebellar ataxia |
| | | | Myositis with weakness |
| | AND/OR | AND/OR | AND/OR |
| | Treatment: any of | Treatment: any of | Treatment: any of |
| | No treatment or analgesic | New/increased prednisone | New/increased prednisone |
| | New/increased | to >7.5 mg/day but <0.5 | >0.5 mg/kg/day (including IV |
| | hydroxychloroquine or | mg/kg/day for >3 days | methylprednisolone) |
| | other antimalarial | Intramuscular | Cyclophosphamide |
| | New/increased prednisone | corticosteroid | Rituximab or other biologic |
| | ≦7.5 mg/day | New or increased dose of | Hospitalization for severe |
| | | immunosuppressive (not | activity |
| | | cyclophosphamide) | Plasmapheresis |
| | | | Intravenous immunoglobulin |

### 8. Gastrointestinal System

| None | Mild | Moderate | Severe |
|---|---|---|---|
| | Clinical: | Clinical: | Clinical: |
| | New/worse LFTs >2× | New/worse LFT ≧4× | New/worse lupus peritonitis |
| | normal but <4× normal | normal | with ascites |
| | AND/OR | New/worse pancreatitis | New/worse enteritis, colitis or |
| | Treatment: any of | with increased amylase, | protein-losing enteropathy |
| | No treatment or analgesic | but no IV therapy | New/worse intestinal pseudo- |
| | New/increased | New/worse clinical | obstruction with hypomotility |
| | hydroxychloroquine or | peritonitis with no ascites | New/worse pancreatitis |
| | other antimalarial | AND/OR | requiring IV therapy |
| | New/increased predni- | Treatment: any of | New/worse GI vasculitis |
| | sone ≦7.5 mg/day | New/increased prednisone | (mesenteric or other GI organ) |
| | | to >7.5 mg/day but <0.5 | AND/OR |
| | | mg/kg/day for >3 days | Treatment: any of |
| | | Intramuscular cortico- | New/increased prednisone ≧0.5 |
| | | steroid | mg/kg/day (including IV methyl- |
| | | New or increased dose of | prednisolone) |
| | | immunosuppressive (not | Cyclophosphamide |
| | | cyclophosphamide) | Rituximab or other biologic |
| | | | Hospitalization for severe |
| | | | activity |

“Antibody exposure” refers to contact with or exposure to the antibody herein in one or more doses administered over a period of time of about 1 day to about 5 weeks. The doses may be given at one time or at a fixed or irregular time intervals over this period of exposure, such as, for example, one dose weekly for four weeks or two doses separated by a time interval of about 13-17 days. Initial and later antibody exposures are separated in time from each other as described in detail herein.

The term “immunosuppressive agent” as used herein for adjunct therapy refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); nonsteroidal antiinflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, and dexamethasone; dihydrofolate reductase inhibitors such as methotrexate (oral or subcutaneous); hydroxycloroquine; sulfasalazine; leflunomide; cytokine or cytokine receptor antibodies including anti-interferon-alpha, -beta, or -gamma antibodies, anti-tumor necrosis factor-alpha antibodies (infliximab or adalimumab), anti-TNF-alpha immunoahesin (etanercept), anti-tumor necrosis factor-beta antibodies, anti-interleukin-2 antibodies and anti-IL-2 receptor antibodies; anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 1990/08187 published Jul. 26, 1990); streptokinase; TGF-beta; streptodornase; RNA or DNA from the host; FK506; RS-61443; deoxyspergualin; rapamycin; T-cell receptor (Cohen et al., U.S. Pat. No. 5,114,721); T-cell-receptor fragments (Offner et al., Science, 251: 430-432 (1991); WO 1990/11294; Ianeway, Nature, 341: 482 (1989); and WO 1991/01133); and T-cell-receptor antibodies (EP 340,109) such as T10B9.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small-molecule toxins or enzymatically active toxins of bacterial, fungal, plant, or animal origin, or fragments thereof.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin, and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids, or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, and H-Ras; vaccines such as gene-therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids, or derivatives of any of the above.

The term “cytokine” is a generic term for proteins released by one cell population that act on another cell as intercellular mediators. Examples of such cytokines are lymphokines, monokines; interleukins (ILs) such as IL-1, IL-1α, IL-2, IL-3, IL-4, IL-5, IL-6, IL-7, IL-8, IL-9, IL-11, IL-12, IL-15; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native-sequence cytokines, including synthetically produced small-molecule entities and pharmaceutically acceptable derivatives and salts thereof.

The term “hormone” refers to polypeptide hormones, which are generally secreted by glandular organs with ducts. Included among the hormones are, for example, growth hormone such as human growth hormone, N-methionyl human growth hormone, and bovine growth hormone; parathyroid hormone; thyroxine; insulin; proinsulin; relaxin; prorelaxin; glycoprotein hormones such as follicle-stimulating hormone (FSH), thyroid-stimulating hormone (TSH), and luteinizing hormone (LH); prolactin, placental lactogen, mouse gonadotropin-associated peptide, inhibin; activin; mullerian-inhibiting substance; and thrombopoietin. As used herein, the term hormone includes proteins from natural sources or from recombinant cell culture and biologically active equivalents of the native-sequence hormone, including synthetically produced small-molecule entities and pharmaceutically acceptable derivatives and salts thereof.

The term “growth factor” refers to proteins that promote growth, and include, for example, hepatic growth factor; fibroblast growth factor; vascular endothelial growth factor; nerve growth factors such as NGF-β; platelet-derived growth factor; transforming growth factors (TGFs) such as TGF-α and TGF-β; insulin-like growth factor-I and —II; erythropoietin (EPO); osteoinductive factors; interferons such as interferon-α, -β, and -γ; and colony-stimulating factors (CSFs) such as macrophage-CSF (M-CSF); granulocyte-macrophage-CSF (GM-CSF); and granulocyte-CSF (G-CSF). As used herein, the term growth factor includes proteins from natural sources or from recombinant cell culture and biologically active equivalents of the native-sequence growth factor, including synthetically produced small-molecule entities and pharmaceutically acceptable derivatives and salts thereof.

The term “integrin” refers to a receptor protein that allows cells both to bind to and to respond to the extracellular matrix and is involved in a variety of cellular functions such as wound healing, cell differentiation, homing of tumor cells, and apoptosis. They are part of a large family of cell adhesion receptors that are involved in cell-extracellular matrix and cell-cell interactions. Functional integrins consist of two transmembrane glycoprotein subunits, called alpha and beta, that are non-covalently bound. The alpha subunits all share some homology to each other, as do the beta subunits. The receptors always contain one alpha chain and one beta chain. Examples include Alpha6beta1, Alpha3beta1, Alpha7beta1, LFA-1 etc. As used herein, the term integrin includes proteins from natural sources or from recombinant cell culture and biologically active equivalents of the native-sequence integrin, including synthetically produced small-molecule entities and pharmaceutically acceptable derivatives and salts thereof.

For the purposes herein, “tumor necrosis factor-alpha (TNF-alpha)” refers to a human TNF-alpha molecule comprising the amino acid sequence as described in Pennica et al., Nature, 312:721 (1984) or Aggarwal et al., JBC, 260:2345 (1985).

A “TNF-alpha inhibitor” herein is an agent that inhibits, to some extent, a biological function of TNF-alpha, generally through binding to TNF-alpha and neutralizing its activity. Examples of TNF inhibitors specifically contemplated herein are etanercept (ENBREL®), infliximab (REMICADE®), and adalimumab (HUMIRA™).

Examples of “disease-modifying anti-rheumatic drugs” or “DMARDs” include hydroxycloroquine, sulfasalazine, methotrexate, leflunomide, etanercept, infliximab (plus oral and subcutaneous methrotrexate), azathioprine, D-penicillamine, gold salts (oral), gold salts (intramuscular), minocycline, cyclosporine, staphylococcal protein A immunoadsorption, including salts and derivatives thereof, etc.

Examples of “nonsteroidal anti-inflammatory drugs” or “NSAIDs” are acetylsalicylic acid, ibuprofen, naproxen, indomethacin, sulindac, tolmetin, including salts and derivatives thereof, etc.

Examples of “integrin antagonists or antibodies” herein include an LFA-1 antibody, such as efalizumab (RAPTIVA®) commercially available from Genentech, or an alpha 4 integrin antibody such as natalizumab (ANTEGREN®) available from Biogen, or diazacyclic phenylalanine derivatives (WO 2003/89410), phenylalanine derivatives (WO 2003/70709, WO 2002/28830, WO 2002/16329 and WO 2003/53926), phenylpropionic acid derivatives (WO 2003/10135), enamine derivatives (WO 2001/79173), propanoic acid derivatives (WO 2000/37444), alkanoic acid derivatives (WO 2000/32575), substituted phenyl derivatives (U.S. Pat. Nos. 6,677,339 and 6,348,463), aromatic amine derivatives (U.S. Pat. No. 6,369,229), ADAM disintegrin domain polypeptides (US 2002/0042368), antibodies to alphavbeta3 integrin (EP 633945), aza-bridged bicyclic amino acid derivatives (WO 2002/02556), etc.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone), dexamethasone triamcinolone, and betamethasone.

“The term “corticosteroid sparing” or “CS” means the decrease in frequency and/or amount, or the elimination of, corticosteroid used to treat a disease in a patient taking corticosteroids for the treatment of the disease due to the administration of another therapeutic agent. A “CS agent” refers to a therapeutic agent that can cause CS in a patient taking a corticosteroid.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, etc.

An exposure not being administered or provided until a certain time “from the initial exposure” or from any prior exposure means that the time for the second or later exposure is measured from the time any of the doses from the prior exposure were administered, if more than one dose was administered in that exposure. For example, when two doses are administered in an initial exposure, the second exposure is not given until at least about 16-54 weeks as measured from the time the first or the second dose was administered within that prior exposure. Similarly, when three doses are administered, the second exposure may be measured from the time of the first, second, or third dose within the prior exposure. Preferably, “from the initial exposure” is measured from the time of the first dose.

A “medicament” is an active drug to treat the lupus or its symptoms or side effects.

### III. Methods

The present invention provides compositions and methods for the diagnosis and/or selection of a population of autoimmune patients that would be good candidates for treatment with interferon inhibitor(s). In one embodiment, the invention provides for methods of treating lupus (e.g., SLE) in a patient, comprising administering an effective amount of an antibody that binds to a type I interferon, wherein the patient is ENA−. In some embodiments, the invention provides methods of treating autoimmune diseases (e.g., SLE) in a patient, comprising administering a certain antibody that binds to a type I interferon according to a particular dosing regimen. In some embodiments, the invention provides methods of treating lupus (e.g., SLE) in a patient, comprising administering an effective amount of an antibody that binds to a type I interferon, wherein the patient's baseline ISM is greater than or equal to the ISM to a healthy individual. In some embodiments, the patient has an IRG status of ISM^{lo}. The antibody may be a naked antibody or may be conjugated with another molecule such as a cytotoxic agent such as a radioactive compound. In one embodiment, the antibody herein is rontalizumab.

The present invention is based partly on the use of specific genes (e.g., one or more of CMPK2, EPSTI1, HERC5, IFI27, IFI44, IFIT1, MX1, OAS1, OAS2, OAS3, and combinations thereof) or biomarkers (e.g., autoantibodies to any ENA or dsDNA) that correlate with efficacy of type I interferon inhibitors (e.g., a type I interferon antibody). Thus, the disclosed methods provide convenient, efficient, and potentially cost-effective means to obtain data and information useful in assessing appropriate or effective therapies for treating patients. For example, a sample can be obtained from a lupus patient, and the sample could be examined by various in vitro assays to determine whether the expression level of one or more biomarkers has increased or decreased as compared to the expression level in a reference sample. In one embodiment, if the patient is ENA−, then the patient is likely to benefit from treatment with a therapy comprising a type I interferon inhibitor (e.g., a type I interferon antibody such as, for example, rontalizumab). In another embodiment, if expression levels of at least 1, 2, 3, 4, 5, 6, 7, or more of CMPK2, EPSTI1, HERC5, IFI27, IFI44, IFIT1, MX1, OAS1, OAS2, or OAS3 in the sample from the patient is less than or equal to the expression level in a healthy individual, then the patient is likely to benefit from treatment with a therapy comprising a type I interferon inhibitor (e.g., a type I interferon antibody such as, for example, rontalizumab).

Expression levels/amount of a gene or a biomarker can be determined based on any suitable criterion known in the art, including but not limited to mRNA, cDNA, proteins, protein fragments and/or gene copy number.

Expression of various genes or biomarkers in a sample can be analyzed by a number of methodologies, many of which are known in the art and understood by the skilled artisan, including but not limited to, immunohistochemical and/or Western blot analysis, immunoprecipitation, molecular binding assays, ELISA, ELIFA, fluorescence activated cell sorting (FACS) and the like, quantitative blood based assays (as for example Serum ELISA) (to examine, for example, levels of protein expression), biochemical enzymatic activity assays, in situ hybridization, Northern analysis and/or PCR analysis of mRNAs, as well as any one of the wide variety of assays that can be performed by gene and/or tissue array analysis. Typical protocols for evaluating the status of genes and gene products are found, for example in Ausubel et al. eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (MSD) may also be used.

In certain embodiments, expression/amount of a gene or biomarker in a sample is increased as compared to expression/amount in a reference sample if the expression level/amount of the gene or biomarker in the sample is greater than the expression level/amount of the gene or biomarker in reference sample. Similarly, expression/amount of a gene or biomarker in a sample is decreased as compared to expression/amount in a reference sample if the expression level/amount of the gene or biomarker in the ample is less than the expression level/amount of the gene or biomarker in the reference sample.

In certain embodiments, the samples are normalized for both differences in the amount of RNA or protein assayed and variability in the quality of the RNA or protein samples used, and variability between assay runs. Such normalization may be accomplished by measuring and incorporating the expression of certain normalizing genes, including well known housekeeping genes, such as ACTB, GAPDH, etc. Alternatively, normalization can be based on the mean or median signal of all of the assayed genes or a large subset thereof (global normalization approach). On a gene-by-gene basis, measured normalized amount of a patient tumor mRNA or protein is compared to the amount found in a reference set. Normalized expression levels for each mRNA or protein per tested tumor per patient can be expressed as a percentage of the expression level measured in the reference set. The expression level measured in a particular patient sample to be analyzed will fall at some percentile within this range, which can be determined by methods well known in the art.

The expression levels of the IRGs of interest and of a housekeeping gene can be measured from a biological sample from an autoimmune patient. The resulting detection data (e.g., Ct data) of the IRGs can be normalized against the detection data for the housekeeping gene resulting in a DCt value (DCt=Ct(ISM gene)—Ct (housekeeping gene). The mean value of the DCt values of the IRGs tested can be calculated (e.g., triplicate DCt values for Herc5, Tyk1 and EPST1 are added and divided by 9). The expression levels of the same IRGs of interest from a biological sample from two or more healthy persons can be detected using the same methods, and the mean value and standard deviation for the healthy persons data can be calculated. Alternatively, if substitute values (e.g., a control(s)) has been developed for the same method, then those values can be used in place of testing healthy persons.

The mean DCt value of the autoimmune patient can be compared against the mean Ct value of the healthy persons as follows: (1) a threshold value can be set, wherein above the threshold value, the patient would be considered to have an ISM hi score (i.e., equal to or greater than 1) and below the threshold value, the patient can be considered to have an ISM low score (i.e., less than 1); (2) in one embodiment, the threshold value is 1.5 times the value of the mean Ct value of healthy persons (or a control) or two standard deviations above the mean value of healthy persons (or control(s)).

Ct is the threshold cycle. The Ct is the cycle number at which the fluorescence generated within a reaction crosses a predefined threshold line.

In one embodiment, all experiments are normalized to a reference RNA, which is a comprehensive mix of RNA from various tissue sources (e.g., reference RNA #636538 from Clontech, Mountain View, Calif.). In another embodiment, the reference RNA is transferrin receptor (TFRC). Identical reference RNA is included in each qRT-PCR run, allowing comparison of results between different experimental runs.

A sample comprising a target gene or biomarker can be obtained by methods well known in the art. See under Definitions. In addition, the progress of therapy can be monitored more easily by testing such body samples for target genes or gene products.

In certain embodiments, the expression of proteins in a sample is examined using immunohistochemistry (“IHC”) and staining protocols. Immunohistochemical staining of tissue sections has been shown to be a reliable method of assessing or detecting presence of proteins in a sample. Immunohistochemistry techniques utilize an antibody to probe and visualize cellular antigens in situ, generally by chromogenic or fluorescent methods.

Two general methods are available; direct and indirect assays. According to the first assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody typically will be labeled with a detectable moiety. Numerous labels are available which can be generally grouped into the following categories:

(a) Radioisotopes, such as ³⁵S, 14C, 125I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991) for example and radioactivity can be measured using scintillation counting.

(b) Colloidal gold particles.

(c) Fluorescent labels including, but are not limited to, rare earth chelates (europium chelates), Texas Red, rhodamine, fluorescein, dansyl, Lissamine, umbelliferone, phycocrytherin, phycocyanin, or commercially available fluorophores such SPECTRUM ORANGE7 and SPECTRUM GREEN7 and/or derivatives of any one or more of the above. The fluorescent labels can be conjugated to the antibody using the techniques disclosed in Current Protocols in Immunology, supra, for example. Fluorescence can be quantified using a fluorimeter.

(d) Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed. J. Langone & H. Van Vunakis), Academic press, New York, 73:147-166 (1981).

Examples of enzyme-substrate combinations include, for example:

(i) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB));

(ii) alkaline phosphatase (AP) with para-Nitrophenyl phosphate as chromogenic substrate; and

(iii) β-D-galactosidase (β-D-Gal) with a chromogenic substrate (e.g., p-nitrophenyl-β-D-galactosidase) or fluorogenic substrate (e.g., 4-methylumbelliferyl-β-D-galactosidase).

Numerous other enzyme-substrate combinations are available to those skilled in the art. For a general review of these, see U.S. Pat. Nos. 4,275,149 and 4,318,980. Sometimes, the label is indirectly conjugated with the antibody. The skilled artisan will be aware of various techniques for achieving this. For example, the antibody can be conjugated with biotin and any of the four broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten and one of the different types of labels mentioned above is conjugated with an anti-hapten antibody. Thus, indirect conjugation of the label with the antibody can be achieved.

Following an optional blocking step, the sample is exposed to primary antibody for a sufficient period of time and under suitable conditions such that the primary antibody binds to the target protein antigen in the sample. Appropriate conditions for achieving this can be determined by routine experimentation. The extent of binding of antibody to the sample is determined by using any one of the detectable labels discussed above. In certain embodiments, the label is an enzymatic label (e.g. HRPO) which catalyzes a chemical alteration of the chromogenic substrate such as 3,3′-diaminobenzidine chromogen. In one embodiment, the enzymatic label is conjugated to antibody which binds specifically to the primary antibody (e.g. the primary antibody is rabbit polyclonal antibody and secondary antibody is goat anti-rabbit antibody).

In some embodiments, the sample may be contacted with an antibody specific for said biomarker (e.g., autoantubody to an ENA antigen) under conditions sufficient for an antibody-biomarker complex to form, and then detecting said complex. The presence of the biomarker may be detected in a number of ways, such as by Western blotting and ELISA procedures for assaying a wide variety of tissues and samples, including plasma or serum. A wide range of immunoassay techniques using such an assay format are available, see, e.g., U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labeled antibody to a target biomarker.

Sandwich assays are among the most useful and commonly used assays. A number of variations of the sandwich assay technique exist, and all are intended to be encompassed by the present invention. Briefly, in a typical forward assay, an unlabeled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of biomarker.

Variations on the forward assay include a simultaneous assay, in which both sample and labeled antibody are added simultaneously to the bound antibody. These techniques are well known to those skilled in the art, including any minor variations as will be readily apparent. In a typical forward sandwich assay, a first antibody having specificity for the biomarker is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g. from room temperature to 40° C. such as between 25° C. and 32° C. inclusive) to allow binding of any subunit present in the antibody. Following the incubation period, the antibody subunit solid phase is washed and dried and incubated with a second antibody specific for a portion of the biomarker. The second antibody is linked to a reporter molecule which is used to indicate the binding of the second antibody to the molecular marker.

An alternative method involves immobilizing the target biomarkers in the sample and then exposing the immobilized target to specific antibody which may or may not be labeled with a reporter molecule. Depending on the amount of target and the strength of the reporter molecule signal, a bound target may be detectable by direct labelling with the antibody. Alternatively, a second labeled antibody, specific to the first antibody is exposed to the target-first antibody complex to form a target-first antibody-second antibody tertiary complex. The complex is detected by the signal emitted by the reporter molecule. By “reporter molecule”, as used in the present specification, is meant a molecule which, by its chemical nature, provides an analytically identifiable signal which allows the detection of antigen-bound antibody. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, -galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker which was present in the sample. Alternately, fluorescent compounds, such as fluorescein and rhodamine, may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA, the fluorescent labeled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest. Immunofluorescence and EIA techniques are both very well established in the art. However, other reporter molecules, such as radioisotope, chemiluminescent or bioluminescent molecules, may also be employed.

Methods of the invention further include protocols which examine the presence and/or expression of mRNAs of the at least 1, 2, 3, 4, 5, 6, 7 or more of CMPK2, EPSTI1, HERC5, IFI27, IFI44, IFIT1, MX1, OAS1, OAS2, or OAS3 and combinations thereof in a sample. Methods for the evaluation of mRNAs in cells are well known and include, for example, hybridization assays using complementary DNA probes (such as in situ hybridization using labeled riboprobes specific for the one or more genes, Northern blot and related techniques) and various nucleic acid amplification assays (such as RT-PCR using complementary primers specific for one or more of the genes, and other amplification type detection methods, such as, for example, branched DNA, SISBA, TMA and the like).

Tissue or other samples from mammals can be conveniently assayed for mRNAs using Northern, dot blot or PCR analysis. For example, RT-PCR assays such as quantitative PCR assays are well known in the art. In some embodiments, the qPCR is performed on a Roche Cobas® system. In an illustrative embodiment of the invention, a method for detecting a target mRNA in a biological sample comprises producing cDNA from the sample by reverse transcription using at least one primer; amplifying the cDNA so produced using a target polynucleotide as sense and antisense primers to amplify target cDNAs therein. In addition, such methods can include one or more steps that allow one to determine the levels of target mRNA in a biological sample (e.g., by simultaneously examining the levels of a comparative control mRNA sequence of a “housekeeping” gene such as an actin family member or GAPDH). Optionally, the sequence of the amplified target cDNA can be determined.

Optional methods of the invention include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlate with increased or reduced clinical benefit of anti-angiogenic therapy may be arrayed on a solid support. Hybridization of a labeled probe with a particular array member indicates that the sample from which the probe was derived expresses that gene. Differential gene expression analysis of disease tissue can provide valuable information. Microarray technology utilizes nucleic acid hybridization techniques and computing technology to evaluate the mRNA expression profile of thousands of genes within a single experiment. (see, e.g., WO 01/75166 published Oct. 11, 2001; (see, for example, U.S. Pat. No. 5,700,637, U.S. Pat. No. 5,445,934, and U.S. Pat. No. 5,807,522, Lockart, Nature Biotechnology, 14:1675-1680 (1996); Cheung, V. G. et al., Nature Genetics 21(Suppl):15-19 (1999) for a discussion of array fabrication). DNA microarrays are miniature arrays containing gene fragments that are either synthesized directly onto or spotted onto glass or other substrates. Thousands of genes are usually represented in a single array. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. Currently two main types of DNA microarrays are being used: oligonucleotide (usually 25 to 70 mers) arrays and gene expression arrays containing PCR products prepared from cDNAs. In forming an array, oligonucleotides can be either prefabricated and spotted to the surface or directly synthesized on to the surface (in situ).

The Affymetrix GeneChip® system is a commercially available microarray system which comprises arrays fabricated by direct synthesis of oligonucleotides on a glass surface. Probe/Gene Arrays: Oligonucleotides, usually 25 mers, are directly synthesized onto a glass wafer by a combination of semiconductor-based photolithography and solid phase chemical synthesis technologies. Each array contains up to 400,000 different oligos and each oligo is present in millions of copies. Since oligonucleotide probes are synthesized in known locations on the array, the hybridization patterns and signal intensities can be interpreted in terms of gene identity and relative expression levels by the Affymetrix Microarray Suite software. Each gene is represented on the array by a series of different oligonucleotide probes. Each probe pair consists of a perfect match oligonucleotide and a mismatch oligonucleotide. The perfect match probe has a sequence exactly complimentary to the particular gene and thus measures the expression of the gene. The mismatch probe differs from the perfect match probe by a single base substitution at the center base position, disturbing the binding of the target gene transcript. This helps to determine the background and nonspecific hybridization that contributes to the signal measured for the perfect match oligo. The Microarray Suite software subtracts the hybridization intensities of the mismatch probes from those of the perfect match probes to determine the absolute or specific intensity value for each probe set. Probes are chosen based on current information from Genbank and other nucleotide repositories. The sequences are believed to recognize unique regions of the 3′ end of the gene. A GeneChip Hybridization Oven (“rotisserie” oven) is used to carry out the hybridization of up to 64 arrays at one time. The fluidics station performs washing and staining of the probe arrays. It is completely automated and contains four modules, with each module holding one probe array. Each module is controlled independently through Microarray Suite software using preprogrammed fluidics protocols. The scanner is a confocal laser fluorescence scanner which measures fluorescence intensity emitted by the labeled cRNA bound to the probe arrays. The computer workstation with Microarray Suite software controls the fluidics station and the scanner. Microarray Suite software can control up to eight fluidics stations using preprogrammed hybridization, wash, and stain protocols for the probe array. The software also acquires and converts hybridization intensity data into a presence/absence call for each gene using appropriate algorithms. Finally, the software detects changes in gene expression between experiments by comparison analysis and formats the output into .txt files, which can be used with other software programs for further data analysis.

Expression of a selected gene or biomarker in a tissue or cell sample may also be examined by way of functional or activity-based assays. For instance, if the biomarker is an enzyme, one may conduct assays known in the art to determine or detect the presence of the given enzymatic activity in the tissue or cell sample.

The IRG status (ISM^{lo} or ISM^{hi}) of a patient based on the test results may be provided in a report. The report may be in any form of written materials (e.g., in paper or digital form, or on internet) or oral presentation(s) (e.g., either in person (live) or as recorded). The report may further indicates to a health professional (e.g., a physician) that the patient may benefit from or is likely to respond to an interferon inhibitor treatment.

The kits of the invention have a number of embodiments. In certain embodiments, a kit comprises a container, a label on said container, and a composition contained within said container; wherein the composition includes one or more primary antibodies that bind to one or more target polypeptide sequences corresponding to an autoantibody to an ENA antigen, the label on the container indicating that the composition can be used to evaluate the presence of one or more target proteins in at least one type of mammalian cell, and instructions for using the antibodies for evaluating the presence of one or more target proteins in at least one type of mammalian cell. The kit can further comprise a set of instructions and materials for preparing a tissue sample and applying antibody and probe to the same section of a tissue sample. The kit may include both a primary and secondary antibody, wherein the secondary antibody is conjugated to a label, e.g., an enzymatic label.

In one embodiment, the subject has never been previously treated with drug(s), such as immunosuppressive agent(s), to treat the lupus and/or has never been previously treated with an antibody to a type I interferon. In another embodiment, the subject has been previously treated with drug(s) to treat the lupus and/or has been previously treated with such antibody. In another embodiment, the type I interferon antibody is the only medicament administered to the subject to treat the lupus. In another embodiment, the type I interferon antibody is one of the medicaments used to treat the lupus. In a further embodiment, the subject does not have rheumatoid arthritis. In a still further embodiment, the subject does not have multiple sclerosis. In yet another embodiment, the subject does not have an autoimmune disease other than lupus. For purposes of this lattermost statement, an “autoimmune disease” herein is a disease or disorder arising from and directed against an individual's own tissues or organs or a co-segregate or manifestation thereof or resulting condition therefrom. In one embodiment, it refers to a condition that results from, or is aggravated by, the production by B cells of antibodies that are reactive with normal body tissues and antigens. In other embodiments, the autoimmune disease is one that involves secretion of an autoantibody that is specific for an epitope from a self antigen (e.g. a nuclear antigen).

The antibody is administered by any suitable means, including parenteral, topical, subcutaneous, intraperitoneal, intrapulmonary, intranasal, and/or intralesional administration. Parenteral infusions include intramuscular, intravenous, intraarterial, intraperitoneal, or subcutaneous administration. Intrathecal administration is also contemplated. In addition, the antibody may suitably be administered by pulse infusion, e.g., with declining doses of the antibody. Preferably, the dosing is given intravenously or subcutaneously, and more preferably by intravenous infusion(s). Each exposure may be provided using the same or a different administration means. In one embodiment, each exposure is by intravenous administration. In another embodiment, each exposure is given by subcutaneous administration. In yet another embodiment, the exposures are given by both intravenous and subcutaneous administration.

In one embodiment, the type I interferon antibody is administered as a slow intravenous infusion rather than an intravenous push or bolus. For example, methylprednisolone (e.g., about 80-120 mg i.v., more preferably about 100 mg i.v.) is administered about 30 minutes prior to any infusion of the type I interferon antibody. The type I interferon antibody is, for example, infused through a dedicated line.

One may administer a second medicament with the type I interferon antibody, such as an anti-malarial agent, immunosuppressive agent, corticosteroid, NSAID, statin, cytotoxic agent, chemotherapeutic agent, cytokine, cytokine antagonist or antibody, growth factor, hormone, integrin, integrin antagonist, or antibody.

For instance, the antibody may be combined with a chemotherapeutic agent, an interferon class drug such as IFN-beta-1a (REBIF® and AVONEX®) or IFN-beta-1b (BETASERON®), an oligopeptide such as glatiramer acetate (COPAXONE®), a cytotoxic agent (such as mitoxantrone (NOVANTRONE®), methotrexate, cyclophosphamide, chlorambucil, and azathioprine), intravenous immunoglobulin (gamma globulin), lymphocyte-depleting therapy (e.g., mitoxantrone, cyclophosphamide, CAMPATH™ antibodies, anti-CD4, cladribine, total body irradiation, bone marrow transplantation), corticosteroid (e.g., methylprednisolone, prednisone such as low-dose prednisone, dexamethasone, or glucorticoid, e.g., via joint injection, including systemic corticosteroid therapy), non-lymphocyte-depleting immunosuppressive therapy (e.g., MMF or cyclosporine), cholesterol-lowering drug of the “statin” class (which includes cerivastatin (BAYCOL™), fluvastatin (LESCOL™), atorvastatin (LIPITOR™), lovastatin (MEVACOR™), pravastatin (PRAVACHOL™), and simvastatin (ZOCOR™)), estradiol, testosterone (optionally at elevated dosages; Stuve et al. Neurology 8:290-301 (2002)), hormone-replacement therapy, an anti-malarial drug such as, e.g., hydroxychloroquine, chloroquine, or quinacrine, treatment for symptoms secondary or related to lupus (e.g., spasticity, incontinence, pain, fatigue), a TNF inhibitor, DMARD, NSAID, anti-integrin antibody or antagonist, plasmapheresis, levothyroxine, cyclosporin A, somatastatin analogue, cytokine, anti-cytokine antagonist or antibody, anti-metabolite, immunosuppressive agent, rehabilitative surgery, radioiodine, thyroidectomy, another B-cell surface antagonist/antibody, etc.

More specific examples of such second medicaments, if the type I interferon antibody is called the first medicament, include a chemotherapeutic agent, cytotoxic agent, anti-integrin, anti-malarial drug such as, e.g., hydroxychloroquine, chloroquine, or quinacrine, gamma globulin, anti-CD4, cladribine, corticosteroid, MMF, cyclosporine, cholesterol-lowering drug of the statin class, estradiol, testosterone, hormone-replacement drug, TNF inhibitor, DMARD, NSAID, levothyroxine, cyclosporin A, somatastatin analogue, cytokine antagonist or cytokine-receptor antagonist, anti-metabolite, and/or an immunosuppressive agent.

These second medicaments are generally used in the same dosages and with administration routes as used hereinbefore or about from 1 to 99% of the heretofore-employed dosages. If such second medicaments are used at all, preferably, they are used in lower amounts than if the IFN antibody were not present, especially in subsequent dosings beyond the initial dosing with antibody, so as to eliminate or reduce side effects caused thereby.

Where a second medicament is administered in an effective amount with an antibody exposure, it may be administered with any exposure, for example, only with one exposure, or with more than one exposure. In one embodiment, the second medicament is administered with the initial exposure. In another embodiment, the second medicament is administered with the initial and second exposures. In a still further embodiment, the second medicament is administered with all exposures.

The combined administration includes co-administration (concurrent administration), using separate formulations or a single pharmaceutical formulation, and consecutive administration in either order, wherein preferably there is a time period while both (or all) active agents simultaneously exert their biological activities. In a preferred embodiment, after the initial exposure, the amount of such agent is reduced or eliminated so as to reduce the exposure of the subject to an agent with side effects such as prednisone and cyclophosphamide, especially when the agent is a corticosteroid. In another embodiment, the amount of the second medicament is not reduced or eliminated.

In one embodiment, an anti-malarial agent, or a chemotherapeutic agent is administered with the initial exposure, more preferably a corticosteroid, methotrexate, cyclophosphamide, hydroxychloroquine, chloroquine, quinacrine, azathioprine, mycophenolate mofetil, or 6-mercaptopurine. In another aspect, the immunosuppressive agent, anti-malarial agent, or chemotherapeutic agent is not administered with the subsequent exposure, or is administered in lower amounts than with the initial exposure. However, such agent is optionally administered with more than one exposure, including all exposures, in the same or similar amounts as with the initial exposure.

A corticosteroid such as methylprednisolone and/or prednisone may be administered to the subject before and/or with the type I interferon antibody. Additionally or alternatively, MMF is preferably administered with the initial antibody exposure, with concomitant administration of MMF and the corticosteroid being particularly preferred. Preferably, the MMF is given initially with the type I interferon antibody at about 1500 mg/day in divided doses (3×/day) and the subject is titrated up to a target dose of about 3 g/day in divided doses (3×/day) by about week 4, as tolerated. If reductions in dose are necessary, decreases will be allowed in about 250-500 mg decrements. In another aspect, cyclophosphamide may be administered to the subject with or without the corticosteroid at the initial antibody exposure. If cyclophosphamide is administered, it is preferably not administered with the second exposure or is administered with the second exposure but in lower amounts than are used with the initial exposure. Also preferred is wherein cyclophosphamide is not administered with third or later exposures.

### IV. Antibodies Directed to a Type I Interferon

Any type I interferon antibodies known in the art may be used in the methods described herein. For example, antibodies that bind multiple subtypes of interferon α are known in the art. Nonlimiting examples of these antibodies can be found in, for example, U.S. Pat. No. 7,087,726 (Genentech, Inc.) and U.S. Patent Appl. Publ. No. 2007/0014724 (Medarex). For example, in one embodiment, the anti-IFNα antibodies that may be used in the invention are any of those disclosed in Example 1 and Example 2 of U.S. Pat. No. 7,087,726, including, for example, those disclosed in Table 3 and Table 4, and/or those disclosed in the table entitled “Deposit of Material” on lines 25-54, column 56, and may comprise SEQ ID Nos 1, 2, 3, 4, 5, 6, 7, 8, 9, 10, 11, 12, 13, and/or 14 as disclosed in U.S. Pat. No. 7,087,726, and may further include chimeric, humanized, or human versions of these antibodies (if not already a chimeric, humanized, or human version), and may further include fragments or derivatives thereof.

In other embodiments, the anti-IFNα antibodies that may be used in the invention are any of those disclosed in U.S. Patent Appl. Publ. No. 2007/0014724, including, for example, those disclosed in Examples 1 and/or 11, and/or those disclosed in SEQ ID Nos. 1 through 30 as disclosed in U.S. Patent Appl. Publ. No. 2007/0014724, and may further include chimeric, humanized, or human versions of these antibodies (if not already a chimeric, humanized, or human version), and may further include fragments or derivatives thereof. For example, in some embodiments, the anti-IFNα antibodies may be the IgG1 antibody isotype of 13H5 and affinity matured variants thereof (See, e.g., U.S. Patent WO 2008/070135 (MedImmune)).

In some embodiments, the anti-human IFN-α monoclonal antibody binds to and neutralizes a biological activity of at least human IFN-α subtypes IFN-α1, IFN-α2, IFN-α4, IFN-α5, IFN-α8, IFN-α10, and IFN-α21. In a further aspect, the anti-human IFN-α monoclonal antibody binds to and neutralizes a biological activity of all human IFN-α subtypes. In certain embodiments, the human IFN-α monoclonal antibody can significantly reduce or eliminate a biological activity of the human IFN-α in question. In one embodiment, the human IFN-α monoclonal antibody is capable of neutralizing at least 60%, or at least 70%, preferably at least 75%, more preferably at least 80%, even more preferably at least 85%, still more preferably at least 90%, still more preferably at least 95%, most preferably at least 99% of a biological activity of the subject human IFN-α. In another embodiment, the human IFN-α biological activity-neutralizing monoclonal antibody does not neutralize the corresponding biological activity of human IFN-β. Binding and neutralization assays are well known in the art. See, e.g., U.S. Pat. No. 7,087,726 for assays useful in screening for antibodies having the desired binding and neutralization properties.

In certain embodiments, it may be desirable to alter the activity of specific subtypes, or combinations of subtypes, of interferon α. Accordingly, in one embodiment, the anti-interferon α antibodies selectively bind to at least interferon α subtype α2, which may include the α2a and/or α2b subtypes. In another embodiment, the anti-interferon α antibodies selectively bind to at least one interferon α subtype selected from α1, α2, α4, α5, α6, α7, α8, α10, α13, α14, α16, α17, or α21.

In one embodiment, the anti-interferon α antibodies selectively bind to at least interferon α subtype α1, α2, α4, α5, α6, α7, α8, α10, α13, α14, α16, α17, and α21. In one embodiment, the anti-interferon α antibodies selectively bind to at least interferon α subtype α1, α2, α4, α5, α6, α7, α8, α10, α13, α14, α16, and α17. In one embodiment, the anti-interferon α antibodies selectively bind to at least interferon α subtype α1, α2, α4, α5, α6, α7, α8, α10, α13, α14, and α16. In one embodiment, the anti-interferon α antibodies selectively bind to at least interferon α subtype α1, α2, α4, α5, α6, α7, α8, α10, α13, and α14. In one embodiment, the anti-interferon α antibodies selectively bind to at least interferon α subtype α1, α2, α4, α5, α6, α7, α8, α10, and α13. In one embodiment, the anti-interferon α antibodies selectively bind to at least interferon α subtype α1, α2, α4, α5, α6, α7, α8, and α10. In one embodiment, the anti-interferon α antibodies selectively bind to at least interferon α subtype α1, α2, α4, α5, α6, α7, and α8. In one embodiment, the anti-interferon α antibodies selectively bind to at least interferon α subtype α1, α2, α4, α5, α6, and α7. In one embodiment, the anti-interferon α antibodies selectively bind to at least interferon α subtype α1, α2, α4, α5, and α6.

In one embodiment, the anti-interferon α antibodies selectively bind to at least interferon α subtype α1, α2, α4, and α5. In one embodiment, the anti-interferon α antibodies selectively bind to at least interferon α subtype α1, α2, and α4. In one embodiment, the anti-interferon α antibodies selectively bind to at least interferon α subtype α1 and α2. In one embodiment, the anti-interferon α antibodies selectively bind to at least interferon α subtype and α1.

In one embodiment, the anti-interferon α antibodies selectively bind to at least interferon α subtype α2, α4, α5, α6, α7, α8, α10, α13, α14, α16, α17, and α21. In one embodiment, the anti-interferon α antibodies selectively bind to at least interferon α subtype α4, α5, α6, α7, α8, α10, α13, α14, α16, α17, and α21. In one embodiment, the anti-interferon α antibodies selectively bind to at least interferon α subtype α5, α6, α7, α8, α10, α13, α14, α16, α17, and α21. In one embodiment, the anti-interferon α antibodies selectively bind to at least interferon α subtype α6, α7, α8, α10, α13, α14, α16, α17, and α21. In one embodiment, the anti-interferon α antibodies selectively bind to at least interferon α subtype α7, α8, α10, α13, α14, α16, α17, and α21. In one embodiment, the anti-interferon α antibodies selectively bind to at least interferon α subtype α8, α10, α13, α14, α16, α17, and α21. In one embodiment, the anti-interferon α antibodies selectively bind to at least interferon α subtype α10, α13, α14, α16, α17, and α21. In one embodiment, the anti-interferon α antibodies selectively bind to at least interferon α subtype α13, α14, α16, α17, and α21. In one embodiment, the anti-interferon α antibodies selectively bind to at least interferon α subtype α14, α16, α17, and α21. In one embodiment, the anti-interferon α antibodies selectively bind to at least interferon α subtype α16, α17, and α21. In one embodiment, the anti-interferon α antibodies selectively bind to at least interferon α subtype α17 and α21. In one embodiment, the anti-interferon α antibodies selectively bind to at least interferon α subtype α21.

In one embodiment, the anti-interferon α antibodies selectively bind to at least interferon α subtype α1, α4, α5, α6, α7, α8, α10, α13, α14, α16, α17, and α21. In one embodiment, the anti-interferon α antibodies selectively bind to at least interferon α subtype α1, α2, α5, α6, α7, α8, α10, α13, α14, α16, α17, and α21. In one embodiment, the anti-interferon α antibodies selectively bind to at least interferon α subtype α1, α2, α4, α6, α7, α8, α10, α13, α14, α16, α17, and α21. In one embodiment, the anti-interferon α antibodies selectively bind to at least interferon α subtype α1, α2, α4, α5, α7, α8, α10, α13, α14, α16, α17, and α21. In one embodiment, the anti-interferon α antibodies selectively bind to at least interferon α subtype α1, α2, α4, α5, α6, α8, α10, α13, α14, α16, α17, and α21. In one embodiment, the anti-interferon α antibodies selectively bind to at least interferon α subtype α1, α2, α4, α5, α6, α7, α10, α13, α14, α16, α17, and α21. In one embodiment, the anti-interferon α antibodies selectively bind to at least interferon α subtype α1, α2, α4, α5, α6, α7, α8, α13, α14, α16, α17, and α21. In one embodiment, the anti-interferon α antibodies selectively bind to at least interferon α subtype α1, α2, α4, α5, α6, α7, α8, α10, α14, α16, α17, and α21. In one embodiment, the anti-interferon α antibodies selectively bind to at least interferon α subtype α1, α2, α4, α5, α6, α7, α8, α10, α13, α16, α17, and α21. In one embodiment, the anti-interferon α antibodies selectively bind to at least interferon α subtype α1, α2, α4, α5, α6, α7, α8, α10, α13, α14, α17, and α21. In one embodiment, the anti-interferon α antibodies selectively bind to at least interferon α subtype α1, α2, α4, α5, α6, α7, α8, α10, α13, α14, α16, and α21.

In one embodiment, the anti-interferon α antibodies selectively bind to at least interferon α subtype α1, α4, α5, α6, α7, α8, α10, α13, α14, α16, α17, and α21. In one embodiment, the anti-interferon α antibodies selectively bind to at least interferon α subtype α1, α5, α6, α7, α8, α10, α13, α14, α16, α17, and α21. In one embodiment, the anti-interferon α antibodies selectively bind to at least interferon α subtype α1, α6, α7, α8, α10, α13, α14, α16, α17, and α21. In one embodiment, the anti-interferon α antibodies selectively bind to at least interferon α subtype α1, α7, α8, α10, α13, α14, α16, α17, and α21. In one embodiment, the anti-interferon α antibodies selectively bind to at least interferon α subtype α1, α8, α10, α13, α14, α16, α17, and α21. In one embodiment, the anti-interferon α antibodies selectively bind to at least interferon α subtype α1, α10, α13, α14, α16, α17, and α21. In one embodiment, the anti-interferon α antibodies selectively bind to at least interferon α subtype α1, α13, α14, α16, α17, and α21. In one embodiment, the anti-interferon α antibodies selectively bind to at least interferon α subtype α1, α14, α16, α17, and α21. In one embodiment, the anti-interferon α antibodies selectively bind to at least interferon α subtype α1, α16, α17, and α21. In one embodiment, the anti-interferon α antibodies selectively bind to at least interferon α subtype α1, α17, and α21. In one embodiment, the anti-interferon α antibodies selectively bind to at least interferon α subtype α1 and α21.

In one embodiment, the anti-interferon α antibodies selectively bind to at least interferon α subtype α1, α2, α6, α7, α8, α10, α13, α14, α16, α17, and α21. In one embodiment, the anti-interferon α antibodies selectively bind to at least interferon α subtype α1, α2, α7, α8, α10, α13, α14, α16, α17, and α21. In one embodiment, the anti-interferon α antibodies selectively bind to at least interferon α subtype α1, α2, α8, α10, α13, α14, α16, α17, and α21. In one embodiment, the anti-interferon α antibodies selectively bind to at least interferon α subtype α1, α2, α10, α13, α14, α16, α17, and α21. In one embodiment, the anti-interferon α antibodies selectively bind to at least interferon α subtype α1, α2, α13, α14, α16, α17, and α21. In one embodiment, the anti-interferon α antibodies selectively bind to at least interferon α subtype α1, α2, α14, α16, α17, and α21. In one embodiment, the anti-interferon α antibodies selectively bind to at least interferon α subtype α1, α2, α16, α17, and α21. In one embodiment, the anti-interferon α antibodies selectively bind to at least interferon α subtype α1, α2, α17, and α21. In one embodiment, the anti-interferon α antibodies selectively bind to at least interferon α subtype α1, α2, and α21. In one embodiment, the anti-interferon α antibodies selectively bind to at least interferon α subtype α1 and α2.

In one embodiment, the anti-interferon α antibodies selectively bind to at least interferon α subtype α1, α2, α4, α6, α7, α8, α10, α13, α14, α16, α17, and α21. In one embodiment, the anti-interferon α antibodies selectively bind to at least interferon α subtype α1, α2, α4, α7, α8, α10, α13, α14, α16, α17, and α21. In one embodiment, the anti-interferon α antibodies selectively bind to at least interferon α subtype α1, α2, α4, α8, α10, α13, α14, α16, α17, and α21. In one embodiment, the anti-interferon α antibodies selectively bind to at least interferon α subtype α1, α2, α4, α10, α13, α14, α16, α17, and α21. In one embodiment, the anti-interferon α antibodies selectively bind to at least interferon α subtype α1, α2, α4, α13, α14, α16, α17, and α21. In one embodiment, the anti-interferon α antibodies selectively bind to at least interferon α subtype α1, α2, α4, α14, α16, α17, and α21. In one embodiment, the anti-interferon α antibodies selectively bind to at least interferon α subtype α1, α2, α4, α16, α17, and α21. In one embodiment, the anti-interferon α antibodies selectively bind to at least interferon α subtype α1, α2, α4, α17, and α21. In one embodiment, the anti-interferon α antibodies selectively bind to at least interferon α subtype α1, α2, α4, and α21. In one embodiment, the anti-interferon α antibodies selectively bind to at least interferon α subtype α1, α2, and α4.

In one embodiment, the anti-interferon α antibodies selectively bind to at least interferon α subtype α1, α2, α4, α5, α7, α8, α10, α13, α14, α16, α17, and α21. In one embodiment, the anti-interferon α antibodies selectively bind to at least interferon α subtype α1, α2, α4, α5, α8, α10, α13, α14, α16, α17, and α21. In one embodiment, the anti-interferon α antibodies selectively bind to at least interferon α subtype α1, α2, α4, α5, α10, α13, α14, α16, α17, and α21. In one embodiment, the anti-interferon α antibodies selectively bind to at least interferon α subtype α1, α2, α4, α5, α13, α14, α16, α17, and α21. In one embodiment, the anti-interferon α antibodies selectively bind to at least interferon α subtype α1, α2, α4, α5, α14, α16, α17, and α21. In one embodiment, the anti-interferon α antibodies selectively bind to at least interferon α subtype α1, α2, α4, α5, α16, α17, and α21. In one embodiment, the anti-interferon α antibodies selectively bind to at least interferon α subtype α1, α2, α4, α5, α17, and α21. In one embodiment, the anti-interferon α antibodies selectively bind to at least interferon α subtype α1, α2, α4, α5, and α21. In one embodiment, the anti-interferon α antibodies selectively bind to at least interferon α subtype α1, α2, α4, and α5.

In one embodiment, the anti-interferon α antibodies selectively bind to at least interferon α subtype α1, α2, α4, α5, α6, α8, α10, α13, α14, α16, α17, and α21. In one embodiment, the anti-interferon α antibodies selectively bind to at least interferon α subtype α1, α2, α4, α5, α6, α10, α13, α14, α16, α17, and α21. In one embodiment, the anti-interferon α antibodies selectively bind to at least interferon α subtype α1, α2, α4, α5, α6, α13, α14, α16, α17, and α21. In one embodiment, the anti-interferon α antibodies selectively bind to at least interferon α subtype α1, α2, α4, α5, α6, α14, α16, α17, and α21.

In one embodiment, the anti-interferon α antibodies selectively bind to at least interferon α subtype α1, α2, α4, α5, α6, α16, α17, and α21. In one embodiment, the anti-interferon α antibodies selectively bind to at least interferon α subtype α1, α2, α4, α5, α6, α17, and α21.

In one embodiment, the anti-interferon α antibodies selectively bind to at least interferon α subtype α1, α2, α4, α5, α6, and α21. In one embodiment, the anti-interferon α antibodies selectively bind to at least interferon α subtype α1, α2, α4, α5, and α6.

In one embodiment, the anti-interferon α antibodies selectively bind to at least interferon α subtype α1, α2, α4, α5, α6, α7, α10, α13, α14, α16, α17, and α21. In one embodiment, the anti-interferon α antibodies selectively bind to at least interferon α subtype α1, α2, α4, α5, α6, α7, α13, α14, α16, α17, and α21. In one embodiment, the anti-interferon α antibodies selectively bind to at least interferon α subtype α1, α2, α4, α5, α6, α7, α14, α16, α17, and α21. In one embodiment, the anti-interferon α antibodies selectively bind to at least interferon α subtype α1, α2, α4, α5, α6, α7, α16, α17, and α21. In one embodiment, the anti-interferon α antibodies selectively bind to at least interferon α subtype α1, α2, α4, α5, α6, α7, α17, and α21. In one embodiment, the anti-interferon α antibodies selectively bind to at least interferon α subtype α1, α2, α4, α5, α6, α7, and α21. In one embodiment, the anti-interferon α antibodies selectively bind to at least interferon α subtype α1, α2, α4, α5, α6, and α7.

In one embodiment, the anti-interferon α antibodies selectively bind to at least interferon α subtype α1, α2, α4, α5, α6, α7, α8, α13, α14, α16, α17, and α21. In one embodiment, the anti-interferon α antibodies selectively bind to at least interferon α subtype α1, α2, α4, α5, α6, α7, α8, α14, α16, α17, and α21. In one embodiment, the anti-interferon α antibodies selectively bind to at least interferon α subtype α1, α2, α4, α5, α6, α7, α8, α16, α17, and α21. In one embodiment, the anti-interferon α antibodies selectively bind to at least interferon α subtype α1, α2, α4, α5, α6, α7, α8, α17, and α21. In one embodiment, the anti-interferon α antibodies selectively bind to at least interferon α subtype α1, α2, α4, α5, α6, α7, α8, and α21. In one embodiment, the anti-interferon α antibodies selectively bind to at least interferon α subtype α1, α2, α4, α5, α6, α7, and α8.

In one embodiment, the anti-interferon α antibodies selectively bind to at least interferon α subtype α1, α2, α4, α5, α6, α7, α8, α10, α14, α16, α17, and α21. In one embodiment, the anti-interferon α antibodies selectively bind to at least interferon α subtype α1, α2, α4, α5, α6, α7, α8, α10, α16, α17, and α21. In one embodiment the anti-interferon α antibodies selectively bind to at least interferon α subtype α1, α2, α4, α5, α6, α7, α8, α10, α17, and α21. In one embodiment, the anti-interferon α antibodies selectively bind to at least interferon α subtype α1, α2, α4, α5, α6, α7, α8, α10, and α21. In one embodiment, the anti-interferon α antibodies selectively bind to at least interferon α subtype α1, α2, α4, α5, α6, α7, α8, and α10.

In one embodiment, the anti-interferon α antibodies selectively bind to at least interferon α subtype α1, α2, α4, α5, α6, α7, α8, α10, α13, α16, α17, and α21. In one embodiment, the anti-interferon α antibodies selectively bind to at least interferon α subtype α1, α2, α4, α5, α6, α7, α8, α10, α13, α17, and α21. In one embodiment, the anti-interferon α antibodies selectively bind to at least interferon α subtype α1, α2, α4, α5, α6, α7, α8, α10, α13, and α21. In one embodiment, the anti-interferon α antibodies selectively bind to at least interferon α subtype α1, α2, α4, α5, α6, α7, α8, α10, and α13.

In one embodiment, the anti-interferon α antibodies selectively bind to at least interferon α subtype α1, α2, α4, α5, α6, α7, α8, α10, α13, α14, α16, α17, and α21. In one embodiment, the anti-interferon α antibodies selectively bind to at least interferon α subtype α1, α2, α4, α5, α6, α7, α8, α10, α13, α14, α17, and α21. In one embodiment, the anti-interferon α antibodies selectively bind to at least interferon α subtype α1, α2, α4, α5, α6, α7, α8, α10, α13, α14, and α21. In one embodiment, the anti-interferon α antibodies selectively bind to at least interferon α subtype α1, α2, α4, α5, α6, α7, α8, α10, α13, and α14. In one embodiment, the anti-interferon α antibodies selectively bind to at least interferon α subtype α1, α2, α4, α5, α6, α7, α8, α10, α13, α14, α16, and α21. In one embodiment, the anti-interferon α antibodies selectively bind to at least interferon α subtype α1, α2, α4, α5, α6, α7, α8, α10, α13, α14, and α16.

In one embodiment, the anti-interferon α antibodies selectively bind to at least interferon α subtype α1, α2, α4, α5, α8, α10, and α21.

In one embodiment, the anti-interferon α antibodies selectively bind to at least interferon α subtype α1. In one embodiment, the anti-interferon α antibodies selectively bind to at least interferon α subtype α2. In one embodiment, the anti-interferon α antibodies selectively bind to at least interferon α subtype α4. In one embodiment, the anti-interferon α antibodies selectively bind to at least interferon α subtype α5. In one embodiment, the anti-interferon α antibodies selectively bind to at least interferon α subtype α6. In one embodiment, the anti-interferon α antibodies selectively bind to at least interferon α subtype α7. In one embodiment, the anti-interferon α antibodies selectively bind to at least interferon α subtype α8. In one embodiment, the anti-interferon α antibodies selectively bind to at least interferon α subtype α10. In one embodiment, the anti-interferon α antibodies selectively bind to at least interferon α subtype α13. In one embodiment, the anti-interferon α antibodies selectively bind to at least interferon α subtype α14. In one embodiment, the anti-interferon α antibodies selectively bind to at least interferon α subtype α16. In one embodiment, the anti-interferon α antibodies selectively bind to at least interferon α subtype α17. In one embodiment, the anti-interferon α antibodies selectively bind to at least interferon α subtype α21.

In certain embodiments, it will be desirable to have the anti-IFNα antibodies of the invention selectively bind and neutralize specific IFNα subtypes, or combinations of IFNα subtypes. Interferon neutralization assays are well known in the art.

In some embodiments, the ability of an anti-IFN-α antibody to block the binding of an IFN-α to receptor is defined as the property or capacity of a certain concentration of the antibody to reduce or eliminate the binding of IFN-α to IFNAR2 in a competition binding assay, as compared to the effect of an equivalent concentration of irrelevant control antibody on IFN-α binding to IFNAR2 in the assay. Preferably, the blocking anti-IFN-α antibody reduces the binding of IFN-α to IFNAR2 by at least about 50%, or at least about 55%, or at least about 60%, or at least about 65%, or at least about 70%, or at least about 75%, or at least about 80%, or at least about 85%, or at least about 90%, or at least about 95%, or at least about 99%, as compared to the irrelevant control antibody.

Accordingly, in one embodiment, the anti-interferon α antibodies selectively bind to and neutralize at least interferon α subtype α2, which may include the α2a and/or α2b subtypes. In another embodiment, the anti-interferon α antibodies selectively bind to and neutralize at least interferon α subtype α1, α2, α4, α5, α6, α7, α8, α10, α13, α14, α16, α17, or α21.

In one embodiment, the anti-interferon α antibodies selectively bind to and neutralize at least interferon α subtype α1, α2, α4, α5, α6, α7, α8, α10, α13, α14, α16, α17, and α21. In one embodiment, the anti-interferon α antibodies selectively bind to and neutralize at least interferon α subtype α1, α2, α4, α5, α6, α7, α8, α10, α13, α14, α16, and α17. In one embodiment, the anti-interferon α antibodies selectively bind to and neutralize at least interferon α subtype α1, α2, α4, α5, α6, α7, α8, α10, α13, α14, and α16. In one embodiment, the anti-interferon α antibodies selectively bind to and neutralize at least interferon α subtype α1, α2, α4, α5, α6, α7, α8, α10, α13, and α14. In one embodiment, the anti-interferon α antibodies selectively bind to and neutralize at least interferon α subtype α1, α2, α4, α5, α6, α7, α8, α10, and α13. In one embodiment, the anti-interferon α antibodies selectively bind to and neutralize at least interferon α subtype α1, α2, α4, α5, α6, α7, α8, and α10. In one embodiment, the anti-interferon α antibodies selectively bind to and neutralize at least interferon α subtype α1, α2, α4, α5, α6, α7, and α8. In one embodiment, the anti-interferon α antibodies selectively bind to and neutralize at least interferon α subtype α1, α2, α4, α5, α6, and α7. In one embodiment, the anti-interferon α antibodies selectively bind to and neutralize at least interferon α subtype α1, α2, α4, α5, and α6. In one embodiment, the anti-interferon α antibodies selectively bind to and neutralize at least interferon α subtype α1, α2, α4, and α5. In one embodiment, the anti-interferon α antibodies selectively bind to and neutralize at least interferon α subtype α1, α2, and α4. In one embodiment, the anti-interferon α antibodies selectively bind to and neutralize at least interferon α subtype α1 and α2. In one embodiment, the anti-interferon α antibodies selectively bind to and neutralize at least interferon α subtype and α1.

In one embodiment, the anti-interferon α antibodies selectively bind to and neutralize at least interferon α subtype α2, α4, α5, α6, α7, α8, α10, α13, α14, α16, α17, and α21. In one embodiment, the anti-interferon α antibodies selectively bind to and neutralize at least interferon α subtype α4, α5, α6, α7, α8, α10, α13, α14, α16, α17, and α21. In one embodiment, the anti-interferon α antibodies selectively bind to and neutralize at least interferon α subtype α5, α6, α7, α8, α10, α13, α14, α16, α17, and α21. In one embodiment, the anti-interferon α antibodies selectively bind to and neutralize at least interferon α subtype α6, α7, α8, α10, α13, α14, α16, α17, and α21. In one embodiment, the anti-interferon α antibodies selectively bind to and neutralize at least interferon α subtype α7, α8, α10, α13, α14, α16, α17, and α21. In one embodiment, the anti-interferon α antibodies selectively bind to and neutralize at least interferon α subtype α8, α10, α13, α14, α16, α17, and α21. In one embodiment, the anti-interferon α antibodies selectively bind to and neutralize at least interferon α subtype α10, α13, α14, α16, α17, and α21. In one embodiment, the anti-interferon α antibodies selectively bind to and neutralize at least interferon α subtype α13, α14, α16, α17, and α21. In one embodiment, the anti-interferon α antibodies selectively bind to and neutralize at least interferon α subtype α14, α16, α17, and α21. In one embodiment, the anti-interferon α antibodies selectively bind to and neutralize at least interferon α subtype α16, α17, and α21. In one embodiment, the anti-interferon α antibodies selectively bind to and neutralize at least interferon α subtype α17 and α21. In one embodiment, the anti-interferon α antibodies selectively bind to and neutralize at least interferon α subtype α21.

In one embodiment, the anti-interferon α antibodies selectively bind to and neutralize at least interferon α subtype α1, α4, α5, α6, α7, α8, α10, α13, α14, α16, α17, and α21. In one embodiment, the anti-interferon α antibodies selectively bind to and neutralize at least interferon α subtype α1, α2, α5, α6, α7, α8, α10, α13, α14, α16, α17, and α21. In one embodiment, the anti-interferon α antibodies selectively bind to and neutralize at least interferon α subtype α1, α2, α4, α6, α7, α8, α10, α13, α14, α16, α17, and α21. In one embodiment, the anti-interferon α antibodies selectively bind to and neutralize at least interferon α subtype α1, α2, α4, α5, α7, α8, α10, α13, α14, α16, α17, and α21. In one embodiment, the anti-interferon α antibodies selectively bind to and neutralize at least interferon α subtype α1, α2, α4, α5, α6, α8, α10, α13, α14, α16, α17, and α21. In one embodiment, the anti-interferon α antibodies selectively bind to and neutralize at least interferon α subtype α1, α2, α4, α5, α6, α7, α10, α13, α14, α16, α17, and α21. In one embodiment, the anti-interferon α antibodies selectively bind to and neutralize at least interferon α subtype α1, α2, α4, α5, α6, α7, α8, α13, α14, α16, α17, and α21. In one embodiment, the anti-interferon α antibodies selectively bind to and neutralize at least interferon α subtype α1, α2, α4, α5, α6, α7, α8, α10, α14, α16, α17, and α21. In one embodiment, the anti-interferon α antibodies selectively bind to and neutralize at least interferon α subtype α1, α2, α4, α5, α6, α7, α8, α10, α13, α16, α17, and α21. In one embodiment, the anti-interferon α antibodies selectively bind to and neutralize at least interferon α subtype α1, α2, α4, α5, α6, α7, α8, α10, α13, α14, α17, and α21. In one embodiment, the anti-interferon α antibodies selectively bind to and neutralize at least interferon α subtype α1, α2, α4, α5, α6, α7, α8, α10, α13, α14, α16, and α21.

In one embodiment, the anti-interferon α antibodies selectively bind to and neutralize at least interferon α subtype α1, α4, α5, α6, α7, α8, α10, α13, α14, α16, α17, and α21. In one embodiment, the anti-interferon α antibodies selectively bind to and neutralize at least interferon α subtype α1, α5, α6, α7, α8, α10, α13, α14, α16, α17, and α21. In one embodiment, the anti-interferon α antibodies selectively bind to and neutralize at least interferon α subtype α1, α6, α7, α8, α10, α13, α14, α16, α17, and α21. In one embodiment, the anti-interferon α antibodies selectively bind to and neutralize at least interferon α subtype α1, α7, α8, α10, α13, α14, α16, α17, and α21. In one embodiment, the anti-interferon α antibodies selectively bind to and neutralize at least interferon α subtype α1, α8, α10, α13, α14, α16, α17, and α21. In one embodiment, the anti-interferon α antibodies selectively bind to and neutralize at least interferon α subtype α1, α10, α13, α14, α16, α17, and α21. In one embodiment, the anti-interferon α antibodies selectively bind to and neutralize at least interferon α subtype α1, α13, α14, α16, α17, and α21. In one embodiment, the anti-interferon α antibodies selectively bind to and neutralize at least interferon α subtype α1, α14, α16, α17, and α21. In one embodiment, the anti-interferon α antibodies selectively bind to and neutralize at least interferon α subtype α1, α16, α17, and α21. In one embodiment, the anti-interferon α antibodies selectively bind to and neutralize at least interferon α subtype α1, α17, and α21. In one embodiment, the anti-interferon α antibodies selectively bind to and neutralize at least interferon α subtype α1 and α21.

In one embodiment, the anti-interferon α antibodies selectively bind to and neutralize at least interferon α subtype α1, α2, α6, α7, α8, α10, α13, α14, α16, α17, and α21. In one embodiment, the anti-interferon α antibodies selectively bind to and neutralize at least interferon α subtype α1, α2, α7, α8, α10, α13, α14, α16, α17, and α21. In one embodiment, the anti-interferon α antibodies selectively bind to and neutralize at least interferon α subtype α1, α2, α8, α10, α13, α14, α16, α17, and α21. In one embodiment, the anti-interferon α antibodies selectively bind to and neutralize at least interferon α subtype α1, α2, α10, α13, α14, α16, α17, and α21. In one embodiment, the anti-interferon α antibodies selectively bind to and neutralize at least interferon α subtype α1, α2, α13, α14, α16, α17, and α21. In one embodiment, the anti-interferon α antibodies selectively bind to and neutralize at least interferon α subtype α1, α2, α14, α16, α17, and α21. In one embodiment, the anti-interferon α antibodies selectively bind to and neutralize at least interferon α subtype α1, α2, α16, α17, and α21. In one embodiment, the anti-interferon α antibodies selectively bind to and neutralize at least interferon α subtype α1, α2, α17, and α21. In one embodiment, the anti-interferon α antibodies selectively bind to and neutralize at least interferon α subtype α1, α2, and α21. In one embodiment, the anti-interferon α antibodies selectively bind to and neutralize at least interferon α subtype α1 and α2.

In one embodiment, the anti-interferon α antibodies selectively bind to and neutralize at least interferon α subtype α1, α2, α4, α6, α7, α8, α10, α13, α14, α16, α17, and α21. In one embodiment, the anti-interferon α antibodies selectively bind to and neutralize at least interferon α subtype α1, α2, α4, α7, α8, α10, α13, α14, α16, α17, and α21. In one embodiment, the anti-interferon α antibodies selectively bind to and neutralize at least interferon α subtype α1, α2, α4, α8, α10, α13, α14, α16, α17, and α21. In one embodiment, the anti-interferon α antibodies selectively bind to and neutralize at least interferon α subtype α1, α2, α4, α10, α13, α14, α16, α17, and α21. In one embodiment, the anti-interferon α antibodies selectively bind to and neutralize at least interferon α subtype α1, α2, α4, α13, α14, α16, α17, and α21. In one embodiment, the anti-interferon α antibodies selectively bind to and neutralize at least interferon α subtype α1, α2, α4, α14, α16, α17, and α21. In one embodiment, the anti-interferon α antibodies selectively bind to and neutralize at least interferon α subtype α1, α2, α4, α16, α17, and α21. In one embodiment, the anti-interferon α antibodies selectively bind to and neutralize at least interferon α subtype α1, α2, α4, α17, and α21. In one embodiment, the anti-interferon α antibodies selectively bind to and neutralize at least interferon α subtype α1, α2, α4, and α21. In one embodiment, the anti-interferon α antibodies selectively bind to and neutralize at least interferon α subtype α1, α2, and α4.

In one embodiment, the anti-interferon α antibodies selectively bind to and neutralize at least interferon α subtype α1, α2, α4, α5, α7, α8, α10, α13, α14, α16, α17, and α21. In one embodiment, the anti-interferon α antibodies selectively bind to and neutralize at least interferon α subtype α1, α2, α4, α5, α8, α10, α13, α14, α16, α17, and α21. In one embodiment, the anti-interferon α antibodies selectively bind to and neutralize at least interferon α subtype α1, α2, α4, α5, α10, α13, α14, α16, α17, and α21. In one embodiment, the anti-interferon α antibodies selectively bind to and neutralize at least interferon α subtype α1, α2, α4, α5, α13, α14, α16, α17, and α21. In one embodiment, the anti-interferon α antibodies selectively bind to and neutralize at least interferon α subtype α1, α2, α4, α5, α14, α16, α17, and \acute{α}21. In one embodiment, the anti-interferon α antibodies selectively bind to and neutralize at least interferon α subtype α1, α2, α4, α5, α16, α17, and α21. In one embodiment, the anti-interferon α antibodies selectively bind to and neutralize at least interferon α subtype α1, α2, α4, α5, α17, and α21. In one embodiment, the anti-interferon α antibodies selectively bind to and neutralize at least interferon α subtype α1, α2, α4, α5, and α21. In one embodiment, the anti-interferon α antibodies selectively bind to and neutralize at least interferon α subtype α1, α2, α4, and α5.

In one embodiment, the anti-interferon α antibodies selectively bind to and neutralize at least interferon α subtype α1, α2, α4, α5, α6, α8, α10, α13, α14, α16, α17, and α21. In one embodiment, the anti-interferon α antibodies selectively bind to and neutralize at least interferon α subtype α1, α2, α4, α5, α6, α10, α13, α14, α16, α17, and α21. In one embodiment, the anti-interferon α antibodies selectively bind to and neutralize at least interferon α subtype α1, α2, α4, α5, α6, α13, α14, α16, α17, and α21. In one embodiment, the anti-interferon α antibodies selectively bind to and neutralize at least interferon α subtype α1, α2, α4, α5, α6, α14, α1, α17, and α21. In one embodiment, the anti-interferon α antibodies selectively bind to and neutralize at least interferon α subtype α1, α2, α4, α5, α6, α16, α17, and α21. In one embodiment, the anti-interferon α antibodies selectively bind to and neutralize at least interferon α subtype α1, α2, α4, α5, α6, α17, and α21. In one embodiment, the anti-interferon α antibodies selectively bind to and neutralize at least interferon α subtype α1, α2, α4, α5, α6, and α21. In one embodiment, the anti-interferon α antibodies selectively bind to and neutralize at least interferon α subtype α1, α2, α4, α5, and α6.

In one embodiment, the anti-interferon α antibodies selectively bind to and neutralize at least interferon α subtype α1, α2, α4, α5, α6, α7, α10, α13, α14, α16, α17, and α21. In one embodiment, the anti-interferon α antibodies selectively bind to and neutralize at least interferon α subtype α1, α2, α4, α5, α6, α7, α13, α14, α16, α17, and α21. In one embodiment, the anti-interferon α antibodies selectively bind to and neutralize at least interferon α subtype α1, α2, α4, α5, α6, α7, α14, α16, α17, and α21. In one embodiment, the anti-interferon α antibodies selectively bind to and neutralize at least interferon α subtype α1, α2, α4, α5, α6, α7, α16, α17, and α21. In one embodiment, the anti-interferon α antibodies selectively bind to and neutralize at least interferon α subtype α1, α2, α4, α5, α6, α7, α17, and α21. In one embodiment, the anti-interferon α antibodies selectively bind to and neutralize at least interferon α subtype α1, α2, α4, α5, α6, α7, and α21. In one embodiment, the anti-interferon α antibodies selectively bind to and neutralize at least interferon α subtype α1, α2, α4, α5, α6, and α7.

In one embodiment, the anti-interferon α antibodies selectively bind to and neutralize at least interferon α subtype α1, α2, α4, α5, α6, α7, α8, α13, α14, α16, α17, and α21. In one embodiment, the anti-interferon α antibodies selectively bind to and neutralize at least interferon α subtype α1, α2, α4, α5, α6, α7, α8, α14, α16, α17, and α21. In one embodiment, the anti-interferon α antibodies selectively bind to and neutralize at least interferon α subtype α1, α2. α4, α5, α6, α7, α8, α16, α17, and α21. In one embodiment, the anti-interferon α antibodies selectively bind to and neutralize at least interferon α subtype α1, α2, α4, α5, α6, α7, α8, α17, and α21. In one embodiment, the anti-interferon α antibodies selectively bind to and neutralize at least interferon α subtype α1, α2, α4, α5, α6, α7, α8, and α21. In one embodiment, the anti-interferon α antibodies selectively bind to and neutralize at least interferon α subtype α1, α2, α4, α5, α6, α7, and α8.

In one embodiment, the anti-interferon α antibodies selectively bind to and neutralize at least interferon α subtype α1, α2, α4, α5, α6, α7, α8, α10, α14, α16, α17, and α21. In one embodiment, the anti-interferon α antibodies selectively bind to and neutralize at least interferon α subtype α1, α2, α4, α5, α6, α7, α8, α10, α16, α17, and α21. In one embodiment, the anti-interferon α antibodies selectively bind to and neutralize at least interferon α subtype α1, α2, α4, α5, α6, α7, α8, α10, α17, and α21. In one embodiment, the anti-interferon α antibodies selectively bind to and neutralize at least interferon α subtype α1, α2, α4, α5, α6, α7, α8, α10, and α21. In one embodiment, the anti-interferon α antibodies selectively bind to and neutralize at least interferon α subtype α1, α2, α4, α5, α6, α7, α8, and α10.

In one embodiment, the anti-interferon α antibodies selectively bind to and neutralize at least interferon α subtype α1, α2, α4, α5, α6, α7, α8, α10, α13, α16, α17, and α21. In one embodiment, the anti-interferon α antibodies selectively bind to and neutralize at least interferon α subtype α1, α2, α4, α5, α6, α7, α8, α10, α13, α17, and α21. In one embodiment, the anti-interferon α antibodies selectively bind to and neutralize at least interferon α subtype α1, α2, α4, α5, α6, α7, α8, α10, α13, and α21. In one embodiment, the anti-interferon α antibodies selectively bind to and neutralize at least interferon α subtype α1, α2, α4, α5, α6, α7, α8, α10, and α13.

In one embodiment, the anti-interferon α antibodies selectively bind to and neutralize at least interferon α subtype α1, α2, α4, α5, α6, α7, α8, α10, α13, α14, α16, α17, and α21. In one embodiment, the anti-interferon α antibodies selectively bind to and neutralize at least interferon α subtype α1, α2, α4, α5, α6, α7, α8, α10, α13, α14, α17, and α21. In one embodiment, the anti-interferon α antibodies selectively bind to and neutralize at least interferon α subtype α1, α2, α4, α5, α6, α7, α8, α10, α13, α14, and α21. In one embodiment, the anti-interferon α antibodies selectively bind to and neutralize at least interferon α subtype α1, α2, α4, α5, α6, α7, α8, α10, α13, and α14. In one embodiment, the anti-interferon α antibodies selectively bind to and neutralize at least interferon α subtype α1, α2, α4, α5, α6, α7, α8, α10, α13, α14, α16, and α21. In one embodiment, the anti-interferon α antibodies selectively bind to and neutralize at least interferon α subtype α1, α2, α4, α5, α6, α7, α8, α10, α13, α14, and α16.

In one embodiment, the anti-interferon α antibodies selectively bind to and neutralize at least interferon α subtype α1, α2, α4, α5, α8, α10, and α21.

In one embodiment, the anti-interferon α antibodies selectively bind to and neutralize at least interferon α subtype α1. In one embodiment, the anti-interferon α antibodies selectively bind to and neutralize at least interferon α subtype α2. In one embodiment, the anti-interferon α antibodies selectively bind to and neutralize at least interferon α subtype α4. In one embodiment, the anti-interferon α antibodies selectively bind to and neutralize at least interferon α subtype α5. In one embodiment, the anti-interferon α antibodies selectively bind to and neutralize at least interferon α subtype α6. In one embodiment, the anti-interferon α antibodies selectively bind to and neutralize at least interferon α subtype α7. In one embodiment, the anti-interferon α antibodies selectively bind to and neutralize at least interferon α subtype α8. In one embodiment, the anti-interferon α antibodies selectively bind to and neutralize at least interferon α subtype α10. In one embodiment, the anti-interferon α antibodies selectively bind to and neutralize at least interferon α subtype α13. In one embodiment, the anti-interferon α antibodies selectively bind to and neutralize at least interferon α subtype α14. In one embodiment, the anti-interferon α antibodies selectively bind to and neutralize at least interferon α subtype α16. In one embodiment, the anti-interferon α antibodies selectively bind to and neutralize at least interferon α subtype α17. In one embodiment, the anti-interferon α antibodies selectively bind to and neutralize at least interferon α subtype α21.

In certain embodiments, the anti-interferon α antibody is capable of reducing tyrosine phosphorylation, due to IFN-α binding, of the IFNAR1/IFNAR2 receptor complex by at least about 60%, or at least 70%, preferably at least 75%, more preferably at least 80%, even more preferably at least 85%, still more preferably at least 90%, still more preferably at least 95%, most preferably at least 99% as determined by a KIRA assay.

In some embodiments, the anti-human IFN-α monoclonal antibody comprises the following HVRs

(SEQ ID NO: 1)
(a) L1 of the formula RASQSVSTSSYSYMH;
(SEQ ID NO: 2)
(b) L2 of the formula YASNLES; and
(SEQ ID NO: 3)
(c) L3 of the formula QHSWGIPRTF; and/or
(SEQ ID NO: 4)
(d) H1 of the formula GYTFTEYIIH;
(SEQ ID NO: 5)
(e) H2 of the formula SINPDYDITNYNQRFKG; and
(SEQ ID NO: 6)
(f) H3 of the formula WISDFFDY;

In certain embodiments, the anti-human IFN-α monoclonal antibody comprises in its heavy and light chain variable domains amino acid sequence of

(SEQ ID NO: 7)
Glu Val Gln Leu Val Glu Ser Gly Gly Gly Leu Val Gln
Pro Gly Gly Ser Leu Arg Leu Ser Cys Ala Thr Ser Gly
Tyr Thr Phe Thr Glu Tyr Ile Ile His Trp Val Arg Gln
Ala Pro Gly Lys Gly Leu Glu Trp Val Ala Ser Ile Asn
Pro Asp Tyr Asp Ile Thr Asn Tyr Asn Gln Arg Phe Lys
Gly Arg Phe Thr Ile Ser Leu Asp Lys Ser Lys Arg Thr
Ala Tyr Leu Gln Met Asn Ser Leu Arg Ala Glu Asp Thr
Ala Val Tyr Tyr Cys Ala Ser Trp Ile Ser Asp Phe Phe
Asp Tyr Trp Gly Gln Gly Thr Leu Val Thr Val Ser Ser
Ala Ser
and
(SEQ ID NO: 8)
Asp Ile Gln Met Thr Gln Ser Pro Ser Ser Leu Ser Ala
Ser Val Gly Asp Arg Val Thr Ile Thr Cys Arg Ala Ser
Gln Ser Val Ser Thr Ser Ser Tyr Ser Tyr Met His Trp
Tyr Gln Gln Lys Pro Gly Lys Ala Pro Lys Val Leu Ile
Ser Tyr Ala Ser Asn Leu Glu Ser Gly Val Pro Ser Arg
Phe Ser Gly Ser Gly Ser Gly Thr Asp Phe Thr Leu Thr
Ile Ser Ser Leu Gln Pro Glu Asp Phe Ala Thr Tyr Tyr
Cys Gln His Ser Trp Gly Ile Pro Arg Thr Phe Gly Gln
Gly Thr Lys Val Glu Ile Lys Arg Thr Val,
respectively.

In some embodiments, the anti-human IFN-α monoclonal antibody has an amino acid sequence that is identical to the anti-human IFN-α monoclonal antibody having the non-proprietary name adopted by the USAN Council designated as Rontalizumab. In other embodiments, the anti-human IFN-α monoclonal antibody has an amino acid sequence identity that is at least 80%, at least 85%, at least 90%, at least 95%, at least 96%, at least 97%, at least 98%, or at least 99% to Rontalizumab. See U.S. Pat. No. 7,087,726. In certain embodiments, the anti-human IFN-α monoclonal antibody is Rontalizumab. In some embodiments, the anti-human IFN-α monoclonal antibody has an amino acid sequence as disclosed in CAS 948570-30-7.

In some embodiments, the anti-human IFN-α monoclonal antibody comprises the HVRs encoded by the following sequences:

(SEQ ID NO: 9)
(a) L1 of the formula
AGGGCCAGTC AGAGTGTTAG CAGCACCTAC TTAGCC;
(SEQ ID NO: 10)
(b) L2 of the formula
GGTGCATCCA GCAGGGCCAC T;
(SEQ ID NO: 11)
(c) L3 of the formula
CAGCAGTATG GTAGCTCACC TCGGACG;
(SEQ ID NO: 12)
(d) H1 of the formula
AGCTATAGTA TCAGC;
(SEQ ID NO: 13)
(e) H2 of the formula
AATGGTAACA CAAACTATGC ACAGAAGTTC CAGGGC; and
(SEQ ID NO: 14)
(f) H3 of the formula
GATCCCATAG CAGCAGGCTA C.

In some embodiments, the anti-human IFN-α monoclonal antibody comprises the HVRs of the following sequences:

(SEQ ID NO: 15)
(a) L1 of the formula RASQSVSSTYLA;
(SEQ ID NO: 16)
(b) L2 of the formula GASSRAT;
(SEQ ID NO: 17)
(c) L3 of the formula QQYGSSPRT;
(SEQ ID NO: 18)
(d) H1 of the formula SYSIS;
(SEQ ID NO: 19)
(e) H2 of the formula WISVYNGNTNYAQKFQG; and
(SEQ ID NO: 20)
(f) H3 of the formula DPIAAGY.

In some embodiments, the anti-human IFN-α monoclonal antibody comprises the HVRs as shown in FIGS. 1 A and B of US2007-0014724 (labeled as CDR therein).

In certain embodiments, the anti-human IFN-α monoclonal antibody comprises in its heavy and light chain variable domains amino acid sequence of

(SEQ ID NO: 21)
Gln Val Gln Leu Val Gln Ser Gly Ala Glu Val
Lys Lys Pro Gly Ala Ser Val Lys Val Ser Cys
Lys Ala Ser Gly Tyr Thr Phe Thr Ser Tyr Ser
Ile Ser Trp Val Arg Gln Ala Pro Gly Gln Gly
Leu Glu Trp Met Gly Trp Ile Ser Val Tyr Asn
Gly Asn Thr Asn Tyr Ala Gln Lys Phe Gln Gly
Arg Val Thr Met Thr Thr Asp Thr Ser Thr Ser
Thr Ala Tyr Leu Glu Leu Arg Ser Leu Arg Ser
Asp Asp Thr Ala Val Tyr Tyr Cys Ala Arg Asp
Pro Ile Ala Ala Gly Tyr Trp Gly Gln Gly Thr
Leu Val Thr Val Ser Ser
and
(SEQ ID NO: 22)
Glu Ile Val Leu Thr Gln Ser Pro Gly Thr Leu
Ser Leu Ser Pro Gly Glu Arg Ala Thr Leu Ser
Cys Arg Ala Ser Gln Ser Val Ser Ser Thr Tyr
Leu Ala Trp Tyr Gln Gln Lys Pro Gly Gln Ala
Pro Arg Leu Leu Ile Tyr Gly Ala Ser Ser Arg
Ala Thr Gly Ile Pro Asp Arg Phe Ser Gly Ser
Gly Ser Gly Thr Asp Phe Thr Leu Thr Ile Ser
Arg Leu Glu Pro Glu Asp Phe Ala Val Tyr Tyr
Cys Gln Gln Tyr Gly Ser Ser Pro Arg Thr Phe
Gly Gln Gly Thr Lys Val Glu Ile Lys,
respectively.

In some embodiments, the anti-human IFN-α monoclonal antibody has an amino acid sequence that is identical to the anti-human IFN-α monoclonal antibody having the non-proprietary name adopted by the USAN Council designated as Sifalimumab. In other embodiments, the anti-human IFN-α monoclonal antibody has an amino acid sequence identity that is at least 80%, at least 85%, at least 90%, at least 95%, at least 96%, at least 97%, at least 98%, or at least 99% to Sifalimumab. In certain embodiments, the anti-human IFN-α monoclonal antibody is Sifalimumab. In some embodiments, the anti-human IFN-α monoclonal antibody has an amino acid sequence as disclosed in CAS 1006877-41-3.

### V. Production of Antibodies

The methods and articles of manufacture of the present invention may use, or incorporate, an antibody that binds to a type I interferon, including, e.g., interferon α. Accordingly, methods for generating such antibodies will be described here.

Type I interferon antigen to be used for production of, or screening for, antibody(ies) may be, e.g., a soluble form of type I interferon, or a portion thereof, containing the desired epitope. Alternatively, or additionally, cells expressing type I interferon at their cell surface can be used to generate, or screen for, antibody(ies). Other forms of type I interferon useful for generating antibodies will be apparent to those skilled in the art.

A description follows as to exemplary techniques for the production of the antibodies used in accordance with the present invention.

(i) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C=NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(ii) Monoclonal Antibodies

Monoclonal antibodies are obtained from a population of substantially homogeneous antibodies, i.e., the individual antibodies comprising the population are identical and/or bind the same epitope except for possible variants that arise during production of the monoclonal antibody, such variants generally being present in minor amounts. Thus, the modifier “monoclonal” indicates the character of the antibody as not being a mixture of discrete or polyclonal antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-SEPHAROSE™ crosslinked agarose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs., 130:151-188 (1992).

In a further embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high-affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin-coding sequence all or part of the coding sequence for a non-immunoglobulin polypeptide.

Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iii) Humanized Antibodies

Methods for humanizing non-human antibodies have been described in the art. Preferably, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable-region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable-region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence that is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light- or heavy-chain variable regions. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

(iv) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain-joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807.

Alternatively, phage-display technology (McCafferty et al., Nature 348:552-553 (1990)) can be used to produce human antibodies and antibody fragments in vitro, from immunoglobulin variable (V)-domain gene repertoires from unimmunized donors. According to this technique, antibody V-domain genes are cloned in-frame into either a major or minor coat-protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

Human antibodies may also be generated by in vitro-activated B cells (see U.S. Pat. Nos. 5,567,610 and 5,229,275).

(v) Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host-cell culture. Other techniques for the production of antibody fragments will be apparent to the skilled practitioner. In other embodiments, the antibody of choice is a single-chain Fv fragment (scFv). See WO 1993/16185 and U.S. Pat. Nos. 5,571,894 and 5,587,458. The antibody fragment may also be a “linear antibody”, e.g., as described in U.S. Pat. No. 5,641,870. Such linear antibody fragments may be monospecific or bispecific.

(vi) Bispecific Antibodies

Bispecific antibodies are antibodies that have binding specificities for at least two different epitopes. Exemplary bispecific antibodies may bind to two different epitopes of the type I interferon antigen. Other such antibodies may bind a first type I interferon and further bind a second type I interferon. Alternatively, an anti-type I interferon-binding arm may be combined with an arm that binds to a triggering molecule on a leukocyte such as a T-cell receptor molecule (e.g. CD2 or CD3), or Fc receptors for IgG (FcγR), such as FcγRI (CD64), FcγRII (CD32) and FcγRIII (CD 16). Bispecific antibodies may also be used to localize cytotoxic agents. These antibodies possess a type I interferon-binding arm and an arm that binds the cytotoxic agent (e.g. saporin, anti-interferon-α, vinca alkaloid, ricin A chain, methotrexate or radioactive isotope hapten). Bispecific antibodies can be prepared as full-length antibodies or antibody fragments (e.g. F(ab′)₂ bispecific antibodies).

Methods for making bispecific antibodies are known in the art. Traditional production of full-length bispecific antibodies is based on the coexpression of two immunoglobulin heavy-chain-light-chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 1993/08829, and in Traunecker et al., EMBO J., 10:3655-3659 (1991).

According to a different approach, antibody variable domains with the desired binding specificities (antibody-antigen combining sites) are fused to immunoglobulin constant-domain sequences. The fusion preferably is with an immunoglobulin heavy-chain constant domain, comprising at least part of the hinge, CH2, and CH3 regions. It is preferred to have the first heavy-chain constant region (CH1), containing the site necessary for light-chain binding, present in at least one of the fusions. DNAs encoding the immunoglobulin heavy-chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy-chain-light-chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 1994/04690. For further details of generating bispecific antibodies, see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 1991/00360, WO 1992/200373, and EP 03089). Heteroconjugate antibodies may be made using any convenient cross-linking methods. Suitable cross-linking agents are well known in the art, and are disclosed, for example, in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

### IV. Conjugates and Other Modifications of the Antibody

The antibody used in the methods or included in the articles of manufacture herein is optionally conjugated to a cytotoxic agent. For instance, the (type I interferon) antibody may be conjugated to a drug as described in WO 2004/032828.

Chemotherapeutic agents useful in the generation of such antibody-cytotoxic agent conjugates have been described above.

Conjugates of an antibody and one or more small-molecule toxins, such as a calicheamicin, a maytansine (U.S. Pat. No. 5,208,020), a trichothene, and CC1065 are also contemplated herein. In one embodiment of the invention, the antibody is conjugated to one or more maytansine molecules (e.g. about 1 to about 10 maytansine molecules per antibody molecule). Maytansine may, for example, be converted to May-SS-Me, which may be reduced to May-SH3 and reacted with modified antibody (Chari et al. Cancer Research 52: 127-131 (1992)) to generate a maytansinoid-antibody conjugate.

Alternatively, the antibody is conjugated to one or more calicheamicin molecules. The calicheamicin family of antibiotics is capable of producing double-stranded DNA breaks at sub-picomolar concentrations. Structural analogues of calicheamicin that may be used include, but are not limited to, γ₁^{I}, α₂^{I}, α₃^{I}, N-acetyl-γ₁^{I}, PSAG and θ^{I}₁ (Hinman et al. Cancer Research 53: 3336-3342 (1993) and Lode et al. Cancer Research 58: 2925-2928 (1998)).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. See, for example, WO 1993/21232 published Oct. 28, 1993.

The present invention further contemplates antibody conjugated with a compound with nucleolytic activity (e.g. a ribonuclease or a DNA endonuclease such as a deoxyribonuclease; DNase).

A variety of radioactive isotopes is available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³² and radioactive isotopes of Lu.

Conjugates of the antibody and cytotoxic agent may be made using a variety of bifunctional protein-coupling agents such as N-succinimidyl-3-(2-pyridyldithiol) propionate (SPDP), succinimidyl-4-(N-maleimidomethyl)cyclohexane-1-carboxylate, iminothiolane (IT), bifunctional derivatives of imidoesters (such as dimethyl adipimidate HCl), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 1994/11026. The linker may be a “cleavable linker” facilitating release of the cytotoxic drug in the cell. For example, an acid-labile linker, peptidase-sensitive linker, dimethyl linker, or disulfide-containing linker (Chari et al. Cancer Research 52: 127-131 (1992)) may be used.

Alternatively, a fusion protein comprising the antibody and cytotoxic agent may be made, e.g. by recombinant techniques or peptide synthesis.

In yet another embodiment, the antibody may be conjugated to a “receptor” (such as streptavidin) for utilization in tumor pretargeting wherein the antibody-receptor conjugate is administered to the subject, followed by removal of unbound conjugate from the circulation using a clearing agent and then administration of a “ligand” (e.g. avidin) that is conjugated to a cytotoxic agent (e.g. a radionucleotide).

The antibodies of the present invention may also be conjugated with a prodrug-activating enzyme that converts a prodrug (e.g. a peptidyl chemotherapeutic agent, see WO 1981/01145) to an active anti-cancer drug. See, for example, WO 1988/07378 and U.S. Pat. No. 4,975,278.

The enzyme component of such conjugates includes any enzyme capable of acting on a prodrug in such a way so as to convert it into its more active, cytotoxic form.

Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases, and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature 328: 457-458 (1987)). Antibody-abzyme conjugates can be prepared as described herein for delivery of the abzyme to a tumor cell population.

The enzymes of this invention can be covalently bound to the antibody by techniques well known in the art such as the use of the heterobifunctional crosslinking reagents discussed above. Alternatively, fusion proteins comprising at least the antigen-binding region of an antibody of the invention linked to at least a functionally active portion of an enzyme of the invention can be constructed using recombinant DNA techniques well known in the art (see, e.g., Neuberger et al., Nature, 312: 604-608 (1984)).

Other modifications of the antibody are contemplated herein. For example, the antibody may be linked to one of a variety of nonproteinaceous polymers, e.g., polyethylene glycol (PEG), polypropylene glycol, polyoxyalkylenes, or copolymers of polyethylene glycol and polypropylene glycol. Antibody fragments, such as Fab′, linked to one or more PEG molecules are an especially preferred embodiment of the invention.

The antibodies disclosed herein may also be formulated as liposomes. Liposomes containing the antibody are prepared by methods known in the art, such as described in Epstein et al., Proc. Natl. Acad. Sci. USA, 82:3688 (1985); Hwang et al., Proc. Natl. Acad. Sci. USA, 77:4030 (1980); U.S. Pat. Nos. 4,485,045 and 4,544,545; and WO 1997/38731 published Oct. 23, 1997. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody of the present invention can be conjugated to the liposomes as described in Martin et al. J. Biol. Chem. 257: 286-288 (1982) via a disulfide-interchange reaction. A chemotherapeutic agent is optionally contained within the liposome. See Gabizon et al. J. National Cancer Inst. 81(19)1484 (1989).

Amino acid sequence modification(s) of protein or peptide antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or position of glycosylation sites.

A useful method for identification of certain residues or regions of the antibody that are preferred locations for mutagenesis is called “alanine-scanning mutagenesis” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody of an enzyme, or a polypeptide that increases the serum half-life of the antibody.

Another type of variant is an amino acid substitution variant. These variants have at least one amino acid residue in the antibody molecule replaced by a different residue. The sites of greatest interest for substitutional mutagenesis of antibodies include the hypervariable regions, but FR alterations are also contemplated. Conservative substitutions are shown in Table 3 under the heading of “preferred substitutions”. If such substitutions result in a change in biological activity, then more substantial changes, denominated “exemplary substitutions” in Table 3, or as further described below in reference to amino acid classes, may be introduced and the products screened.

TABLE 3

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (I), Pro (P), Phe (F), Trp (W), Met (M)

(2) uncharged polar: Gly (G), Ser (S), Thr (T), Cys (C), Tyr (Y), Asn (N), Gln (O)

(3) acidic: Asp (D), Glu (E)

(4) basic: Lys (K), Arg (R), His(H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g. 6-7 sites) are mutated to generate all possible amino acid substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine-scanning mutagenesis can be performed to identify hypervariable region residues contributing significantly to antigen binding. Alternatively, or in additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. Such altering includes deleting one or more carbohydrate moieties found in the antibody, and/or adding one or more glycosylation sites that are not present in the antibody.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered. For example, antibodies with a mature carbohydrate structure that lacks fucose attached to an Fc region of the antibody are described in US 2003/0157108 (Presta, L.). See also US 2004/0093621 (Kyowa Hakko Kogyo Co., Ltd.). Antibodies with a bisecting N-acetylglucosamine (GlcNAc) in the carbohydrate attached to an Fc region of the antibody are referenced in WO 2003/011878, Jean-Mairet et al. and U.S. Pat. No. 6,602,684, Umana et al. Antibodies with at least one galactose residue in the oligosaccharide attached to an Fc region of the antibody are reported in WO 1997/30087, Patel et al. See, also, WO 1998/58964 (Raju, S.) and WO 1999/22764 (Raju, S.) concerning antibodies with altered carbohydrate attached to the Fc region thereof.

The preferred glycosylation variant herein comprises an Fc region, wherein a carbohydrate structure attached to the Fc region lacks fucose. Such variants have improved ADCC function. Optionally, the Fc region further comprises one or more amino acid substitutions therein that further improve ADCC, for example, substitutions at positions 298, 333, and/or 334 of the Fc region (Eu numbering of residues). Examples of publications related to “defucosylated” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; Okazaki et al. J. Mol. Biol. 336:1239-1249 (2004); and Yamane-Ohnuki et al. Biotech. Bioeng. 87: 614 (2004). Examples of cell lines producing defucosylated antibodies include Lec13 CHO cells deficient in protein fucosylation (Ripka et al. Arch. Biochem. Biophys. 249:533-545 (1986); US 2003/0157108, Presta, L; and WO 2004/056312, Adams et al., especially at Example 11), and knockout cell lines, such as alpha-1,6-fucosyltransferase gene, FUT8,-knockout CHO cells (Yamane-Ohnuki et al. Biotech. Bioeng. 87: 614 (2004)).

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

It may be desirable to modify the antibody of the invention with respect to effector function, e.g. so as to enhance ADCC and/or CDC of the antibody. This may be achieved by introducing one or more amino acid substitutions in an Fc region of an antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and ADCC. See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered that has dual Fc regions and may thereby have enhanced complement lysis and ADCC capabilities. See Stevenson et al. Anti-Cancer Drug Design 3:219-230 (1989).

WO 2000/42072 (Presta, L.) describes antibodies with improved ADCC function in the presence of human effector cells, where the antibodies comprise amino acid substitutions in the Fc region thereof. Preferably, the antibody with improved ADCC comprises substitutions at positions 298, 333, and/or 334 of the Fc region. Preferably, the altered Fc region is a human IgG1 Fc region comprising or consisting of substitutions at one, two, or three of these positions.

Antibodies with altered C1q binding and/or CDC are described in WO 1999/51642 and U.S. Pat. Nos. 6,194,551, 6,242,195, 6,528,624, and 6,538,124 (Idusogie et al.). The antibodies comprise an amino acid substitution at one or more of amino acid positions 270, 322, 326, 327, 329, 313, 333, and/or 334 of the Fc region thereof.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule. Antibodies with substitutions in an Fc region thereof and increased serum half-lives are also described in WO 2000/42072 (Presta, L.).

Engineered antibodies with three or more (preferably four) functional antigen-binding sites are also contemplated (US 2002/0004587 A1, Miller et al.).

### V. Pharmaceutical Formulations

Therapeutic formulations of the antibodies used in accordance with the present invention are prepared for storage by mixing an antibody having the desired degree of purity with optional pharmaceutically acceptable carriers, excipients, or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low-molecular-weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™, or PEG.

Exemplary anti-type I antibody formulations are described in U.S. Pat. Nos. 7,087,726 7,741,449, and U.S. Patent Publication No, 2009/0214565.

Lyophilized formulations adapted for subcutaneous administration are described, for example, in U.S. Pat. No. 6,267,958 (Andya et al.). Such lyophilized formulations may be reconstituted with a suitable diluent to a high protein concentration and the reconstituted formulation may be administered subcutaneously to the mammal to be treated herein.

Crystallized forms of the antibody are also contemplated. See, for example, US 2002/0136719A1 (Shenoy et al.).

The formulation herein may also contain more than one active compound (a second medicament) as necessary for the particular indication being treated, preferably those with complementary activities that do not adversely affect each other. For example, it may be desirable to further provide a cytotoxic agent (e.g. mitoxantrone (NOVANTRONE®), methotrexate, cyclophosphamide, chlorambucil, or azathioprine), chemotherapeutic agent, immunosuppressive agent, cytokine, cytokine antagonist or antibody, growth factor, hormone (e.g., testosterone or hormone replacement therapy), integrin, integrin antagonist or antibody (e.g., an LFA-1 antibody such as efalizumab/RAPTIVA® commercially available from Genentech, or an alpha 4 integrin antibody such as natalizumab/ANTEGREN® available from Biogen, or others as noted above), interferon class drug such as IFN-beta-1a (REBIF® and AVONEX®) or IFN-beta-1b (BETASERON®), an oligopeptide such as glatiramer acetate (COPAXONE®), intravenous immunoglobulin (gamma globulin), lymphocyte-depleting drug (e.g., mitoxantrone, cyclophosphamide, CAMPATH™ antibodies, anti-CD4, or cladribine), non-lymphocyte-depleting immunosuppressive drug (e.g., MMF or cyclosporine), cholesterol-lowering drug of the “statin” class, estradiol, drug that treats symptoms secondary or related to lupus (e.g., spasticity, incontinence, pain, fatigue), a TNF inhibitor, DMARD, NSAID, corticosteroid (e.g., methylprednisolone, prednisone, dexamethasone, or glucorticoid), levothyroxine, cyclosporin A, somatastatin analogue, anti-metabolite, another B-cell surface antagonist/antibody, etc., in the formulation. The type and effective amounts of such other agents (called herein second medicaments, wherein the first medicament is the type I interferon antibody) depend, for example, on the amount of antibody present in the formulation, the type of lupus being treated, and clinical parameters of the subjects.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug-delivery systems (for example, liposomes, albumin microspheres, microemulsions, nano-particles and nanocapsules) or in macroemulsions. Such techniques are disclosed, e.g., in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

### VI. Articles of Manufacture

Various articles of manufacture are contemplated within the scope of the invention. In another embodiment of the invention, an article of manufacture containing materials useful for the treatment of lupus described above is provided. Preferably, the article of manufacture comprises (a) a container comprising a composition comprising a type I interferon antibody and a pharmaceutically acceptable carrier or diluent within the container; and (b) a package insert with instructions for treating lupus in a subject, wherein the instructions indicate that an amount of the antibody is administered to the subject that is effective to provide an initial antibody exposure of about 0.5 to 4 grams followed by a second antibody exposure of about 0.5 to 4 grams, wherein the second exposure is not provided until from about 16 to 54 weeks from the initial exposure and each of the antibody exposures is provided to the subject as a single dose or as two or three separate doses of antibody.

The package insert is on or associated with the container. Suitable containers include, for example, bottles, vials, syringes, etc. The containers may be formed from a variety of materials such as glass or plastic. The container holds or contains a composition that is effective for treating the lupus and may have a sterile access port (for example, the container may be an intravenous solution bag or a vial having a stopper pierceable by a hypodermic injection needle). At least one active agent in the composition is the antibody. The label or package insert indicates that the composition is used for treating lupus in a subject eligible for treatment with specific guidance regarding dosing amounts and intervals of antibody and any other drug being provided. The article of manufacture may further comprise a second container comprising a pharmaceutically acceptable diluent buffer, such as bacteriostatic water for injection (BWFI), phosphate-buffered saline, Ringer's solution, and dextrose solution. The article of manufacture may still further comprise a second or third container comprising a second medicament, wherein the type I interferon antibody is a first medicament, where the article further comprises instructions on the package insert for treating the subject with the second medicament. Exemplary second medicaments include a chemotherapeutic agent, an immunosuppressive agent, an anti-malarial agent, a cytotoxic agent, an integrin antagonist, a cytokine antagonist, or a hormone. In some embodiments, the second medicament is a chemotherapeutic agent, an anti-malarial agent, or an immunosuppressive agent, including, e.g., hydroxychloroquine, chloroquine, quinacrine, cyclophosphamide, prednisone, mycophenolate mofetil, methotrexate, azathiprine, or 6-mercaptopurine; a corticosteroid such as prednisone (along with optionally methotrexate, hydroxychloroquine, chloroquine, quinacrine, MMF, or azathioprine with or without 6-mercaptopurine); or a corticosteroid such as prednisone as well as MMF or cyclophosphamide. The article of manufacture may further include other materials desirable from a commercial and user standpoint, including other buffers, diluents, filters, needles, and syringes.

In another aspect, the invention contemplates an article of manufacture that includes, but is not limited to, a computerized system comprising a bio-assay module for detecting a gene expression of one or more IRGs from a biological sample and a processor module to calculate expression of the gene and to score the calculation of the gene or protein synthesis against a cutoff value to provide a diagnosis, wherein the cutoff value is (1) less than 1.5 times the value of the expression levels of the IRGs of a healthy person (or control) or (2) less than two standard deviations over the median value of the expression levels of the IRGs in a healthy persons (or control).

In another aspect, the invention provides an article of manufacture comprising a subcutaneous administration device, which delivers to a patient a fixed dose of an anti-interferon α antibody, wherein the fixed dose is in the range of about 50 mg to about 2000 mg of the anti-interferon α antibody. In some embodiments, the fixed dose is about 100-500 mg weekly, about 200-1000 mg biweekly, or about 400-2000 mg monthly. In some embodiments, the fixed dose is about any of 150 mg, 200 mg, 250 mg, 300 mg, 350 mg, 400 mg, 450 mg, or 500 mg. In some embodiments, the fixed dose is about 150 mg or about 300 mg weekly, about 300 mg or about 600 mg biweekly, or about 600 mg, about 750 mg or about 1200 mg monthly. In some embodiments, the concentration of the antibody in the device is from about 50 to 250 mg/mL. In another aspect, the invention provides an article of manufacture comprising an anti-interferon α antibody in a concentration from about 50 to 250 mg/mL. In some embodiments, the anti-interferon α antibody comprises a light chain comprising HVR-L1 comprising the amino acid sequence RASQSVSTSSYSYMH (SEQ ID NO:1), HVR-L2 comprising the amino acid sequence YASNLES (SEQ ID NO:2), and HVR-L3 comprising the amino acid sequence QHSWGIPRTF (SEQ ID NO:3); and/or a heavy chain comprising HVR-H1 comprising the amino acid sequence GYTFTEYIIH (SEQ ID NO:4), HVR-H2 comprising the amino acid sequence SINPDYDITNYNQRFKG (SEQ ID NO:5), and HVR-H3 comprising the amino acid sequence WISDFFDY (SEQ ID NO:6). In some embodiments, the antibody comprises a heavy chain variable region sequence of at least 95% sequence identity to the amino acid sequence of SEQ ID NO:7; and/or a light chain variable region sequence of at least 95% sequence identity to the amino acid sequence of SEQ ID NO:8. In some embodiments, the antibody comprises a heavy chain variable region comprising the amino acid sequence of SEQ ID NO:7; and/or a light chain variable region comprising the amino acid sequence of SEQ ID NO:8. In some embodiments, the antibody is rontalizumab having CAS registration number 948570-30-7. In some embodiments, the subcutaneous administration device is a pre-filled syringe, an autoinjector, or a large volume infusion device. For example, MyDose product from Roche, a single use infusion device that enables the subcutaneous administration of large quantities of liquid medication, may be used as the administration device.

### VII. Exemplar Embodiments

1. A method of treating an autoimmune disease in a patient, the method comprising administering an effective amount of an interferon inhibitor to a patient, wherein the patient has been diagnosed with the autoimmune disease and has been determined to be ISM^{lo} or has been selected for treatment based on being ISM^{lo}.
2. The method of embodiment 1, wherein the ISM^{lo} is determined by measuring mRNA expression levels of one or more interferon response genes (IRGs) in a sample from the patient.
3. The method of embodiment 2, wherein the mRNA expression levels in the sample are determined by RT-PCR.
4. The method of embodiment 2 or 3, wherein the mRNA expression levels of one or more IRGs are normalized against mRNA expression level of a house keeping gene (such as transferring receptor (TFRC), ribosomal protein L19, glyceraldehyde-3-phosphate dehydrogenase (GAPDH), etc.).
5. The method of any one of embodiments 2-4, wherein the sample is a blood sample.
6. The method of any one of embodiments 2-5, wherein the mRNA expression levels of one or more of IRGs selected from the group consisting of CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP, ZBP1, IFI27, SIGLEC1, DNAPTP6, USP18, IFI6, HSXIAPAF1, and LAMP3 (one, combination or all IRGs listed in Table A) are determined.
7. The method of embodiment 6, wherein the mRNA expression levels of one or more of IRGs selected from the group consisting of CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP, ZBP1, IFI27, SIGLEC1, DNAPTP6, USP18, IFI6, HSXIAPAF1, and LAMP3 are normalized against mRNA expression level of transferrin receptor (TFRC).
8. The method of any one of embodiments 2-5, wherein the mRNA expression levels of one or more of IRGs selected from the group consisting of CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP, and ZBP1 (one, combination or all IRGs of the 24-gene ISM signature) are determined.
9. The method of embodiment 8, wherein the mRNA expression levels of one or more of IRGs selected from the group consisting of CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP, ZBP1 are normalized against mRNA expression level of transferrin receptor (TFRC).
10. The method of any one of embodiments 2-5, wherein the mRNA expression levels of EPSTI1, HERC5 and/or TYK1 (CMPK2) are determined.
11. The method of embodiment 10, wherein the mRNA expression levels of EPSTI1, HERC5 and/or TYK1 (CMPK2) are normalized against mRNA expression level of transferrin receptor (TFRC).
12. A method of treating an autoimmune disease in a patient, the method comprising administering an effective amount of an interferon inhibitor to a patient, wherein the patient has been diagnosed with the autoimmune disease and has been determined to have a pre-treatment anti-double stranded DNA antibody titer (anti-dsDNA) that is less than or equal to 200 IU as measured by immunoassay or selected for treatment based on having a pre-treatment anti-double stranded DNA antibody titer (anti-dsDNA) that is less than or equal to 200 IU as measured by immunoassay.
13. The method of embodiment 12, wherein the immunoassay is an ELISA.
14. The method of embodiment 12 or 13, wherein the patient has an anti-dsDNA titer that is less than or equal to 200 IU and is ISM^{hi}.
15. The method of embodiment 14, wherein the ISM^{hi} is determined by measuring mRNA expression levels of one or more IRGs in a sample from the patient.
16. The method of embodiment 15, wherein the mRNA expression levels are determined by RT-PCR.
17. The method of embodiment 15 or 16, wherein the mRNA expression levels of one or more IRGs are normalized against mRNA expression level of a house keeping gene (such as transferring receptor (TFRC), ribosomal protein L19, glyceraldehyde-3-phosphate dehydrogenase (GAPDH), etc.).
18. The method of any one of embodiments 15-17, wherein the sample is a blood sample.
19. The method of any one of embodiments 15-18, wherein the mRNA expression levels of one or more of IRGs selected from the group consisting of CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP, ZBP1, IFI27, SIGLEC1, DNAPTP6, USP18, IFI6, HSXIAPAF1, and LAMP3 (one, combination or all IRGs listed in Table A) are determined.
20. The method of embodiment 19, wherein the mRNA expression levels of one or more of IRGs selected from the group consisting of CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP, ZBP1, IFI27, SIGLEC1, DNAPTP6, USP18, IFI6, HSXIAPAF1, and LAMP3 are normalized against mRNA expression level of transferrin receptor (TFRC).
21. The method of any one of embodiments 15-18, wherein the mRNA expression levels of one or more of IRGs selected from the group consisting of CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP, and ZBP1 (one, combination or all IRGs of the 24-gene ISM signature) are determined.
22. The method of embodiment 21, wherein the mRNA expression levels of one or more of IRGs selected from the group consisting of CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP, ZBP1 are normalized against mRNA expression level of transferrin receptor (TFRC).
23. The method of any one of embodiments 15-18, wherein the mRNA expression levels of EPSTI1, HERC5 and/or TYK1 (CMPK2) are determined.
24. The method of embodiment 23, wherein the mRNA expression levels of EPSTI1, HERC5 and/or TYK1 (CMPK2) are normalized against TFRC.
25. The method of any one of embodiments 1-24, wherein the autoimmune disease is selected from the group consisting of lupus, rheumatoid arthritis, psoriasis, psoriatic arthritis, insulin-dependent diabetes mellitus (IDDM), multiple sclerosis (MS), myositis, dermatomyositis, vasculitis, atherosclerosis, ankylosing spondylitis, and Sjogren's syndrome.
26. The method of embodiment 25, wherein the patient has systemic lupus erythematosus (SLE).
27. The method of embodiment 25, wherein the patient has moderately to severely active lupus.
28. The method of embodiment 25, wherein the patient has moderately to severely active SLE.
29. The method of embodiment 25, wherein the patient has lupus nephritis.
30. The method of any one of embodiments 1-13, wherein the patient has Class III-V lupus nephritis and is ISM^{lo}.
31. The method of embodiment 25, wherein the patient has pediatric lupus.
32. The method of any one of embodiments 1-31, wherein the interferon inhibitor is an anti-interferon type I antibody.
33. The method of embodiment 32, wherein the antibody specifically binds an interferon selected from the group consisting of: interferon α, interferon β, interferon ω, interferon λ, and combinations thereof.
34. The method of embodiment 32, wherein the antibody specifically binds interferon α.
35. The method of embodiment 34, wherein the antibody binds to at least IFNα subtypes 1, 2, 4, 5, 8, 10 and 21.
36. The method of embodiment 34, wherein the antibody comprises a light chain comprising HVR-L1 comprising the amino acid sequence RASQSVSTSSYSYMH (SEQ ID NO:1), HVR-L2 comprising the amino acid sequence YASNLES (SEQ ID NO:2), and HVR-L3 comprising the amino acid sequence QHSWGIPRTF (SEQ ID NO: 3); and/or a heavy chain comprising HVR-H1 comprising the amino acid sequence GYTFTEYIIH (SEQ ID NO: 4), HVR-H2 comprising the amino acid sequence SINPDYDITNYNQRFKG (SEQ ID NO:5), and HVR-H3 comprising the amino acid sequence WISDFFDY (SEQ ID NO:6).
37. The method of embodiment 34, wherein the antibody comprises a heavy chain variable region sequence of at least 95% sequence identity to the amino acid sequence of SEQ ID NO:7; and/or a light chain variable region sequence of at least 95% sequence identity to the amino acid sequence of SEQ ID NO:8.
38. The method of embodiment 34, wherein the antibody comprises a heavy chain variable region comprising the amino acid sequence of SEQ ID NO:7; and/or a light chain variable region comprising the amino acid sequence of SEQ ID NO:8.
39. The method of embodiment 34, wherein the antibody is rontalizumab having CAS registration number 948570-30-7.
40. The method of any one of embodiments 32-39, wherein the antibody is administered intravenously.
41. The method of any one of embodiments 32-39, wherein the antibody is administered subcutaneously.
42. The method of any one of embodiments 32-39, wherein the antibody is administered at a flat dose of 100 to 2000 mg.
43. The method of embodiment 42, wherein the antibody is administered at a flat dose of 100-500 mg weekly, 200-1000 mg biweekly, or 400-2000 mg monthly.
44. The method of embodiment 42 or 43, wherein the antibody is administered at a flat does of 150 mg or 300 mg weekly, 300 mg or 600 mg biweekly, or 600 mg, 750 mg or 1200 mg monthly.
45. The method of any one of embodiments 32-44, wherein the administration of the antibody is effective in one or more of the following: (1) reduction of the number and/or severity of lupus flares, (2) prevention of lupus flares, (3) reduction in lupus nephritis flares, (4) prevention of lupus nephritis flares, (5) induction of remission in lupus nephritis, (6) maintenance of lupus nephritis remission, (7) reduction in the number and/or severity of pediatric lupus flares, (8) prevention of pediatric lupus flares, (9) reduction in pediatric lupus nephritis flares, (10) prevention of pediatric lupus nephritis flares, (11) induction of remission in pediatric lupus nephritis, and (12) maintenance of pediatric lupus nephritis remission.
46. The method of any one of embodiments 32-44, wherein the administration of the antibody is effective in lowering the anti-dsDNA antibody titer in the patient.
47. The method of any one of embodiments 32-44, wherein the administration of the antibody is effective in reduction of flare(s) in the patient.
48. The method of embodiment 47, wherein said flare(s) are moderate or severe.
49. The method of any one of embodiments 32-44, wherein the administration of the antibody is effective in reduction of Selena Flare Index (SFI) score or Selena Flare Index-Revised (SFI-R) score in the patient.
50. The method of any one of embodiments 32-44, wherein the administration of the antibody is effective in decreasing all pre-treatment BILAG A and B domains.
51. The method of any one of embodiments 32-44, wherein the patient has no new BILAG A organ domain score or no more than one new BILAG B organ domain score after the administration of the antibody.
52. The method of any one of embodiments 32-44, wherein the administration of the antibody is effective in decreasing in SELENA-SLEDAI score by at least four points from the patient's pre-treatment score.
53. The method of any one of embodiments 32-44, wherein the patient has no more than 0.3 points increase in Physician Global Assessment (PGA) from the pre-treatment score after the administration of the antibody.
54. The method of any one of embodiments 32-44, wherein said patient has a post-treatment decrease in disease activity in those organ systems with moderate or severe disease activity prior to treatment as measured by any one of the following assessment tools: SRI, BILAG, SELENA-SLEDAI, or Physician Global Assessment (PGA).
55. The method of any one of embodiments 32-44, wherein the patient has an SRI-4, SRI-5, SRI-6, or SRI-7 response to the administration of the antibody.
56. The method of any one of embodiments 1-55, further comprising administering a second medicament to the patient.
57. The method of embodiment 56, wherein the second medicament is selected from the group consisting of: a corticosteroid, a non-steroidal anti-inflammatory drug (NSAID), an immunosuppressive, an anti-malarial agent, a statin, and combinations thereof.
58. The method of embodiment 56, wherein the second medicament is a standard of care for lupus.
59. The method of any one of embodiments 32-44, wherein the administration of the antibody results in corticosteroid sparing (CS) in a patient taking a corticosteroid prior to said administration of said antibody.
60. The method of any one of embodiments 32-44, wherein the administration of the antibody results in a decrease in the requirement for therapy with steroids and/or immunosuppressive regimens.
61. The method of any one of embodiments 32-44, wherein the patient has tapered their corticosteroid dose to a prednisone equivalent of 10 mg/day after the administration of the antibody.
62. The method of any one of embodiments 32-44, wherein the administration of the antibody results in reduction in corticosteroid use by at least 50% after about 24 to about 52 weeks of the administration of the antibody.
63. The method of any one of embodiments 32-44, wherein the administration of the antibody results in one or more of the following: reduction in the incidence of moderate and/or severe flares as measured by SELENA SLEDAI scores and/or Physicians Global Assessment; significantly delaying time to severe flare; reduction in the number of swollen or tender joints; and significantly reducing the risk of one BILAG A (severe) organ flare or more than one BILAG B (moderate) organ flare.
64. A therapeutic regimen for the treatment of an ISM^{lo} lupus patient in need thereof comprising the administration of an interferon inhibitor.
65. The regimen of embodiment 64, wherein the patient has SLE or lupus nephritis.
66. The regimen of embodiment 64 or 65, wherein the interferon inhibitor is an anti-IFNα antibody.
67. The regimen of embodiment 66, wherein the antibody is administered at a flat dose of 100-2000 mg.
68. The regimen of embodiment 66, wherein the antibody is administered at a flat dose of 100-500 mg weekly, 200-1000 mg biweekly, or 400-2000 mg monthly.
69. The regimen of embodiment 66, wherein the antibody is administered at a flat dose of 150 mg or 300 mg weekly, 300 mg or 600 mg biweekly, or 600 mg, 750 mg or 1200 mg monthly.
70. The regimen of any one of embodiments 66-69, wherein the antibody is administered intravenously or subcutaneously.
71. The regimen of any one of embodiments 66-70, wherein the antibody comprises a light chain comprising HVR-L1 comprising the amino acid sequence RASQSVSTSSYSYMH (SEQ ID NO:1), HVR-L2 comprising the amino acid sequence YASNLES (SEQ ID NO:2), and HVR-L3 comprising the amino acid sequence QHSWGIPRTF (SEQ ID NO:3); and/or a heavy chain comprising HVR-H1 comprising the amino acid sequence GYTFTEYIIH (SEQ ID NO:4), HVR-H2 comprising the amino acid sequence SINPDYDITNYNQRFKG (SEQ ID NO:5), and HVR-H3 comprising the amino acid sequence WISDFFDY (SEQ ID NO:6).
72. The regimen of any one of embodiments 66-70, wherein the antibody comprises a heavy chain variable region sequence of at least 95% sequence identity to the amino acid sequence of SEQ ID NO:7; and/or a light chain variable region sequence of at least 95% sequence identity to the amino acid sequence of SEQ ID NO:8.
73. The regimen of any one of embodiments 66-70, wherein the antibody comprises a heavy chain variable region comprising the amino acid sequence of SEQ ID NO:7; and/or a light chain variable region comprising the amino acid sequence of SEQ ID NO:8.
74. The regimen of any one of embodiments 66-70, wherein the antibody is rontalizumab having CAS registration number 948570-30-7.
75. A method of identifying a lupus patient who may benefit from an interferon inhibitor treatment, the method comprising determining the IRG status in a sample from the patient, wherein a patient who is ISM^{lo} is identified as a patient who may benefit from the interferon inhibitor treatment.
76. A method of identifying a lupus patient who may benefit from an interferon inhibitor treatment, the method comprising determining the IRG status in a sample from the patient, and providing a report regarding the IRG status of the patient, wherein the report indicates that the patient is ISM^{lo} or ISM^{hi}.
77. The method of embodiment 76, wherein the report further indicates that the patient may benefit from the interferon inhibitor treatment if the patient is ISM^{lo}.
78. A method of predicting responsiveness of a lupus patient to an interferon inhibitor treatment, the method comprising determining the IRG status in a sample from the patient, wherein a patient who is ISM^{lo} is identified as a patient who is likely to respond to the interferon inhibitor treatment.
79. The method of any one of embodiments 75-78, wherein the patient is considered as ISM^{lo} if the IRG expression levels are less than 1.5, 1.4, 1.3, 1.2, or 1.1 times the mean value of the expression levels of the same IRGs of a healthy person or control.
80. The method of any one of embodiments 75-78, wherein the patient is considered as ISM^{lo} if the IRG expression levels are less than 2, 1.9, 1.8, 1.7, 1.6, 1.5, 1.4, 1.3, 1.2, or 1.1 standard deviations over the mean value of the expression levels of the same IRGs of healthy persons or control.
81. A method of predicting responsiveness of a lupus patient to an interferon inhibitor treatment, the method comprising determining the expression levels of IRGs in a sample from the patient, and comparing the patient's IRG expression levels to a mean value of the expression levels of the same IRGs in a healthy person or healthy persons, wherein the patient is identified as a patient who is likely to respond to the interferon inhibitor treatment if the patient's IRG expression levels are (1) less than 1.5, 1.4, 1.3, 1.2, or 1.1 times the mean value of the expression levels of the same IRGs of a healthy person or control, or (2) less than 2, 1.9, 1.8, 1.7, 1.6, 1.5, 1.4, 1.3, 1.2, or 1.1 standard deviations over the mean value of the expression levels of the same IRGs in healthy persons or control.
82. The method of any one of embodiments 75-81, wherein the ISM^{lo} or IRG expression levels are determined by measuring mRNA expression levels of one or more interferon response genes (IRGs) in a sample from the patient.
83. The method of embodiment 82, wherein the mRNA expression levels of one or more IRGs in the sample are measured by RT-PCR.
84. The method of embodiment 82 or 83, wherein the mRNA expression levels of one or more IRGs are normalized against mRNA expression level of a house keeping gene (such as transferring receptor (TFRC), ribosomal protein L19, glyceraldehyde-3-phosphate dehydrogenase (GAPDH), etc.).
85. The method of any one of embodiments 75-84, wherein the sample is a blood sample.
86. The method of any one of embodiments 75-85, wherein the mRNA expression levels of one or more of IRGs selected from the group consisting of CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP, ZBP1, IFI27, SIGLEC1, DNAPTP6, USP18, IFI6, HSXIAPAF1, and LAMP3 (one, combination or all IRGs listed in Table A) are determined.
87. The method of embodiment 86, wherein the mRNA expression levels of one or more of IRGs selected from the group consisting of CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP, ZBP1, IFI27, SIGLEC1, DNAPTP6, USP18, IFI6, HSXIAPAF1, and LAMP3 are normalized against mRNA expression level of transferrin receptor (TFRC).
88. The method of any one of embodiments 75-85, wherein the mRNA expression levels of one or more of IRGs selected from the group consisting of CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP, and ZBP1 (one, combination or all IRGs of the 24-gene ISM signature) are determined.
89. The method of embodiment 88, wherein the mRNA expression levels of one or more of IRGs selected from the group consisting of CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP, ZBP1 are normalized against mRNA expression level of transferrin receptor (TFRC).
90. The method of any one of embodiments 75-85, wherein the mRNA expression levels of EPSTI1, HERC5 and/or TYK1 (CMPK2) are determined.
91. The method of embodiment 90, wherein the mRNA expression levels of EPSTI1, HERC5 and/or TYK1 (CMPK2) are normalized against mRNA expression level of transferrin receptor (TFRC).
92. A method of identifying a lupus patient who may benefit from an interferon inhibitor treatment, the method comprising determining the anti-dsDNA antibody status in a sample from the patient, wherein a patient who has an anti-dsDNA antibody titer that is less than or equal to 200 IU as measured by immunoassay is identified as a patient who may benefit from the interferon inhibitor treatment.
93. A method of identifying a lupus patient who may benefit from an interferon inhibitor treatment, the method comprising determining the anti-dsDNA antibody status in a sample from the patient, and providing a report indicating that the patient may benefit from the interferon inhibitor treatment if anti-dsDNA antibody titer that is less than or equal to 200 IU as measured by immunoassay.
94. A method of predicting responsiveness of a lupus patient to an interferon inhibitor treatment, the method comprising determining the anti-dsDNA antibody status in a sample from the patient, wherein a patient who has an anti-dsDNA antibody titer that is less than or equal to 200 IU as measured by immunoassay is identified as a patient who is likely to respond to the interferon inhibitor treatment.
95. The method of any one of embodiments 92-94, wherein the immunoassay is an ELISA.
96. A method for predicting the likelihood of a flare in a lupus patient, the method comprising determining the IRG status of the patient, wherein a significant increase of expression levels of IRGs indicates that the patient is likely to have a flare in the next 3 to 5 weeks.
97. The method of embodiment 96, wherein said IRGs are selected from the group consisting of EPSTI1, HERC5, TYK1 (CMPK2), IFI27, IFI44, IFIT1, MX1, OAS1, OAS2, OAS3, and a combination thereof.
98. The method of embodiment 96, wherein the flare is determined by the SELENA-SLEDAI Flare Index (SFI) and/or SFI-Revised.
99. The method of embodiment 96, wherein the flare is mild, moderate or severe based on the SELENA-SLEDAI Flare Index (SFI) and/or SFI-Revised.
100. The method of any one of embodiments 75-95, wherein the interferon inhibitor is an anti-interferon type I antibody.
101. The method of embodiment 100, wherein the antibody specifically binds an interferon selected from the group consisting of: interferon α, interferon β, interferon ω, interferon λ, and combinations thereof.
102. The method of embodiment 100, wherein the antibody specifically binds interferon α.
103. The method of embodiment 100, wherein the antibody binds to at least IFNα subtypes 1, 2, 4, 5, 8, 10 and 21.
104. The method of embodiment 102, wherein the antibody comprises a light chain comprising HVR-L1 comprising the amino acid sequence RASQSVSTSSYSYMH (SEQ ID NO:1), HVR-L2 comprising the amino acid sequence YASNLES (SEQ ID NO:2), and HVR-L3 comprising the amino acid sequence QHSWGIPRTF (SEQ ID NO:3); and/or a heavy chain comprising HVR-H1 comprising the amino acid sequence GYTFTEYIIH (SEQ ID NO:4), HVR-H2 comprising the amino acid sequence SINPDYDITNYNQRFKG (SEQ ID NO:5), and HVR-H3 comprising the amino acid sequence WISDFFDY (SEQ ID NO:6).
105. The method of embodiment 102, wherein the antibody comprises a heavy chain variable region sequence of at least 95% sequence identity to the amino acid sequence of SEQ ID NO:7; and/or a light chain variable region sequence of at least 95% sequence identity to the amino acid sequence of SEQ ID NO:8.
106. The method of embodiment 102, wherein the antibody comprises a heavy chain variable region comprising the amino acid sequence of SEQ ID NO:7; and/or a light chain variable region comprising the amino acid sequence of SEQ ID NO:8.
107. The method of embodiment 102, wherein the antibody is rontalizumab having CAS registration number 948570-30-7.
108. An article of manufacture comprising a subcutaneous administration device, which delivers to a patient a flat dose of an anti-interferon α antibody, wherein the flat dose is in the range of 50 mg to 2000 mg of the anti-interferon α antibody.
109. The article of manufacture of embodiment 108, wherein the flat dose is 100-500 mg weekly, 200-1000 mg biweekly, or 400-2000 mg monthly.
110. The article of manufacture of embodiment 108, wherein the flat dose is 150 mg or 300 mg weekly, 300 mg or 600 mg biweekly, or 600 mg, 750 mg or 1200 mg monthly.
111. The article of manufacture of embodiment 108, wherein the concentration of the antibody in the device is from about 50 to 250 mg/mL.
112. An article of manufacture comprising an anti-interferon α antibody in a concentration from about 50 to 250 mg/mL.
113. The article of manufacture of any one embodiments 108-112, wherein the antibody comprises a light chain comprising HVR-L1 comprising the amino acid sequence RASQSVSTSSYSYMH (SEQ ID NO:1), HVR-L2 comprising the amino acid sequence YASNLES (SEQ ID NO:2), and HVR-L3 comprising the amino acid sequence QHSWGIPRTF (SEQ ID NO:3); and/or a heavy chain comprising HVR-H1 comprising the amino acid sequence GYTFTEYIIH (SEQ ID NO:4), HVR-H2 comprising the amino acid sequence SINPDYDITNYNQRFKG (SEQ ID NO:5), and HVR-H3 comprising the amino acid sequence WISDFFDY (SEQ ID NO:6).
114. The article of manufacture of any one of embodiments 108-112, wherein the antibody comprises a heavy chain variable region sequence of at least 95% sequence identity to the amino acid sequence of SEQ ID NO:7; and/or a light chain variable region sequence of at least 95% sequence identity to the amino acid sequence of SEQ ID NO:8.
115. The article of manufacture of any one of embodiments 108-112, wherein the antibody comprises a heavy chain variable region comprising the amino acid sequence of SEQ ID NO:7; and/or a light chain variable region comprising the amino acid sequence of SEQ ID NO:8.
116. The article of manufacture of any one of embodiments 108-112, wherein the antibody is rontalizumab having CAS registration number 948570-30-7.
117. The article of manufacture of embodiment 108, wherein the subcutaneous administration device is a pre-filled syringe, an autoinjector, or a large volume infusion device.
118. An article of manufacture comprising a computerized system comprising a bio-assay module for detecting a gene expression of one or more IRGs from a biological sample and a processor module to calculate expression of the gene and to score the calculation of the gene against a cutoff value to provide a diagnosis, wherein the cutoff value is (1) less than 1.5, 1.4, 1.3, 1.2, or 1.1 times the value of the expression levels of the IRGs of a healthy person or control or (2) less than 2, 1.9, 1.8, 1.7, 1.6, 1.5, 1.4, 1.3, 1.2, or 1.1 standard deviations (over the median value of the expression levels of the IRGs in healthy persons or control.
119. The article of manufacture according to embodiment 118, wherein the bio-assay module is the cobas z480 analyzer.
120. A kit for identifying an autoimmune patient who may benefit for an interferon inhibitor treatment, comprising a vial for collecting a blood sample from an autoimmune patient and instructions for determining whether the autoimmune patient is ISM^{lo}.
121. The kit of embodiment 120, wherein the expression level of at least one gene selected from the group consisting of EPSTI1, HERC5, TYK1 (CMPK2), IFI27, IFI44, IFIT1, MX1, OAS 1, OAS2, and OAS3 is used to determine whether the autoimmune patient is ISM^{lo}.
122. The kit of embodiment 121, wherein the autoimmune disease is lupus.
123. The kit of embodiment 120, wherein the interferon inhibitor is an anti-interferon α antibody.
124. A stable liquid composition comprising an anti-interferon α antibody in an amount of about 50 to about 250 mg/mL, arginine-HCl in an amount of about 50 to about 200 mM, histidine in an amount of about 5 to about 100 mM, polysorbate in an amount of about 0.01 to about 0.1%, wherein the composition has a pH from about 5.5 to about 7.0.
125. A method of treating lupus in a patient, the method comprising administering an effective amount of an interferon type I antibody to a patient diagnosed with lupus, wherein the patient is ENA−.
126. The method of embodiment 125, wherein the antibody specifically binds an interferon selected from the group consisting of: interferon α; interferon β; interferon ω; interferon λ; and combinations thereof.
127. The method of embodiment 125, wherein the antibody specifically binds interferon α.
128. The method of embodiment 127, wherein the antibody is rontalizumab.
129. The method of embodiment 125, wherein the ENA status of the patient is determined by detecting autoantibodies in a sample from the patient wherein the autoantibodies selected from the group consisting of anti-Ro, anti-La, anti-SM, anti-RNP, and combinations thereof.
130. The method of embodiment 129, wherein the sample is selected from the group consisting of: whole blood, blood-derived cells, plasma, serum, and combinations thereof
131. The method of embodiment 125, wherein the antibody is administered intravenously.
132. The method of embodiment 125, wherein the lupus is systemic lupus erythematosus.
133. The method of embodiment 125, wherein the patient has a baseline interferon signature metric (ISM) that is greater than or equal to the ISM of a healthy individual.
134. The method of embodiment 125, wherein the patient has an ISM that is lower following administration of the antibody as compared to the patient's baseline ISM.
135. The method of embodiment 133 or 134, wherein the ISM is determined by measuring the expression level of at least one gene selected from the group consisting of: CMPK2, EPST1, HERC5, and combinations thereof.
136. The method of embodiment 133 or 134, wherein the ISM is determined by measuring the expression level of at least one gene selected from the group consisting of: IFI27, IFI44, IFIT1, MX1, OAS1, OAS2, OAS3, and combinations thereof.
137. The method of embodiment 125, further comprising administering a second medicament to the subject.
138. The method of embodiment 137, wherein the second medicament is elected from the group consisting of: a corticosteroid, a non-steroidal anti-inflammatory drug (NSAID), an anti-malarial agent, a statin, and combinations thereof.
139. A method of identifying a lupus patient who may benefit from treatment with an interferon type I antibody, the method comprising determining ENA status of the patient, wherein a patient who is determined to have an ENA status of ENA− is identified as a patient who may benefit from treatment with the interferon type I antibody.
140. A method of optimizing therapeutic efficacy for treatment of lupus, the method comprising determining ENA status of a lupus patient, wherein a patient who is determined to have an ENA status of ENA− has increased likelihood of benefit from treatment with the interferon type I antibody.
141. A method of predicting responsiveness of a lupus patient to treatment with an interferon type I antibody, the method comprising determining ENA status of the patient, wherein a patient who is determined to have an ENA status of ENA− is identified as a patient who is likely to respond to treatment with the interferon type I antibody.
142. A method for determining the likelihood that a lupus patient will benefit from treatment with an interferon type I antibody, the method comprising determining ENA status of the patient, wherein a patient who is determined to have an ENA status of ENA− is identified as a patient who is likely to respond to treatment with the interferon type I antibody.
143. The method of any one of embodiments 139-142, wherein the ENA status is determined by detecting autoantibodies in a sample from the patient wherein the autoantibodies selected from the group consisting of anti-Ro, anti-La, anti-SM, anti-RNP, and combinations thereof.
144. The method of any one of embodiments 139-142, wherein the sample is selected from the group consisting of: whole blood, blood-derived cells, plasma, serum, and combinations thereof.
145. The method of any one of embodiments 139-142, wherein the lupus is systemic lupus erythematosus.
146. The method of any one of embodiments 139-142, further comprising administering an effective amount of the interferon type I antibody to the patient.
147. The method of embodiment 146, wherein the antibody is administered intravenously.
148. The method of embodiment 146, wherein the antibody specifically binds interferon α.
149. The method of embodiment 148, wherein the antibody is rontalizumab.
150. The method of embodiment 146, wherein the antibody is administered for at least 24 weeks.
151. The method of embodiment any one of embodiments 139-142, wherein the patient has a baseline interferon signature metric (ISM) that is greater than or equal to the ISM of a healthy individual.
152. The method of any one of embodiments 139-142, wherein the patient has an ISM that is lower following administration of the antibody as compared to the patient's baseline ISM.
153. The method of embodiment 151 or 152 wherein the ISM is determined by measuring the expression level of at least one gene selected from the group consisting of: CMPK2, EPST1, HERC5, and combinations thereof.
154. The method of embodiment 151 or 152 wherein the ISM is determined by measuring the expression level of at least one gene selected from the group consisting of: IFI27, IFI44, IFIT1, MX1, OAS1, OAS2, OAS3, and combinations thereof.
155. The method of embodiment 146, further comprising administering a second medicament to the subject.
156. The method of embodiment 155, wherein the second medicament is elected from the group consisting of: a corticosteroid, a non-steroidal anti-inflammatory drug (NSAID), an anti-malarial agent, a statin, and combinations thereof.
157. A method of treating lupus in a patient, the method comprising administering an effective amount of an interferon type I antibody to a patient diagnosed with lupus, wherein the patient has a baseline interferon signature metric (ISM) that is greater than or equal to the ISM of a healthy individual. In some embodiments, the ISM has been determined by measuring mRNA expression levels of one or more IRGs in a sample (e.g., blood sample) from the patient.
158. A method of treating lupus in a patient, the method comprising administering an effective amount of an interferon type I antibody to a patient diagnosed with lupus, wherein the patient has an ISM that is lower following administration of the antibody as compared to the patient's baseline ISM. In some embodiments, the ISM is determined by measuring mRNA expression levels of one or more IRGs in a sample (e.g., blood sample) from the patient.
159. The method of embodiment 157 or 158 wherein the antibody specifically binds an interferon selected from the group consisting of: interferon α; interferon β; interferon ω; interferon λ; and combinations thereof.
160. The method of embodiment 157 or 158, wherein the antibody specifically binds interferon α.
161. The method of embodiment 160, wherein the antibody is rontalizumab.
162. The method of embodiment 157 or 158, wherein the ISM is determined by measuring the expression level of at least one gene selected from the group consisting of: CMPK2, EPST1, HERC5, and combinations thereof.
163. The method of embodiment 157 or 158 wherein the ISM is determined by measuring the expression level of at least one gene selected from the group consisting of: IFI27, IFI44, IFIT1, MX1, OAS1, OAS2, OAS3, and combinations thereof.
164. The method of embodiment 157 or 158 wherein the antibody is administered intravenously.
165. The method of embodiment 157 or 158 wherein the lupus is systemic lupus erythematosus.
166. The method of embodiment 157 or 158, further comprising administering a second medicament to the subject.
167. The method of embodiment 166, wherein the second medicament is elected from the group consisting of: a corticosteroid, a non-steroidal anti-inflammatory drug (NSAID), an anti-malarial agent, a statin, and combinations thereof.
168. A method of identifying a lupus patient who may benefit from treatment with an interferon type I antibody, the method comprising determining the baseline ISM status of the patient, wherein a patient who has a baseline ISM greater than or equal to the ISM of a healthy individual is identified as a patient who may benefit from treatment with the interferon type I antibody.
169. A method of optimizing therapeutic efficacy for treatment of lupus, the method comprising determining the baseline ISM status of the patient, wherein a patient who has a baseline ISM greater than or equal to the ISM of a healthy individual has increased likelihood of benefit from treatment with the interferon type I antibody.
170. A method of predicting responsiveness of a lupus patient to treatment with an interferon type I antibody, the method comprising determining the ISM status of the patient, wherein a patient who has an ISM greater than or equal to the ISM of a healthy individual is identified as a patient who is likely to respond to treatment with the interferon type I antibody.
171. A method for determining the likelihood that a lupus patient will benefit from treatment with an interferon type I antibody, the method comprising determining the ISM status of the patient, wherein a patient who has an ISM greater than or equal to the ISM of a healthy individual is identified as a patient who is likely to respond to treatment with the interferon type I antibody.
172. The method of any one of embodiments 168-171, wherein the lupus is systemic lupus erythematosus.
173. The method of any one of embodiments 168-171, further comprising administering an effective amount of the interferon type I antibody to the patient.
174. The method of embodiment 173, wherein the antibody is administered intravenously.
175. The method of embodiment 173, wherein the antibody specifically binds interferon-α.
176. The method of embodiment 175, wherein the antibody is rontalizumab.
177. The method of embodiment 173, wherein the antibody is administered for at least 24 weeks.
178. The method of any one of embodiments 168-171, wherein the patient has an ISM that is lower following administration of the antibody as compared to the baseline ISM.
179. The method of any one of embodiments 168-171, wherein the ISM is determined by measuring the expression level of at least one gene selected from the group consisting of: CMPK2, EPST1, HERC5, and combinations thereof.
180. The method of any one of embodiments 168-171, wherein the ISM is determined by measuring the expression level of at least one gene selected from the group consisting of: IFI27, IFI44, IFIT1, MX1, OAS1, OAS2, OAS3, and combinations thereof.
181. The method of embodiment 173, further comprising administering a second medicament to the subject.
182. The method of embodiment 181, wherein the second medicament is elected from the group consisting of: a corticosteroid, a non-steroidal anti-inflammatory drug (NSAID), an anti-malarial agent, a statin, and combinations thereof.

Further details of the invention are illustrated by the following non-limiting Examples. The disclosures of all citations in the specification are expressly incorporated herein by reference.

### EXAMPLES

### Example 1

### SLE Clinical Study

### 1. Summary

A two-part Phase II, randomized, double-blind, placebo-controlled multicenter study was carried out to evaluate the efficacy and safety of rontalizumab compared with placebo in patients with moderately to severely active SLE. The primary efficacy endpoint was evaluated at 24 weeks.

In Part 1, patients were randomized at a 2:1 ratio (active:placebo) to receive either rontalizumab 750 mg, administered via monthly intravenous (IV) infusion, or matching placebo. Part 2 was initiated upon completion of recruitment for Part 1. In Part 2, patients were randomized at a 2:1 ratio (active:placebo) to receive either rontalizumab 300 mg, administered via subcutaneous (SC) injection every 14 days, or matching placebo.

A summary of the trial design is in Table 4.

TABLE 4

| | |
|---|---|
| Design | Randomized, double-blind, placebo controlled, multiple |
| | dose study to evaluate effect of Rontalizumab vs placebo |
| | on SLE disease score |
| Population | Patients had a diagnosis of moderately to severely active |
| | SLE according to the current ACR criteria (Hochberg, |
| | Arthritis Rheum. 40:1725, 1997), including an anti-nuclear |
| | antibody (ANA) titer of 1:80 or greater at the time of |
| | screening, and a British Isles Lupus Assessment Group |
| | index (BILAG) A score in one or more domains, or a |
| | BILAG B score in two or more domains. |
| Sample Size | 238 patients were enrolled; 235 were in the efficacy- |
| | evaluable population |
| Study Duration | 24 weeks (and 48 week safety follow-up) |
| Schedule, Dose | subcutaneous formulation (SC), Q2w, 1 active dose level |
| | (300 mg) OR intravenous (IV), Q4w, 1 active dose level |
| | (750 mg) |
| 1° endpoint | BILAG response index at week 24 |
| | Improvement: step-down in all baseline BILAG A and B |
| | domains. |
| | No worsening: no new BILAG A or more than one |
| | BILAG B; not classified as treatment failure (e.g., |
| | failure to achieve steroid taper, rescue medication). |
| 2° endpoint | SRI (SLE Response Index) at Week 24 |
| | Improvement: reduction in baseline SELENA SLEDAI ≧ 4 |
| | points |
| | No worsening: no new BILAG A or more than one new |
| | BILAG B; no worsening in PGA by >10%; not classified |
| | as treatment failure. |

Patient population in the study included 94% female and had a mean age of 39. Racial/ethnicity distribution of the study population was 46% white, 37% American Indian/Alaska native, 14% black race, and 49% Hispanics. Demographics, baseline characteristics were generally balanced within each cohort, reflecting moderate to severe disease activity (mean SELENA SLEDAI of ˜10, & ≧1 BILAG A, or 2 B domains, mean disease duration ˜6.5 yrs). 76% of all patients were ISM^{hi} at baseline (ISM score ≧1). Lower ISM scores were not associated with lower disease activity at baseline (i.e., pre-treatment). ISM^{lo} patients and ISM^{Hi} patients had comparable mean SELENA-SLEDAI, BILAG, CLASI Activity scores, swollen/tender joint counts, morning stiffness duration.

Starting at the screening period, and for the first 14 days following randomization, patients might receive a steroid regimen ranging from 0.25 to 0.5 mg/kg prednisone equivalent. Investigators were to select the lowest prednisone dose within this range that, in the investigator's judgment, was likely to result in adequate control of disease symptoms. Beginning on study Day 15, this regimen was tapered to a target dose of 10 mg/day prednisone equivalent or less by the end of Week 6 (see Section 3.3.2). This initial steroid regimen was not required if investigators or patients believe that its risks would outweigh the potential benefits.

Where clinically appropriate, a faster steroid taper was encouraged. In cases of documented steroid toxicity or intolerance, the steroid taper would start prior to Day 15. In cases where the screening period was prolonged and steroid exposure during screening exceeded 20 mg prednisone (or equivalent) for 14 or more days, the steroid taper would begin prior to Day 15. After reaching a daily steroid dose of 10 mg, patients continued to taper steroids as tolerated, aiming to discontinue steroids whenever possible.

Patients discontinued all immunosuppressive regimens (e.g., azathioprine, methotrexate, mycophenolate mofetil, cyclosporine, tacrolimus) during screening, but no later than Day 1 (see Section 3.3.2). Use of anti-malarials (e.g., hydroxychloroquine) was permitted but was not introduced or altered in dose during the study period. If patients experienced persistent or increasing disease activity at any time during the study, steroids could be increased and/or immunosuppressive regimens resumed or initiated according to the investigator's clinical judgment. Investigators were free to administer additional treatments/rescue therapy according to their clinical judgment at any time if required, due to persistent disease activity or flare. Patients who required increases in their steroid regimen exceeding a certain threshold (see Section 3.3.2) or who required initiation or resumption of immunosuppressive regimens would, under most circumstances, be permitted to remain in the study and continued to receive investigational product, but would be classified as having experienced treatment failure during analysis. Exceptions are detailed in the Section 3.3.2.

SLE disease activity was assessed monthly through Week 36 and then every 12 weeks through Week 72 using the BILAG 2004 index, Safety of Estrogen in Lupus Erythematosus National Assessment-Systemic Lupus Erythematosus Disease Activity Index (SELENA-SLEDAI), the SELENA Flare Index-Revised (SFI-R), and Physician's Global Assessment (PGA). For patients with arthritis at the time of randomization or at any time prior to Week 24, monthly (through Week 36) 28 joint counts were performed and the duration of morning stiffness was recorded. For patients with mucosal or cutaneous disease at randomization or at any time prior to Week 24, monthly (through Week 36) assessments using the Cutaneous Lupus Erythematosus Disease Area and Severity Index (CLASI) were performed, and digital photographs obtained. In addition, certain patient-reported outcomes (PROs) were measured.

The primary efficacy endpoint for this trial was the proportion of patients who achieve a reduction of all BILAG A domains present at randomization to BILAG B or better, and of all BILAG B domains present at randomization to BILAG C or better at Week 24 without having one or more new BILAG A or two or more new BILAG B manifestations at Week 24 and without being classified as Treatment failure (e.g., due to additional treatments) prior to Week 24. Additional secondary and exploratory endpoints are listed in the Section 2.2.

Safety data, including adverse events, serious adverse events, and laboratory abnormalities were monitored throughout the study.

### 2. Outcome Measures

2.1 Primary Outcome Measure

The primary endpoint for this trial was the proportion of patients who achieve a reduction of all BILAG A domains present at randomization to BILAG B or better, and of all BILAG B domains present at baseline to BILAG C or better at Week 24 without having one or more new BILAG A or two or more new BILAG B manifestations at Week 24 and without being classified as treatment failure (e.g., due to additional treatments) prior to Week 24. Patients who dropped out of the study prior to Week 24 or whose response status could not be determined were considered to be non-responders for the purpose of the primary analysis. The BILAG 2004 disease activity index was used as the primary instrument to capture changes in disease activity in this trial.

Additional instruments that were utilized to capture SLE disease activity in this study include the SELENA-SLEDAI, the SFI-R, and the Physician's Global Assessment. Organ system-specific endpoints such as 28 joint counts (for patients with arthritis) and the CLASI (for patients with mucosal or cutaneous manifestations) were used to supplement the SLE disease activity instruments. In addition, where possible, digital photographs of representative mucocutaneous lesions should be obtained at Weeks 28, 32, 36, 40, 44, 48, 52, 56, 60, 64, 68, and 72 (+/−7 days) (this was optional and subject to availability of equipment).

2.2 Secondary Outcome Measures

The secondary outcome measures included the following: 1) time-adjusted area under the curve (AUC) of the BILAG index global score over 24 weeks; 2) treatment failure status (see Reference Drugs, Section 3.3.2); 3) time to treatment failure; 4) BILAG index global score at Week 24; 5) time to sustained reduction (for at least two consecutive visits) in all baseline BILAG A scores to B or better, and all baseline BILAG B scores to C or better over 24 weeks; 6) time-adjusted SELENA-SLEDAI AUC over 24 weeks; 7) SELENA-SLEDAI score at Week 24; 8) combined SELENA-SLEDAI, PGA, and BILAG response at Week 24, defined by the following criteria: reduction from baseline in SELENA-SLEDAI score of at least four points; no worsening in PGA (with worsening defined as an increase in PGA of more than 0.3 points from baseline); no new BILAG A organ domain score, and no more than one new BILAG B organ domain score any time between baseline and Week 24.

2.3 Additional Outcome Measures

Additional exploratory outcome measures included the following: a) steroid burden (average corticosteroid burden measured by the time-adjusted AUC of corticosteroid dose between Week 8 and Week 24); b) SLE Flare (proportion of patients with mild, moderate, and severe disease flares between Week 8 and Week 24, using the SELENA-SLEDAI flare index and the SFI-R; time to mild, moderate, and severe disease flare using the SELENA-SLEDAI flare index and the SFI-R); c) PROs (change from baseline to Week 24 in the Physical Component Summary of the Short Form-36 (SF-36) Health Survey; change from baseline to Week 24 in the Functional Assessment of Chronic Illness Therapy (FACIT)-Fatigue score; change from baseline to Week 24 in the Subject's Global Assessment of Disease Activity; d) diagnostic (response at Week 24, as defined by the primary outcome measure, in patients classified as ISM signature positive at baseline; treatment failure status in patients classified as ISM-signature positive at baseline).

2.4 Pharmacokinetic and Pharmacodynamic Outcome Measures

The PK and PD outcome measures were as follows: Incidence of anti-rontalizumab antibodies; Change in the expression of selected interferon regulated genes (IRGs) over time; PK parameters of rontalizumab; Change in the levels of interferon-induced proteins and other serum/plasma analytes.

2.5 Safety Plan

IFNalpha is an important cytokine contributing to innate and acquired immune defenses. Therefore, it is possible that antagonizing this pathway may result in increased susceptibility to infections, particularly viral infections. In addition, patients with SLE are at increased risk for infections due to the underlying disease as well as concomitant treatments such as corticosteroids and immunosuppressants.

The safety plan for this trial consisted of three key elements: patient selection, treatment and monitoring.

Patient Selection:

Patients at high risk of infection (e.g., those with multiple recurrences of herpes virus reactivations, recent infections or a history of multiple serious infections requiring hospitalization and/or administration of IV antibiotics, or immunodeficiency) were excluded from this trial. Patients with a history of malignancy or carcinoma in situ within 5 years of screening were excluded, except for basal or squamous cell carcinoma of the skin that had been excised and was considered cured. Patients with a history of chronic myelogenous leukemia, hairy cell leukemia, melanoma, renal cell carcinoma, or Kaposi sarcoma were excluded irrespective of the duration of time before screening. Female patients should have had a cervical smear within the period recommended by the American Cancer Society (Saslow et al. 2002) or applicable ex-U.S. local/national guidelines, but it could not have been more than 3 years preceding randomization and results must be available prior to randomization for review (Patients who have had a hysterectomy did not require a cervical smear). Patients were excluded from the trial if the cervical smear had indicated infection with human papilloma virus, the presence of adenocarcinoma in situ (AIS), squamous intraepithelial lesions (HSIL), or cervical intraepithelial neoplasia (CIN) Grade >1.

Treatment:

This trial was designed to minimize patient exposure to concomitant treatment regimens that increased the risk of infection, particularly high-dose corticosteroids and immunosuppressants. The protocol specified a rapid steroid taper (achieving daily prednisone equivalent doses of 10 mg or less by the end of Week 6), as well as discontinuation of concomitant immunosuppressive regimens. Patients with recent exposure to high-dose steroids and certain immunosuppressive regimens were excluded. Patients who required rescue treatment during the study that involved very high doses of steroids or cytotoxic treatment were not be permitted to receive additional doses of investigational product, although they were encouraged to remain in the study for safety and efficacy assessments if deemed safe and appropriate by the investigator.

These provisions were expected to reduce the risk of infection and facilitate attribution of any infectious events that were observed during the course of the trial.

Monitoring:

Any signs or symptoms that could be suggestive of malignancy were promptly and aggressively evaluated and reported to the Sponsor. Incident hematologic abnormalities (e.g., new or worsening neutropenia, anemia, thrombocytopenia, macrocytosis, atypical cells in the WBC differential) were carefully evaluated. Patients who develop malignancy or serious or life-threatening infections during the study must not receive additional doses of investigational product.

### 3. Materials and Methods

3.1 Patients

The following patient selection criteria in 3.1.1 and 3.1.2 applied to initial enrollment (i.e., Parts 1 or 2) in this trial.

3.1.1 Inclusion Criteria

Patients must meet the following criteria for study entry:
1. Ability and willingness to provide written informed consent and comply with study procedures as defined in the protocol.
2. Age 18-65 years.
3. Diagnosis of SLE according to current ACR criteria (Hochberg et al., Arthritis Rheum 40:1725, 1997). At least four criteria must have been met at any time prior to screening. Presence of four criteria at the time of screening was not required.
4. Positive ANA with a minimum titer of 1:80 at the time of screening. Patients with a previously documented ANA titer of at least 1:80 but with a titer of less than 1:80 at the time of screening could not be enrolled only if it was determined during the enrollment adjudication process that based on all available data (including clinical, serological, or other diagnostic findings), active lupus was present at the time of screening.
5. Active disease at the time of screening, defined by the presence of a BILAG A score in at least one organ system or BILAG B scores in at least two organ systems. For BILAG B scores in the following 3 domains, additional criteria applied:

Constitutional Domain:
BILAG B scores to which anorexia contributed did not count towards entry requirements.

Musculoskeletal Domain:
BILAG B scores to which arthritis (moderate)/tendonitis/tenosynovitis contributed did not count towards entry requirements unless objective signs of inflammation (i.e., tenderness, swelling or effusion) were observed in three or more joints. A patient-reported history of arthritis was not sufficient.

Neuropsychiatric Domain:
BILAG B scores to which lupus headache contributed did not count towards entry requirements. Cognitive dysfunction did not contribute to a B score unless it had been established using appropriate cognitive testing and was documented in the source document.

It should be noted that these additional requirements apply to determining eligibility only and do not represent changes to the BILAG 2004 instrument. Disease manifestations qualifying for entry into this study included Pyrexia (>37.5° C./>99.5° F.), unintentional weight loss, lymphadenopathy, splenomegaly, skin eruption, angioedema (severe), mucosal ulceration (severe), panniculitis/bullous lupus, cutaneous vasculitis, alopecia (severe), digital infarcts, nodular vasculitis, aseptic meningitis, mononeuropathy, plexopathy, polyneuropathy, cranial neuropathy, cognitive dysfunction, seizure disorder, movement disorder, autonomic disorder, cerebellar ataxia, intracranial hypertension, (see exclusion criteria, section 3.1.2), myositis, arthritis, tendonitis/tenosynovitis (see exclusion criteria, section 3.1.2), myocarditis, pericarditis, cardiac valvular dysfunction (new), pleuritis, pleural effusion with dyspnea, interstitial alveolitis/pneumonitis, cardiac tamponade, pulmonary hemorrhage/vasculitis, arrhythmia, shrinking lung syndrome, endocarditis, aortitis, coronary vasculitis, abdominal serositis, ascites, lupus enteritis/colitis, intestinal pseudo-obstruction, malabsorption, lupus hepatitis, protein-losing enteropathy, acute lupus pancreatitis, acute lupus cholecystitis, orbital inflammation, myositis, proptosis, keratitis vaso-occlusive disease of retina or choroid, optic neuritis, anterior or posterior uveitis, retinal vasculitis, scleritis, proteinuria, accelerated hypertension, elevated creatinine (refer to BILAG 2004 manual for quantitative cut-offs; and sle-related exclusion criteria, section 3.1.2), cytopenias, anemia, and hemolysis. In each case the manifestation must, in the considered opinion of the Investigator, be attributable to the patient's SLE rather than to a concomitant medical condition. It should be noted that certain manifestations were not counted towards a BILAG B score for the purposes of satisfying the Inclusion Criteria: anorexia, moderate arthritis/tendonitis/tenosynovitis (unless objective inflammation is observed in three or more sites), lupus headache, and cognitive impairment (unless documented by formal cognitive testing).

Patients with certain severe manifestations that mandated urgent standard of care therapy were expressly excluded (active proliferative nephritis, unstable severe neuropsychiatric lupus, and severe anti-phospholipid syndrome; see Section 3.1.2).

Patients of reproductive potential (males and females) agreed to use of a reliable means of contraception (e.g., hormonal contraceptive, patch, vaginal ring, intrauterine device, physical barrier, surgical sterilization, abstinence) throughout their study participation and for at least 24 weeks following the last administration of investigational product. For all female patients except those who had been postmenopausal for at last 1 year and those who had had a hysterectomy, a negative serum pregnancy test was documented at screening. In addition, a negative urine pregnancy test was documented prior to each administration of study drug.

3.1.2 Exclusion Criteria

Patients who met any of the following criteria were excluded from study entry:

a. SLE-Related Exclusions
1. Active lupus nephritis as evidenced by the presence of proteinuria >1 g/24 hr or a urine protein/creatinine ratio (UPC)>1 or the presence of >10 RBC/HPF or RBC casts in the absence of menstrual hematuria or urinary tract infection at the time of screening. Patients with proteinuria characterized by a UPC >1.0 or protein >1 g/24 hr but a UPC <3.0 or protein ≦3 g/24 hr that, in the investigator's opinion, was not due to active proliferative lupus nephritis, were eligible after prior consultation with the Medical Monitor.
2. Unstable neuropsychiatric SLE, including poorly controlled seizure disorder psychosis or acute confusional state, transverse myelitis, stroke or stroke syndrome.
3. History of severe anti-phospholipid antibody syndrome (stroke, arterial or venous thromboembolism, disseminated intravascular coagulation) within 1 year of screening and not on an adequate and stable anticoagulation regimen at the time of screening.

Aspirin alone was generally not considered to be an adequate regimen. Presence of anti-phospholipid antibodies alone (without a history of thromboembolism) was not exclusionary.

b. Exclusions Related to General Health
4. Pregnancy or breastfeeding
5. Lack of peripheral venous access
6. History of severe allergic or anaphylactic reactions to monoclonal antibodies or IV immunoglobulin
7. Significant, uncontrolled medical disease in any organ system not related to SLE (e.g., poorly controlled chronic obstructive pulmonary disease or asthma, cardiovascular disease, accelerated hypertension, major depression) that in the investigator's opinion would preclude patient participation
8. Concomitant conditions (e.g., asthma, Crohn's disease, etc.) that required systemic corticosteroid use within 1 year prior to screening. Use of topical, intraarticular, or inhaled corticosteroids was not exclusionary.
9. History of cancer, including hematologic malignancy, solid tumors, and carcinoma in situ, within 5 years of screening. Basal or squamous cell carcinoma of the skin that had been excised and was considered cured was not exclusionary. A history of chronic myelogenous leukemia, hairy cell leukemia, melanoma, renal cell carcinoma, or Kaposi sarcoma is exclusionary irrespective of the duration of time before screening. Female patients must have had a cervical smear within the period recommended by the American Cancer Society or applicable ex-U.S. local/national guidelines, but it could not be more than 3 years preceding the randomization visit. Patients are excluded from the trial if the cervical smear has indicated infection with human papilloma virus, the presence of adenocarcinoma in situ (AIS), squamous intraepithelial lesions (HSIL), or cervical intraepithelial neoplasia (CIN) Grade >1.
10. History of alcohol or drug abuse within 1 year prior to screening
   c. Exclusions Related to Infectious Disease
11. Any current or recent (within 4 weeks of screening) signs or symptoms of infection, except for the following:
   Minor infections (e.g., common cold, viral gastroenteritis) that had, in the investigator's judgment, completely resolved prior to randomization
   Fungal infections of the nail beds
   Oral or vaginal candidiasis that has resolved with or without treatment prior to randomization
12. History of severe systemic bacterial, fungal, viral, or parasitic infections (two or more hospitalizations or two or more courses of IV antibiotics) within 6 months prior to screening
13. History of severe and/or disseminated viral infections, particularly herpes viruses such as HSV-1, HSV-2, VZV, CMV (e.g., herpes encephalitis, ophthalmic herpes, disseminated zoster, CMV colitis) per the investigator's judgment
14. Episode of zoster (shingles) within 3 months of screening, or more than two episodes within 2 years prior to screening
15. Positive test for HIV, hepatitis B (HBsAg, anti-HBc), hepatitis C. In cases of a positive screening test, patients could be enrolled if a confirmatory test was negative.
16. History of active tuberculosis or positive screening test for latent mycobacterium tuberculosis infection (purified protein derivative [PPD] skin test and QuantiFERON®-TB Gold were acceptable screening assays). If a negative tuberculosis screening test had been documented within the 3 months of screening, no new test was needed. Patients with a history of Bacille Calmette-Guerin (BCG) vaccination were screened using the QuantiFERON®-TB Gold test only. This test was available through the central laboratory. Indeterminate QuantiFERON®-TB Gold test results were followed up by checking for signs and symptoms of tuberculosis infection, a chest X-ray, or other investigations to rule out infection with mycobacterium tuberculosis as appropriate in the investigator's judgment. Patients with a history of latent TB infection who had received an appropriate and documented course of therapy could be included if the screening examination and a chest X-ray performed within 3 months of screening, revealed no evidence of current active infection. In this case, a TB screening test was not be required.
17. History of congenital or acquired immunodeficiency
   d. Exclusions Related to Medications
18. Received any of the following medications within the indicated period of time:
   B cell-depleting therapy (e.g., anti-CD20, anti-CD22) or anti-BLYS therapy within 12 months prior to screening
   Cyclophosphamide or other alkylating agents within 3 months prior to screening
   Thalidomide or thalidomide derivatives within 6 months prior to screening
   Tumor necrosis factor (TNF)-antagonists within 3 months prior to screening
   Any investigational drug within 3 months or 5 half-lives (whichever is longer) of screening
   Transfusion with blood, packed red blood cells, platelets, or intravenous immunoglobulins, or treatment with plasmapheresis or plasma exchange within 6 weeks prior to screening
   Azathioprine >200 mg/day within 3 months prior to screening
   Methotrexate >25 mg/wk within 3 months prior to screening
   Mycophenolate >3 g/day within 3 months prior to screening
   Live vaccines within 30 days prior to randomization
   Pulse IV methylprednisolone (≧500 mg) within 30 days prior to screening
   Oral prednisone (or equivalent systemic corticosteroid) at a dose of >0.5 mg/kg/day for more than 7 days within the 30 days prior to screening or >0.25 mg/day for more than 30 days within the 90 days prior to screening. Patients on chronic moderate to high doses of steroids as defined above were less likely to achieve the protocol-defined steroid taper goal and therefore were not eligible for this trial. For corticosteroid regimens that involved every other day dosing, intermittent administration of parenteral corticosteroids etc., the closest daily prednisone equivalent was calculated according to the conversion table below to determine eligibility.

TABLE 5
Steroid Equivalency Table for Equivalent Doses of Steroid

| Drug | Dose (mg) |
|---|---|
| Hydrocortisone | 20 |
| Cortisone acetate | 25 |
| Prednisone | 5 |
| Prednisolone | 5 |
| Deflazacort | 6 |
| Methylprednisolone | 4 |
| Dexamethasone | 0.75 |
| Betamethasone | 0.75 |
| Triamcinolone | 4 |
| Beclometasone | 0.75 |
| (Eight puffs beclometasone of 4 times a day | |
| is equivalent to 14 mg of oral prednisone | |
| once a day) | |

e. Exclusions Related to Laboratory Tests
19. AST or ALT>2.5× the upper limit of normal (ULN). If the elevated transaminases were due to lupus (e.g., lupus hepatitis) and other causes had been ruled out, the patient could be eligible after discussion with the Medical Monitor.
20. Lipase>2× the ULN. If the elevated lipase was due to lupus (e.g., lupus pancreatitis) and other causes had been ruled out, the patient could be eligible after discussion with the Medical Monitor.
21. Calculated glomerular filtration rate <30 mL/min
22. Hemoglobin <8 g/dL. If the hemoglobin was <8 g/dL but >7 g/dL and anemia was attributed to SLE, patients could be eligible after discussion with the Medical Monitor.
23. Neutrophil count <1500/μL or platelet count <50,000/μL. If the neutrophil count was <1,500/μL but >500/μL or the platelet count was <50,000/μL but >15,000/μL and was attributed to SLE, patients could be eligible after discussion with the Medical Monitor.

3.3 Study Treatment

3.3.1 Trial Drug

Rontalizumab or matching placebo was supplied as a sterile liquid solution that contains no preservatives. Each single-use, 2-cc vial nominally contained 180 mg rontalizumab in 30 mM histidine, 200 mM arginine hydrochloride, pH 5.5 with 0.04% polysorbate 20. In Part 1 of the trial, rontalizumab 750 mg or matching placebo was given by IV infusion, diluted in a 100-cc normal saline bag and administered over approximately 60 minutes once every 4 weeks for a total of 6 doses. Rontalizumab must not be administered by undiluted, rapid IV injection.

In Part 2 of the trial, rontalizumab 300 mg or matching placebo was given as two SC injections into the back of the arm, thigh or abdomen of 1 mL each once every 2 weeks for a total of 12 doses.

3.3.2 Reference Drugs

a. Corticosteroids

Patients who had been on daily systemic (oral or parenteral) corticosteroid doses of >0.5 mg/kg/day of prednisone or equivalent for more than 7 days (cumulative) in the 30 days prior to screening and patients on daily corticosteroid doses of >0.25 mg/kg/day of prednisone or equivalent for more than 30 cumulative days within the 90 days prior to screening were ineligible for this trial.

During the screening period and for up to 14 days after randomization, a daily prednisone (or equivalent) regimen of up to 0.5 mg/kg (maximum, 40 mg/day) could be initiated to treat the moderate to severe disease activity present at screening. Patients with BILAG A manifestations were treated with prednisone up to 0.5 mg/kg/day (maximum 40 mg/day or equivalent). Patients with BILAG B manifestations (and without BILAG A manifestations) could be treated with prednisone ≦0.25 mg/kg/day (or equivalent). The initial steroid regimen was not required if investigators or patients believed that the risks would outweigh the potential benefits. Patients who received steroid doses exceeding 40 mg/day for >7 days during screening and prior to randomization were randomized.

Steroids were tapered to a target dose of no more than 10 mg/day of prednisone (or equivalent) by the end of Week 6 (Day 41). The steroid regimen was tapered as quickly as safely possible. The optimal steroid taper schedule was determined by the investigator, provided that the target dose of 10 mg/day or less is reached by the end of Week 6. After Week 6, patients continued to taper steroids by increments of 1-2.5 mg/wk as tolerated, with the goal of discontinuing steroid treatment. Patients who were unable to reach a daily prednisone dose of 10 mg or less by the end of Week 8 (Day 62) because of disease activity were classified as treatment failures (see below). These patients were allowed to remain in the study if this is, in the investigator's judgment, safe and clinically appropriate.

### Increase in Steroid Dose for Flare/Treatment Failure

Patients who exceed the following steroid doses were considered treatment failures.
Patients unable to complete the steroid taper (not reaching target dose of 10 mg/day or less by the end of Week 8).
Prior to Week 20
   Any increase in steroids exceeding the lowest achieved dose by 20 mg or more for at least 14 days
   Any increase in steroids exceeding the lowest achieved dose by 10 mg or more for at least 28 days
From Week 20 to Week 24
   Received 20 mg or more of prednisone equivalent on any day during this 4-week period
   Received more than 10 but less than 20 mg/day prednisone equivalent for more than 7 days (cumulative)

Patients who received steroid rescue treatment and were classified as treatment failures could remain in the study and continued to receive investigational treatment if this was, in the investigator's judgment, safe and clinically appropriate, except in the following circumstances:

Patients receive daily prednisone (or equivalent) doses of >60 mg for more than 14 days

Patients receive IV pulse steroids of >1000 mg methylprednisolone

3.3.3 Immunosuppressive Regimens

Eligible patients who were on immunosuppressive regimens (e.g., azathioprine, mycophenolate mofetil, methotrexate) at the time of screening discontinued these regimens during the screening period, but no later than Day 1 (the day of randomization). No immunosuppressive medication was administered on Day 1 or thereafter unless required for rescue treatment as specified below. To treat disease activity, a steroid regimen will be initiated as described above.

a. Changes to the Immunosuppressive Regimen

If at any time during the study patients experienced persistent disease activity or a disease flare that did not respond to treatment with steroids, immunosuppressive regimens could be initiated or resumed according to the investigator's discretion. Patients who required resumption/initiation of an immunosuppressive regimen prior to Week 24 were classified as treatment failures.

Patients who required resumption/initiation of an immunosuppressive regimen remained in the study and continued to receive investigational product if, in the investigator's judgment, this was both safe and clinically appropriate except in the following circumstances: Patients received cyclophosphamide or another alkylating agent at any dose; Patients received a biological agent (e.g., anti-CD20, anti-TNF); Patients received mycophenolate mofetil at a dose of >2 g/day; Patients received azathioprine at a dose of >200 mg/day; Patients received methotrexate at a dose of >25 mg/wk; Patients received systemic calcineurin inhibitors (e.g., cyclosporin); Patients received another investigational product.

In these cases, patients did not receive additional doses of investigational drug but remained in the study and completed study assessments per protocol if this was, in the investigator's judgment, safe and clinically appropriate.

3.4 Other Treatments

3.4.1 Non-Steroidal Anti-Inflammatory Drugs

The use of NSAIDs/Cox-2 inhibitors was permitted in this trial. The combination of more than 1 NSAID or Cox-2 inhibitor was not permitted (except for low-dose aspirin given for cardiovascular prophylaxis). For patients on chronic treatment with NSAIDs/Cox-2 inhibitors, the dose remained stable throughout the trial until Week 24 whenever possible, except in case of toxicity or intolerance. Any change in the use of NSAIDs was carefully documented in the source documents. It was essential to document the reason for use and whether or not it was related to lupus (e.g., lupus pericarditis vs. menstrual cramps). The use of H₂-receptor antagonists or proton pump inhibitors for gastrointestinal prophylaxis was permitted.

3.4.2 Antimalarials

Patients were permitted to enter the study on one antimalarial drug (e.g., hydroxychloroquine, chloroquine, quinacrine). The antimalarial regimen was not changed until Week 24 except if mandated by observed or suspected toxicity or intolerance. Antimalarials were not initiated during the trial unless clinically mandated and after prior discussion with the Medical Monitor. Changes were documented in the appropriate source documents.

3.4.3 HMG-CoA Reductase Inhibitors (Statins)

Use of statins was permitted. Doses were kept stable whenever possible (except in cases of toxicity/intolerance). Changes were documented in the appropriate source documents.

3.4.4 Osteoporosis Prophylaxis

All patients receiving steroids received appropriate regimens to help prevent steroid-induced osteoporosis, including calcium and vitamin D supplements and/or bisphosphonates as appropriate in the investigator's opinion. Regimens were kept stable during the study whenever possible.

3.4.5 Other Concomitant Treatments

It was essential that any initiation, discontinuation, or change in dose of any other concomitant medication (prescribed or over-the-counter) be carefully documented in the appropriate source documents. Any concomitant treatments might cause adverse events, and it was critical that their use was documented so that proper attribution of adverse events to investigational treatment vs. concomitant treatments was attempted.

3.5 Study Assessments

The BILAG 2004 index was used as the primary lupus disease activity instrument used in this study. BILAG assessments were conducted for each patient at screening, at randomization, and then once monthly through Week 36 and every 12 weeks thereafter. For patients with arthritis, 28 joint counts were performed. For patients with cutaneous manifestations at baseline or at any timepoint prior to Week 24, the CLASI was completed for each patient at screening, at randomization, and then once monthly through Week 36. In addition, where possible, digital photographs of representative mucocutaneous lesions was obtained. To capture PROs, the Subject's Global Assessment, SF-36 Health Survey, v2, and FACIT-Fatigue Scale were collected at screening, at randomization, at about day 14, and then once monthly through Week 36.

3.5.1 Definitions of Study Assessments

Study assessments are detailed below and were undertaken at various study visits.

a. SLE Disease Activity Assessments

Whenever possible, all applicable SLE disease activity assessments (BILAG index, Selena-SLEDAI, SELENA Flare Index-Revised (SFI-R), PGA, CLASI and joint counts) were performed on the same day.

### BILAG 2004 Index

The BILAG disease activity index (2004 version), herein referred to as BILAG 2004 index, was used as the primary method to assess disease activity in this study.

The BILAG 2004 index assesses 97 clinical signs, symptoms, and laboratory parameters across nine organ system domains: constitutional, mucocutaneous, neuropsychiatric, musculoskeletal, cardiorespiratory, gastrointestinal, ophthalmic, renal, and hematological. The 97 symptoms are rated with respect to severity over the previous month (4 weeks) and with respect to any change from the previous examination (new, improving, stable, worsening, absent). A single alphabetic score (A through E) for each of the nine domains is then derived from the examination results in each organ category.

Investigators were required to score the activity in each domain only at screening to determine patient eligibility. Thereafter, investigators were only required to record the presence or absence of lupus specific items on the BILAG worksheets and eCRFs provided to sites. The Sponsor was responsible for scoring (i.e., A, B, C, D, and E) after randomization.

### SELENA-SLEDAI

The SELENA-SLEDAI was used as an additional instrument to measure disease activity. In this trial, the SELENA-SLEDAI was used to assess disease activity over the preceding 28 days, and the SELENA-SLEDAI flare tool and SFI-R were utilized.

### Physician's Global Assessment

The PGA is a visual analog scale. This is also part of the SELENA flare tool. Physicians were to rate the patient's disease activity over the past 28 days and place a vertical tick mark on a 100-mm analog scale that was gradated from 0 to 3. Patient history, results of the physical examination, as well as pertinent laboratory values should be taken into account when rating the patient's disease activity. Physicians could also refer to the value recorded at the previous visit and move the tick mark as appropriate.

### CLASI

The CLASI is an instrument designed to capture SLE-specific mucocutaneous disease manifestations. It comprises a score for the activity of the disease and a score for the damage caused by the disease). The CLASI was completed at appropriate intervals for any patient who had mucocutaneous manifestations of SLE at a given study visit and at all subsequent visits. The CLASI was used to capture mucocutaneous disease in any patient with mucocutaneous disease manifestations beginning on the visit that the mucocutaneous manifestation was first observed and at monthly intervals thereafter.

It was important that only SLE-specific lesions were included in this assessment. Wherever possible, digital images of the lesions were obtained after obtaining appropriate informed consent. Follow-up photography of the same regions was obtained. Wherever possible the lighting and other conditions that may influence the interpretation of the photographic images should be precisely replicated for photography at each follow-up visit.

### SELENA Flare Index-Revised (SFI-R)

The 2009 revision of the SELENA Flare index evaluates increases in SLE disease activity within eight organ systems: mucocutaneous, musculoskeletal, cardiopulmonary, hematological, constitutional, renal, neurological, and gastrointestinal. Within each organ system, the investigator assessed clinical manifestations and treatment recommendations to arrive at a flare categorization as no flare, mild flare, moderate flare, or severe flare. In the event that the assessment of a clinical manifestation and the recommendation for a treatment change were discrepant, the treatment choice took precedence (in the direction of a higher flare definition). Treatment changes recommended because of intolerance, toxicity, or safety did not count towards a flare definition.

### Joint Counts

For all patients with arthritis, 28-joint counts were performed at appropriate intervals The duration of morning stiffness was recorded (in minutes) at each of these visits.

e. Laboratory Assessments

A posteroanterior chest X-ray (CXR) was performed for all patients as soon as possible after the screening visit. An X-ray was not required if a patient had an X-ray within the previous 6 months and the results were documented and showed no clinically significant abnormality, or if a patient had undergone other chest imaging modalities (e.g., CT, MRI) and no clinically significant abnormality was revealed.

A 12-lead electrocardiogram (ECG) was obtained at various timepoints during the study.

Other laboratory assessments were performed at various timepoints during the study. With the exception of the erythrocyte sedimentation rate (ESR), urine dipstick, urine microscopy examinations and urine pregnancy tests, all laboratory investigations were performed by the central laboratory. Hematology laboratories included hemoglobin, hematocrit, red blood cells, automatically calculated red cell indices (mean cell volume, mean cell hemoglobin, mean cell hemoglobin concentration, red cell distribution width), platelets, white blood cells, and white blood cell differential with percentage and absolute counts of neutrophils, monocytes, lymphocytes, basophils, eosinophils, and bands. Presence of immature white blood cells would be reported. Presence of abnormal red blood cell morphologies would be reported. The ESR was measured in the local laboratory.

The chemistry panel included electrolytes (sodium, potassium, calcium, chloride, bicarbonate and phosphate), urea, creatinine, estimated glomerular filtration rate (eGFR), glucose, triglycerides, total cholesterol, HDL, LDL, alanine aminotransferase, aspartate aminotransferase, amylase, lipase, total and direct bilirubin, alkaline phosphatase, gamma glutamyl transpeptidase, creatine phosphokinase, lactate dehydrogenase, uric acid, albumin, globulin, and total protein concentrations. C-reactive protein was measured by immunoassay using an assay validated for high sensitivity CRP.

A coagulation panel was done at the screening visit and repeated at various timepoints during the study, and included the prothrombin time, partial thromboplastin time, and international normalized ratio. If clinically indicated (e.g., in the case of suspected anti-phospholipid antibody syndrome), additional functional coagulation assays should be performed in the local laboratory or in specialty laboratories as clinically indicated and determined by the investigator. The results of these investigations should be recorded in the source documents.

The serum level of total immunoglobulins (Ig), IgG, IgM, and IgA was determined by immunoassay at various timepoints during the study.

All women of childbearing potential (unless surgically sterilized or at least 1 year post-menopausal) had a serum pregnancy test at screening. Urine pregnancy tests were performed at various timepoints during the study. If a urine pregnancy test was positive, it was confirmed by a serum pregnancy test.

Urinalysis was performed by the local laboratory at the screening visit and at various timepoints during the study. Wherever possible, a first morning void was obtained. Urine was analyzed by dipstick for the presence of blood, protein, glucose, and nitrites, and leucocyte esterase; if present these were reported semi-quantitatively. If protein with a level of 2+ or greater was detected on dipstick analysis a urine sample was submitted for quantitative measurement of protein and creatinine reported as UPC on this specimen and on all subsequent urinalysis samples. If deemed necessary by the Principal Investigator a 24-hour collection was done for a more precise determination of UPC. If blood, nitrites, or leucocyte esterase were detected on dipstick examination, or if WBC or RBC (>5 cells/HPF) were present on microscopy, the urine was sent for bacterial culture and sensitivities. Urine microscopy was done at the local lab and the numbers of red blood cells and white blood cells per high power field reported together with the number and type of casts.

Hepatitis and human immunodeficiency virus (HIV) serology were done at the screening visit. The presence of the hepatitis B surface antigen (HBsAg) and of antibodies against the hepatitis B core antigen, hepatitis C virus antibody, HIV-1 and HIV-2 were determined by immunoassay. In the event that a screening assay was reported as positive but the confirmatory assay is negative, the assay was regarded as negative.

Screening for latent infection with Mycobacterium tuberculosis was performed by the central laboratory using the QuantiFERON® Gold in vitro IFNgamma release assay with whole blood drawn at the screening visit. Sample processing was required by the local laboratory prior to shipping to the central laboratory; details were provided in the Laboratory Manual. Alternatively, for patients who had not received BCG vaccination, a PPD skin test was performed at the site.

The levels of serum complement C3 and C4 components were determined by immunoassay. The 50% total hemolytic capacity of complement (CHSO) was determined by lysis of sensitized red blood cells. Processing instructions for the complement sample were provided in the Laboratory Manual.

The presence and titer of ANA at the screening visit were assessed by indirect immunofluorescence microscopy at the central laboratory. Other pertinent autoantibodies were measured by the central laboratory by immunoassay at screening and at various timepoints during the study, and included antibodies directed against dsDNA and at the extractable ribonucleoproteins Ro (SSA), La (SSB), Sm, and RNP. The anti-phospholipid autoantibody panel included measurement of antibodies directed against cardiolipin (with IgG and IgM specificities reported individually) and anti-beta-2-glycoprotein (IgG and IgM).

For PK analysis, the serum levels of rontalizumab were measured at various timepoints during the study using a validated immunoassay. Samples were drawn before the administration of study drug. Antibodies against rontalizumab were measured at various timepoints during the study using a validated immunoassay.

Stabilized whole blood was collected for RNA extraction and measurement of expression of key IRG at various timepoints during the study, to assess the in vivo pharmacodynamic effect of the investigational product. There were two samples collected at each time point to ensure sufficient RNA for the assay (PD sample RNA). An additional two whole blood samples were obtained for RNA extraction at various timepoints during the study, for measurement of expression of other genes which might be related to SLE or to the mechanism of action of rontalizumab (exploratory RNA). PD biomarker serum and plasma samples were taken at various timepoints during the study for subsequent protein analysis. Instructions for phlebotomy and for the processing of the RNA, serum and plasma samples by the investigator site and by the local laboratory were provided in the Laboratory Manual.

For patients who consented to participate in the DNA Repository Substudy in Association with Rontalizumab Study IFN4575g, a whole blood sample was obtained at the randomization visit and used for extraction of genomic DNA. Further details of the use of sample can be found in the sub-study Protocol. Collection of this sample was subject to an additional Informed Consent process.

Whole-blood samples were collected to determine the potential effect of rontalizumab on peripheral blood lymphocyte subsets by flow cytometry. For standard immunophenotyping blood was collected for T cell counts (CD3⁺, CD4⁺, CD8⁺), B-cell counts (CD19⁺), and NK cell counts (CD16⁺, CD56⁺) in TruCount vacutainers at various timepoints during the study and was analyzed by flow cytometry at the central laboratory. The results were reported as percentages of the total lymphocyte count and as absolute cell numbers per volume of blood. At these same time intervals assessment of additional subsets and activation markers will be performed by specialist laboratories contracted by the Immune Tolerance Network. These additional flow cytometry studies were limited to patients at investigator sites located within the continental United States, for which the blood sample could be couriered to the specialist laboratory within 24 hours of venipuncture. In the event that no pre-treatment baseline sample was collected (e.g., due to error, sample mishandling, etc.), subsequent exploratory flow cytometry samples were not obtained.

3.5.3 Assessments During Treatment

Visits for administration of the investigational product occurred every 4 weeks during Part 1 of the study (IV administration) and every 2 weeks during Part 2 of the study (SC administration). If it was necessary to exceed the specified intervals because of scheduling issues (for example, patient or investigator site staff vacation), this was permitted with prior approval of the Sponsor's Medical Monitor or designee.

3.5.4 Post-Treatment and Early Termination Assessments

Patients who completed the treatment phase of the study (Week 24) returned for additional assessments of safety and efficacy at three subsequent visits at four-weekly intervals (Weeks 28, 32, and 36). After Week 36, these patients returned to the clinic every 12 weeks for three additional visits at Weeks 48, 60, and 72 for assessment of serious adverse events, concomitant medications, continued disease activity measurements, safety laboratory monitoring, ATAs, and PK and PD sampling. Patients who discontinued from the treatment period prior to Week 24 were asked to return to the clinic within 4 weeks after the last administration of investigational product; at this visit the assessments for early termination were done. Except in cases for which consent to participate in this study was withdrawn, patients who discontinued the treatment phase before Week 24 returned for six follow-up visits at which various assessments will be done.

3.9 Assay Methods

In addition to routine safety laboratory samples, the following samples were collected for all patients at various timepoints throughout the study:
Serum and plasma samples for PK, ATA, and biomarker analysis. Rontalizumab levels were measured by an ELISA. ATA levels were measured by a bridging immunoassay. Exploratory biomarkers were measured using an immunoassay.
Whole blood samples were collected for RNA extraction for PD and biomarker analysis. RNA were extracted from whole blood Paxgene Samples. Complementary DNA were generated via reverse transcription followed by quantitative PCR, which could be performed on custom made TaqMan® Low Density Array cards containing primer-probes for each IRG.
Whole blood samples for DNA extraction for pharmacogenomic analysis (optional; applies to patients consenting to participate in DNA Repository Substudy in Association with Rontalizumab [rhuMAb IFNalpha] Study IFN4575g).
Whole blood samples for flow cytometry analysis

3.10 Statistical Methods

Efficacy analyses included all patients who were included in the randomization, received at least one dose of study treatment, and had at least one post-baseline efficacy assessment, with patients allocated to the treatment arm to which they were randomized.

Safety analyses included all patients who were included in the randomization and received at least one dose of study treatment, with patients allocated to the treatment arm associated with the regimen actually received.

3.10.2 Analysis of Treatment Group Comparability

Demographic and baseline characteristics such as age, sex, race/ethnicity, weight, height, duration of SLE, BILAG 2004 index score, SELENA-SLEDAI score, were summarized by treatment group. Continuous data (e.g., age, body weight, and height) were summarized using descriptive statistics (mean, standard deviation, median, minimum, and maximum). For categorical data (e.g., race/ethnicity, and sex), the number and percentage of participants in each category were presented by treatment group.

3.10.3 Efficacy Analyses

a. Primary Efficacy Endpoint

The ability of the two dose regimens of rontalizumab, administered intravenously and subcutaneously, respectively, to improve signs and symptoms in moderate to severe SLE patients was assessed by the BILAG index response at Week 24 as defined in the Primary Endpoint section (see Section 2.1).

A point estimate of the treatment effect size, defined as the difference in the proportion of responders to treatment with either a dose of rontalizumab or placebo at Week 24, was determined, accompanied by a 90% two-sided confidence interval. Exploratory hypothesis testing was performed, with statistical significance judged at the α=0.1 level.

As an exploratory analysis of the primary endpoint, a logistic regression model was used to compare the rontalizumab arms with the combined (IV and SC) placebo arm. The model included treatment, race, previous use of an immunosuppressant (yes/no), and baseline ISM score as covariates. Statistical significance was judged at the α=0.10 level.

b. Secondary Efficacy Endpoints

The secondary efficacy endpoints included the following:
Time-adjusted AUC of the BILAG index global scores from baseline to Week 24 or study discontinuation. The adjusted treatment difference was estimated using an ANCOVA model.
Treatment failure status
   95% confidence intervals for the unadjusted difference between the proportions of patients who were classified as treatment failure in the active arms vs. the combined placebo arm were calculated. Adjusted differences in the proportion of patients classified as treatment failure were also be estimated, using a logistic regression model.
Time to treatment failure
   A stratified Cox proportional hazards model was used to calculate a point estimate of the hazard ratio for each rontalizumab arm relative to the placebo group, along with a 95% confidence interval.

Other secondary or exploratory endpoints were analyzed by methods similar to those presented above.

3.10.5 Pharmacokinetic and Pharmacodynamic Analyses

Serum samples were obtained from all patients for determination and characterization of the pharmacokinetics of rontalizumab and ATAs. Samples were obtained at various timepoints during the study. If a patient decided to withdraw prematurely from the study, a blood sample was obtained for ATA and PK determination.

Measurement of IFN regulated genes was assessed at pre-dose and post-dose timepoints from whole blood RNA preparations and using quantitative RT-PCR analyses. Changes in gene expression were assessed in relation to PK data and clinical data. Additional PK and PD analyses were conducted as appropriate.

3.10.6 Handling of Missing Data

For the determination of treatment response at Week 24, a patient who discontinued from the study early (prior to Week 24) was considered a non-responder. Missing values for continuous endpoints were imputed by carrying the last available observation forward or through the use of other imputation techniques. Patients with an insufficient amount of post-baseline measurements may be excluded from the analysis of the Week 24 efficacy endpoints.

### Example 2

### Interim Analysis

Treatment effects at 24 weeks from patients who received rontalizumab or placebo intravenously are set forth in Tables 6-12 below. Table 6 sets forth the treatment effect (by % responders) according to ISM status using the SRI index. Table 7 sets forth the treatment effect (by % responders) according to ENA status using the SRI index. Table 8 sets forth the treatment effect (by change in BILAG Global Score from baseline) according to ISM status. Table 9 sets forth the treatment effect (by change in SELENA-SLEDAI Score from baseline) according to ISM status. Table 10 sets forth the treatment effect based on the presence of arthritis and according to ENA status using SELENA-SLEDAI and BILAG indices. Table 11 set forth the treatment effect based on the change in swollen joint count and according to ENA status. Table 12 sets forth the treatment effect based on the change in mucutaneous rash and according to ENA status using SELENA-SLEDAI and BILAG indices.

TABLE 6

| | | | | Delta | |
|---|---|---|---|---|---|
| | Statistic | PLC IV | 750 mg IV | (90% CI) | p-value |
| All | N | 41 | 79 | | 0.184 |
| N = 120 | n (%) | 14 (34.2%) | 37 (46.8%) | 12.8% | |
| | 90% CI | 22.0%, 46.3% | 37.6%, 56.1% | −2.3%, | |
| | | | | 28% | |
| ISM hi | N | 30 | 62 | | 0.78 |
| N = 92 | n (%) | 13 (43.3%) | 25 (40.3%) | −3.1% | |
| | 90% CI | 28.5%, 58.2% | 30.1%, 50.6% | −21.1%, | |
| | | | | 14.9% | |
| ISM lo | N | 11 | 17 | | 0.0021 |
| N = 28 | n (%) | 1 (9.1%) | 12 (70.6%) | 61.5% | |
| | 90% CI | 0.5%, 36.4% | 47.8%, 87.6% | 38.4%, | |
| | | | | 84.6% | |

TABLE 7

| | | | | Delta | |
|---|---|---|---|---|---|
| | Statistic | PLC IV | 750 mg IV | (90% CI) | p-value |
| All | N | 41 | 79 | | 0.184 |
| | n (%) | 14 (34.2%) | 37 (46.8%) | 12.8 % | |
| | 90% CI | 22.0%, 46.3% | 37.6%, 56.1% | −2.3 %, | |
| | | | | 28% | |
| ENA+ | N | 18 | 55 | | 0.80 |
| | n (%) | 6 (33.3%) | 20 (36.4%) | 3.4% | |
| | 90% CI | 15.1%, 51.6% | 25.7%, 47.0% | −18.0%, | |
| | | | | 24.7% | |
| ENA− | N | 23 | 24 | | 0.019 |
| | n (%) | 8 (34.8%) | 17 (70.8%) | 34.6% | |
| | 90% CI | 18.5%, 51.1% | 55.6%, 86.1% | 12.3%, | |
| | | | | 57.0% | |

TABLE 8

| | | | | Delta | ANCOVA |
|---|---|---|---|---|---|
| | Statistic | PLC IV | 750 mg IV | (90% CI) | p-value |
| All | N | 41 | 79 | | 0.59 |
| | mean (SD) | −5.1 (5.0) | −4.6 (5.9) | 0.5 | |
| | 90% CI of mean | −6.4, −3.8 | −5.7, −3.5 | −1.1, 2.1 | |
| | median | −4 | −4 | | |
| ISM hi | N | 30 | 62 | | 0.51 |
| | mean (SD) | −5.4 (5.2) | −4.0 (6.0) | 0.7 | |
| | 90% CI of mean | −7.0, −3.8 | −5.3, −2.8 | −1.1, 2.6 | |
| | median | −5.0 | −3.0 | | |
| ISM lo | N | 11 | 17 | | 0.66 |
| | mean (SD) | −4.4 (4.8) | −6.5 (5.1) | −0.8 | |
| | 90% CI of mean | −7.0, −1.8 | −8.6, −4.3 | −4.0, 2.4 | |
| | median | −2.0 | −8.0 | | |

TABLE 9

| | | | | Delta | ANCOVA |
|---|---|---|---|---|---|
| | Statistic | PLC IV | 750 mg IV | (90% CI) | p-value |
| All | N | 41 | 79 | | 0.56 |
| | mean (SD) | −2.7 (4.3) | −3.5 (4.4) | −0.5 | |
| | 90% CI of | −3.9, −1.6 | −4.4, −2.7 | −1.8, 0.9 | |
| | mean | | | | |
| | median | −3 | −4 | | |
| ISM hi | N | 30 | 62 | | >0.99 |
| | mean (SD) | −2.9 (4.4) | −3.3 (4.8) | −0.0 | |
| | 90% CI of | −4.3, −1.6 | −4.3, −2.3 | (−1.7, 1.7) | |
| | mean | | | | |
| | median | −4 | −4 | | |
| ISM lo | N | 11 | 17 | | 0.076 |
| | mean (SD) | −2.2 (4.1) | −4.4 (2.5) | −2.1 | |
| | 90% CI of | −4.4, 0.1 | −5.4, −3.3 | −4.0, −0.2 | |
| | mean | | | | |
| | median | −2.0 | −4.0 | | |

TABLE 10

| | | SELENA ARTHRITIS | | BILAG A or B→C or | |
|---|---|---|---|---|---|
| | | Present→Not Present* | | better | |
| | | PLC (in %) | ACT (in %) | PLC (in %) | ACT (in %) |
| IV Cohort | All | 52.6 | 56.3 | 56.1 | 52.8 |
| Response | ENA- | 41 | 66.7 | 56.5 | 60 |
| at Week 24 | | | | | |

TABLE 11

| ENA | Treatment | | | | |
|---|---|---|---|---|---|
| Group | Group | Variable | N | Mean | Median |
| ENA− | 750 mg IV | # swollen joints at BL | 21 | 4.0 | 4.0 |
| | | # swollen joints at Week 24 | 20 | 1.8 | 1.0 |
| | | Change from BL | 20 | −2.3 | −2.0 |
| | Placebo IV | # swollen joints at BL | 21 | 6.0 | 6.0 |
| | | # swollen joints at Week 24 | 21 | 2.8 | 1.0 |
| | | Change from BL | 21 | −3.2 | −3.0 |
| ENA+ | 750 mg IV | # swollen joints at BL | 50 | 4.7 | 4.0 |
| | | # swollen joints at Week 24 | 49 | 2.4 | 1.0 |
| | | Change from BL | 49 | −2.3 | −2.0 |
| | Placebo IV | # swollen joints at BL | 15 | 4.7 | 5.0 |
| | | # swollen joints at Week 24 | 15 | 2.1 | 1.0 |
| | | Change from BL | 15 | −2.7 | −2.0 |

TABLE 12

| | | SELENA RASH | | BILAG A or B→C or | |
|---|---|---|---|---|---|
| | | Present→Not Present* | | better | |
| | | PLC (in %) | ACT (in %) | PLC (in %) | ACT (in %) |
| IV Cohort | All | 12.5 | 14.8 | 39.4 | 27.7 |
| Response | ENA- | 15.8 | 17.7 | 40 | 35 |
| at Week 24 | | | | | |

### Example 3

### ISM Signature in Patients

The following protocol is provided as an example of the steps that can be taken to measure the various IRG genes for determination of which patients are ISM^{lo} or ISM^{hi}.

### Materials

In this example, primers for RT-PCR of IRG genes Herc5, Tyk1 and EPSTI1 and housekeeping gene transferrin receptor (TRFC) can be used.

### Oligonucleotide Sequences

Oligonucleotide sequence of forward and reverse primers, and dye-conjugated probes that were used are shown below.

All sequences shown in 5′-3′ orientation.

| EPSTI-1 (NM_001002264) | |
|---|---|
| (SEQ ID NO: 23) | |
| Probe: | TGCTCTTGCTGCTGCCGTTTCAGT |
| (SEQ ID NO: 24) | |
| Forward: | AGGCAGAAGAAAACAGAAAATTGC |
| (SEQ ID NO: 25) | |
| Reverse: | GTGTTCAGTCTGGTGGATTTTGG |
| HERC5 (NM_016323) | |
| (SEQ ID NO: 26) | |
| Probe: | CTGCCGGAGAAGCCCACAGCATGG |
| (SEQ ID NO: 27) | |
| Forward: | ACCTCGCAGGAGTACCCTTG |
| (SEQ ID NO: 28) | |
| Reverse: | GCCACCACAAGCGACAAATTC |
| TYKI or CMPK2 (NM_207315) | |
| (SEQ ID NO: 29) | |
| Probe: | CGAAGGACTGGATGCCACGGGTAAA |
| (SEQ ID NO: 30) | |
| Forward: | GAAAGTTCCAGGTTGTTGCCA |
| (SEQ ID NO: 31) | |
| Reverse: | TGAATCTGCCACTGACTGGG |

### RNA Isolation

SLE patient RNA can be isolated from either PBMCs using the RNeasy Mini Kit (Qiagen, #74124) or from whole blood collected in PAXgene tubes using the PAXgene Blood RNA Kit (Qiagen, #762164) following the manufacturer's protocols. On-column DNase treatment can be used in both protocols. RNA can be quantified using the NanoDrop® ND-1000 spectrophotometer.

cDNA Synthesis

1 ug input RNA can be used for first strand synthesis with the iScript cDNA Synthesis Kit (Bio-Rad, #170-8890) following the manufacturer's protocol.

qPCR (RT-PCR) Protocol:

10 ul PCR reactions can be performed in duplicate using a 384-well plate format on the ABI PRISM® 7900HT Sequence Detection System. cDNA can be diluted to a concentration of 5 ng/ml based on a starting RNA concentration and 10 ng can be used per 10 ul reaction (final template concentration of 1 ng/ul). TaqMan® Universal PCR Master Mix (ABI, #4304437) can be used following the manufacturer's protocol with default thermal cycling conditions. Primers and Taqman probe can be designed with Beacon Designer 6.0 and used at a final concentration of 100 nM.

### Analysis:

IRG detection data can be normalized against the housekeeping gene. In this example, delta Ct (DCt) can be calculated for each gene relative to TFRC. The ISM score can be calculated as described elsewhere herein.

### Alternative Method:

Alternatively, whole blood can be collected from lupus patients using PAXgene® blood tubes and extracted using the PAXgene® Blood RNA Kit WD (PreAnalytix). Then the RNA samples can be analyzed by real-time PCR thermocycling using the cobas z 480 analyzer (Roche Diagnostics). For example, the mRNA for Tyk1, EPSTI1 and HERC5 can be analyzed in this way with the mRNA levels of the housekeeping gene TRFC being used to normalize against each IRG reaction.

### Example 4

### Results of Anti-Ds DNA and ISM Analysis

Prior to the start of the rontalizumab treatment cycle, biological samples from lupus patients participating in the Phase II studies (ROSE Trial) described in Example 1 were obtained and analyzed with regard to the IRG gene expression of TYK1, HERC5 and EPST1 and the housekeeping gene TRFC and with regard to anti-dsDNA antibodies. Biological samples from healthy patients were similarly processed.

Anti-ds DNA IgG testing was performed on serum samples by using the AtheNA Multi-Lyte® ANA-II PLUS Test System Kit (manufactured by Inverness Medical Inc., Raritan, N.J.). The IRGs were analyzed in triplicate by qPCR using the cobas z 480 analyzer (Roche Diagnostics). The resulting IRG Ct data was normalized against the resulting Ct data for the housekeeping gene (e.g., TFRC) resulting in a DCt value. (DCt=Ct(ISM gene)-Ct(housekeeping gene) per reaction. A mean value of all the DCts was calculated for that patient.

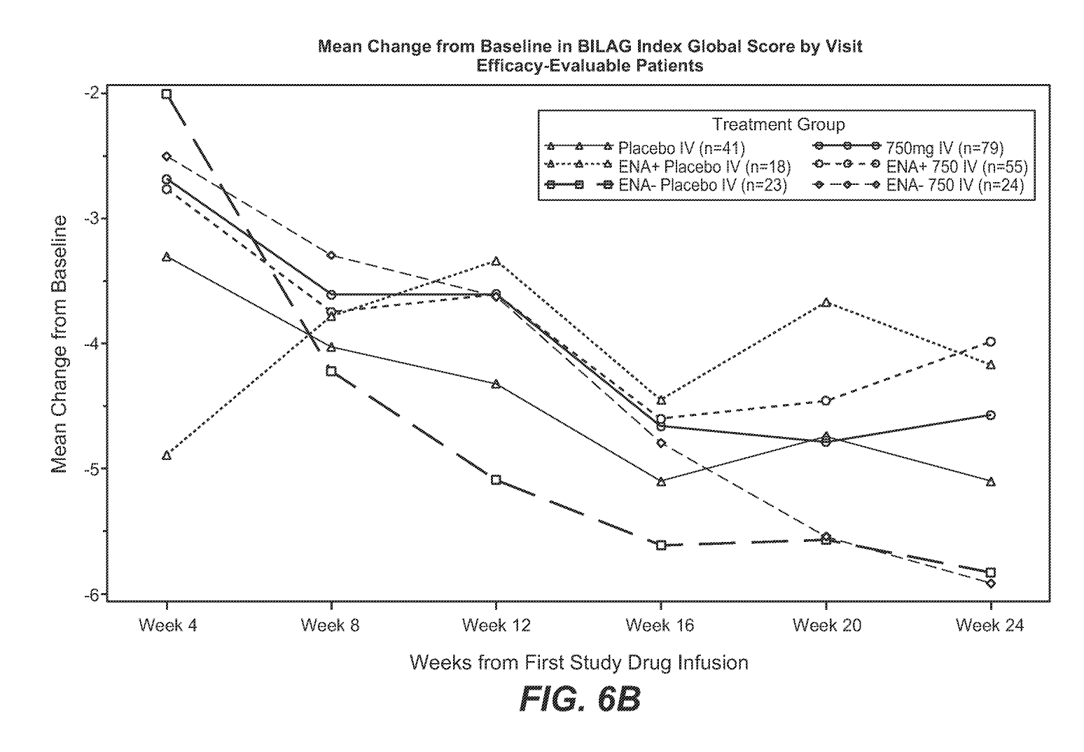

A bimodal distribution of patients was observed based on the amount of the IRG gene expression (mean DCt) observed, which distribution was further characterized relative to a cutoff (represented by “1”) that differentiated between the lower and higher IRG expressing groups of patients (FIG. 9). The lower expressing IRG population in this instance could be generally described as having a mean DCt that was (1) less than 1.5 times the value of the expression levels of the IRGs of a healthy person or (2) less than two standard deviations over the median value of the expression levels of the same IRGs in healthy patients. The lower expressing IRG population was generally referred to as “ISM^{lo}” while the higher expressing ISM population was generally referred to as “ISM^{hi}.”

Irregardless of the ISM^{lo} or ISM^{hi} status of the patient prior to treatment with an interferon alpha inhibitor, it was observed that ISM^{lo} or ISM^{hi} patients generally have a similar SRI, BILAG and/or SLEDAI clinical score.

Individual response rates to rontalizumab treatment based on BILAG Response Index/SRI scores were matched with the individual's IRG expression status (i.e., ISM^{lo} or ISM^{hi}) to result in the following observations.

TABLE 13

| IV | | SC | | Pooled | |
|---|---|---|---|---|---|
| P | R 750 mg | P | R 300 mg | P | R (IV + SC) |
| (n = 41) | (n = 79) | (n = 38) | (n = 77) | (n = 79) | (n = 156) |

BILAG Response Index

| | | | | | | |
|---|---|---|---|---|---|---|
| All | 34% | 38% | 47% | 53% | 41% | 46% |
| ISM | 37% | 40% | 40% | 46% | 38% | 43% |
| high | | | | | | |
| ISM | 27% | 29% | 62% | 81% | 46% | 55% |
| low | (3/11) | (5/17) | (8/13) | (13/16) | (11/24) | (18/33) |

SRI

| | | | | | | |
|---|---|---|---|---|---|---|
| All | 41% | 48% | 47% | 53% | 44% | 51% |
| ISM | 50% | 42% | 44% | 48% | 47% | 45% |
| high | | | | | | |
| ISM | 18% | 71% | 54% | 75% | 38% | 73% |
| low | (2/11) | (12/17) | (7/13) | (12/16) | (9/24) | (24/33) |

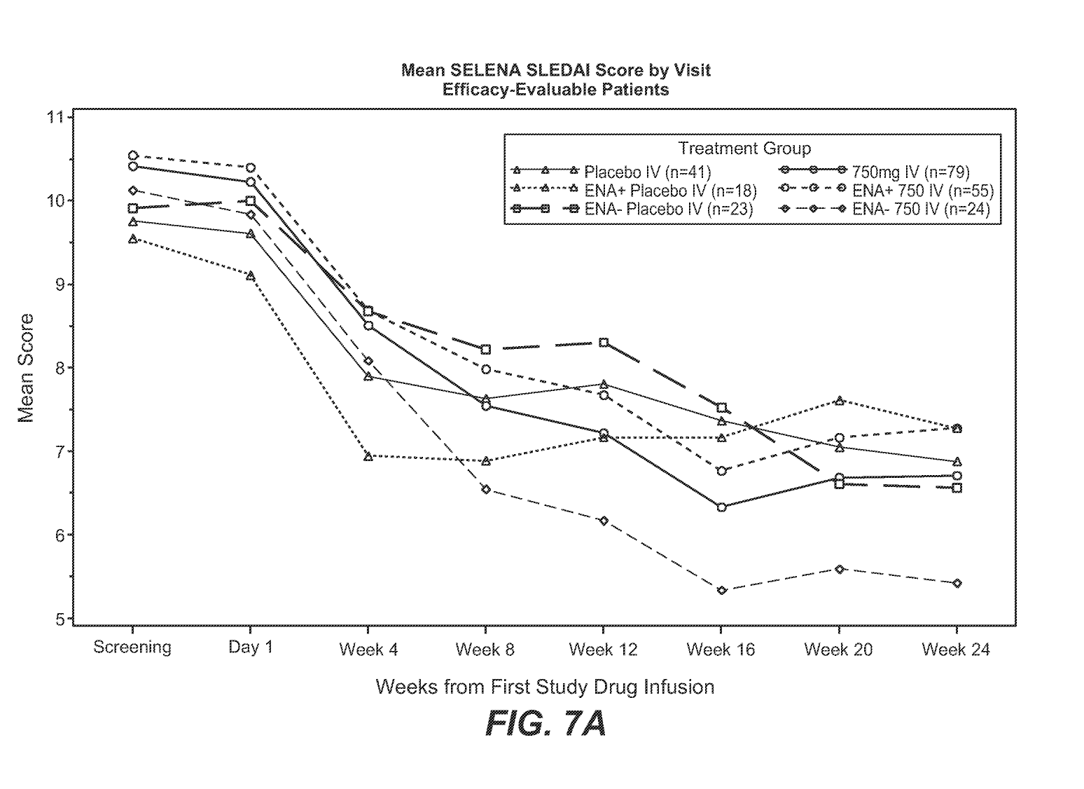

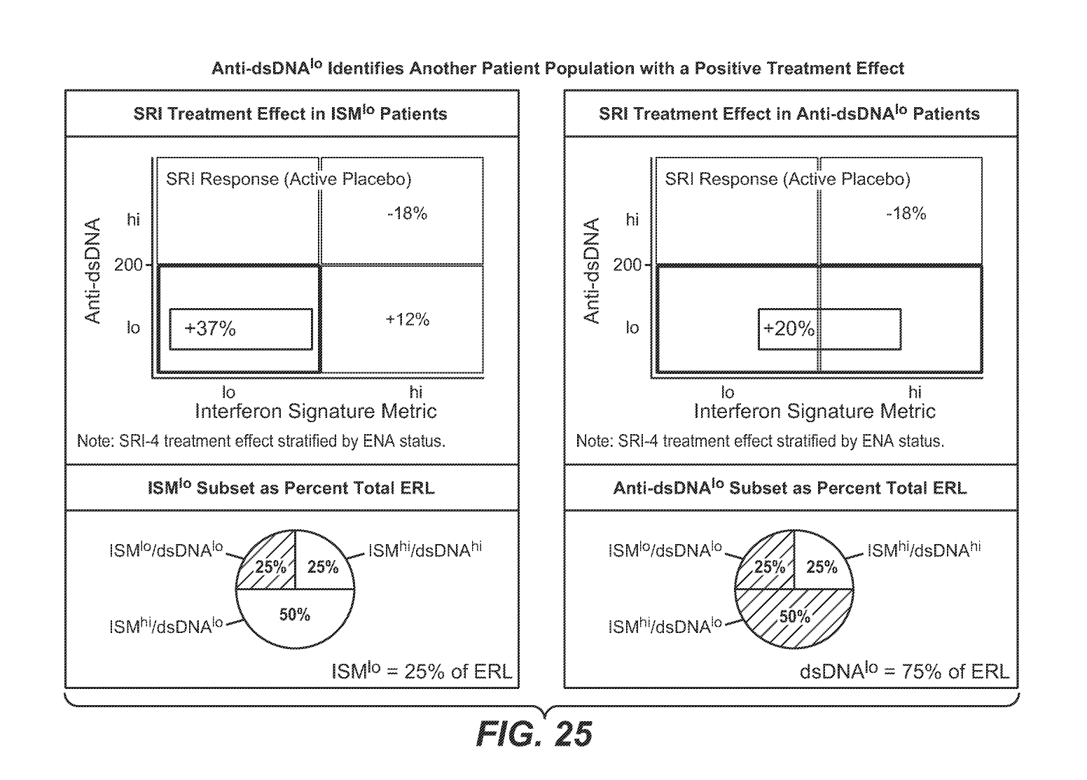

The results suggested a possible increased SRI response rate for IV and SC in ISM low patients, which result was further analyzed in more detail below. Furthermore, additional analysis of the patients based on their ISM score and dsDNA antibody titer indicated that there are three patient populations—ISM^{lo}, ISM^{hi}/dsDNA lo≦2001U), or ISM^{hi}/dsDNA hi (FIGS. 10 and 25). Further analysis of those patient populations suggest that, in addition to ISM^{lo}, ISM^{hi}/dsDNA lo (≦200 IU) signatures may be predictive of responsiveness to rontalizumab and other interferon inhibitors. See FIG. 25 and Table 14.

Seventy eight percent of the patients were classified as having an anti-dsDNA titer of less than 200 IU or ISM^{lo}. These patients responded better than patients that were ISM hi and had an anti-dsDNA titer of greater than 200 IU. See Table 14.

TABLE 14

| | ISM Low | ISM Hi, dsDNA ≦ 200 | ISM Hi, dsDNA > 200 |
|---|---|---|---|
| SRI-4 | | | |
| Rontalizumab (IV + SC) | 73% (24/33) | 53% (47/88) | 23% (8/35) |
| Pbo (IV + SC) | 38% (9/24) | 46% (18/39) | 50% (8/16) |

SRI-4 (excluding serologic items from SELENA SLEDAI)

| | | | |
|---|---|---|---|
| SRI4 | | | |
| Rontalizumab (IV + SC) | 76% (25/33) | 64% (56/88) | 29% (10/35) |
| Pbo (IV + SC) | 42% (10/24) | 46% (18/39) | 56% (9/16) |

SRI-4 Response rates at week 24 from the Rose study are shown in tables below.

| | | | | | | | Treatment | |
|---|---|---|---|---|---|---|---|---|
| | Pbo | 750 mg | Pbo | 300 mg | Pbo | Ront | difference | |
| | IV | IV | SC | SC | (IV + SC) | (IV + SC) | (90% CI) | p-value* |
| Observed/ | | | | | | | | |
| Unadjusted Rates | | | | | | | | |
| ISM Low | 18% | 71% | 54% | 75% | 38% | 73% | 35% | 0.0066 |
| | (2/11) | (12/17) | (7/13) | (12/16) | (9/24) | (24/33) | (15%, 56%) | |
| ISM Low/ | 43% | 53% | 42% | 66% | 42.9% | 58.7% | 16% | 0.0341 |
| dsDNA ≦ 200 IU | (13/30) | (36/68) | (14/33) | (35/53) | (27/63) | (71/121) | (3%, 28%) | |
| anti-ENA- | 43% | 75% | 64% | 68% | 51.4% | 70.9% | 20% | 0.0734 |
| | (10/23) | (18/24) | (9/14) | (21/31) | (19/37) | (39/55) | (3%, 36%) | |
| Rates adjusted by | | | | | | | | |
| anti-ENA status** | | | | | | | | |
| ISM Low | 17.5% | 73.6% | 54.4% | 73.9% | 36.9% | 73.7% | 37% | 0.0055 |
| | | | | | | | (18%, 56%) | |
| ISM | 41.4% | 60.4% | 43.3% | 64.8% | 42.4% | 62.7% | 20% | 0.0088 |
| Low/dsDNA ≦ 200 | | | | | | | (8%, 33%) | |
| IU | | | | | | | | |

*CMH p-value with stratification by administration mode (IV/SC).
**Rates for Pbo/Ront IV + SC groups are also stratified by IV vs. SC; CMH p-value with stratification by anti-ENA status and administration mode (IV/SC).

### Example 5

### Reduction in SRI after Rontalizumab therapy

Patient response rate from phase 2 study described in Example 1 was evaluated with different response criteria. Tables 15 and 16 below show response criteria defined as SRI-4, SRI-5, SRI-6, and SRI-7 and response rate in 300 mg/2w SC treatment group as compared to placebo. As shown below, the response for SC treatment group was detected clearer with the stricter response criteria.

TABLE 15
Response criteria SRI-4, SRI-5, SRI-6 and SRI-7

| | SRI-4 (SRI) | SRI-5 | SRI-6 | SRI-7 |
|---|---|---|---|---|
| Reduction in SELENA- | 4 | 5 | 6 | 7 |
| SLEDAI score | | | | |
| New BILAG A or | 0 | 0 | 0 | 0 |
| New BILAG B | ≦1 | ≦1 | ≦1 | ≦1 |
| Worsening in PGA (%) | ≦10 | ≦10 | ≦10 | ≦10 |
| Treatment failure | No | No | No | No |

TABLE 16
Response rate of phase 2 study at 24 wk endpoint

| | Pbo SC | 300 mg SC | Adjusted Tx Difference* | |
|---|---|---|---|---|
| Endpoint | (n = 38) | (n = 78) | (90% CI) | |
| SRI-4 | 47% | 53% | 6.5% | (−9.2, 22.2) |
| SRI-5 | 34% | 48% | 14.6% | (−0.1, 29.2) |
| SRI-6 | 32% | 48% | 17.2% | (2.6, 31.7) |
| SRI-7 | 21% | 35% | 14.6% | (0.1, 28.0) |

*Adjusted by stratification factors: prior use of an immunosuppressant and race/ethnicity.

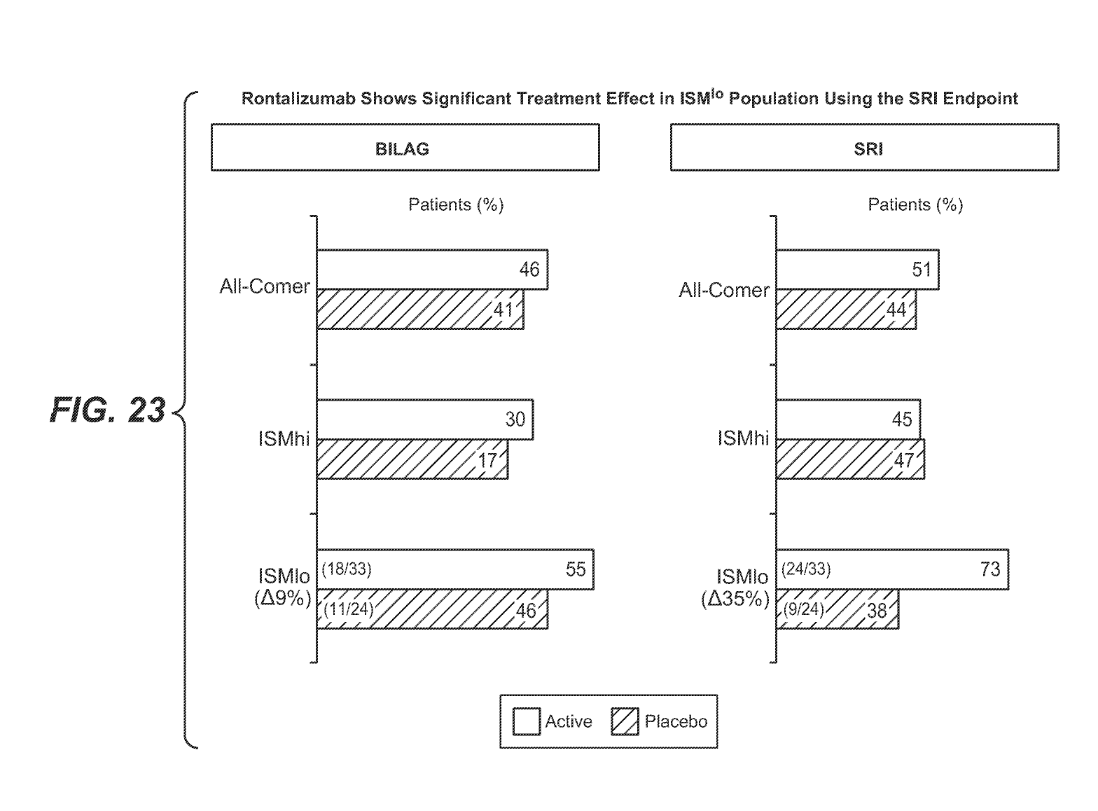

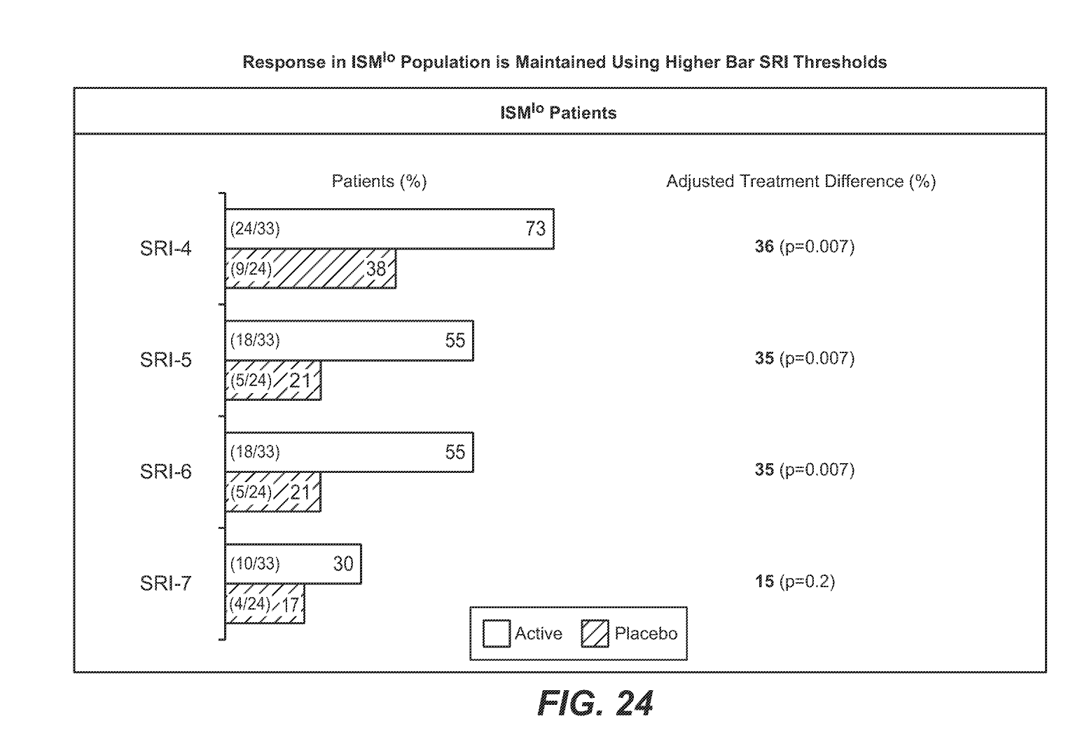

In addition, Table 17 below shows that ISM low subgroup showed higher response rate. See also, FIGS. 23 and 24.

TABLE 17
Response rate of Phase 2 study at 24 week endpoint.

| End- | Pbo (IV + SC) | Tx (IV + SC) | Absolute Tx | Adjusted Tx |
|---|---|---|---|---|
| point | n = 24 | n = 33 | Difference | Difference* |
| SRI-4 | 37.5% (9) | 72.7% (24) | 35.2% | 36.2% |
| | | | p** = 0.014 | p** = 0.007 |
| SRI-5 | 20.8% (5) | 54.6% (18) | 33.8% | 35.1% |
| | | | p** = 0.014 | p** = 0.007 |
| SRI-6 | 20.8% (5) | 54.6% (18) | 33.8% | 35.1% |
| | | | p** = 0.014 | p** = 0.007 |
| SRI-7 | 16.7% (4) | 30.3% (10) | 13.6% | 14.8% |
| | | | p** = 0.35 | p** = 0.20 |

**Fisher exact p-value.

### Example 6

### Prediction of Flares

Flares can be identified by an acute measurable increase in disease activity in one or more organ systems involving new or worse clinical signs and symptoms and/or laboratory measurements. It must be considered clinically significant by the assessor and usually there would be at least consideration of a change or an increase in treatment. (See Ruperto et al., International consensus for a definition of disease flare in lupus. Lupus (2011) 20: 453-462.) Thus, flare refers to onset of disease activity in a patient diagnosed with an immune disorder; in clinical intervention trials of SLE, flares are classified as mild, moderate or severe based on criteria published in Lupus [1999] 8(8):685-91 as the SELENA-SLEDAI Flare Index (SFI) and in a revised form (SFI-R) in Arthritis & Rheumatology [2011] 63(12): 3918-30. Following the protocols described in Example 1, SELENA-SLEDAI was used for a composite assessment of disease activity based on 16 clinical manifestations and eight laboratory measures including two immunological tests with a possible range of overall score from 0 to 105.

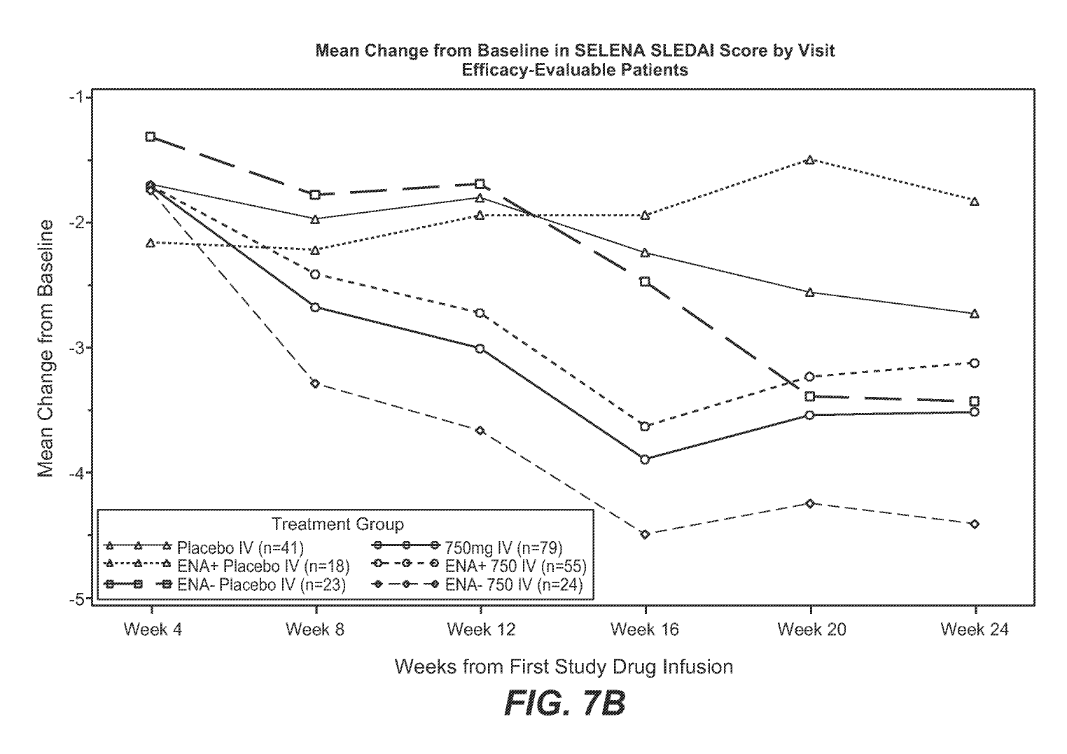

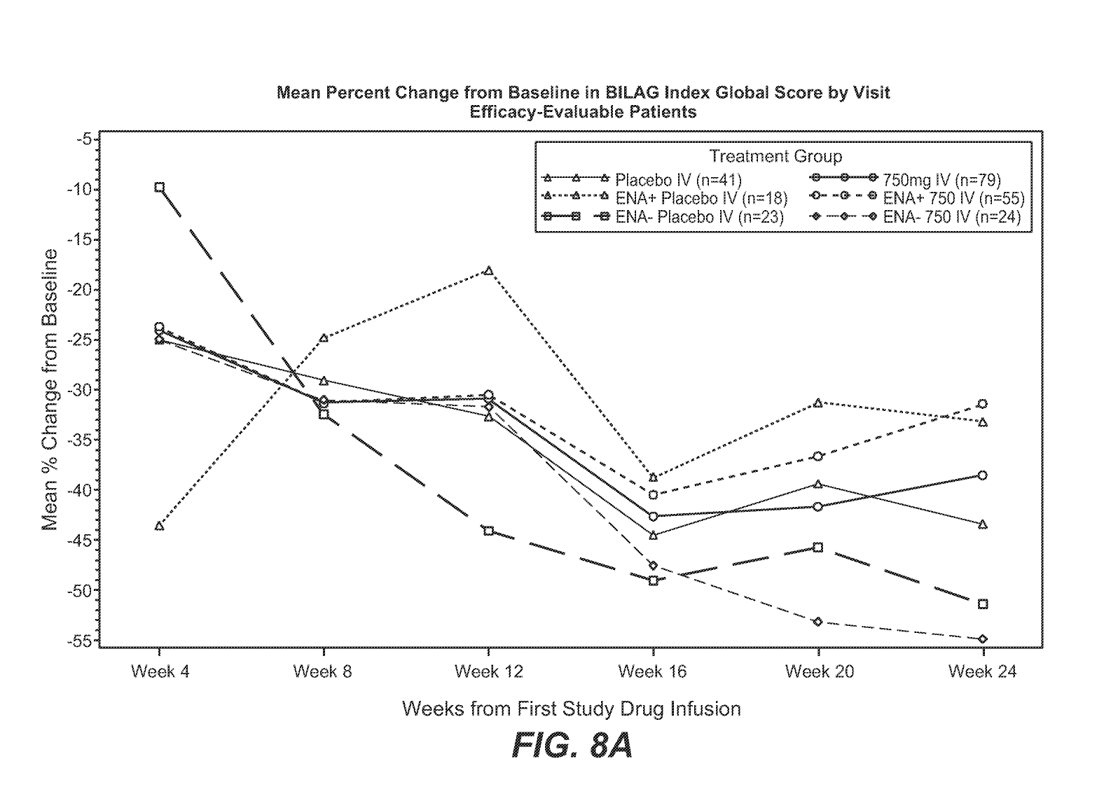

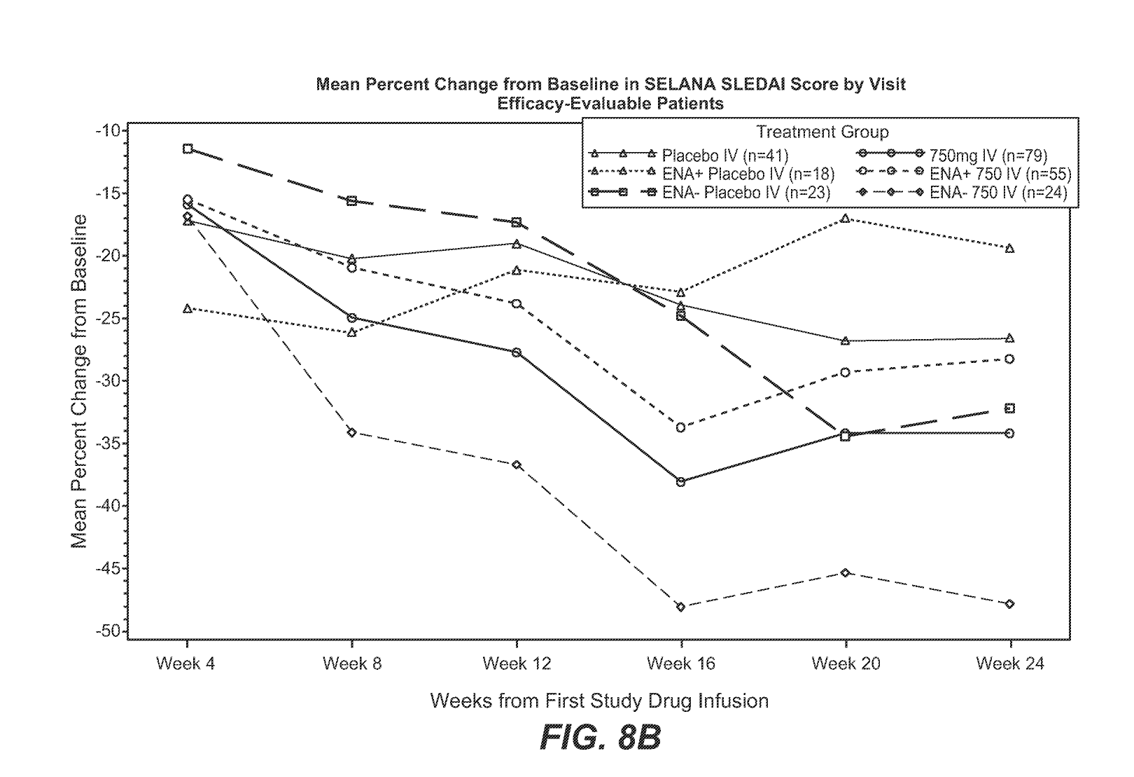

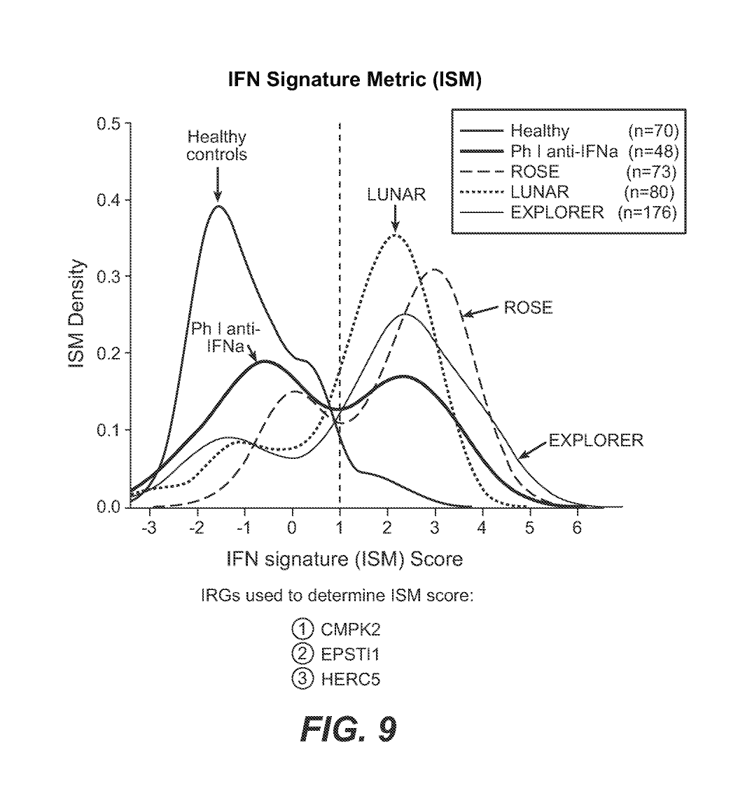

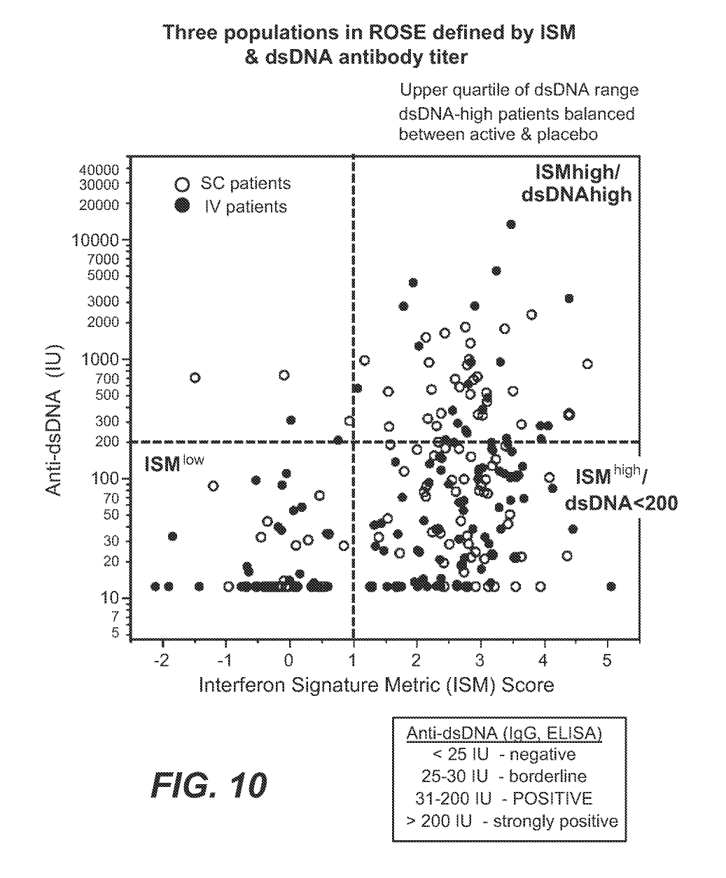

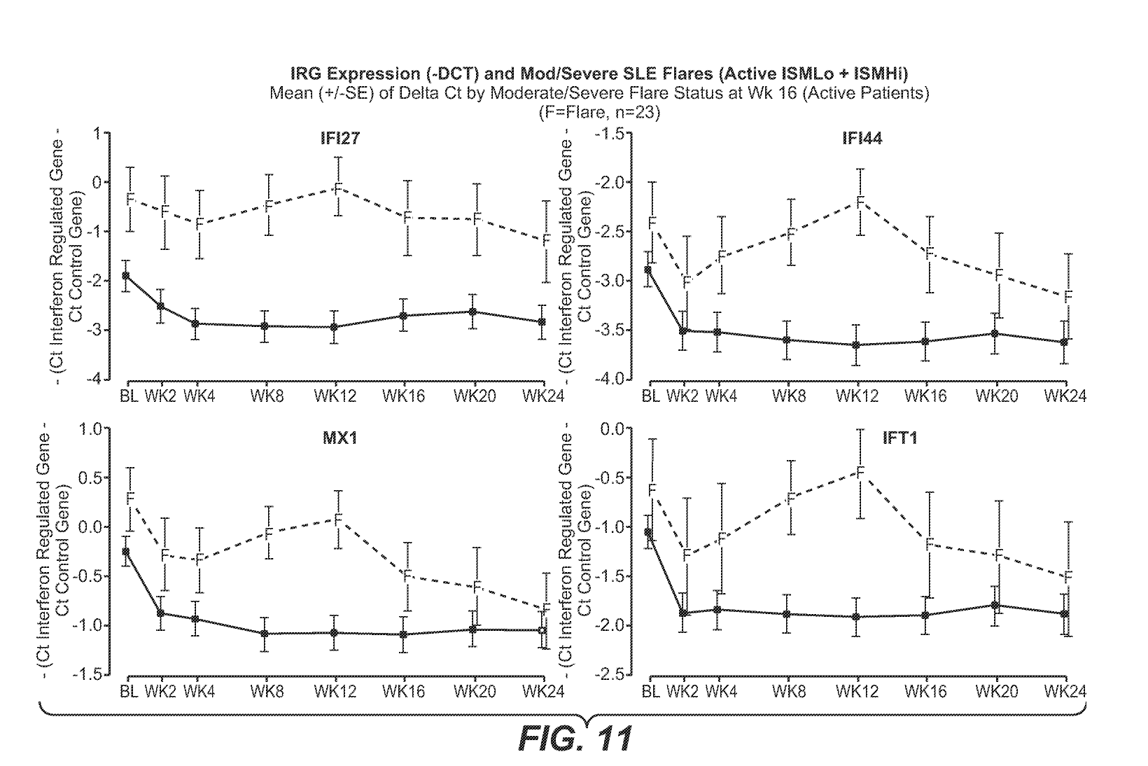

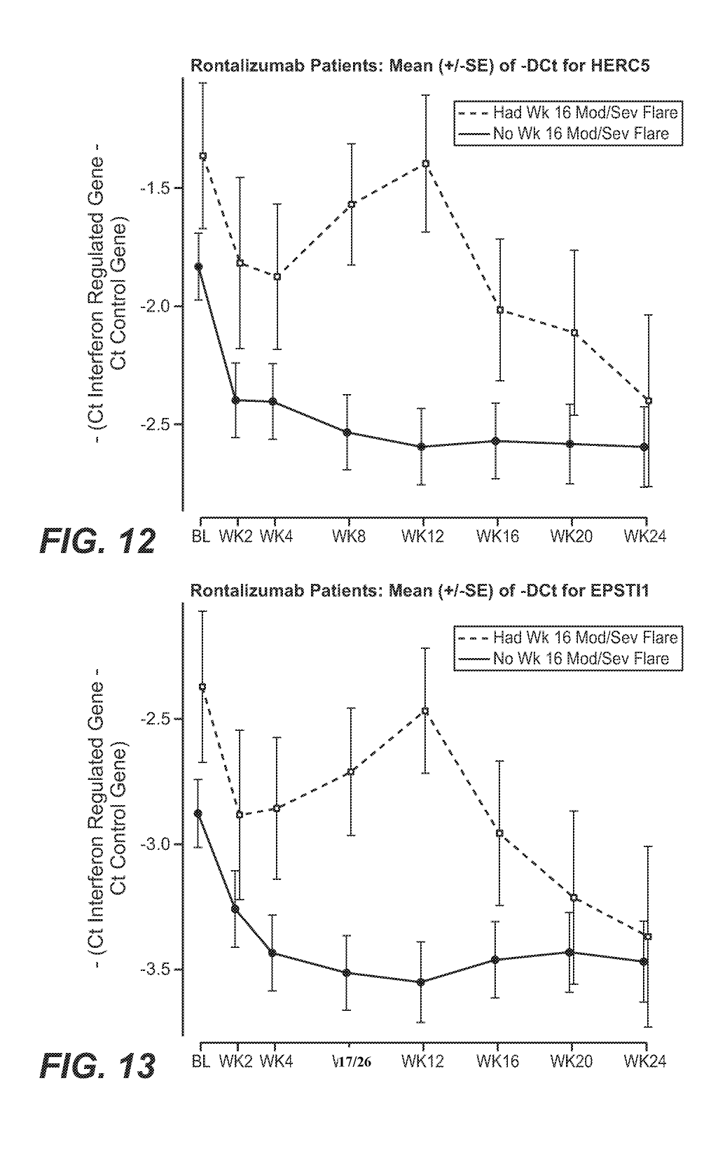

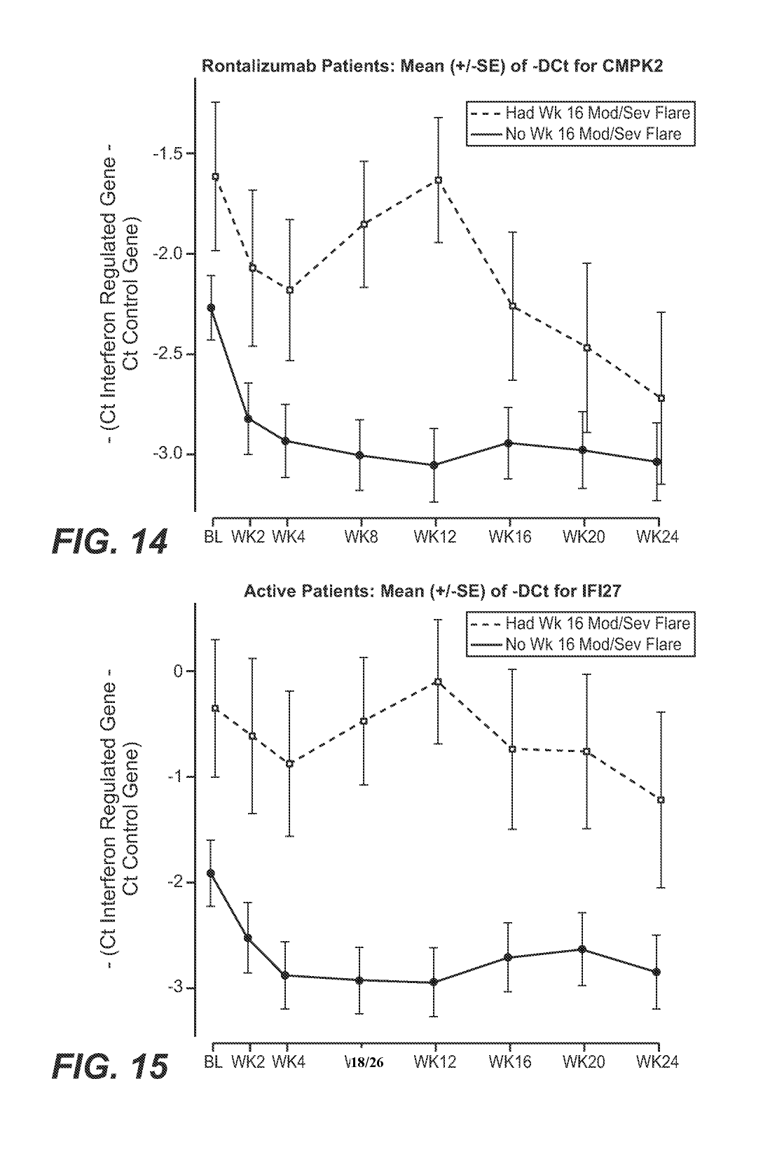

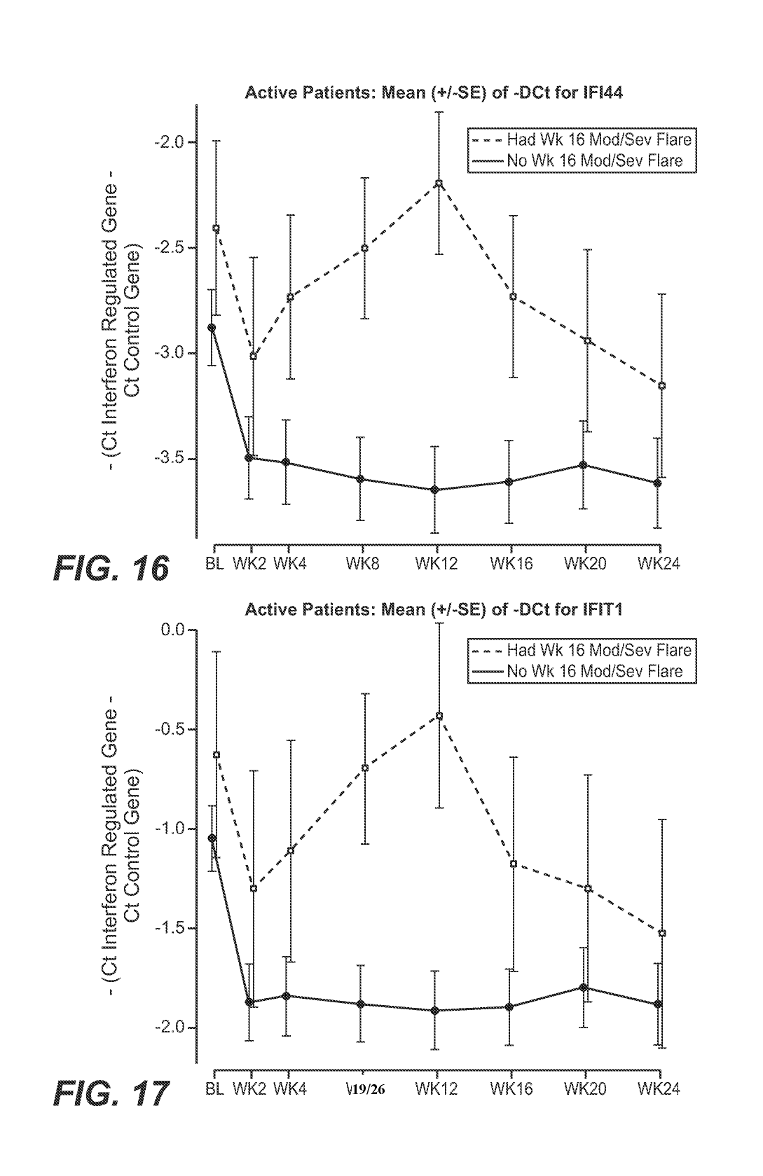

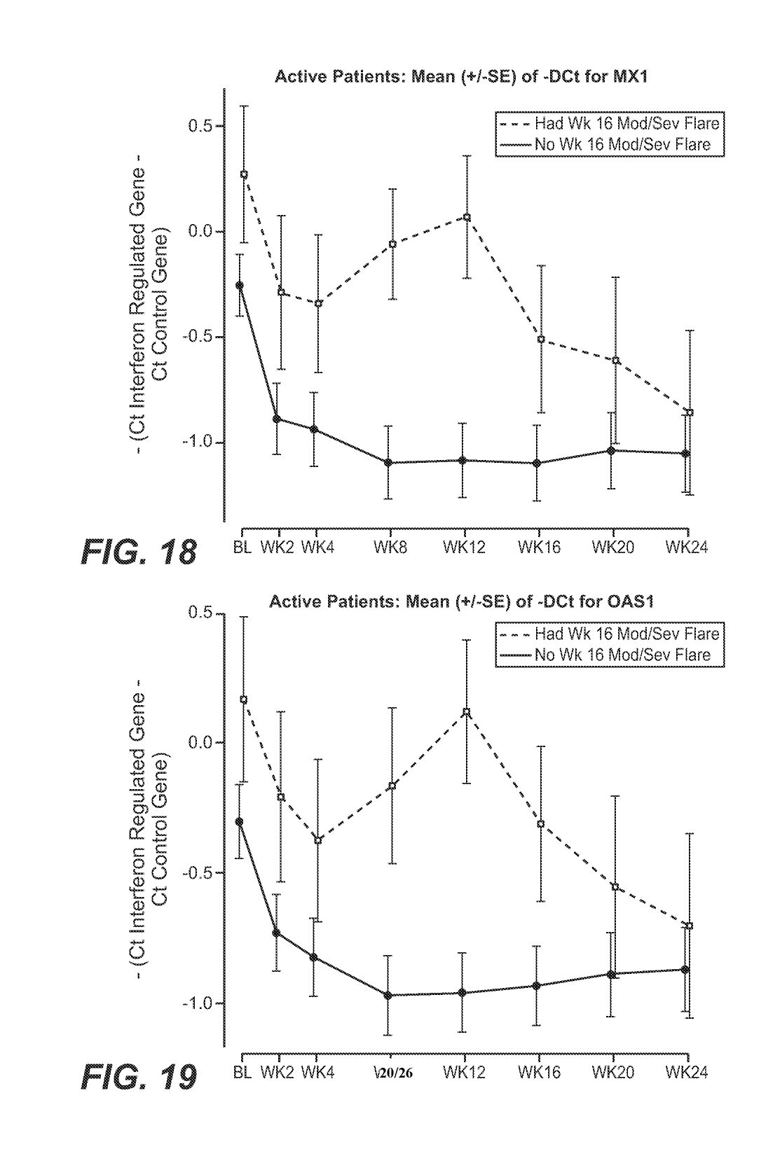

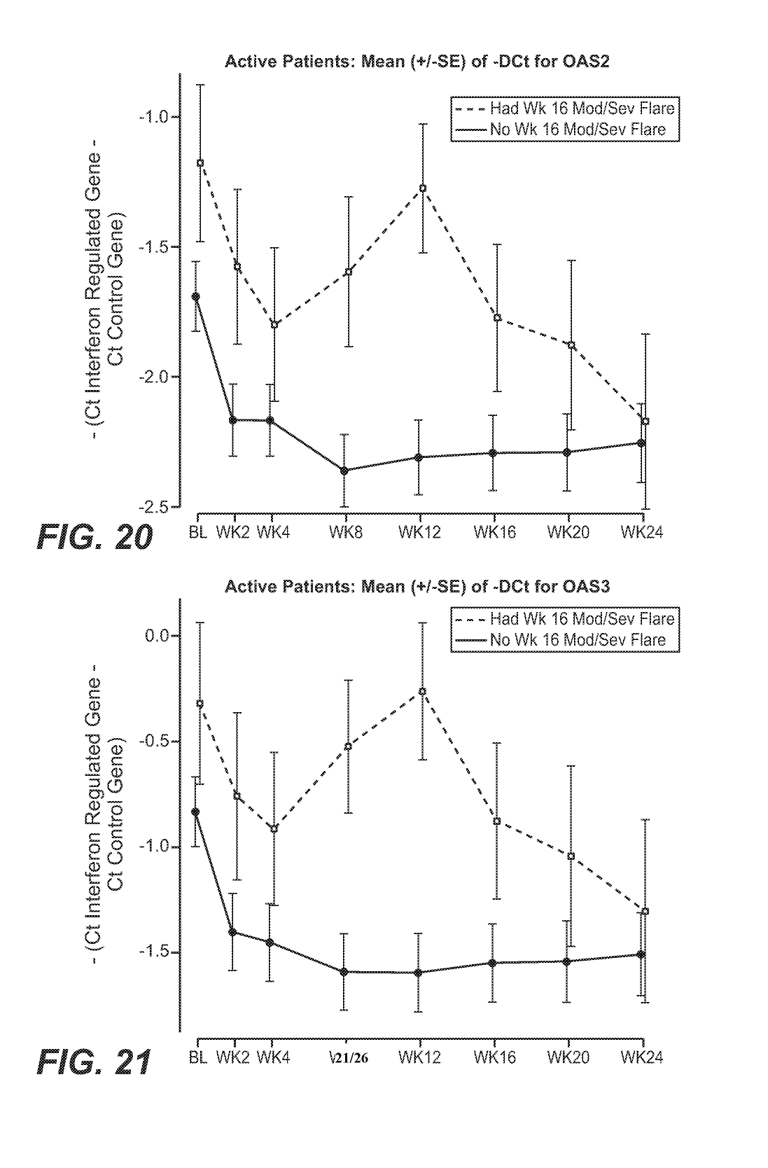

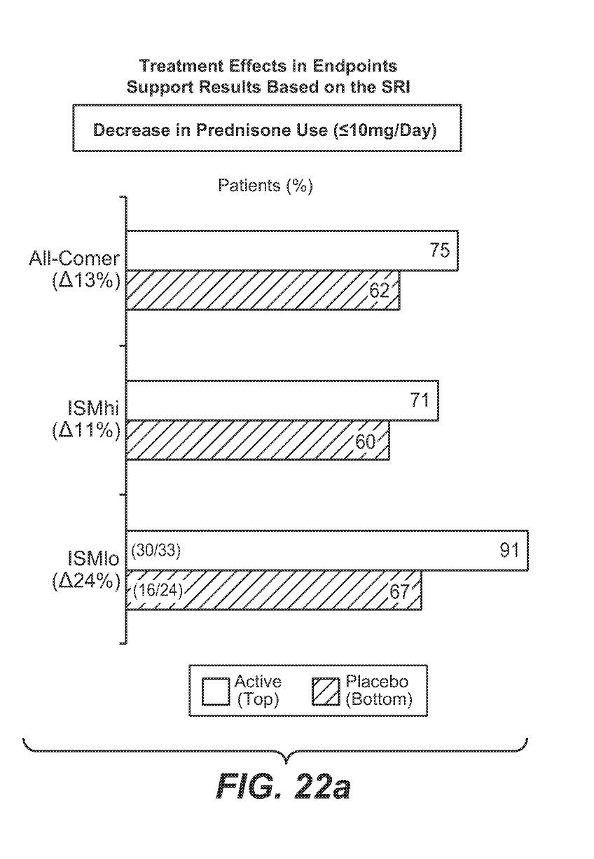

As shown in FIGS. 11-21, interferon regulated genes expression levels (−delta CT or −DCT units) were measured at pre-dose and post-dose time points by quantitative PCR. Patients were separated into two categories; those that had a moderate to severe flare at week 16 (n=23, red line) and the remainder of patients that did not present flares (black line). Preceding the flare at week 16, the mean IRG expression levels were elevated and these genes include but are not limited to HERC5, EPSTI1, CMPK2, IFI27, IFI44, MX1, IFIT1, OAS1, OAS2, and OAS3. Lines shown represent the mean and standard errors of the mean of expression from baseline onwards and include patients from IV and SC cohorts of active groups only. Thus, this example shows that the IRGs can be used to predict the occurrence of flares in patients.

### Example 7

### Secondary Outcomes

This example illustrates the benefits of rontalizumab with respect to the corticosteroid sparing and reduction of flares.

Corticosteroid Sparing

It is often difficult to taper patients with moderate or severe disease completely off corticosteroids, which cause long-term morbidity and may contribute toward early cardiovascular mortality. (Hahn, B H. Systemic lupus erythematosus and accelerated atherosclerosis. N Engl J Med 2003 Dec. 18: 349(25):2379-80). The morbidity associated with prolonged corticosteroid treatment includes osteoporosis, avascular necrosis of bone, Cushingoid features, cutaneous changes, muscle wasting and weakness, easy bruising and many other complications. Thus, the corticosteroid sparing effect of rontalizumab treatment was investigated.

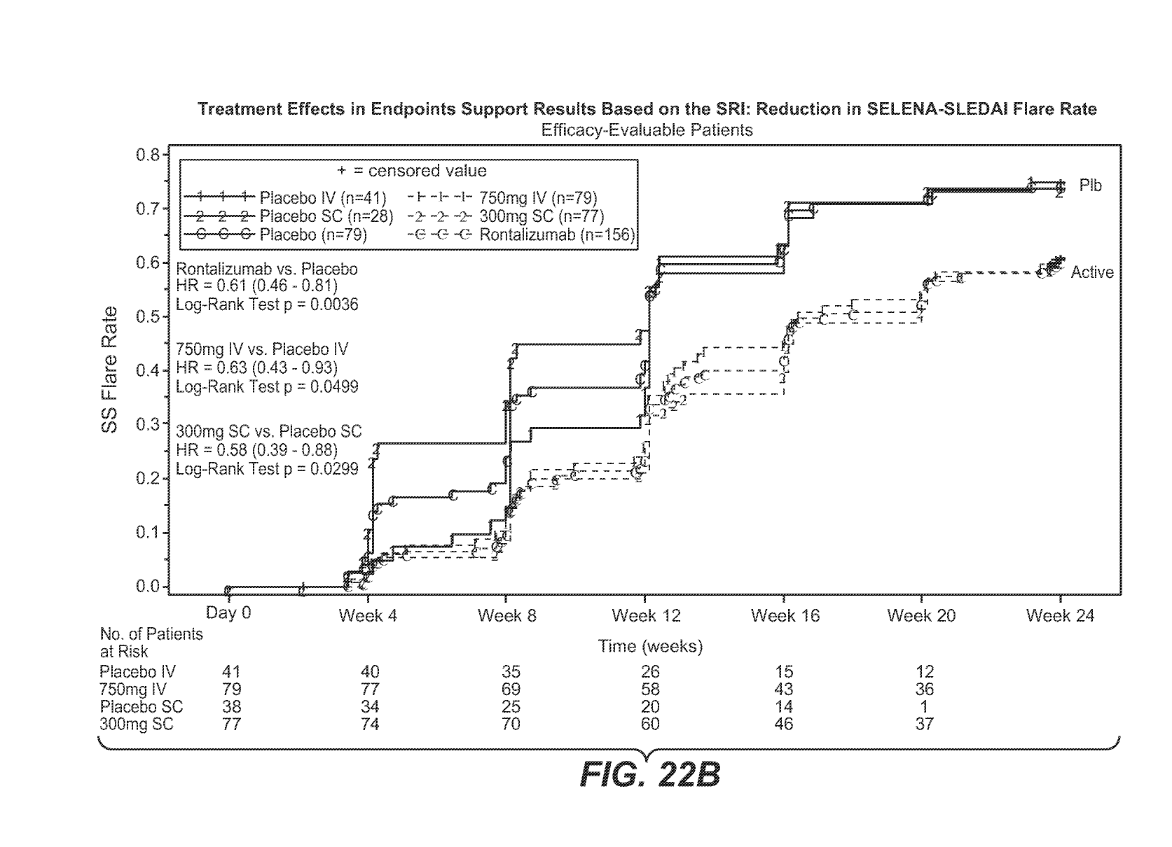

Corticosteroid-sparing in subjects receiving rontalizumab, as measured, e.g., by the proportion of subjects who achieve a meaningful clinical response with <10 mg prednisone per day until the end of the study. Patients were tapered their used as described elsewhere herein. As shown in FIG. 22a, the percent of patients achieving a SRI-4 response while using <10 mg prednisone or prednisone equivalent per day was achieved in all-corners and was pronounced in the ISM^{lo} group relative to placebo.

Reduction of Flares

Reductions in the flare rate or time to flare are considered to be clinically important outcomes. For example, an increase in the frequency and severity of flares of lupus nephritis is correlated with worse outcomes. Thus, a reduction in the rate of flares and/or time-to-flare was evaluated as an efficacy endpoint. Patients were evaluated for flares as described elsewhere herein. As shown in FIG. 22b treatment with rontalizumab (either IV or SC) resulted in a reduction in SELENA-SLEDAI flare rate and a longer time to flare as compared to placebo over the treatment period.

### Example 8

### Adverse Events

The most common adverse reaction with this anti-IFNα antibody was injection site reactions with subcutaneous administration.

The proportion of patients who discontinued treatment due to adverse events was 4% for patients taking this anti-IFNα antibody and 5% for placebo-treated patients. A summary of adverse events is shown in Table 18. Overall adverse events between the placebo and active groups were comparable.

TABLE 18

| Adverse Events | Placebo | Active |
|---|---|---|
| N | 79 | 159 |
| AEs | 84% | 79% |
| Serious AE | 11% | 10% |
| Gr ≧ 3 AEs | 15% | 15% |
| Infection AEs | 49% | 52% |
| Gr ≧ 3 infections | 5% | 2% |
| Drug Discon AEs | 5% | 4% |

1. A method of treating an autoimmune disease in a patient, the method comprising administering an effective amount of an interferon inhibitor to a patient, wherein the patient has been diagnosed with the autoimmune disease and has been determined to be ISM^{lo} or has been selected for treatment based on being ISM^{lo}.

2. The method of claim 1, wherein the ISM^{lo} is determined by measuring mRNA expression levels of one or more interferon response genes (IRGs) in a sample from the patient.

3. The method of claim 2, wherein the mRNA expression levels in the sample are determined by RT-PCR.

4. The method of claim 2, wherein the mRNA expression levels of one or more IRGs are normalized against mRNA expression level of transferring receptor (TFRC).

5. The method of claim 2, wherein the sample is a blood sample.

6. The method of claim 2, wherein the mRNA expression levels of one or more of IRGs selected from the group consisting of CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP, ZBP1, IFI27, SIGLEC1, DNAPTP6, USP18, IFI6, HSXIAPAF1, and LAMP3 are determined.

7. The method of claim 6, wherein the mRNA expression levels of one or more of IRGs selected from the group consisting of CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP, ZBP1, IFI27, SIGLEC1, DNAPTP6, USP18, IFI6, HSXIAPAF1, and LAMP3 are normalized against mRNA expression level of transferrin receptor (TFRC).

8. The method of claim 2, wherein the mRNA expression levels of one or more of IRGs selected from the group consisting of CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP, and ZBP1 are determined.

9. The method of claim 8, wherein the mRNA expression levels of one or more of IRGs selected from the group consisting of CHMP5, CIG5, EPSTI1, G1P2, HERC5, IFI44, IFI44L, IFIT1, IFIT4, IFIT5, IRF7, MX1, OAS1, OAS2, OAS3, OASL, PARP9, RIG1, RIGE, SAMD9L, SP110, TYK1 (CMPK2), XIAP, ZBP1 are normalized against mRNA expression level of transferrin receptor (TFRC).

10. The method of claim 2, wherein the mRNA expression levels of EPSTI1, HERC5 and/or TYK1 (CMPK2) are determined.

11. The method of claim 10, wherein the mRNA expression levels of EPSTI1, HERC5 and/or TYK1 (CMPK2) are normalized against mRNA expression level of transferrin receptor (TFRC).

12. A method of treating an autoimmune disease in a patient, the method comprising administering an effective amount of an interferon inhibitor to a patient, wherein the patient has been diagnosed with the autoimmune disease and has been determined to have a pretreatment anti-double stranded DNA antibody titer (anti-dsDNA) that is less than or equal to 200 IU as measured by immunoassay or selected for treatment based on having a pre-treatment anti-double stranded DNA antibody titer (anti-dsDNA) that is less than or equal to 200 IU as measured by immunoassay.

13-24. (canceled)

25. The method of claim 1, wherein the autoimmune disease is selected from the group consisting of lupus, rheumatoid arthritis, psoriasis, psoriatic arthritis, insulin-dependent diabetes mellitus (IDDM), multiple sclerosis (MS), myositis, dermatomyositis, vasculitis, atherosclerosis, ankylosing spondylitis, and Sjogren's syndrome.

26. The method of claim 25, wherein the patient has systemic lupus erythematosus (SLE).

27. The method of claim 25, wherein the patient has moderately to severely active lupus.

28. The method of claim 25, wherein the patient has moderately to severely active SLE.

29. The method of claim 25, wherein the patient has lupus nephritis.

30. The method of claim 1, wherein the patient has Class III-V lupus nephritis and is ISM^{lo}.

31. The method of claim 25, wherein the patient has pediatric lupus.

32. The method of claim 1, wherein the interferon inhibitor is an anti-interferon type I antibody.

33. The method of claim 32, wherein the antibody specifically binds an interferon selected from the group consisting of: interferon α, interferon β, interferon ω, interferon λ, and combinations thereof.

34. The method of claim 32, wherein the antibody specifically binds interferon α.

35. The method of claim 34, wherein the antibody binds to at least IFNα subtypes 1, 2, 4, 5, 8, 10 and 21.

36. The method of claim 34, wherein the antibody comprises a light chain comprising HVR-L1 comprising the amino acid sequence RASQSVSTSSYSYMH (SEQ ID NO:1), HVR-L2 comprising the amino acid sequence YASNLES (SEQ ID NO:2), and HVR-L3 comprising the amino acid sequence QHSWGIPRTF (SEQ ID NO:3); and/or a heavy chain comprising HVR-H1 comprising the amino acid sequence GYTFTEYIIH (SEQ ID NO:4), HVR-H2 comprising the amino acid sequence SINPDYDITNYNQRFKG (SEQ ID NO:5), and HVR-H3 comprising the amino acid sequence WISDFFDY (SEQ ID NO:6).

37. The method of claim 34, wherein the antibody comprises a heavy chain variable region sequence of at least 95% sequence identity to the amino acid sequence of SEQ ID NO:7; and/or a light chain variable region sequence of at least 95% sequence identity to the amino acid sequence of SEQ ID NO:8.

38. The method of claim 34, wherein the antibody comprises a heavy chain variable region comprising the amino acid sequence of SEQ ID NO:7; and a light chain variable region comprising the amino acid sequence of SEQ ID NO:8.

39. The method of claim 34, wherein the antibody is rontalizumab having CAS registration number 948570-30-7.

40. The method of claim 32, wherein the antibody is administered intravenously.

41. The method of claim 32, wherein the antibody is administered subcutaneously.

42. The method of claim 32, wherein the antibody is administered at a flat dose of 100 to 2000 mg.

43. The method of claim 42, wherein the antibody is administered at a flat dose of 100-500 mg weekly, 200-1000 mg biweekly, or 400-2000 mg monthly.

44. The method of claim 42, wherein the antibody is administered at a flat does of 150 mg or 300 mg weekly, 300 mg or 600 mg biweekly, or 600 mg, 750 mg or 1200 mg monthly.

45. The method of claim 32, wherein the administration of the antibody is effective in one or more of the following: (1) reduction of the number and/or severity of lupus flares, (2) prevention of lupus flares, (3) reduction in lupus nephritis flares, (4) prevention of lupus nephritis flares, (5) induction of remission in lupus nephritis, (6) maintenance of lupus nephritis remission, (7) reduction in the number and/or severity of pediatric lupus flares, (8) prevention of pediatric lupus flares, (9) reduction in pediatric lupus nephritis flares, (10) prevention of pediatric lupus nephritis flares, (11) induction of remission in pediatric lupus nephritis, and (12) maintenance of pediatric lupus nephritis remission.

46. The method of claim 32, wherein the administration of the antibody is effective in lowering the anti-dsDNA antibody titer in the patient.

47. The method of claim 32, wherein the administration of the antibody is effective in reduction of flare(s) in the patient.

48. The method of claim 47, wherein said flare(s) are moderate or severe.

49. The method of claim 32, wherein the administration of the antibody is effective in reduction of Selena Flare Index (SFI) score or Selena Flare Index-Revised (SFIR) score in the patient.

50. The method of claim 32, wherein the administration of the antibody is effective in decreasing all pre-treatment BILAG A and B domains.

51. The method of claim 32, wherein the patient has no new BILAG A organ domain score or no more than one new BILAG B organ domain score after the administration of the antibody.

52. The method of claim 32, wherein the administration of the antibody is effective in decreasing in SELENA-SLEDAI score by at least four points from the patient's pre-treatment score.

53. The method of claim 32, wherein the patient has no more than 0.3 points increase in Physician Global Assessment (PGA) from the pre-treatment score after the administration of the antibody.

54. The method of claim 32, wherein said patient has a post-treatment decrease in disease activity in those organ systems with moderate or severe disease activity prior to treatment as measured by any one of the following assessment tools: SRI, BILAG, SELENA-SLEDAI, or Physician Global Assessment (PGA).

55. The method of claim 32, wherein the patient has an SRI-4, SRI-5, SRI-6, or SRI-7 response to the administration of the antibody.

56. The method of claim 1, further comprising administering a second medicament to the patient.

57. The method of claim 56, wherein the second medicament is selected from the group consisting of: a corticosteroid, a non-steroidal anti-inflammatory drug (NSAID), an immunosuppressive, an anti-malarial agent, a statin, and combinations thereof.

58. The method of claim 56, wherein the second medicament is a standard of care for lupus.

59. The method of claim 32, wherein the administration of the antibody results in corticosteroid sparing (CS) in a patient taking a corticosteroid prior to said administration of said antibody.

60. The method of claim 32, wherein the administration of the antibody results in a decrease in the requirement for therapy with steroids and/or immunosuppressive regimens.

61. The method of claim 32, wherein the patient has tapered their corticosteroid dose to a prednisone equivalent of 10 mg/day after the administration of the antibody.

62. The method of claim 32, wherein the administration of the antibody results in reduction in corticosteroid use by at least 50% after about 24 to about 52 weeks of the administration of the antibody.

63. The method of claim 32, wherein the administration of the antibody results in one or more of the following: reduction in the incidence of moderate and/or severe flares as measured by SELENA SLEDAI scores and/or Physicians Global Assessment; significantly delaying time to severe flare; reduction in the number of swollen or tender joints; and significantly reducing the risk of one BILAG A (severe) organ flare or more than one BILAG B (moderate) organ flare.

64. A therapeutic regimen for the treatment of an ISM^{lo} lupus patient in need thereof comprising the administration of an interferon inhibitor.

65-74. (canceled)

75. A method of identifying a lupus patient who may benefit from an interferon inhibitor treatment, the method comprising determining the IRG status in a sample from the patient, wherein a patient who is ISM^{lo} is identified as a patient who may benefit from the interferon inhibitor treatment.

76. A method of identifying a lupus patient who may benefit from an interferon inhibitor treatment, the method comprising determining the IRG status in a sample from the patient, and providing a report regarding the IRG status of the patient, wherein the report indicates that the patient is ISM^{lo} or ISM^{hi}.

77. (canceled)

78. A method of predicting responsiveness of a lupus patient to an interferon inhibitor treatment, the method comprising determining the IRG status in a sample from the patient, wherein a patient who is ISM^{lo} is identified as a patient who is likely to respond to the interferon inhibitor treatment.

79-80. (canceled)

81. A method of predicting responsiveness of a lupus patient to an interferon inhibitor treatment, the method comprising determining the expression levels of IRGs in a sample from the patient, and comparing the patient's IRG expression levels to a mean value of the expression levels of the same IRGs in a healthy person or healthy persons, wherein the patient is identified as a patient who is likely to respond to the interferon inhibitor treatment if the patient's IRG expression levels are (1) less than 1.4 times the mean value of the expression levels of the same IRGs of a healthy person or (2) less than two standard deviations over the mean value of the expression levels of the same IRGs in healthy persons.

82-91. (canceled)

92. A method of identifying a lupus patient who may benefit from an interferon inhibitor treatment, the method comprising determining the anti-dsDNA antibody status in a sample from the patient, wherein a patient who has an anti-dsDNA antibody titer that is less than or equal to 200 IU as measured by immunoassay is identified as a patient who may benefit from the interferon inhibitor treatment.

93. A method of identifying a lupus patient who may benefit from an interferon inhibitor treatment, the method comprising determining the anti-dsDNA antibody status in a sample from the patient, and providing a report indicating that the patient may benefit from the interferon inhibitor treatment if anti-dsDNA antibody titer that is less than or equal to 200 IU as measured by immunoassay.

94. A method of predicting responsiveness of a lupus patient to an interferon inhibitor treatment, the method comprising determining the anti-dsDNA antibody status in a sample from the patient, wherein a patient who has an anti-dsDNA antibody titer that is less than or equal to 200 IU as measured by immunoassay is identified as a patient who is likely to respond to the interferon inhibitor treatment.

95. (canceled)

96. A method for predicting the likelihood of a flare in a lupus patient, the method comprising determining the IRG status of the patient, wherein a significant increase of expression levels of IRGs indicates that the patient is likely to have a flare in the next 3 to 5 weeks.

97-107. (canceled)

108. An article of manufacture comprising a subcutaneous administration device, which delivers to a patient a flat dose of an anti-interferon α antibody, wherein the flat dose is in the range of 50 mg to 2000 mg of the anti-interferon α antibody.

109-111. (canceled)

112. An article of manufacture comprising an anti-interferon α antibody in a concentration from about 50 to 250 mg/mL.

113-117. (canceled)

118. An article of manufacture comprising a computerized system comprising a bio-assay module for detecting a gene expression of one or more IRGs from a biological sample and a processor module to calculate expression of the gene and to score the calculation of the gene against a cutoff value to provide a diagnosis, wherein the cutoff value is (1) less than 1.4 times the value of the expression levels of the IRGs of a healthy person or (2) less than two standard deviations over the median value of the expression levels of the IRGs in healthy persons.

119. (canceled)

120. A kit for identifying an autoimmune patient who may benefit for an interferon inhibitor treatment, comprising a vial for collecting a blood sample from an autoimmune patient and instructions for determining whether the autoimmune patient is ISM^{lo}.

121-123. (canceled)

124. A stable liquid composition comprising an anti-interferon α antibody in an amount of about 50 to about 250 mg/mL, arginine-HCl in an amount of about 50 to about 200 mM, histidine in an amount of about 5 to about 100 mM, polysorbate in an amount of about 0.01 to about 0.1%, wherein the composition has a pH from about 5.5 to about 7.0.

125. A method of treating lupus in a patient, the method comprising administering an effective amount of an interferon type I antibody to a patient diagnosed with lupus, wherein the patient is ENA−.

126-138. (canceled)

139. A method of identifying a lupus patient who may benefit from treatment with an interferon type I antibody, the method comprising determining ENA status of the patient, wherein a patient who is determined to have an ENA status of ENA− is identified as a patient who may benefit from treatment with the interferon type I antibody.

140. A method of optimizing therapeutic efficacy for treatment of lupus, the method comprising determining ENA status of a lupus patient, wherein a patient who is determined to have an ENA status of ENA− has increased likelihood of benefit from treatment with the interferon type I antibody.

141. A method of predicting responsiveness of a lupus patient to treatment with an interferon type I antibody, the method comprising determining ENA status of the patient, wherein a patient who is determined to have an ENA status of ENA− is identified as a patient who is likely to respond to treatment with the interferon type I antibody.

142. A method for determining the likelihood that a lupus patient will benefit from treatment with an interferon type I antibody, the method comprising determining ENA status of the patient, wherein a patient who is determined to have an ENA status of ENA− is identified as a patient who is likely to respond to treatment with the interferon type I antibody.

143-156. (canceled)

157. A method of treating lupus in a patient, the method comprising administering an effective amount of an interferon type I antibody to a patient diagnosed with lupus, wherein the patient has a baseline interferon signature metric (ISM) that is equal to the ISM of a healthy individual.

158. A method of treating lupus in a patient, the method comprising administering an effective amount of an interferon type I antibody to a patient diagnosed with lupus, wherein the patient has an ISM that is lower following administration of the antibody as compared to the patient's baseline ISM.

159-167. (canceled)

168. A method of identifying a lupus patient who may benefit from treatment with an interferon type I antibody, the method comprising determining the baseline ISM status of the patient, wherein a patient who has a baseline ISM equal to the ISM of a healthy individual is identified as a patient who may benefit from treatment with the interferon type I antibody.

169. A method of optimizing therapeutic efficacy for treatment of lupus, the method comprising determining the baseline ISM status of the patient, wherein a patient who has a baseline ISM equal to the ISM of a healthy individual has increased likelihood of benefit from treatment with the interferon type I antibody.

170. A method of predicting responsiveness of a lupus patient to treatment with an interferon type I antibody, the method comprising determining the ISM status of the patient, wherein a patient who has an ISM equal to the ISM of a healthy individual is identified as a patient who is likely to respond to treatment with the interferon type I antibody.

171. A method for determining the likelihood that a lupus patient will benefit from treatment with an interferon type I antibody, the method comprising determining the ISM status of the patient, wherein a patient who has an ISM equal to the ISM of a healthy individual is identified as a patient who is likely to respond to treatment with the interferon type I antibody.

172-182. (canceled)


SEQUENCE LISTING (31 sequences)

SEQ ID NO: 1 - protein, 15 aa, Artificial Sequence
  Synthetic Construct
RASQSVSTSSYSYMH

SEQ ID NO: 2 - protein, 7 aa, Artificial Sequence
  Synthetic Construct
YASNLES

SEQ ID NO: 3 - protein, 10 aa, Artificial Sequence
  Synthetic Construct
QHSWGIPRTF

SEQ ID NO: 4 - protein, 10 aa, Artificial Sequence
  Synthetic Construct
GYTFTEYIIH

SEQ ID NO: 5 - protein, 17 aa, Artificial Sequence
  Synthetic Construct
SINPDYDITNYNQRFKG

SEQ ID NO: 6 - protein, 8 aa, Artificial Sequence
  Synthetic Construct
WISDFFDY

SEQ ID NO: 7 - protein, 119 aa, Artificial Sequence
  Synthetic Construct
EVQLVESGGGLVQPGGSLRLSCATSGYTFTEYIIHWVRQAPGKGLEWVASINPDYDITNYNQRFKGRFTISLDKSKRTAYLQMNSLRAEDTAVYYCASWISDFFDYWGQGTLVTVSSAS

SEQ ID NO: 8 - protein, 114 aa, Artificial Sequence
  Synthetic Construct
DIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIKRTV

SEQ ID NO: 9 - DNA, 36 bp, Artificial Sequence
  Synthetic Construct
agggccagtcagagtgttagcagcacctacttagcc

SEQ ID NO: 10 - DNA, 21 bp, Artificial Sequence
  Synthetic Construct
ggtgcatccagcagggccact

SEQ ID NO: 11 - DNA, 27 bp, Artificial Sequence
  Synthetic Construct
cagcagtatggtagctcacctcggacg

SEQ ID NO: 12 - DNA, 15 bp, Artificial Sequence
  Synthetic Construct
agctatagtatcagc

SEQ ID NO: 13 - DNA, 36 bp, Artificial Sequence
  Synthetic Construct
aatggtaacacaaactatgcacagaagttccagggc

SEQ ID NO: 14 - DNA, 21 bp, Artificial Sequence
  Synthetic Construct
gatcccatagcagcaggctac

SEQ ID NO: 15 - protein, 12 aa, Artificial Sequence
  Synthetic Construct
RASQSVSSTYLA

SEQ ID NO: 16 - protein, 7 aa, Artificial Sequence
  Synthetic Construct
GASSRAT

SEQ ID NO: 17 - protein, 9 aa, Artificial Sequence
  Synthetic Construct
QQYGSSPRT

SEQ ID NO: 18 - protein, 5 aa, Artificial Sequence
  Synthetic Construct
SYSIS

SEQ ID NO: 19 - protein, 17 aa, Artificial Sequence
  Synthetic Construct
WISVYNGNTNYAQKFQG

SEQ ID NO: 20 - protein, 7 aa, Artificial Sequence
  Synthetic Construct
DPIAAGY

SEQ ID NO: 21 - protein, 116 aa, Artificial Sequence [sequence omitted]

SEQ ID NO: 22 - protein, 108 aa, Artificial Sequence [sequence omitted]

SEQ ID NO: 23 - DNA, 24 bp, Artificial Sequence
  Synthetic Construct
tgctcttgctgctgccgtttcagt

SEQ ID NO: 24 - DNA, 24 bp, Artificial Sequence
  Synthetic Construct
aggcagaagaaaacagaaaattgc

SEQ ID NO: 25 - DNA, 23 bp, Artificial Sequence
  Synthetic Construct
gtgttcagtctggtggattttgg

SEQ ID NO: 26 - DNA, 24 bp, Artificial Sequence
  Synthetic Construct
ctgccggagaagcccacagcatgg

SEQ ID NO: 27 - DNA, 20 bp, Artificial Sequence
  Synthetic Construct
acctcgcaggagtacccttg

SEQ ID NO: 28 - DNA, 21 bp, Artificial Sequence
  Synthetic Construct
gccaccacaagcgacaaattc

SEQ ID NO: 29 - DNA, 25 bp, Artificial Sequence
  Synthetic Construct
cgaaggactggatgccacgggtaaa

SEQ ID NO: 30 - DNA, 21 bp, Artificial Sequence
  Synthetic Construct
gaaagttccaggttgttgcca

SEQ ID NO: 31 - DNA, 20 bp, Artificial Sequence
  Synthetic Construct
tgaatctgccactgactggg